SEQUENCE LISTING (163 sequences)

SEQ ID NO: 1 - DNA, 1487 bp, Aspergillus nidulans
  source 1..1487: mol_type = genomic DNA
aatgactggtccgtccgtacttagaaagggtgtttctgtccggcagttatttaatgtcggctgtctgctcttgcaatttctcttttgatttatctttcgtggtgtatctcgccggaacgaatggccacggttcgcgtttgcgttcatgttcatgttcatagagcagctgcgaagtttcaaatgttcgttcgttcggctcggcttggctaggcgtatgatggtgttatgtttaggttgagaaggtattcttagttgggagctagagaaaagattatttgttccctgcaattttgctgtaccccggaaacatagaactgttactgtaccaatactctgcgttccctccccaatgcaccccatacatatggagttggagcctgtacctttgtcgataagcttattctccaatcaactctgctattgcagcttttcacttgagctttcttattcgtatgtgctctacggacgaaaaataagctttgttgcctgcagatcaccttggcagctgtgctgcgcctagacttataatgcaacgtttttaactttttgtttttcttttttctttcttttttaaactagttttcacatgagctacccgttcattataaccatcagctctagctaggacaggatcgcatgagtatatacctatttatattccttccctcccaactcggactcacgctttatatatatgtctactattactcgtgggtgaagagaagtttacgactatttagcctagatgaaggataggttgtgcaatgctcgatagcgtagcatttaaccctacctagtaatgagctacttgggctgctagaataaatctcccaatccaagctaatgtagtcagagctgaacgcaagtctcgtacatggccctacgaggcatcacaatagccctaaagagtatcacgtgaccatactagcaccgcaatgagttcaggatccgacaatagcgaggctgtatccaagtgcgccgaataatgtctatcactgtagaaatatatctgattcgctcagctggtcgataggcgaagcatcggagttggcggagttggcggagttgcaggacttgctggattagggctgaggtcagacggactctcactctccgctatagacactgggcgatgttgtaggcagcgatgggagaatgtgcattgcacatggtccggagatttctggagtcaggtcatgcagtctagatcctgactgcagtagaatgtgcagattccggagcttggggagttaacctgcagtaagctcagctcaagcaatgatcggtaggtaggcctggtggccatatcagctatagatgcgatccgcgcctcaagcgcatttcaagccctccctcttcaatacgtttgcgataccttagagaaacaaatcaacatccatcaactggcacagattcatctaccaactcaacgtgattacccgtccagctttgacctaaacctccataatccccatccacaaggcacc

SEQ ID NO: 2 - DNA, 8049 bp, Aspergillus nidulans
  source 1..8049: mol_type = genomic DNA
atgggcagcacatcttccgagcccacatacgacagtgagcccatcgcgattattggcctttcgtgcaagttcgctgggtccgcagacagccccgagaaactatgggagatgcttgcggaagggcggaatgcatggtcagagatccctgagtcgcggtttaaccacaaggccgtgtatcatcctgatagtgagaagctggggacggtacgtctttccttctagacttgagtttcagtggtgaagtggatgggaagcaagaacctggccagactaacgcggaatcttcgcagacgcatgtcaaaggggcacattttctcgagcaagatgtcgggctcttcgacgcggcattcttcaattattcggcggagacagctgctgtacggtccctatgaacgatttcaggatgaatggccaggctaactgagcatgatgtacggatagaccctcgatccgcaattccgcttccagctcgagtccgtctatgaggctcttgaaaatggtaccaccctccccccaacagcccttgcgcaaggctgaacagagagtacagctggcctgacgattccatccatcgccggcaccaacacctccgtctacgccggcgtcttcacgcatgactaccacgaaggtctgattcgcgacgaagacaaactgccccggttcctccccatcggaaccctctccgccatgtcctcgaaccgcatcagccacttcttcgacctcaaaggagcaagcgtgactgtagacaccggctgctcgacggccctggtggccctgcaccaggccgtcctcggcctgcgcacgcgcgaagcagacatgagcatcgtctctggatgcaacatcatgctgtcgccggatatgttcaaggtgttttcaagtttgggaatgctaagccctgatgggaagagctacgcctttgactcaagggcgaatggatacggacggggcgagggcgtagcgacgattatcgtgaagcgactcgcggatgcgctgagggacggggatcccgtgcgcggcgtgatccgcgagagctatctgaatcaggatggaaaaacagagactatcacctcgccgtcacaggaagcgcaggaggcactgatcaaagaatgttatcggcgcgcggggctgtcgccgtcggatacacagtacttcgaagcgcatgggacaggcacccccactggagatccgattgaggcgcgctcaatcgcgtcagtatttggaaagaatcgagagcagccgttgcggattggctctgtcaagacgaatatcgggcatactgaggcggccagtggtcttgccgggctgatcaaggtcgtgctggccatggagaaggggttcatcccgcccagcgtaaactttgagaagccgaatccgaagctgaagctggatgaatggaggctaaaggtggcagatactttggaaaagtggcctgcaccggcggagcggccatggagggcgagcgtgaacaactttgggtatgggggtacgaacagccatgtcattgtggaaggggtgccgaagagattatacacaccggcaaatggaaatgagaccggccagataaagcatgagacagagagcaaagtgctcctcttctctggccgcgacgaacaagcctgccagcgcatggttgccagcacgaaggagtacctgaagaagcgcagggagcaggatcctcccatgacacctgaacaagtcaagaccctcatgcaaaatctcgcctggacattaacgcagcaccgcactcgcttctcctgggtctccgcacacgcggtcaagtactcgacctccctggacaccgtcattgacgccctcgagtctccgccgccggcctcaagacccgttcgcatccctgactctccattccgtattggcatggtcttcacggggcaaggtgcgcagtggcacgccatgggccgcgagctgatcgccgcgtacccggtattcaaggcaaccctagacgaagcggaacagtatttgcgccaactgggggccggctggtccctcatcgaagagctgatgaaggatgcagccacgacaagagtcaacgacaccggcctcagcatccctatctgtgtcgccgtgcagatcgctctcgtccgcctgctcaaggcatgggggatcactgcctcggccgtgacatcccactcgtccggtgagatcgccgccgcgtatacggttggcgctctctcgctgcgccaggccatggccgccgcctactaccgcgctgccatggcagcagacaagacgctgaagagcgcagaggggccccaaggcgcaatggttgccgtgggtgttgacaaggctgccgcgcaggcatacctggaccgcgttgagaaatcggcaggccgcgctgtggtggcatgcatcaacagccccagcagcatcaccattgccggcgacgaggcagccgtcgtcgcggtcgagaagttggccactgaggagggcgtctttgcgcgccgactcagggtcgagacgggatatcactcgcaccatatggagccaattgcgagcccgtaccgggaggcgcttcgcgccgcattggcccaggaagatgctgagtctggtaccaaggaccagactgatgtcccgggctttgcggatgccactaaaccgggcagcctagaccacaccgtcttctcctcccccgtcacgggcggccgtgtcacagatgccaaagtcctctctgacccggagcactgggtccgcagtctgctccagccagtgcggttcgtcgaggccttcactgatatggtgcttggctccacagatagcagcaatattgacctgatcctcgaggtcgggccgcatacagcccttggcggaccgatcaaggagatccttgccctgcctgacttcagcagcaggaatgtcagcctcccctacatgggctgcctcgttcgtaaagaagatgcgcgcgactgcatgctcactgctgccttaaaccttttctccaagggccacagtatcgacctgctcagactcagcttctcgtctggcatcccagagttgcaagtcctgaccgacctcccctcatacccgtggaaccacagcatcagacactggtctgagtctcgccgcaatgccgcgtaccgtaagcgcagccaggagccgcatgagctgctgggcgtgctggaaccgggcacgaacccggacgctgcctcgtggaggcatatcatcaagctctccgaggcgccgtggctgcgcgaccacgttgtccaggggaacatcctctaccccggtgcaggattcgtgtgtctcgccattgaggcaatcaagatgcagtctgccatgagcgggacgaatgatgtgaccggtttcaggctgcgcgatgtcgagatccatcaggcgctcgtgattgcggacagtgcagacggcgtcgaagtgcagacgaccctccggtccgtaggaggcaaggtcatcggcgccagaggctggaagcagtttgagatctggtcggtcagcgcagacagcgagtggacagagcacgcgaggggtctaatcaccgtcgacactgagaccaaggcatccacgctcgtggcaagcactctcgatgaatccggctacacgcgccgcatcgacccgcaagacatgtttgctagcctgcgcgcaaaggggctcaaccacgggcccatgttccagaatacgctgagaatcctgcaggacggaagggccaaggagccgcagtgcgtcgtcgatatcaagatcgccgacgtatcgagcagcaaggacagcggccggatgagtcttctgcacccgacgacgctcgactcaatcgttctctcctcatacgccgcagtacccagctcggatccgtccaacgacgacagcgcgcgcgttccccggtccatccgcagcctgtgggtgtcgagcatgatcagcagcgccccgggccatacgttcacctgtaatgtgaagatgccgcatcacgatgcgcagagttacgaagcgaacgtgacagtcgtggacgaggccggagccagagctgagagcatggtcgagatgcagggtcttgtctgccagtctctcggccgcagcgcaccagcagaggaccgagaaccctggacgaaggagctatgcgcgaacgtcgaatgggcgcctgatctctccctctctctcggccttccgggctcgtcagacgccatcgacaggcgcctcaacaccctccgcgaccagaatccagacgagaggagcatcgaagtgcagacggtcctgcgccgcgtctgcgtctacttcagccacgatgccctttcctccctgacagaaaacgacgtggcaaatctcgcattccaccatgtcaagttctacaagtggatgcaggataccgtcaacctggcactcgcgcgccgctggagtgccgacagcgacacctggattcatgacagtcccgccgtacgggaaaagtacatttcccttgctgggtcgcagacggtggacggagagctgatctgccagctaggcccattgctgctgccggtccttcgcggggaacgagcgccgctggaggttatgatggagggacgcctgctgtacaagtactacgccaacgcataccggctggagcccgccttcgagcagctcaagtcattgctgggcgcgatcctgcataagaaccctcgtgccagggttctcgagatcggagccggcaccggcgctgccacacgacacgcgctcaagaccctagggactgatgaggatggcggtcctcgctgcgagagctggcactttactgacatctcctccgggttcttcgaggcagcccgcgctgaattcgccacctggggcggcctgctggagtttaataagctggatatcgagcaggaccccgaagcgcaggggttcaagctcggttcttacgatgtcgtggtcgcctgccaggttctgcacgccacgaagagcatgcaccggactatgaccaatgtccggtccctgatgaaacccggcggcacgctgctccttatggagacgacacaggaccagattgacttgcagttcatctttggtctcctgccgggttggtggctgagcgaagagcctgagcgccacgcgagccccagcctgagcattgacatgtgggatcgggtgctcaagggggccggctttacgggagtcgagattgacctgagagatgtgaacgttgatgctgagagtgatctgtacggcatcagcaatatcatgagcacggctgtcggcacggcgggttcgagccctgagaaggtggatgccgcccaggtggtgatcgtgacgggcaacaagacgggctttcaggacgattgggtcaggggactgcaggcagccattgctcaggactccggtagcgatgcccttccagagattatatccctcgagtctccctcgctcggggcagaggccttccagtcccggctggtcgtcttcgtcggcgagcttgacagacccgttctggcgtctcttgactccacagagctcgagggaatcaagaccatggccctcgcctgcaaaggtcttctctgggtcacccgcggcggcgcggttgagtgtacggaccccgactctgcgcttgcatctgggttcgtccgcgttctgcgcaccgagtatctcggccggcgcttcttgactctcgacctggacccagcagcccattcgcctgcgtctgatatctcagtcattgtgcacctcctctcctcgcgcctacagccggccgttgagacagcggccccggccgacagcgagttcgctctgcgagacggcctcctccttgtgccgcgcctttacaaagacgttgtctggaatgcactgctggagcctgaggtccccgactgggcctctccagagagtattcccgaaggcccccttcttccaagccaagcggccgcttaaactcgaggttgggatccctggtctgctcgatacactcgccttcggcgacgaccccgacgcgctggacgccgccgggcccatgcccgacgagatggtcgagatagagcctcgcgcttatggcctcaacttccgcgacgtcatggtggccatgggccagctcaaagagcgcgtcatgggtctagagtgcgcaggcgtcatcacgcgcgtcggcgctgaagctgcggcgcaaggcttcgccgtgggtgaccgggtcatggccctgctgctgggcccgttcagctctcgtgcacgggtgagctggcacggagtcgccagtatgcccgcggggatggggtttgcagatgctgcctctatcccgatgatcttcaccacggcgtacgtcgctctcgtgcaagcagcgcgactgtcgcaggggcagacagtgcttattcacgccgctgcaggaggtgtagggcaagcagccgtgatactggccaaggaatatctcggagcagaagtctttgcaaccgtgggctcgcaggagaagcgagacctactgatcaaggagtacggaatccccgacgaccacatcttcaactctcgcgacagttcctttgcaccggctgccctggccgcaacagccggacggggcgtggactgcgtccttaactcgctaggtggcgccctcctccaagccagctaatcgaggttctcgcgccctttggccactttgtcgagatcggcaagcgcgatctcgagcagaacagcctgctcgagatggccaccttcacgcgcgctgtctccttcacttcgctcgacatgatgaccctcctccgccagcgcggcgacgaggcgcaccgcgtcctgagcgagctcgcccggctggccggccaggggatcgtcaagcccgtccaccctgtgtccgtatacccaatgcgccaggttgacaaggccttccgtctgctgcagacggggaagcatctcggcaagctggtactgtccaccgagcctgacgaagaggttagagttcttccccggccggccacgcccaaattgcgcgccgatgcatcttacctccttgtcggcggcgtgggaggtctcggccgctccctcgccagctggatggtcgaacacggcgcaaaacaccttatcctcctctcgcggagtgcaggcaagcaggacagcagcgcattcgttaatggcctacgggacgcaggatgccgcgtcgccgcaatctcctgcgacgtcgccgacagggccgacctcgaccgcgcgatcgcggccgcctcagagttggggttcccgcatgtccgcggcgtcatccagggcgcgatggtcttgcaagactcgatcattgagcagatgagcattgcagactggaatgcggcaatcaagcccaaggttgccgggacacgcaacctccatgaccgcttctcccagcgcaacagcctcgacttcttcgtcatgctctcttccctatccgcgatcctgggttgggccagtcaggcctcctacgcggctggcggaacgtaccaggatgcgctggcgcgctggcgctgctccaagggtctgcctgccgtatccctcgatatgggcgtaatcaaagatgtcggctacgtcgccgagtcgcggtcagtctcagaccggctgcgcaaagttggccagtccctccgcctctctgaagagtcgatcctccagaccctggcaacggcggtcttgcacccattcggccggccccagctcctcctgggcctgaactccggcccaggcagccactgggacccttccagcgacagccagatggggcgtgacgcccgcttcgcacctctccgctaccgtaagcccgcatctacgaagtccgctcagacatcttccagcggcgacggcgaagagcccctttcatccaagctcaagtcagccgattcccccgatgcggcggcgaactatgtcgggggtgcaattgccaccaagctcgcagacatcttcatggtccctgtggccgatatcgatctgaccaagccgccaagtgcgtacggggtcgactcgttggttgctgtcgagctgaggaatatgctggtgctccaggcggcgtgtgatgtgagtatctttagtatcctgcagagtgtgagccttgcggcgctggcggggatggtggtcgaaaagagtgcgcatttcgagggaagtgccacgggaactgtcgttgttgcttga

SEQ ID NO: 3 - DNA, 1768 bp, Aspergillus nidulans
  source 1..1768: mol_type = genomic DNA
gctgcatcggtcatgttgttcttctatagagttgaagcaaggtttgtagtttgctctgggtgtctggagttgtctggagttgtctggagttttgttatgatgttgatgggtacttcttcatactagcattttggcatgttataagaacatattatcagttaaatgtctttcaatttaatcaatttgtttttagaatgatgttgtctgcctggctatgtatctagatcctatacaagctctatcgactcgacctaactactacgacttgaaagtcaagcgagaagtgatgatatgaacccatatgtcagacccgctaaatttattagtgataacaactatattactcagagcttttctttctagagtatgttagaattgccctttctggctcagtgggaagctcgagacctagtccttagtcacgtgctgctacatcatgtaaatataagccctacatggctgtcttgtgcatgaggctaacaccattatctgtcactggtccttttatttggttcttttctttactttctcgggcgggggggaaagccgctaacactgtctatcgcttggacagaaactcaccagtttgttcgcaatcctgaagcgtatgggaagcttacagttaaggagtagctcgagtctggaccctgttttcgacttgtacctttgatttggatgactggttaacctcagcttatgtatgatgtgctctcatggtgtcaatatctggtagtctgattctgagcaatttgatagtatctgatggctggcgagtaaggccagggcgatgactggtataaagtcagccctaaaacttccatccgagatgtaaaaccatcgattcccctccaagatctcctgacgagactaaacaaagatcaagtggccttgtagtaactctagcaagcagcgacaaaatgcctcaacacgagatgaccaagtcagactcggaacgaatccagtcctcgcaggtaagagcatcaggacatttgctaataccattccgccccgctaatctgcttgaatgcacacaggctaaaagcggaggggacatgtctcttggaggattcgcctcgcgcgccctgtctgccgggactgctgggtcaattcccagtcctcggccactgcttccggccacgcggactcgggtgccggatctgcaggcggatctcattcggccgcacctggcggtgatgcggggcagggaagaagataaaagtaccctgttgtctttggggcgttgaggtataatggcatcgtggtagaccgactgggcttttttttttgatatagttgatcctgaagcggaggacagttggtaggataaatgaaagatactgaaccatgcccggattttgtgctcaaggacctaaaactgagaagctgaatctgttcttgtctgggagaaggcctgccagctgcatccgagtatctatcttgccaggaccaaaccgggtctgggctcagttcttctaacttcttagtggagttttgcagtgtagattcctttgcactatctggtatcctagtagcagcctaccaggaaataagagataaataaagtcttaattggcattattatgtttctcagaactatatatctcggaacaaagctgagcagacagaagtttaccctcacatatggacaaattgcgtgctcaggcataagtcggaaacagccttagccaggtcaacacttgtagccttcgctagacgacgccccagcttttcataatggccggcctggagggagatacggctatccacc

SEQ ID NO: 4 - DNA, 1593 bp, Aspergillus nidulans
  source 1..1593: mol_type = genomic DNA
ctagaacctcggaataggtgtccccttcccaaagacccccttgggatcccactttctcttgagatacgacagctttggcagattctccttgctccaccacgcctccggcccctcatcaccaaatgcataattgacgtagatatgtggctggtcagcaggaaacccgctggtggcatggagcttttcgcgcagtgagaccagcagctcgttcgttggagcctccagttccggattcaagaatatattctcgtgcagccaaaacatcttcgtgtcgcgccagggatacacggccgtgtgcgcaggcgttttgagcgtgttgttgttcgcgtatcgctggaacagactctgccccagataccccgggtactgctcgtagaacgcggtcatgtcgtcgaacacctcctgcatggtggccgcgtctgttcggcctagtcctacggtaccgccggagacgtaggctcccgtctggcagggtccgtcgaggccagcgtacagctcgacaagagtgacgttcgatacgttccggctgatcgggccgagcgcctcggcgtgctcccagtggtcgacgaaagtggcccagggggcgaagtgcttgatgtccacggtcaagagggtctcgttgatggtgcggtcgtacccgatcgagagctgcactcccagttcaggagggaggacattatcgaggacagagaggtactcgaagacgccgagactcttggatgagttatacacaaacgtgccgatcacggcgtcgccgttgttcggctggtcgaacatcttgaatgtggcggcggtgatgatgccgaagtttgcaccggcgccgcggatagcccagaggagatcgctattgcaggtctcattcgcagtgatcagctcgcccgtcgcagtgataatgcggacagagacgagtgcgtccacgccgaggccgaagagccctgtttcgtacccaattccgccgccgatagtggcgccaataaccccgacgcagggagagttgccgcgggctgtctctattagacggcatgcttaagaaggagaaggagagaatgagggggcatacggatggccttgcccgctttatagagcggctcagtgatatctcccagctttgcgcccgcaccaacggtgacggtgttggactccagatcgatgtccacgttgttaaagttggccaggttgatatcaagccctttgacggtgccgtaaatcagactagtgccgtggccaccgctggtggccatgaagctgacattgttcgcgacggcgatgcggacctgcctcgtcagtacactatttccttaagaagcaacactacaaaggcaaacagagaacaagaggcataagaagaagaagaagaagaagggggtatacaatctcctgtaaatcctcctcggtctgcggcttgatcgcgcctgtccaggtcggaggcctccattcggaccatctgggtgatacgacctcgtcaaaatccgcgtcgccaacctcggcgatctctgtttcaggcgagacgtatgggccgaaaagagattcgaggtcgatgcttgccgcgcgcgccgcagcgactagtgttattgactgaagcagaaaccgcat

SEQ ID NO: 5 - DNA, 527 bp, Aspergillus nidulans
  source 1..527: mol_type = genomic DNA
cctggtgtgattgggctgattaggacaggccggatgggtgtgcaagataggaggagaggactggtacggcgaatgagctttaatagccggtcagagattgcgcgtggctgcgcccagatccagcagctccagccatactccagcatactccggccagccgggggcatatggcgtggtcactggagctggttaggatcaactgctggttaaggcttactgtgttgccatgcttacggtgcaccgagagggaaggttggagttaacggagttgtaactccggggatccaattagggcttacagtctgcaaatccatgcaaagtccgctgcgcccctgacacagcaaggaacagtgtagagtccgattggatagcggagttgaggtgactggctggttcctgttagcccctgcatcgacctgcaatgtattgcatcaaattagggctagcctctaactccgttagactatccgcaacgcctgtcacacacgtggctaggcagcagatgatatacttttgaaagcagtact

SEQ ID NO: 6 - DNA, 8931 bp, Aspergillus nidulans
  source 1..8931: mol_type = genomic DNA
ctaaatttgtggggtatatggtgtggctatgctggatcgtcgtctaaggcccattgttaccagcactatttaagttgtcgacaagatctagtcacatactaccagcgagtgcatgcagggccgcaggatatagaccggactcagcattgagccatgtctttacgtaccactgtagttagccactgagtgatagacacattgcagcttctctagactgatcagtaatgacgatctcgcttgatactgtctgcttatgcagtatttatatagtatagtgtagactacggacagattgcatctattccgtgaggaaagggtcttcaagcatctataaggaataaaaactcgctgtcactgtacatgctctagctacctaaaagagatattgcaggtgcattgataaaggactatgcagagagctagatctcatgtttctactcaagttacagggcatggcctagcctaatatgcagttgtcctatatgtgagctagctggagccgatgggaagtgtgtttgatgaaactgattggaataatatggaattgtaagcaaagtaacaacagtctagatacaatgaatcattcccaacaccagaatacgccagactaaaaccagagttagcgaaacaaagaatatctgtaagctcaagcaatcaggcgaggtagcccatatccttccaagcctgcacatacaacctcgcaagctccgtgccaacaggcccaacccccgccatagtggtcgagtgctccttcgccttgcttgtgtcaagcaccaggccgccacagctcatgcgctcgaaatggtcgtcgaggaaatccaccagccgcgccgccggattctccgtctccatcggcagcggagaccgccgcacccttgagatccacgtcttgaatgggatgatattcgatgcgggaatatcgagtgctgacgcaagcacatggttcatggcttgccagttctgaccgacaggattgtccatatggtacactgggtatgcctcgtcgcctcgtgaggtgagatggagcaggtccacaacaccagcagcgcagtaatccacaggaatccactgcatctggccctgcaggtccggccaagcacgcagcgactgcgaagacttgactaagaaagcaaagtgctcgaccgggttccagaaaccgctcgtcgacgagcccgagatctggccgggccgcacgaccatcgcccggaagagaccgggatgccggtgaagggtctcatcaaccatgcgctcacaaatccatttcgcctcgccatatccggacggcagtgctgcagatagcgggacgcggtcctcgctcacgcgggactgcccgcagaatccgacgacgccgatggaggagatgaattggaagcccacgcggctggaaccattgaagggccgttctgcaatgtcacgggcaagatcaagaagattccgcattgcctgtagctggggctcgaatgcggacactggccgtgtcccgctcatgggccaggcgttgtggatgatatccgtcgcgttctcgaggagccagccgtactcaagcggcgggaggcccagctgtggcttagaagtgtctgtctctaaaacgcggagctttgcccgtgcgccgggggacagggtgatgccgcgggctgttagggctgcctgttggcgcttctctggggtggtgctgctgctgcgacggttgaggcacaccaccgtcgcaaccgacggtgtctcggcgagtctctgaacgatatgtgagcctaggctgccagtcgcaccagtgacgatgacgacggcctcgtgcgctctgcgtcctggtgctgcgtgcggcgcctgtgttttgccagactccttctcggcccggctcgctaaagcacggagtttgggcgtctcccagccagccgtgtactttgcaactaggctctctgctgtcgctgtgcgcgcctcaacattctcccggttcaactcggggatgagggtctgcacgggccctggcttgggcagacgggctccctgagcccccgacgcgagcgcgataatgactttctggaaggtattttcaggcaggttgccgtctgtccagtcgacgtggccaaacccggccctgtgcagctcactctcccagtgctcggccggtacgacggcgtggtgccgcccgtcatcgaacagccaccacccctcgagcaggccgaaaacaagatcgacaaaggggaccacctcggtcatttccagcatcatcaaaaacccatcggggcggagtgcctgatggatgttggacagcgagaccccgagattgtgcgtggcatggatggcattgctggcgagcaccagatgctggttcctgagctcgtcggccgggggcttctcgatatcgtgcacggcgaaacgcataaacgggtattgcttgctgaaccggcgacgggcgttggcgaccatgctgggggaaatgtctgtgaaagtgtattcaatgggcagggcgcccgattcagccagggtcgccaggaacggcgccatgatgagcgtggtgcctcctgtgccggcgcccatctcgagaaccttgagcgtctctccggtgcggccaatccgctcagcgaggaggttcgtgacttcacgcatctgtgcgtaactcatgcagttgaaggtatgctcgcagtacatggccgcggtcagctctcttccctcagggctgccaaacagcacgcggatgccgtccgtcgagccgctcaagacgcccgccagctgctgcccggcgtagtaggctagtctgttggggactgcaaacccggggtctgatgccaggacttcctgcaggatcacctggctggtcttgcgcggggccgtgatgtgcgtgcgtgtaatctggccgctggccgggtcgatgttgataaggcgtgcgtcacgctcaaggaattcgtagacccattgcatgaggcggccatgctgagggaggaaggcgacgcgggcgaggggctggcctggtgatgccgtgcgaagggggcatccgagttcatccatcgcctcgacgacgagggcagtacagagtctgttgcttccagagagcatgacgccctcggtcttgtcgactccgtactccttcatgagggtgtcggtctgcatcttgacctgcccaaaggaggctagaatgtcggaagaggatagggcaagccgagactcgacgggaggtgcaatcgctgcgagcccagcagacttgtgaatggccactgccttgagaggcaaaggctgctcctcttcgccagtgggcgtgaggatgcccgtgtctgagctttcggagccggcgtcgtcgctctcagaggcagactcgctggacgagttgtcgctcttctcttcgtcttcgtcatcttctgcctccgcaggacctgcgtttggaccaaagagcgcattcgagacgcactgcacgaacttgcgtaaactggttgcttccatctgctcgttctggtcgagagtgcacttgaacgcggcctcgacctccttgcccagttccatgcccatcagactatcgatgccaaagtccgccatctcggcgtccagctcgagctcgctggcatcaatgccagagactgtagccacaaggttgcgcacttcctcggtaatatctcgccaaccagagggcttgctggacttggatttggccttcgtaacgggcttcttctctttcttctctttcttcgaggtcttgctggcctttaccttcgccccaggttcagagctagctcttacctcaggagcagtctttagagcagcctggaaggctgccgctggtgttggtcctggcaccagcgctttcgttctcaggaccgagtcgtccttggtcatccgtgcgagcatcatgctcatcgacgcctttgcgacacgcatatactgcacgcccagcatgatctccacgagctggccgcttaccgcatcaaatacaaacaggtccgtcatgatcgctttgtcgccttgtcttgaatggcgggcataaacatgccagacgtccgcatcctctctcggcggtgctctaggcgagcgcatgctcagctcgcagcccgtcgcgatgaacatgtcgctgctcggcaggtccgtcatcaagtttacccagacaccgccgacctggctgaaactgtcgctgagcgggacatcgagccatgtatccccgcgactggatctggggagttgcacacggcctgcgcactcagttcccttgccgacgacatacttgaccccgcggtagacctcgccgtagtcgacgatcgagctgaatgcacggtagacattgcggccctgcaggacctcgacaccttcgtcggtgtcctgatcgaggcttagacggagaagatcggtgcattgcttgtgtgagacgagccgctcaaagttggcgaactcgcggacgtgcgcttggtcagaagaggagcgcatttcgaccgtggcttcggcgtgaatttctggtgttttcttggtcgcgtcatcatcaaggctgaagatcctgaccgtccagtttgtccgtctcttgtttgtcgccgtcaaatcgaggtatacgacccggctgggatccttgcagatagggctgtggttgatcatctctcggacaacgggctgcaccccatcttgcctccaccctggctcgagactgaagagggcctcgataacgatgtcgcactcgagcgtccccgggcaaatgggcgcagtctgtgcgatgacgtgactgagcacgtagcggttgtacttgtccgcggaggtattaacccggaatcgggcctgccttgtctcgtcgtcttgatagccgacgaactcccacaccggcagcgtccgggggtcctgcggcgtgccggcctgctgaccctgcagccctgccccagcgagggagccgccgttggcagcgatcaaggcgagagcggcttccttaactttctcaacgggggacttcatcgggagccagtggcgggaagaagtatcgaactggtatgggggtaggaggaggtgggcatactcagcggtctggacagcatcatgcgcccagaaggtaacgcggagaccctgcttccagagcgcggttgtggtatcggcgagagagtctagggctgtctcgttggtgatgctgacagcctggaagtagtggctctctgacgacgcctggccctgagcaatggcccggccggccatgacggtgatggtcgagctagagccggcttcgaggaagatcgcctgcgggtgtctctttgcgagacgctgcactgcgtggttgaagaagacgggttggcgcatgtgctgcgagacgaaggaggcatctgtcgctctggcagaggccacctcagtggctcgctcgacggggatgagggggctgttgaaggtcagcgtcttgccgatagagtccagcccgtcactgatcttgtcaacgagcgaggagtggaaggcgttcgtgacattgagacgcttgcccttgatcgagccgaattcgggccgcgagatcgtctgctggacctgatcgacagcactggtggacccagcaatcgtgaagctgcgcgggccattatagcaggcgatactcgcagagccatcagaccctgaagctccgttggcctcggacagtagctggtggactagtccctcatcgccttccagagccatcatggcgccccggtcagcgccccagctgtcccggacgagcttcgcacgcgccgcaaccaaacggacggtctcatccaggctcagggtcccggcaacgcatagggccgtgatctctccaaagctgtggcccactagggcctggaccttgccgttgaggccgcagtctatccaggtctgagcgcaggcgtactgcatcgcaaagagcatcgtctgaagcttaacggtatcttcaatgggctcgcggctgaatatatcgggcgcggcgtagatactgaccagcccctgcgccttaacaacagtatccaccgcatctagatgcttgcgaaagagggcaactgcgtcaaagaggccccgatccagcccgacaaagcgcgagatctggccgccgaagcagaggatgacgggtcgttcggccttgacgggggcaatgcccacactcgcggcggcatccttgctgctcggagccgcggcaacggcctgttcgatcttctcgtggagttcggccagcgagcgggcattgaagatgaatccctgaggcagaccgcggttggattggcgactgaggttgaaggagatgtccgccagggtcggctcttcggcgcgcgagcgcaaccagggcccgagtttggcacaatacgccgttattgctcgagtatcgagcccaggaatccaaaaggggtagcgtgctcctgcaacagcgtggcttctcgagtgagggcctcggagatcgggctgggtgacgatcatgcttgcattcgacccgcaagcgccgtagttgttcagcaaggccgtcttcctctcctcctcccaggcccgtagtcttgtcacaacctcgatattgtcgtccgccttgacggggatcttcttgttcatcgtcttgaaactcgcttgcggggggatgaacccctcgcgcatcatcatgattatcttgacgagcgcaatcgccccggacgcgccctctgtatgcccaatatggcctttgacagacccaattggcagcttcttcttgcggcttggtccacccagtgcagcaaggatgctctcgtactctgcaggatcgccgacgggcgttccggtgccgtgggcctcgaccagcgagacgtcgttagcagtgaccttggcctggcgcatgacgtccttgaacaggtgcgacagggacggcgagttcgggacgaacaggggcgtgcagttctcgttttggtacacggcgctcgcggcaatggttgcaataacctggttcccatcgcggagggcatcagacagacgcttgaggtagacgaatgcagcgccctcagcgcggcagtatccatcagcatcgtcgtcaaagggcttgcactggccagtaggagacacaaagctgcccgccgcgaggttctggaaccagttcatgtttgtgaccgtattggacccgcctgcaagcgcagccgtgcactctccagagagcaggttcctgcaggctgtatggatagccaccgccgaggaggaacacgccgtatcaaaggtcatacaggggcccgtccacccgaaatggtggctgactcggccggtaatgaaactcttgagtgcaccagtcgccgtgaacgcgttcgggtcgtagcacgagatgttatgctcgtagtcgacaccgcatgaacccaagtagacaccaacatgcatcttgtcacgcccgtccggggtatacccgttatggtcttcgacaaagtacccagactgctcaacagcctgatacgcagcctgcaggacgatgcgactctgcggatccatcgctgccgactcccgcggcgagcgcttgaagaatttgtggtcaaaggcatcgccgtcgcggaagaagcacccgtagaatttgcgcttcgggtcggcatctgcgttctcgcggaagagcatgtcgtgcatgagtctgtcccgggtgatggggatatgctgcgactggcccgtcttgagcatggcgacgaactcatctagatcgtcggctccggcggtcttgacggacatgccgacgatggcgatgggctcagactggggcgagacgggcatgactggctcgacgcgggtggtctgctgctgctgcagttgcaggaccggttgaagctggggttgtgggggaggtgatgattgcggtgtaagccagaatgaaggcttctcagggtctttgggaaggtcttcgtaaaagacctgtcttcctccgagagttctcatcagagttggagggacacatctctccaggccaaaggtgaccacgtaagggtctgggagggcatccgccacggccgagaaggtgtcaaaccaccggcattgctgcaccaggatcgaccgcaccaccatctcagtcatgttccctgagccagaaaccggaatgcccgatccctggttgtcgtaagtctgcagagcgagcttcgacacctctgcatactgcagcccaggcagagaggcgcacagctccaccagggcattcgtatgttgtttccgatcagcattggggctatggatctggcccttgattccaacctcggccaccgtgactcctgcagctctgaggcgcttcatgagcagtggcgcaattgtctctgaggccgtcaccgttgcccgcgcctggtcataccggacagcaacatacgcgtcgtttgacagatccccaatgattcggttcatctcgtcctcctgtttctggccgcgccaggcgacggcgtaggacgctgaactgcccttgccggatgccttgtcccatacttcttgcgcgtcgatgagagcgccgatgagcatcgccagccggacggcgacggctccgtattcctcgaacccggcctggtttctggcgctagccactgaaagcgcagcgagcaggccagcgcagaagcccaggatgaccgtcggcctgctgccggactgtgtctgctgcaccagctccgcctgcagatctacggctggggcactgccgtccctgatcatctccagatgccgccagtactgcgtcagctggattaacaccactaacgggccaaccaagatgctcggcagagactcgtcgtcagaaaccgagagcccggccgtgtcgaggctgtgccgaagccatctgtccagttcagacaaggaggtcggcccgtcgatatcgcgggctatatcaggcatcttggctgccaaggcatcccagtatgttggtaggtcggcgattgtgcgcaaaatccagtcgcgttgtggcgattgtgagagtggacgaacgagcttgtccatggatgcctttgtgaatgtaccgacatgcgggccaaataggaagactgttgaggcctcgtggcctgacccagaggcgcttgctcgggtcat

SEQ ID NO: 7 - DNA, 849 bp, Aspergillus nidulans
  source 1..849: mol_type = genomic DNA
tgcgggagggtaggagggtaggagggtagctaggtagttgatagtgctaagtgctctgccgggtcaactgtgaatgaatgaggtgtagttgagacacttgaggttgactttccaggcgagcgagcgggtcaagagagcagagagaatatgatagactgggtgtctgtagtagatagacaagatgtatgtctgtcccttggggaagtagggctaatacttctaccttagcacatgttgcgggaagccacgcactgaggaaacactgacatcgttggggcactctgattggagccggagattaaggtaagatggaatccttctggctgcagcgctgtaagccctaagcctggtggcgcttctggcggacttttcggactacaggactccatccaagactccagatcgagactcagcttcgctagtccggaagtccgctggctgatgcttgtctcagcttttcgtctcagctttgtcgtcttctgtagagcctttagggaaaccccaactcagcatatggatgcagggctggttgggctgattgggcgttgtctggacttgtatctgggtatggctgccgtctggggatcaaaggtaaatggggcagaaattgcctgttgaaatagttattgcggaggccaatgcaatatcccaagaatttcccaaaatgcaagctactatagatgctacatagccagatagaggttgataatgccacattttcaatatatacacatacgtttgtgtgtataagtacataacacgactacagtggctgatatatatgcagtggacgcctttagacatgtttccatttatgattatagagcgatcctcaggcaagtggttata

SEQ ID NO: 8 - DNA, 1452 bp, Aspergillus nidulans
  source 1..1452: mol_type = genomic DNA
ctagaccttcactacagcacgctcatacgcttctctcgcctggtcgaccatgccctgcacatcgaaatcccaaatcaccctgctcctcctctcccattcagccttacactttaccccgtcacccccaatgtcttcataccgccattcgtagagatctcccatctcccttgagctcttcacaagccactggctacgctcaatcctcacatcgctgtaagtcttcagggcaagctcaatattagacttcttctccttgaacgcggagccgttctggaccttctcaagcaactcagcgagaacaagcgcgtcctcaacgcccatacaggccccagccccgtggaacggactggacgcgtgcgcggcatcaccggccagcgccacccggccagcggcatagtaaggaagcgggtggtccgcctgatcgaagatggcgtacttgcttagctgttccgggaagaggctggcaagttccttgatatgcgggccccagttctcgaccgccgagagtatctcctccttcgagctgggcactgtcatggtgtggccgtgagtccactcgttcgagtcgtgcgtgaagaggaaaacattatagatctgggcgttgtttacctaggcacaatcagcgccttcttgcagaatagatgcggcatgctaggcctggaggtaaggtagggtaccggaaaagagacaatgtgcgcgtccggcccgcaatgtgcgatctggacatgcgccttttcggtccccagcgcatcaattgctgctggcataggcacgagagcgcggtagacagctttgcgagagtacctggcgtttgcagcagggtgttctgcgccgaggaggactctgcgggccgtggagtggacgccatcgcatgcgatcacttcacccaccgcattagcattatgaaacgtccaatacccagctcagggaagaaaaccaaccaatatctgcctcctccacctccccgtcctcgaacctcagcaccactttctggtccccaccatcctcatatgccaccagcctcttgccaaacctcacaaccctctcgggcagcaaccgcgccatctccgcatgaaaaacacccctcaagcaagcccagtacgccatattcttctcctcgatctcaaacagcacgctcttctctggatcctgtgcctcctctttgcttttcgggtggaatccgtcccagtaccgcactttatcatgcggattgcgctgcgcaactttggagagagcggatagaattgcgggatcaaggcgctgcatgcactcgcgggcgattccggtgaaggcaaatgcggccccaatgtcgggccaagctgaggcgcgctcgtagattgtcaccttgccgatgttgcggtggagaagccccagggctgtcataaggccgatgatgccgccgcctatgatggcgatggagaggggttcctgttcctgctcgtggtctgccat

SEQ ID NO: 9 - DNA, 605 bp, Aspergillus nidulans
  source 1..605: mol_type = genomic DNA
cctgtttagagtggccagaaggtgtgtgtgttatctgcaggatgccggtaccagtagggctgtatgtaaatacggctgcagtagtttcaagttctgcttcgatcaagcgttagacctaggattgagcgcggctctggcaatggcggcttttctcatggtatagcatggcatagcctgaggatataggtactccataccgaggtacgagtacatctatactaagaatagtgactcccagcttgcctatcccctgcttatcccggagtttgcatctccgccaggaagcacgcggactgaggcggagtaattaacagaaggcatggcaatgcttactgcgtggggcttaaaacctgacctgacctggcctggcctggcctgatctgatgtgaaactggttctccttctctatctccctctgtcagattgatcgtcaaaacctaaccctaagtcaaatttaaacgccacgcaccggatactctcaactctgaatacggccttgatcagccaatcacagaagattgcgagctgacagttcgtattgattactttaaagcctggcatagacgatctgccattgatttgcaattctccggcccagttgcata

SEQ ID NO: 10 - DNA, 893 bp, Aspergillus nidulans
  source 1..893: mol_type = genomic DNA
tgccggcgctcgatatcgcctcggccccggccgcagtctatcaacagcaactccatctcccacgcatcctctgcctccacggtggcggcaccaacgcccgaatttttaccgcgcaatgccgcgctctgcgaagacagctgacagacagctatcgtctcgtttttgccgacgcgccatttctctcgtccgccgggccggatgtgacgtctgtctatggcgaatggggcccgtttaggagctgggttcctgttcctgcgggcgtggatatcagtgcatgggccgctgccggtgccgctagtaggatcgatatcgacgtggaggcgatcgatgagtgcatcgcagctgccatagcgcaggatgaccgggccggcgcgacaggggattgggtcggcctgctggggttcagtcagggggcgagggtcgctgccagtctgttgtaccggcagcagaaacagcagcgcatgggtctgaccagttggagtaggggtagggatcgcaagcgaggtgcgacctctagcaccaattatcgcttcgctgtcttatttgccggccgcggaccgctcctggacctaggctttgggtctggctctttagccggctcgagtgctgcttcttcgtctgcgtctgcgtctgtatctggatctgaatctgcgggtgaagaggaagaggacgggcacctcttaagcatcccaaccatacacgtccacgggctgcgagatccaggcctcgagatgcaccgggatctagtccggtcttgccggccctcgtctgtgaggattgtcgagtgggaaggcgcccaccggatgccaataacgacgaaagatgtgggagcggtagtagcggagcttcgacacttggcgataagccggaaatatgaaagcttgagatgttga

SEQ ID NO: 11 - DNA, 384 bp, Aspergillus nidulans
  source 1..384: mol_type = genomic DNA
attcagcctattgagattacagccacggaagtaatcctgtaaggatcaggatgcaactccatgcaaggcgctaaggatcaggatccttttcttcaggattgtggcaacggcgccagcggccagcgggcgctatcgcgtcggtggtgatggcgttatttggatttcggaggatagaatccggtcagcctaatcaagccaactccgtcggacttcggcgggactgtccggtcagttagagctagagaaggaaggaggtagagtcccagatagacaaaagacttggctgctatatatcttattattcaatcctcaatcccgctagctgtcaatagaatgatcctcagccgcacttgaagtcttgtctacatcccgaatccaggcgca

SEQ ID NO: 12 - DNA, 2033 bp, Aspergillus nidulans
  source 1..2033: mol_type = genomic DNA
atggctgagacggattcctcccacacccgtgggcccgtagactcaatccagaagaacgacgcctcaagcgacgatgccgaggcagagaccaagatccagtatccctcgggctggagggtcacgatgatcctgacttcggtgacattggcgtactttcttttctttcttgacctagccgtgctgtcgaccgcgactcctgccattacctcgcagtttgactcgttagtcgatgttggatggtgcgttatgtcccctactgcgctcttccctaggtacatatgtgctggatgctaaaacccaccttgccggcaggtatggaggcgcctaccagcttggaagcgcagcgttccagcccctgacgggcaaaatctacagccagttctcgatcaaggtagttctccctcaaccatttgacgcagttggaggcttgggtgctcatgaatagcagtggacattccttgtcttcttcattgtctttgaactcggctctgtcctgtgcgccgcagcacgcaactcgcccatgttcatcgttggtcgggtcattgcaggcgtagggtcggccggcatgtccaacggcgccgtaaccacaatctccgcggtcctgccaacgcagaaacaggcgctcttcatgggcctgaacatgggtatgggccagctcggtcttgcgacgggaccgattatcggaggcgcgttcacaacgaacgtttcgtggcggtggtgttcgtccccctgctccctcctttcaaatcccacctactaggcgaccatgcagagaagatgcaccagctgatgacgacgcaggcttctacatcaacctccccctcggcgccgttgtcggcggcttcctcctcttcaacacgatccccgagccgaaaccaaaggcccctccgttgcagatcctcggcaccgcaatcaggtccctcgatctgccgggattcatgctaatctgccctgccgtggttatgttcctcctgggtctgcaattcgggggcaatgagcacccctgggacagctccgtcgtgatcggcctcattgtcggaggaggtgccaccttcggtgtcttcctcgtgcaccagtggtggcgtggcgatgaggcaatggtcccgtttgccctcttgaagcacaaggttatctggtctgcggccatgaccatgttcttctccctgtccagtgtgctcgtcgcggacttctatatcgcgatatacttccaggctatccgggacgactcgccactcatgagtggtgtgcacatgttgcccatcaccctaggtctggtcttgtttactgttgtttcaggggcgctgagtatggtcttttctcctgcgtgcttgaacaatggctaaccgtccagtctccgtactgggctactacctgcccttccttcttgcaggcggcgccatctccgccgtcggctacggcctcctctcgacgctgagcccgaccacctctgtcgcgaaatgggtgggataccagatcctctacggcgtagccagtggctgcaccaccgccgctgtatgtcttcagttttacatacccccggaaccctttgccttcacctttaccaggtagaatgccgctgacaaggccgaatgcagccctacgtcgcaatccagaacctcgttcccgcgccccaaatcccgcaagcaatggcaattatcatcttttggcagaacattggcgccgccatatctctcattgcggcaaacgccatcttctccaactccctccgcgaccagctagcccagcgcgcgagtcagatcaccgtctccccgggcgcgattgttgcggccggtgtccggtccatccgggacctcgtctccggctctgcgcttgcggctgttctggaggcgtatgcggaggccatcgacagggtcatgtacttgggcatcgcggttagcgtgatggttattgtgttctcgcctggtctagggtggaaagatattcggaagacaaaagatctgcaagctctaactagcgatggagcgcagggtgaagcgacggagaaggagactgttccggttgccctgggttaa

SEQ ID NO: 13 - DNA, 591 bp, Aspergillus nidulans
  source 1..591: mol_type = genomic DNA
ggcatcgtctacaagcagatgctaggcacacatttctttctgccgctaaaaattgggtaatgcagagccacctcgctttttttttttcgaacattttccatcttgtggtatttctgggttcatttcgctccatataacgaagattggccttggtacgggctagggttcgcgggtgggatagttatagaatgagaaataatacttttatatgtaacaatttcaacttctcaagatgaatataccattcggatagagcagcttctgagtatcgacagacttaggtaggcttatgggtatgctctgttgaatatcttgtagatgtgacaggcaatagattgttagattatagcctacaatccacagctcagctcagcacgagtttgattttttcattataattggaataagcactgagctcagaatgaaaccaatagattactagggctatgcgtagacgttgaacgggatccatcaccaagcgcagtattagggcaccttttgtcgtgggtatatagcaactaaacacattctcttcggtcctgttcggccctcttcggcctccattagccagtcaaaataaacagtaaccag

SEQ ID NO: 14 - DNA, 1218 bp, Aspergillus nidulans
  source 1..1218: mol_type = genomic DNA
ctacaaagtgacaacaagcttctttcccgaaaccccctttcgctggatatccagcgcctcctggatcttctcgagcccctttccgacaacgagcggcggcggtgcaggcacaaactgccctctctcgagcgcttggggcagaaagtccatgtaaacccggctgaccacactgtccgggtccaccagcccgtcaacaaggataaacttggcgatgacgcctgtgcggcgctgccggatgctcgatttcaccattcctcccagcatcccaatgaggtaagtccccttgccgacgaaggtggttagcttctcaggcgggatgatctcaccggcgacggcgatgaactttctcgtcagcgcaggatcatgcttgcgcatcacgagggtgcaggcttccaccgcaccggcgccaatggtatatgcgccgacgagctctctgcccttgagggcggataagagatccttggccaggaacttgctccggtagtcaaagacgtggctcgccccgagccccttgacatagtcgaagttcttgggcgacgaggtcgaaaggacctcgtagcctgctgcgacagcgagctggatcgcattgctgccaacgctgctggcgccgcccgtgatgatcaccgcgcgcggggaccccgacctgccccgctgcacctctcccctgcccttttccgcaagctgcggcatatcgagggccagatagtccttgtggaagagaccaaatgcggccgtacccagcccgagtccgagcacagatgcctgcgcatcgctgatcccagcgggcaccggcgtgagcatatgcactcgcaggacggtatacagctggaacccaccctcggccgggtcgttcacctctttcgcaatcgccgtcgcgcttccacagacgcggtcgcccacggcgaaccgggtgacgcccggtccgacctcgacgacctcgcccgcaacatcagtcccaaagatgaacgggtagtggatatacccggccagcgcgggcccgatgaactgcaagacccagtcgaacgggttgatagctacggcgccgttcttgacgaccacctggccagggccagggcgcgtgtagggggcgtcgccgactttgaaggggatcacctttttggcggggatccacgcggcgcggtttttgggtttgggggtcccgttgccgttggtagccggcgctgctgcggttgctgcggttgtatcttgagttgccat

SEQ ID NO: 15 - DNA, 1221 bp, Aspergillus nidulans
  source 1..1221: mol_type = genomic DNA
aacgaggtccaggtgacggtaacgtggttcagtgcagttccaatgtatggtagcgttgtaagctgacacggcgacggctgcgagaggggttggggggacggaaccagctgaaacaggactggcgaaagaaagctgctgtgttatatgtaggcagagctaaagaaccttgtggagcgacagaaccaaagtcagtctgggccatgggctatcttccataattttgggagctcgaggtccggattgcccgttaatactccgccagactagggcaagatagggctacgcggagttttaggtggacggatttcaaccctccgaagtccgctcgaacttttgtcgacgagattaagccactagcctaaaggaatcagacctttaattcctcaggccgagtcgggatcattgaaggcgagaatgaggtgaggttgtcagccacatcgtcagctcaatcctttagaccacgttcttatctcgcggccgttctccaatcgacgggcccgctggcccccagcgtgcagattacaccgtctcgctccgactgcaggatctggcgtcttccatgcgcggacgtttcggacggcgatgactgtctgagtggttggcagggatgcacccctacctacccctgatcgaagctaatggtaatgcagaatacgaggttggttagactaagcgcttctgcagctgcagcgcatggaagctgttctgtctggtggagagactaagcagtgctctgtgctcctctgtgctgctctgcattgcactgcactgtactgcattgtactgcattgctgttctgcacggatcattcatccatctaccatggatccactactaacctcgcttactctagtcgatctggtcaagacgaccaagacctcggagaattagatggccaaccaaggatagatgcgagatcaactgatccaccgctggcaaacttagttgtgaatgtcgcgaacgcaaataccacggagatggcatgcagccgcacccgaaatggaatgctgtaggcctaatcaagctcatcgattctcgcccccaaatctgggctgcgcggtcctgcaggtgagacggatcctggaggctccatgctggctggctctgcctcctcgtggacgagggtacgatggcagccagtctgctggcgtgctggcgccgctggtagcacggccacgagcctattgattgcacgggcaaacgttcgtaactcgctcgtaa

SEQ ID NO: 16 - DNA, 404 bp, Aspergillus nidulans
  source 1..404: mol_type = genomic DNA
ctgaaaagctgattgtgatagttcccacttgtccgtccgcatcggcatccgcagctcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataaagacccccaaggttctcagtctcaccaacatcatcaaccaacaatcaacagttctctactcagttaattagaactcttccaatcctatcacctcgcctcaaa

SEQ ID NO: 17 - DNA, 2354 bp, Aspergillus nidulans
  source 1..2354: mol_type = genomic DNA
atggcgtgtcccaccagacgaggacgacagcagcccggctttgcatgcgaggagtgtcgccgccgcaaagcgcgctgtgatcgcgtgcgtccgaaatgcgggttctgcactgagaatgagctgcagtgtgtgttcgttgacaagaggcagcagaggggtccgatcaaagggcagatcacctcgatgcagtcgcagctgggtaggtgtttgtcttgtctcattgtatctcgtctcgtctgcgcttttgtgattatggggctgccatgtttccggtccggacacaggcatctgcaaggcccgccgctgtgctcccccgatctgcagggaccaatgcagctggttctggagcttgtgctgtgctgcttccctgtctttccacatggtcgagtcgagcgagctagctaacatgggatgcctcatgctttcagcaacgcttcgatggcagcttgatcgatacctgcgacatcgacctcccccgtccataaccatggccggcgagctcgatgagccaccagcggatatccagacgatgctggatgactttgatgtacaggtcgccgcgctgaagcaggatgccacggcaaccaccacaatgtcgacgtcgacagctctcatgcctgccccagccatctcatctaaagatgctgctcctgctggtgctggtttatcgtggcctgacccaacctggctggatcgccagtggcaggatgtcagcagtaccagcctcgtccctccatcagacctgacagtctcgtcggccactaccctaaccgaccctctcagcttcgaccttttgaacgagactcctcctcctccttctacgacgacaacaacgtcgacgacgaggcgagactcatgtactaaggtcatgttaactgacctcatccgggctgaattgtacactacctaactgatttgtctaccatgacacctgactgacaatgtgcagagaccaactctacttcgaccgggtccacgccttctgccccatcatccaccggcgacggtactttgcgcgggtcgcccgagatagccataccccagcacaggcatgtctgcagttcgccatgcgaacgctcgcagcggcaatgtctgctcactgccatcttagcgagcatctctatgccgagaccaaggccctcttggagacgcacagccagacgcccgccacaccgcgagacaaggtcccgctcgagcacatccaggcctggctgttgttaagccactacgagctgctgcggatcggcgtgcaccaggctatgctcacggctggccgggcctttcgtctcgtgcagatggcacgactgtcagagctggatgccgggtcagatcgacagctctcgccgccgtcttcgtcgccgccgtcttcgctaaccctatctccttcgggggagaatgctgagaacttcgtcgacgccgaagaaggccggcggacgttctggcttgcttattgctttgatcgtttgctttgcttgcagaatgagtggccgttaacgttacaagaagagatggtacgtcgcgcttcttttattctatttacctcagaatttatattcagttattttttattctaaccctgctagatattaacccgcctcccctccctcgaacacaactaccagaacaatctccccgcacgcacgccctttctcactgaagccatggcccagaccgggcagagcacaatgtccccgtttgccgaatgcattatcatggccacccttcacggccgatgtatgacgcaccgccgcttctacgcaaacagcaactcgactgcgtccggctccgagttcgagtctggcgccgcgacgcgagacttctgtatccgccagaattggctgtcgaatgcagtggaccggcgagtccagatgctacagcaggtctcctcgcccgctgttgacagcgacccgatgctgctcttcacgcagacgctcggctaccgcgcgaccatgcacctgagcgataccgtccagcaagtctcctggcgggctctcgccagctcgcccgttgaccagcagctactgagcccgggcgcgacgatgtcgctgtcggccgccgcgtaccaccagatggccagccacgcagccggcgagatcgtccgcctggcgaaggccgtcccctcgctgagtccgttcaaggcgcacccgttcctacccgatacgttggcgtgcgccgccacgttcctctcgacgggcagtcccgatcccacgggcggcgagggggtgcagcatctgctacgagtgttaagcgagctgcgcgatacacacagcctggcgcgggattatttgcaggggttgtcggtgcagacgcaggacgaagatcatagacaggatacgaggtggtattgtacatag

SEQ ID NO: 18 - DNA, 7527 bp, Aspergillus terreus
  source 1..7527: mol_type = genomic DNA
atggcacccatggagccgattgccatcgttggcactgcctgccgatttgccggctcgtcatccactccttccaggctttgggaacttctcttaaaccccaaggacgtggcatcagagccacccgcagatcgattcaatatcgatgctttctatgacccggaaggctccaaccccatggcgaccaatgcccgccaggggtatttcctttctgacaacgtcaaagccttcgatgccccgttcttcaatatctccgcagccgaagcactggcactcgacccacagcagcggatgctgctggaagtcgtctatgaatcactggagactgctggcctgcgcttagacactctccgcggctcctcgacgggggtctactgcggtgtgatgaactccgactgggagggcatattcagcgtctcatgtgcagcaccgcagtatgggagtgttggggttgcccggaataacctcgctaaccgcatctcctacttcttcgactggcaaggcccgtccatgtccatcgataccgcctgctcagcgagcatggtagcattgcatgatgccgtctccgcactcactcgccacgactgcgacatggctgcagctctaggtgccaacctcatgttgtctccccagatgttcatcgctgcatccaatttgcagatgttgtccccaaccagccgcagccgtatgtgggatgcgcaggctgatggttatgcgcgtggcgagggggtcgcatccgtgctcttgaaacggctttcagatgcagtggccgacggcgaccctatcgaatgtgttatccgagctgtcggcgtgaaccatgatggccgtagcatgggtttcaccatgccgtcgagtgatgcacaagtgcaactgatcaggtctacttatgcaaaagccggattggatcctcgctgcgcggaagatcgaccccaatatgtcgaggcccatggtacaggcacgttggcgggtgatccccaggaagcatccgcccttcatcaggccttcttcagttcctcggacgaggacactgtactgcatgtcggttccatcaagacagtggtaggccacgcggaagggactgctggtctcgcgggtctcatcaaggcatccctgtgcattcagcatggcataatacccccgaatcttcttttcaatcgcttgaacccggctctggagccatatgcacggcaattgcgagttccagtagacgtgatcccctggccctcccttcctccaggcgttccccgacgtgtttcagtgaactccttcggctttggtggcaccaatgctcatgttattctggagagctatgaacctgctagagacctcaccaaggacggcttcaatcagaatgcggtgcttccgtttgtcttctctgcggagtcggattatagtcttgggtcggttctggagcagtattccagatatctctccagattttctgacgtggacgtacacgatctggcatggacgctaatcgagcgccgttccgcgctgatgcaccgtgtcgctttttgggcgccagatattgcacacctcaaaagaaggatccaggatgaggtcgccctccggaaagcagggacaccctcgacagtcatctgccggccacatggcaagactaggaagcacattctgggcgtcttcactggtcagggtgcccaatgggcgcagatgggacttgaactaatcaccgcgtccaccattgcgcgaggctggctggatgagctgcaacagtctctcgatactttgccggaggcgtatcgtccagagttctcgctctttcaagagcttgctgcggatccggccgcatcacgactatcggaggcccttctgtcgcagaccctctgcacagcaatgcagattatctgggtgaaggtgctctgggctctgaacatccacttggaagctgtggtcggtcactcatctggcgagattgctgcggcctttgcggctggctttctgacagctgaggatgccattcgcattgcctaccttcgaggtgtgttttgctcggcttcaggcagctcgggggaaggtgcgatgctggccgctggtctttcgatggacgaagcgactgcactctgtgacgacgtatcctcgtctggggggcgaatcaacgtggcagcgtccaactcgcctgaaagcgtcacgctctctggagaccgagatgcaattctgcgagctgagcagcagttgaaggataggggagtctttgcccgtctacttcgtgtcagtaccgcctaccactcccatcacatgcagccatgttcgcagccctatcagaacgcattgagtagttgcaacattcagattcaggccccggtgcccaccaccacctggtattcaagcgtctatgctgggtgccccctggaggagccttcggtcatagagacgctcggtacaggagaatactgggcggaaaatctagtcagtcctgtgttgttctcgcaggcactaacggctgccatatccaccacaaacccttccctggtcgtcgaagttggacctcatccagctctgaaaggacctgccttacagacgatctcaggaataacgtcaggggagatcccttatatcggggtatcagcccggaacaattgtgcacttgagtccatagccacagccattggatctttctggacgcatcttggtccacaagtcatcaatccgcgagggtacctggctcttttccggccgaatgtgaggtcttcagttgtccgtgggctgcctttgtatccctttgaccatcgccaagagcacggttatcagacccgcaaggctaatggttggctgtaccgacggtacacaccacaccctctgctgggttctctgagtgaagacctcggggagggcgagttgcggtggaatcattacctctccccccgacggctcccatggctcgatggccaccgcgtccagggccaaatcgtggtccctgccacagcttatatcgtgatggctctcgaggccgctcgcatactgaccgctgagaaacaaaagagcttgcatctaatccgtatagacgacctagtcatcggtcaagctatctccttccaggatgaacgagatgaggttgagactctgttccacctcgcccctatggtggagaccaaggatgacaacacagcagtcggccggttccgctgtcagatggctgcttccgggggtcacgtcaagacatgtgcggagggcatcctcacggtaacctggggctcgccgctggatgatgtcctcccataccctaggtctccagcgcccgcagggctagcccatgtagccgacatagacgagtactatgcgtcgctccgaagcttgggttacgagtacaccggcgccttccagggaattttttctctctcccggaagatgggtatcgccacgggccaattgtgtaaccctgcattaaatggctttctgatccatccagcagttctcgacactggattacagggtcttctggccgcggtgggggagggacacctcacgagcctacatgttccaacccgcattgatgcattcagcgtaaaccctgcagcctgtagtagcggttcgctagcctttgaggctgccgtgactcggacaggattagacggtctcgtgggcgacgtggagttgtatacggataccaacggccctggtgccgtcttctttgaaggagtgcacgtctccccactagtgccgccatccgcagcggatgatccgtcagtattttgggtgcagcattggacaccccttagcctggatgtcaaccgttccaaatctcgactgtcgccggaatggatggccatgttagaagggtatgagcgccgggcgttccttgcactgaaggacatcctccagcaggtcacaccagagcttcgtgccacttttgactggcatcgtgaaagcgttgtcagttggattgagcacattatggaggaaacccgcgtgggtcggcacgccgtctgcaagcctgagtggctagaccaagagctagagaatctcggacacatatgggggcggccagacgcgcgcattgaggatcgaatgatgtatcgagtttaccggaacctgctacccttcctccgcggggaagcgaagatgctagatgctcttcggcaggacgaattgcttacacagttctatcgcgacgagcacgagctgcgcgatatcaaccgtcgactgggtcagttggttggtgacctagccgtgcgctttccacgtatgaaactccttgaagtcggcgccgggacaggctctgccactcgagaggtactcaaacatgtcggccgggcctaccattcctacacgttcacagacatctcggttggcttttttgaagacatgttggaaacaattcccgagcacgcggaccgtctgctattccagaagctcgatgtcgggcaagacccattgcagcagggctttggtgaacacacttacgatgtaatcatcgccgctaacgtacttcatgccacaccgacgctgcaagagactctgcgaaacgtgcgtcgtctactcaagccaggagggtatctgatcgctctggagatcactaacattgatacaatccgcatcggcttcttgatgtgtgcctttgacggctggtggcttggccgggaggatggccgtccatggggtccggtggtctctgcatcacagtgggatagcctactccgggagacgggattcggtggcatagacactatcactgatcgcgccgctgaccagctcaccatgtactctgtctttgccgcccaagcggtggacgaccagatcactcgatgtcgagaacctctgacgccgctccctcctcaacctcctttctgccggggagtgatcatcggaggctcgcctagtctggtgacaggcataagagtcattattcatcctttcttctcgactgttgaacatgtttctaccatcgagaacctgacggagggagcaccagctgttgtgttgatgttggctgacctgagcgacatcccctgcttcgaaaatctcaccgagtcaagactggccggactcaaagcactggtgcaaatggccgagaagacgctctgggtgaccacgggctctgaagcggacaacccttatctctgcctcagcaagggctttctcacttcgatgaattatgaacatccagctatcttccaatatctgaacatcatcgactcggctgacgtccaacccgtggtcttggccgagcatcttctgcgattggcctataccaaccaaaacaatgacttcgccctcacgaattgcgtccacagcacagagcttgagctgcgtctctaccagggcgggattctgaagttcccacgcattaacgcgagcgatgtcctgaacagtcggtacgcggcagctcggcgcccagtcacccattctgtcaccaacatgcaggacagcgtggttgtacttgaccaaagcccaagtgggaagcttcgactcgtgtttggggaggagcttgcaggtgatcgcgcaaccgtcaccattaacgtccgatactcgacctctcgtgcaatccgcatcaatggtgctggatatctggtccttgttctcgggcaggataaagttaccaaagcgcgtctggtggctctggcaggtcagtctgcgagcgtcgtctcgtcctcctgttattgggaggtcccagcagatatcttcgaggagcaggagcccgcgtatctgtacgccacagcaacagctttgctcgctgccagtttggtgcagtccaacggcaccacaatcctggtacatggcgctgacatggtcctacgccatgcaatcgccatagaggccgcttcacgggtcattcagcctatattcactaccacatctccctccgcagcatcatccgcgggtcttgggaagagcatcctcgtgcatgagaacgacacccggcgacaactggttcatcttctccctcgatatttcacagctgctgtgaatttcgaccctagtgcccgccgactcttcgaccgaatgatgacagtcggtcatcaatcgggtgtcacagaagaacaccttcttaccactttgacagctgccctccctcgtccgtcagcatctctgctgccggcccagcctcaggctgccatggacactcttcgcaaagcctcattgactgcttatcagttcaccgtccagttgacagcaccaggacccatcatcgcaccaatcgccgacatccaatcctgttcacaacagttagcagtcgtagactggaaaccatcttgcggctcggttccagtacacctccaaccagccactgagctggttcgtctctctgctcaaaagacatatctcctggtgggtatgactggtgccctcggccaatccatcacgcaatggctggtcacccgcggcgctcgcaatatcgtcctcaccagccgcaagccatcagtggaccccgcatggatcgcagagatgcagaccacaacaagcgcgcgtgtcctcgttacgccaatggatgtgacaagccgcgactcgatccttgtggtggcacacgccctgaaggccgactggccgccgctcggcggcgtcgtcaacggtgccatggtgctctgggaccgtctcttcgtcgacgcacccctgtccgttctgacgggacagctcgccccaaaagtccaggggagccttctcctcgatgagatttttggccatgaaccgggccttgatttctttatcctcttcggtagcgctatcgccactattggaaatctgggtcagtctgcctacacagccgccagtaacttcatggtcgcgcttgcggcgcaacgccgcgcccgagggcttgtcgcaagcgtcctccagccggcgcaggtcgccggtgccatgggttatctcagggataaagacgacagcttctgggctcggatgtttgatatgattgggcgacatctcgtctccgaaccagatctgcacgaacttttggcccatgctatcttgtcgggtcgtggccctccagctgacgttggatacggaccaggcgaggatgagtgcatcattggcggactccgcgtccaagaccctgctgtatacccagatatcctctggttccgtacgcccaaagtctggccattcatccactatcaccacgagggaactggcccttcatctggggcggctggttcgatatcgctggtcgatcagctgaagtgtgcgactagcttagcccaagttggggacatggtggaagctggcgttgcggccaaactgcaccatcgactccatctcccaggcgaggttggaggcgtcactggcgacacgcgtttgaccgagctgggggtggactcgttaattgcggtggacttgcgtcggtggtttgcgcaggagttggaggttgatattcccgttctgcagatgctgagtgggtgttcagtaaaggagctggctgcttccgcgacggcgttgttgcatccgaaattctatccggaggtggtggccgattctgacgtggggagtgagagggatggttcctcggactcccgtggtgatacctcttcctcctcgtatcagctgatcactccggaggagggggaccatgactga

SEQ ID NO: 19 - DNA, 687 bp, Aspergillus terreus
  source 1..687: mol_type = genomic DNA
ctagcgacgaggcttccgcgccttgaacataaggaccgttccaataatcacgctctccacctcctcgaactcgtcctccagcgcacggacaaagtcgtctggatagtctgaccgattctgaaacatgtccacagcattgtagatgcgctgaaggagccaactgaaccaattctgccgaactccacggcacagcagcgtagacccgaagagagtgccattgtccttgaggagcggcttcaagttggcaaacacgcgtcctttgtccttagaagtccccgggagacagtgcaggacgtacataagggatatggagtcgaactgccgttcaggttgtatagggatgggctccaggatattggccagcacacactccgtgcgatccgctactccaacgcggttggcagccttcctcaggcatcggatgtgaaaatccactagcgtcagcttctccggccaggacggccgacgcttccgcacagcagagagatagtagcccgtgcccacgccaacatcacagtgccgagatccaatgttggacaggaaaaaagggagaagaatgtccttagacgaacacttccaggcaaagagcgcgctgacccaatgaacccagaagtcgtaccaccacaagagaagtggattgtagtagtngtcggcgccttcggcatcggaaagctggtaggaggtcat

SEQ ID NO: 20 - DNA, 1611 bp, Aspergillus terreus
  source 1..1611: mol_type = genomic DNA
tcatacttccttgacattgaacaccacccagctaatccacaaaactatcacaagtccagagcaatacatcaccctctccccaaattcctgccacttggacagaccccaagtattccaccactccaacttcggccagtacttccccgaacgttcagtcaacggcaagaactgcagtacctcaccgttgtatgagttacgcaccacccccaacgcaaaaagatcgccacagtgtggcgccacatatcgcgtgaaggccgtgaccatccgtccctggcaagcctccaatcgcgtgatccagcgcgactctagatggatagcatccagtcgtttgcggtttttctcgctgaaggcagcgagcgcctggtgaatggtgtcacttgacggctggtcatggttttgtgcaatcgcatatatcacgttggccagtccagccgccgcttcaatggctgtattggcgccttgaccgatgttgggggtcatctagagtcccagctatcagtatatgaaggggaaaaaaaggctccatgcaagacataccttgctgatactatccccgatgcagatgatccggccatgatgccacgtacggaagagattctcctccaacgcaaccatccggaatccccggcgttgagcccagatatcccggaattgtacttcctcccagataggctggctggcggctgcctcacagcgcgcaatggcgtcctcctgcgagaatcgcggaacgtcagggtagatatacttgtgtggtagcttttcaatcagcacccagaaaaggctctccccggtcgcagggaatatcaggatcgtgaaaccgggcccgatgcggatcacatgctgccaacgcttccgtccggggatggggttggacatgccgaagacgcagctgaactcgacggacatgcctagccgcagagcatcatctattaatattggacggaatcgataaggagtgtagcaacatactggcctgttctttgagcggaatcagccccggctgctctatattagcaatgcgccacatctctcgccgcgtcacactgtgcacgccgtccgcaccgacaaccagatctccctggaactcgtccccatctgcggtggtgaccgtcatcttgctgccatggggagtgatccggacgacgcctttgctcgtgaggaccctggacttgtcaggcaaatgggcgtacaggatctcgagtagctgagtccgttccaggcacgcgaatttcaagccaaacctgcgtacccgccgtgttagcgtcttcttacgactttgttccaccctctcggggaccaaggaagcaccgacctcttcaagacgacactaggcgaaaggctatcatagtagaacccatcctgaaagcaaagatgcaccctttgaaatggctggcagcggtcttcaatgtgccggaagatccccagctgctccatgatccgccctccattcggcaggatggccaccgcggcgccaatcggcggatggacttcgtgatgcttctccagcaccacgtagtctattccggcccgatgcagacagtgggcgagggtcagacccgtgacggatgccccgacgatgacgaccttgaactgagggtgctttccttccat

SEQ ID NO: 21 - DNA, 1132 bp, Aspergillus terreus
  source 1..1132: mol_type = genomic DNA
tcagaattgagattcctcccgcagcaaccaaacagccgcaccgcagggccctgagatcagacaaagacctccaactttcagcgctagatagcaagtctgtgtgaatgacgactgcctctcaactgtccgccgcatatgcagtgcccacaggagaaagctccccattccaatgaggtgatcccactgtaggaaccacagcgccccttgggccatggattgaacctggaccgtacgacccccagcaaactgccaaggggatacatccgccaagagatttgtaggagccaaggtcagggaaagaccccagctgattacatgggggataatcgcgcatacaaacgcaaaggtatacgcggtccggcatgcactcctcgtggagatacccgtgctcgctcttggtcggaaaaaggcccgtagaccccagtgacacagagctgcatagattggccagagctgccatgcggcaatggccatctgtttgccgaacaagtcctggtgcgcggattccggaaggaccatggcgatagtcgggactccaaatcccaggatcatgctgatggggatgaggcgtatggaatgagctgctgatgccgagataacgcgcgccactgggcgtgatgatgatgatgacgacgacgacgacgacgaccagatgtggatcgcgcaccagaggggtacgacgacggcgatggccacgacctgggatagcatggcgaaaagggttggactatgacggttggttcggaacatggccgacagatgaagaatagggatacgagggtagagacactcacgatagcagaacgctggtgcgggttggcgatctccagctctggacctggatcgccacccacacggccacgattgcaccagagaagtgaaaggcctggacactgagaccaggatggcgtccgtccagcacgggccagtagaagacaattagatttcccaagagttcgtcaaatcccgttccggtgatgttgccttgcaagggctctgccgtgccggatagctttcgctctcggtagctgttggccatgagctcgaggaagccattccggaatcngaagccatagatggcgtctagtccaagtacggacaagcagagaagtatgtaggctgaaagggccat

SEQ ID NO: 22 - DNA, 1885 bp, Aspergillus oryzae
  source 1..1885: mol_type = genomic DNA
caatgctcttcaccctcttcgcgggtctgaaataccctcacctggcaacagcaattggcgcttcatggctgtttttccgatctctctacttgtacggctatgtgtactcgggtaagccacaaggcaagggcagattgctgggaggtttcttctggttttctcaaggcgctctgtgggctctgagtgtgtttggtgttgccaaagacatgatctcttactgagagttattctgtgtctgacgaaatatgttgtgtatatatatatatgtacgttaaaagttccgtggagttaccagtgattgaccaatgttttatcttctacagttctgcctgtctaccccattctagctgtacctgactacagaatagtttaattgtggttgaccccacagtcggaggcggaggaatacagcaccgatgtggcctgtctccatccagattggcacgcaatttttacacgcggaaaagatcgagatagagtacgactttaaatttagtccccggcggcttctattttagaatatttgagatttgattctcaagcaattgatttggttgggtcaccctcaattggataatatacctcattgctcggctacttcaactcatcaatcaccgtcataccccgcatataaccctccattcccacgatgtcgtccaagtcgcaattgacttacggtgctcgagccagcaagcaccccaatcctctggcaaagagactttttgagattgccgaagcaaagaagacaaacgttaccgtctctgctgatgtgacgacaacccgagaactcctggacctcgctgaccgtacggaagctgttggatccaatacatatgccgtctagcaatggactaatcaacttttgatgatacaggtctcggtccctacatcgccgtcatcaagacacacatcgacatcctcaccgatttcagcgtcgacactatcaatggcctgaatgtgctggctcaaaagcacaactttttgatcttcgaggaccgcaaattcatcgacatcggcaataccgtccagaagcaataccacggcggtgctctgaggatctccgaatgggcccacattatcaactgcagcgttctccctggcgagggcatcgtcgaggctctggcccagaccgcatctgcgcaagacttcccctatggtcctgagagaggactgttggtcctggcagagatgacctccaaaggatcgctggctacgggcgagtataccaaggcatcggttgactacgctcgcaaatacaagaacttcgttatgggtttcgtgtcgacgcgggccctgacggaagtgcagtcggatgtgtcttcagcctcggaggatgaagatttcgtggtcttcacgacgggtgtgaacctctcttccaaaggagataagcttggacagcaataccagactcctgcatcggctattggacgcggtgccgactttatcatcgccggtcgaggcatctacgctgctcccgacccggttgaagctgcacagcggtaccagaaagaaggctgggaagcttatatggccagagtatgcggcaagtcatgatttcctcttggagcaaaagtgtagtgccagtacgagtgttgtggaggaaggctgcatacattgtgcctgtcattaaacgatgagctcgtccgtattggcccctgtaatgccatgttttccgcccccaatcgtcaaggttttccctttgttagattcctaccagtcatctagcaagtgaggtaagctttgccagaaacgccaaggctttatctatgtagtcgataagcaaagtggactgatagcttaatatggaaggtccctcagggacaagtcgacctgtgcagaagagataacagcttggcatcacgcatcagtgcctcctctcagacag

SEQ ID NO: 23 - DNA, 1053 bp, Clitopilus passeckerianus
  source 1..1053: mol_type = genomic DNA
atgagaatacctaacgtctttctctcttacctgcgacaagtcgccgtcgacgccactctgtcatcttgctctggagtgaagtcacgaaagccggtcattgcctatggctttgacgactcgcaagactctcgcgtcgatgagaatgacgaaaaaatattggagccctttggctactatcgtcatcttctgaaaggcaagagcgccaggacggtgttgatgcactgcttcaacgcgttccttggactgcccgaagattgggtcattggcgtaacaaaggccattgaagaccttcataatgcatccctactaattgatgacatcgaagacgagtctgccctccgtcgtggttcaccagctgcccacatgaagtacgggattgcgctcaccatgaacgcggggaatcttgtctacttcacggtccttcaagacgtctatgaccttggcatgaagacaggtggcacacaggttgccaacgcaatggctcgcatctacactgaagagatgattgagctccatcgcggtcagggcatcgaaatctggtggcgtgaccagcggtcccctccctccgtcgatcaatacattcacatgctcgagcagaaaaccggcggcctgctcaggcttggcgtacggctcttgcaatgccatcccggtgtcaatagcagggccgacctctccgacattgcgctccgtattggtgtctactaccaacttcgcgacgactacatcaacctcatgtccacaagctaccacgacgagcgtggatttgctgaggacattaccgaaggaaagtataccttcccgatgttgcactctctcaagaggtcacccgactctggactgcgtgaaatcttggaccttaagccggccgacatcgccctgaaaaagaaagctatcgctatcatgcaagagacaggatcgcttgttgcaacccggaaccttctcggtgcagtcaggaatgatctcagtggattggttgctgaacagcgtggagacgactacgctatgagcgcgggtcttgaacgattcttggaaaagttgtacatcgcagagtag

SEQ ID NO: 24 - DNA, 2880 bp, Clitopilus passeckerianus
  source 1..2880: mol_type = genomic DNA
tcaatggtggattccattgctcccgtttgctgtgaccttgatcccatttgtcgccgacccattagctttcttaaccccattggtacctttggaaacctcctggttggcgttgctgatatcagcgcgagtgagacgaccaaggtcatcgtagagtgccgtgtgcaggtaggtgacccggatgatattgatataatcccgtgcacgtttggcaccgacatgtggagtgagttgcttgaccaagtactcgaacccatcgtcggtggccttgcgttcgaatttggtcaattcaagcagagcagcttcacgagccttctctgtatctgtaccagactttggtcctgtgaattcggagaacatgatggagttgagattgacttcgttgaagtcgcgggagatactgtgaagatcgttggcgagccttgagaatgtaccgaagtgcatgacgcagtcgttgaacaagtacttcaggactggggaggggaaaacgtccaccaaatcgcgagagcctcgttcttcattgatctgatgaccaagaagacaaagggcgaagacgagggcgatggtcccggcgacgttgtcagcgccaacgacatgtgtccagcgatagtgagaggttccgatgcgctccttgtcgagtccacgttcacgaaggagaatgttttcttcgcactgaccaatacctgccaggaaatagtgctcgatttcggagcggaggagagccttatcgttatcgctggcgagctgtgcacggggatggttcaacagggaataggcaaagcgctcaatgacctcgatgtgcgtaggcatccggtcatccgggacctcgctgagggtcgagaacgacttcggatccgcgaacaggtcgcggatcttcttcttgaggtcgttcaagtcgtcattggtggccttgatgagggtcatatcgaggtagtcgtcggtgttgtaaagaccgcggatgagaacgagcacgtccagcatcccttgtgaactgataggagtgccttccaagctgctcggagcgatggtcatgtatggcaagaactcgaaccatttgcccgccgctcccttctctaccttggcgaacgtggacgatgggacacggttgagctcggggcccatgagagtggcctcaatgccccacgtaagcttgcgccattcgggagcaggcttgaacatgtcaagacgcccgaagaacttagagagttcctttggcaaggtttgcgagatagtaggaagagtgctgatggaagatggatcgaagcgggggatgggtacgttgagagcagaaacgaggtaggcatcgcggaatgactcgacgctatatgtaaccttgtcaatccagacacggtcctccggtttggcagcagggcgggcgtagaagatggaggtgaggtatgccttcgcggattcaatgactttgtacaggtggtcgcggatgaggtcgcaagtgggaagagaagcgacgttggcgagtgtaatgagagcgtatgaggtttcttcagcgcatccccaagagccatcgggcttctggctctggagaatacgactgatcattgtgaagcaggcgatggacaccctggacagaagctcctcagatatggatttaaggttgccctttccgtgctcgaaaaggagacggacaagcgcctgtgaagacagcatagaggagtaccattctgatacattccatttgtctttgacgacacctgctgatgtccaccagacatcggcgacgtaggtggcgatcttgacgatttgggattcgtacatgttgacatcaggggcgtggaggagcgacataaggcagttggagttgacggtcacgcttgcgttcctttcgaaagagtagcaacggaagtaggtaggtgcctcaaactctgtgacgaattcgtcatgggcatatgggtggttgagaacttgcaagagcatcagggtcttcgagctcatgtcagcgtcgtgagtggtgccgggaacgaagcctaagacaccttttcctgccacaaggaattcacgtagtttgagggcaatgcgatccaagcattccggatccatttgtgcaaactccaggttgttgtcataaagggagctgagcgaccatacgatctcgaagaaggtcatcggccagaggttaggaacaacatctcggccatggggtgcgtagacctcgataacgtggcgaaggtaatcctccgctcggtcatcccacttggtggccttcatgaggtatgcagcggtggtagatggcgtagccatgaagttaccatcacgtaggagatgaggcatgcgatcgaagtcgcagacaccaacgaatgcctccatgcagtgaagcaaggagctgttcttggcgtagatagcctcccagttaagcttcgccagttttccggcgtacatgttgtacagaaggtcatgatgggggaagctgaaggatacgccaaaggcatcgagttgtttgaggaggcagggtacgatcatctcgtacgcgacacgctcagtctccatgatgtcccagcgctttagggcatcgtcgagataattttgagcggctctggcacgggcaggtatgtcgggttttgaggcgttgctctcgtgcatcttgagagcgacaaggcaggccagagtgttgacgatggagtcgatgagtgacccatcccctgaccaactgccgtcggcctcctggtgctcgtagatgtaggtgaaggtctccgggaagacgaagacttgcttgccgtcgatctcacgggagaccatggctacccaagcagtgtcgtagatagtcggattcgcggtgccaatacccctagaacctggcgtattgagcgcagactcgagagtctgcatgagggttcgggcgcgtgcatgaagatcttcagatagacccat

SEQ ID NO: 25 - DNA, 1572 bp, Clitopilus passeckerianus
  source 1..1572: mol_type = genomic DNA
ctacaacgcagcgaacgcttccttaatcaagtcttccttcatcttatctcgaggttcaattttgcatgcgaacggaagtggaagagtctcaagagaaaccgacttgtcgtaacagtcctccatgttcatattcttcacagtgtccttgtttgaagaatctgggtaaaaattgaatgcccaacagagcctcatgatgaagagaccagttgatcttttcgcgagcttatcgcctgggcagactctacgtccagcaccgaaaaggaaatcgggattgacgtcttcagataagcctggcttcgtgccgtttggcgacaagaaatagcgttcaggcttgaaggcctcaggttcgtcgaagagctcggggtcgtggcccattccccagatgttcatgaagatcatacttccctctggtagtacgtaaccgccataagacaagctctcccgcgagacgtggggaagggctacagggccgactggccgaatccgaaggacctcctgtaggaacgccttgagataaggcaaccgctctaaatcattgaagcacggcatggtttcggtccccaaaacattatccagctcgtcctgtatcttgcgctggcagtccgggtgggcgataagagcaagaatacacgattcgatgtacgatatcgtggtcttcgcgccggcatccaagaagccaccgctaaggtttgataactcaatccagctacgaccatccggatggtcaatcacggactctgcaaaacatccggtcctgacaccggaatccatcgccttcttggcaccgtccaagagagaattgtagacaccattacgaaaatccttgaattcgtccacaatagtcttccagccggccccggggaaaccgcgaggaatgtagtctaagaaggggaaagcgtcgaccgctgcaccattgtgagcgatttgaccaattctggtggcagcttcgtatgcattctcgataattgtgccatagtaactctcgcagcgtggctggccatacacaatgtgtaggagcagcgacatcatagcgcgcctaatatggatcggccgattaggagcgtccatcaatagatcgcgcatgaggttcacagattcctcttcttgtcgcgctatgtagccactcaaggcacttggcgttaggtaattgtggatacctttgcgaccagtcttccatacagaagtgtccatgctttccaccgtgagattcaagccttcagtataccgggcaatcatgggcgaaaatggccggtctcctgtgatattaccctgcttgtcaagaatagtccgaacagcctttggactgttcaaaacaatcacagtgcgattcatcaatttgagagagtacacttcgccatactccctggcccactgtgtcaattgcattggaagccacatcttcgtcatgagatgagcatttccgagaacaggcttggtaggtggcccgggaggcaagaagttctccctggagcctagctgaaggagcttatagacggcaacagcggatcctgcagcagcagccacgatcacgggatccaagttcgcaacagacgggaggtcgacggacagcat

SEQ ID NO: 26 - DNA, 1578 bp, Clitopilus passeckerianus
  source 1..1578: mol_type = genomic DNA
ctaatagtctgcaacatcgtggatcacctgcacaactgactgactacgtggtaccatctcgcattcaaacggttttggcatcgagaccggaccgggtacaacgacatcgtccttcattgacttggggctgttaggcaggggcttgatgtcgaatccccagatgatgttcaaagatacagtgcgcttgaaaatttcagccatcttgagtccaggacagagcctgcgcccagcgccgaaagtgaaggtatgacggtagccagtcaggtcaacgcttggttttgtgccaaattcagactccatgtaccgttcggggcggaaatcgtctggggcctcgaaaacatttgggtctcgttggatgccataaaggttcatcacgatgacggtacccttcgggatgaagtagccattgtattcgaaatcctctgtcgagtaatgaggcggtacgatgggactcggaggccagatgcgagttacctctctgacgacgcaattgaagtatttcatcttcaatgcatcttgataagttggcaaacgcgagtcgtattcatcgcccatgacctccttcagctcatcacgaatcttctgctggcattcggggtgcatcgtcatcatgagcacgaagacacgagtgaacatagcgagggtatcagttcctccgtcaatcatgacgcctccgtgataggcaataagatccctatccttgaatccaaactcatccttcctctgaagaatggtctgcatgtgagacccgtcgaagacgccagcttccattctcttctcaacccttccgaggaaatcattaaagataccaagttgcttgtccttgataccttgagccatgaccctccagccggccagactatcaggaagccacttggcgagccaaggaattagagcggtgaagtgaacacctcggagacccatcatgttttcgaagtcgtgaagatattcttcgtggtagggaatgaatgggtctgaggaggtgaggacgcgttcaccataagcgatagcaacaatactggacatgctggtgcggacgagatgcctaaagaattccttgggctcagccaacagctccttcatcagcacgatggtctccgtctcaatgttctctgcatatcgatcaatactgtcgttgctaatgagcaacttaaaggccttgtggttgattcggaattcgtcggatttgtaggaggcgataggaaggaaacggtcgtctttgataggagcagggaggaaaccagtgggtctttcagcagtcttggcattcagcttgtcaagaatgccagtaacggaggctgagtctgttaggacgataacgttcttgaagaagatcttcaagctgtatattcctccatattcttgtgcccatcggctaagctgaaggtgcatgtcgtccattgctggcatctggtggagattacccaacaccggcttcgtaggtggcccaggaggtaacgtcttctccctcgaccccatacgaagcagcttgtagaccaagtagcatgccaaagggatggccacaggtgcgatcatgttgctgtcaagcagagcagccttcagagcagaaagattcat

SEQ ID NO: 27 - DNA, 762 bp, Clitopilus passeckerianus
  source 1..762: mol_type = genomic DNA
atggaaggcaaggtcgcaatcgtcactggcgcatccaatggtattggactcgccaccgtcaatctcctcctcgcagcaggagcgtctgtctttggtgtagacctcgctccagcaccgccctcggtgacctccgagaaattcaaattcctacaactcaacatctgcgacaaggatgcacccgctaggatcgtatccggctccaaagaggcctttggcatcgagaggattgatgccctcttgaatgtcgctggtatttcggactacttccagactgcgttgaccttcgaggacgatgtatgggaccgagtcctcgatgtcaacctggctgcacaagtgaggttgatgagagaggtattaaaggtcatgaaggtgcagaaatcggggagtatcgtgaatgtcgtcagcaagctggccctcagcggtgcttgtggtggtgttgcatacgttgcgagtaaacatgccttgcttggcgtgacgaagaacacagcctggatgttcaaggatgacggcattcgatgcaatgcagtcgcacctggttcgactgacaccaacatccgaaacacgacagacccgtccaaaatagattacgacgccttctctcgagccatgcctgttatcggcgtacactgcaacttgcaaacaggtgagggcatgatgagccctgagcctgcagcccaagcgatcttcttcctagcttcagacttgagtaagggcacgaacggtgtcgttattccagtcgataacgggtggagtgtcatttag

SEQ ID NO: 28 - DNA, 2088 bp, Aspergillus fumigatus
  source 1..2088: mol_type = genomic DNA
caatgctcttcaccctcttcgcgggtctgaaataccctcacctggcaacagcaattggcgcttcatggctgtttttccgatctctctacttgtacggctatgtgtactcgggtaagccacaaggcaagggcagattgctgggaggtttcttctggttttctcaaggcgctctgtgggctctgagtgtgtttggtgttgccaaagacatgatctcttactgagagttattctgtgtctgacgaaatatgttgtgtatatatatatatgtacgttaaaagttccgtggagttaccagtgattgaccaggacatcagatgctggattactaaggtaatgtaaggtcagttcgagaccatctgatattaccacaaatacaatggcgagagagtttttcgtaaaagccaatccttggcgtttccagctgttcctgacggttgtaggcccaagtccgcgggaaaccgcccacaaagcggcgtttttgcagattggcagatttatgctggaaacttactggggagatggaggggcacaagcgctgtgattggttttcaaagccgcggccggatggaacgaagacataattcggcggggacatgaaaatgtgggtgatcgatacggaatttttggttcttcggaggcgacaaagggcgcaacggtcgaggttagtagttatcttgactcacacttacagggcccgtcttcggtcttcttaagaactgggttttgctgggacttcccccccacctctcttttctactgtgtctcgtatctatttctatactcattctttcacttctcttagtaccaccattcccttctaaatacacagaatggcttccaacggtaccaatggcgcctccgcctccaacagcttcactgtgaaggccggcttggctcagatgctgaagggtggtgtgattatggacgtcgtcaacgcggagcaggtatgagcgattgtcatcaggatacttccagccctttgacgctaacatgacttctacaacaggcccgcattgcggaggaggccggtgccgctgccgtgatggccctggagagagtccccgccgacatcagagcccagggtggcgttgcccgcatgtctgaccccagcatgatcaaggagatcatggctgctgttaccattcctgtcatggccaaagctcgtatcggacacttcgttgagtgccaggtaaggctgcctttctcccgtggaaagcctgcattgcagctaacatgtgtaattgttagatcctcgaagccattggcgttgactacatcgacgagtccgaagtccttacccctgccgatgatgtctaccacgtgaagaagcacgactacaaggttcctttcgtctgtggttgccgcaacctgggcgaggcccttcgtcggatcgccgagggtgccgctatgatccgtaccaagggtgaggccggtaccggagatgttgttgaagccgtcaagcacatgcgcacggtcaactcccagatcgcccgcgcccgctccatcctccagaattccaccgaccccgagattgagctgcgtgcctacgctcgtgagcttgaggtcccttatgagcttctgcgcgagaccgccgagaagggccgtcttcccgttgtcaacttcgccgccggcggtgttgccactcccgctgatgccgcactcatgatgcagctgggctgcgacggagtgttcgtcggctctggtattttcaagtctggtgatgcgaagaagcgcgccaaggctattgtccaggccgtgactcactacaaggaccccaaggtcctcgctgaagtcagcgagggtctgggtgaggccatggttggtatcaatgtctctcagatgcccgaggccgaccgattggccaagagaggatggtaattgcactactatctctacttgtgattcttcttatgttcttgtcatgatatgggcgttggaaaagttgatatagcgttctttgatgcattttgcattcaagactttcaggttcattcttgttagggtgttctgtgcatttgtccttcattatgtagacactcgcgaattctgaaaagctgattgtgagcatcagtgcctcctctcagacag

SEQ ID NO: 29 - DNA, 1134 bp, Clitopilus passeckerianus
  source 1..1134: mol_type = genomic DNA
atgaagcccttctcaccagaacttctggttctatctttcattctattggtactatcttgtgccatccggcctgctagaggacgatgggttctctgggtcattattgttgggctcaacacctacctcaccctgactccgaccggcgattcgaccttggattatgacattgccaataacctcttcgttattaccctcacggccacagattatattctcttgacggacgtccagagagagttacaattccgcaaccagaaaggtgtcgagcaagcctcgttgcttgaacgcatcaagtgggcgacctggctggtgcaaagtcggcgtggtgtgggctggaattgggagccgaagattttcgtccacaagtttgacccaaagacttcacgcctttcattcctcctccagcaactcgtcacaggttttcggcattaccttatttgcgatctagtctcgctatatagccgcagtccagtcgccttcatcgaacctcttgcttctcgccctctgatctggcggtgtgcagatattaccgcatggctcctgttcacgacgaaccaagtatcaattcttcttacggcattgagtgtcatgcaagttctctcaggttactcagaaccacaggactgggtccccgtgtttggccgctggagagatgcttataccgttaggcggttctggggtcgatcgtggcatcaattggttcgcagatgcctatcagccccaggaaaacatctttccacgaagattctaggcttgaagtctggctctaacccggcgctttacgtacaactgtacaccgcattcttcctctcgggagttttgcatgcgattggggacttcaaggttcacgcagattggtacaaagccgggactatggagttcttctgtgttcaagcggcgatcatacagatggaggatggggttctctgggtcggaaggaagcttggtatcaagccgacttcgtactggaaggcccttggacatctttggactgtggcatggttcgtctacagctgcccgaattggctgggggcaactgtctcgggaaggggaaaggcctcaatgtcgttggagagtagtctcattcttggtctgtaccggggggaatggaatccccctcgtgtagcacagtag

SEQ ID NO: 30 - DNA, 1569 bp, Clitopilus passeckerianus
  source 1..1569: mol_type = genomic DNA
ctagccactagcaggcttcgtgaacgtcaacgggcaagcacggatgacctcctcagcttccttacttcttggcttgatgcggcaagggaaatctagtggacgtgagatcatagcctggtgatactctctagtaggctcaatttctttgccattttcgtcaactgcttttaagagatcaaacagcgacagaacagaggccgcagccaaggtgatggtggaatgagcgaggtaacgaccagcgcaaattcttctaccgaagccgaatgcgatatcaaaggggtctctgacagccttgttaggcttaccgtcttcggtcaagtatcgctcaggccggaattcgtctggctgggggtaatcggtctcgtcgttggacatcgcccattggttggcaaacacgatggatcccttagggatgtggtattccctgtaaacgtcatctgagatggtttgatgaggtacgcccataggagtcacaggtctccagcggtaaacctccttgatcacagcgttgaggtatgggaaagaggggaagtcggcgtgctcgggcatccttccattgagaacactatctaattctcgttgtgctttcttctgtacttcggggaaacagaccatggcgaggaagaaagtccccaaggcggatgcagtcgtatcagcaccagcaatgtagacttgaccagcaacatccttgaggtgctccaaatctgcctcctggttttccgagttctgaagatctcggagagcgtcagatncaaaggagggctcataatcgccagttttaatcatctcctgggcaactttgaatggctgttcacgaacatagtacgcatgacctcgcattaaggcagccttttgatggaagatagtccctgggacccatggaggaatgtgtttcatcgcagggatgatgtcaacaagaaaggcgccagacgtcataatctcagacgctgcaaggacagctttctcgaccaggtcaacataggggtcgttataaggttcagtctcaaggccataggtcattgaaagcgtcgtagagccgaccaagttccgtacatgatcgagaacgtcgtcgggcttctcgtaaagctgcttgaggaaccgtttcacatatcgcaactcacgaggttggtttataccggggtttgaagagttgaagtgcttggtgaagcttcttcgaccagcccgccatgactcgccgtatggcattaaggcccacgtaaagccccatcctgacagctcgtggtgcatcgtgctgtgtggtctgctcgagtagatcgccgacctcttcagcaacaagtcattggcggcgttggcagaattcagtattacgatcgaggttcccatggcgctaacatgtatgatatcagagttgtactctttaccccagcgagcataggtttcccattcgaccttcgctggtaggtccatgacgttgccaataattggaagtttctttggcccaggcggcaggtgctgctttttcttcttctgagaatctatccagtaggccaagcctatagcagtccatattacaaggactggtagagcacgttccgttgacggagccat

SEQ ID NO: 31 - DNA, 1885 bp, Aspergillus fumigatus; identical to SEQ ID NO: 22
  source 1..1885: mol_type = genomic DNA

SEQ ID NO: 32 - DNA, 1233 bp, Aspergillus fumigatus
  source 1..1233: mol_type = genomic DNA
atggaccgtgtgctatcgctggggaaactccccatcagttttttgaagacgttatatctgttcagcaagtctgacatcccagcagcgactttaccttctgtatgtctggcgttcactctcgccccacgcaccggaagggtcactggctaatactgagagcagatggctgtagctcttgtgcttgctgccccgtgtagctttcacctaattataaagggatttctgtggaaccaattgcatcttctcacatttcaggtgcgtctagaagcattctccttgaaccgaggccatcaagcgttgacctgagcaggtgaaaaatcaggttcgttagtccgagacacgacaggcaggtcgacaacgacatgcaatgcttaccgcagccgttagatcgatggtatcgacgaggatagcatagcaaagccacatcgacccttgccctctggccggatcacacctggacaagctaccctcctctatcgcgtcctcttcttcctgatgtgggttgccgccgtgtacaccaacacgatctcctgcacgttggtctattcgattgccatcgtagtgtacaatgagggtgggctggcagctattccggtagtcaagaatttgatcggagctatcggtctcggctgttactgctggggaaccacgatcatctttggtatttagtctggcacggtccttctttttgtcaaggtacgcgctgacagatgatggttcaagatggcggcaaagagttgcatggactgaaagccgtcgcggtactgatgatcgttggcattttcgctactacggtgagttcatccggtagagaggcaactacctgctaatatctttgtcacacctgcttagggccatgctcaagacttccgtgaccggactgcagacgcaacacgaggccgcaaaacaatcccgctactgctctcccagcctgtggctcgctggtcactagccacgataacagcggcgtggactataggcttgattgccttgtggaagcccccggctatcgttactctggcatatgttgctgcgagtctccgctgtctggacgggtttctctccagctatgacgaaaaggacgattatgtgtcttattgctggtatggggtacgtctatgctttttttcctatgtacgcctggcccatgtccgttgacccagattacagttctggcttcttgggagtaatatcctacccatcttccctcgtttgagaggcgagcttccttag

SEQ ID NO: 33 - DNA, 1665 bp, Aspergillus fumigatus
  source 1..1665: mol_type = genomic DNA
ctagactgtactcggtttgagaaggcttgcatggctgacctcgggtatctgctccgactcgatgcggcgcagaagatcgatgtggtgctgactgcgaggctcgaccttgaactggaagggagcagggcgattgaccatgcccgggatcgcctggagagtgacggggatctcgttgccttggtcatctcgagccttgcggacgttgaaaacagccagcagctggaccacggtgatgtagacactggcgtccgcaaagtaccgacccgcacaagatcggcggccgtaaccaaaagcaatttcgctcggatcagggtggttgaaaggctccatgtagcgctccggcttgaacactcgcggctctgggtactctttggggtcgttcaggaaccaccatagagaaggcaggagataggaacccttggggatgagatattctccgcacactaaatcttcctcggacttgtgcgtcaatcccatgggtcccacgggattccatcgccaggcttccttgataatgccgtcgacataaggcaggttggttcgatcgtcaaagttggggagccgatcggagccgacaactcggtcgatttcttcctgcgcccttgtcacaacctcggggaacatgacaagaccacagatgacgctgtggatgatggcgacggtactgtccgagccggcggcgtacaggctcacggcggtccacttgatcgcctcttcgtcagccgcggaaacgttgatcttgttgtcctccgacttgatcatgtgcttctcgagaagattggacacgtatgacggctggtgggctttgtgcgccatctggcgtttaacaaaatcgtaagggagttccgcagcggcctcattgatagccctccatttccgcgccgtcttccggtacgacatgccggggaaccagtctggaaggtacttgatcgcaggtacggagtccacggcccaagcgagaggcacaaatgcttgggacaggttttccatggcgtgttcgatcaactcgaccaacgggtcctggccctttcgctcaatggagtatccataggtaattttcaaaacgatggcggcagccaacctacaacccatgagacagtgtagaagacatattaccacgtcgtagggcacttacgttttcaggtgctgcaagatgtcgtccggccggttgaacgtctgtaggatgaaccgaatggattcttgctcctgaatggggcggaaaccagcagagagccctttcgtcccaatctcctggtgcaccattttccggtgcaggcggtacttgtcattgtactgatgggtaatgagaaagttctcgaacccacatagctgggcaaagttgagctggggtctcgcggatgtcttttgggccttttttcccatcaccgcgtgggccgcgtccttgtcatggaagatgacgagcgttgtccccatgacattgatcgaactgacgggaccataggcatctttgtgcttgaaccagtgcagatactcgggctgccccttgggggggagatcaaagaaattcccaataattggcaatggccttggcccaggcgggacgttctgtttgaggtttctggtacgagtccggaataccagaacggccatgaaggccacaaaggccacgcagctaagctgaagggtagatagctcgtaggccat

SEQ ID NO: 34 - DNA, 930 bp, Aspergillus fumigatus
  source 1..930: mol_type = genomic DNA
tcaagcgctcaccgcagttgtacccttttcggaagggtatttctgagccatatacgtcagatcgcccttgacgacgtatccaatatggctgagtgcgagcagttccttcaactgcggactaagtgtctcttgaagctcctttggcagctttgcagaccagtttgtcaggggtcgcatgtgaggcgccgaataataggcaaacagaatggctcgatcttcatcctcagtcacgttggaaccagaggtgtgccacaggcgcccgtcaataacgacaatgtcgcccgcatccgcttcaaacgggaccagcagatccggtgcgttatcgggcacgtcctcccaggtggtccacttgttcgaaccggggatatacaaggtcgcaccgttctccttggtcatcctcgtcaggcaccagatcacgttgactgcccagacatccaaccacggcgctggaagaacgatgctctggtccgagtgcagggccatgctctccgcgccaggacgagcaatgttggccgagaagttgctgaccagcagctggtcgcccaggagggacttggccaggtctagtgcggtcgggttgaccagcatgtcgcgccagtatgcgtccaactcggggagatagaagacgcgcacgttcgccgggttgggatccaagatcggctggaaagtgcactcgccacgagcctccgaggcagctttcgcctcccagagacggctgagtgcatcctcagcttcagctttggagagaacggcagggatcttgacccagccatgctcttttagatgagcttgggcgtcttccatgtttagtgtcatgtctcgaacaaggtcccttgatgttgagggtacaagggtgtattcaggctcttgagccgtaggatcaagagcgctgactgactcgctaatagtgcattcatgcctacccagcat

SEQ ID NO: 35 - DNA, 1379 bp, Aspergillus fumigatus
  source 1..1379: mol_type = genomic DNA
ctatggcagctctgcctcaatcacgctctcgtagccacgaccatcagggtaatacttgaccagcttgagcccggcatccttgatcaccttgctccacacggcttcggttctttcattagctgaagcctgcaacacaagacagtccatggccgcttggtagcaactggcacctgtcgatgggatcacaatgtcgttgatcagcaccttggagtagccgggcttcatcacagcggcaatctgccgaagaatcttgacggatgtctcatccgaccagtcatgcaggacggcatgcataaaatacgctcgcgctcctacatatcgctgttagtcccctaggcacctgtagtggcagcagaaccggtagcctacctttgatgggctgctcaactccctcctcaaagaggtcatgcgccacagttcggatcttgtccgtggtaaggtggacagcaccgacaacgtcgggcaggtcctcgagcacaagggacccagcggggagatcggggtgcttctcggccacgcgcatcaagtcgatgccgtggtgtccgccaacgtccacaacgaaagggcttccattactgaggtcggcgccatcgagcagtgcttgggtgtcgtagaactccggccacggtctctttcctttggcccacacgtccatgaaagatgagaagctctcctggtgcacggggttcgcgctgcaacgctcgaaaaagctcttcttttctgggaaagtatcaatgtaacaactcgccttgtcgtcccgcggcttccgatagttggtcttggccaggaaatcgggccagtgcatggcacatggtgcgacatgatccgttctcatcggtgctcgttagtatggctcgcgttgatatcgaccttgtgcctaaagggggcagctcaccgaatgcgaagcgctggggcaacctttgtgctcttgtcgccgatagcgagggcatacggcgtaggtgcatagcggtcgttggccgtttccaggataatgtggttggcagccatcagtcgcagttgatgacctgggatcatcagcatccacgtgggatggcactctcagcagagagaaacctacgtaatagctcgggttccacgtctctcttgctgagcttggccaactcggtcacatctctctcgccgcccccggccgcagcccagccttcgaacagaccggtgtcgatgagagcttggagcacggagaacatgactggttcctcgatagcgagccgcatggtcttttcttctttcgtttccagcgtgtggaagagtttgcgggccgccagagccagcttctgtcgcgtggcatcctggccctcaaagatgctcgtctccaacgtctggagcttttcgattaattgttcggcaatgtcagccat

SEQ ID NO: 36 - DNA, 3155 bp, Aspergillus fumigatus
  source 1..3155: mol_type = genomic DNA
atgctaggattgcctaacgagctgtcggggagccaagtcccaggtgctacagaatatgagccaggatggcgacgcgtcttcaaggtagaagacttgcctgggctaggggattaccacatagacaatcaaaccgctgtccctacgtctatagtctgcgtgattgcccttgcagccgccatggatatcagcaatggcaaacaagcaaacagcatcgagctctatgacgttaccatcggacgaccgatccacttaggaacatctccagtggagattgagaccatgatcgccatagagcctggtaaggatggagctgactccatccaggccgagttcagtctgaacaagagcgccgggcatgacgaaaacccggtcagtgtagccaacggacggttacgcatgactttcgcaggccacgagctagaattattgtcctccagacaagcgaagccgtgcgggttgaggcctgtgagcatcagcccattctatgattccctcagggaagtcgggctgggatacagtggacctttccgagctttaacttctgctgagcggcgaatggactatgcatgcggcgtcatcgcgccgacgactggtgaagcatcaaggacaccagccctacttcaccccgccatgctcgaggcctgcttccagacgcttcttcttgccttcgccgcccctcgagatggttcgttatggacgattttcgtgcctacccagatcggtcgactcacgatatttccgaattcatccgttggcatcaatacgccagcctcggtaactatcgatacgcacctacatgaatttactgcagggcataaagcagatttacccatgatcaaaggagacgtcagcgtctacagctcagaggctgggcagttgcggatacgcctcgaaggcctcacgatgagccccatagcgccctctaccgagaagcaggacaaacggctgtacttgaaaaggacatggctgccagatattctctcgggcccagtactcgagcgagggaagccagttttctgttacgaactcttcggcctgtcgctcgctcctaagtcgatactggccgccacccgactgctctcgcatcgctacgcaaagttaaaaattctccaggttggaacttcttccgtacatctggtacattctttatgtcgcgagctaggaagttccatggactcttacacgattgcctgtgaatcggacagttccatggaagatatgaggcggaggttgctatcggacgccctgcctatcaagtatgtagtcctcgacatcggaaagagtcttacagaaggggacgaacctgccgccggtgagccaaccgacctcggctctttcgacttgataattcttctaaaagcctctgccgatgattctcccattttgaaacgtacccgaggtctcataaagccaggggggtttctactgatgactgtggcggcaacagaggccattccgtgggaagcaagagacatgacccgaaaggcaatacatgatacgctgcagagcgttgggttttcgggagtcgatttattgcagagggacccagaaggcgattcgtctttcgtgatcctgtcacaggccgtcgatcatcaaatcagatttcttagggctccgtttgactcgactccaccatttccgactcgagggacgcttcttgttataggcggcgcctcgcacagggccaaacggcccattgagacgatccagaatagtttgaggcgtgtctgggctggggagatcgtcttaattaggtccctgaccgacttgcagacccggggccttgaccacgtggaagctgtgctgagcctgaccgagcttgatcagtcggtcctggaaaatctcagtcgcgatacctttgacggcctacatcgactgctccaccagtccaagatagtcctgtgggtcacatacagcgcaggaaatctgaacccccaccaaagcggtgcaattgggctggttcgagccgtccaggctgaaacccccgacaaggttctgcagctccttgatgtggatcagattgatggcaacgacggtcttgtggcggagagcttccttcggcttatcgggggcgtcaagatgaaggatggcagctcgaatagcttgtggacggtcgaaccagagctctccgtccaaggagggagacttcttatcccgagggtgcttttcgacaagaagcgcaacgatcgtctcaactgtttacgccggcagctgaaagcaaccgattcctttgagaagcagtcggctctggctcgtcccattgatccttgcagcctgttctcgccgaacaagacgtatgttctcgccggtctgagcgggcagatgggccagtccatcaccagatggatagtacagagtggtgggcgccacattgtgatcacaagccggtgcgaacagacacgtctgtgatgtggataagtactgacagtaatagcaatcccgacaaggacgatctctggacaaaagagctagaacagcgcggtgctcacattgagatcatggccgctgatgtgaccaagaagcaagaaatgatcaacgtccgcaaccagatcctaagtgctatgccccccatcggaggcgtggcaaacggtgcaatgcttcagtcgaattgtttcttctctgatctgacgtacgaggccctacaggatgtcctgaagcccaaggtggatgggtcgctggttctcgatgaggtcttctctagtgatgacctcgacttttttctgttgttctcgtccatctcggcggtggttgggcagccattccaagcaaactacgatgcggcgaataacgttaagtttggccaatctgccgcagtgcggacctactgactgaccactttgtagtttatgaccggcttggtgttgcagagacgcgctcgtaacctgcctgcgtcggtcatcaaccttggcccgatcatagggctcgggttcattcagaacatagatagtggtggaggttccgaggctgtgattgctacattgcgaagtctggattacatgcttgtctccgagcgtgagcttcatcacatattggccgaagcaatcctcatcggcaagagcgatgagactccggaaataatcactgggttagagacggtctcggacaatccagcacctttctggcacaagagcttgctcttttcacatatcatatag

SEQ ID NO: 37 - DNA, 2145 bp, Aspergillus fumigatus
  source 1..2145: mol_type = genomic DNA
atggcgcaacttgacacgctcgatattgttgtcctggtagtgctcttggtgggtagcgttgcctacttcaccaagggctcctactgggccgttcctaaagacccctatgccgcagcgaattccgcaatgaatggcgccgccaaaacaggtaaaactcgggacatcatccaaaagatggaagaaaccgggaagaattgtgttattttctacggttctcagactggaactgccgaagattacgcgtcccggctagcaaaggaaggttcccagcgtttcggcttgaagaccatggtcgctgatctcgaagattacgactatgaaaatcttgataagttccccgaggataagatcgctttctttgttttggctacctacggtgagggcgagccaaccgataacgccgtcgagttttaccagtttatcaccggtgaggacgtcgctttcgagagtggtgcctccgctgaggaaaagccactctcctccctcaagtatgttgctttcggccttggtaacaatacctacgagcactacaatgctatggttcgccacgtcgatgctgctcttacaaagcttggtgcgcaacgcatcggaaccgctggtgagggtgatgacggcgctggtacaatggaggaggacttcttggcatggaaggagcccatgtgggccgcgctgtcggaatctatgaacctgcaagagcgcgaggctgtctatgaacctgttttctctgtgattgaagatgaatctttgagccccgaggacgatagcgtctaccttggcgagccgactcagggtcatctcagcggcagccccaagggtccctactcggcacacaatccttacatcgctcccatcgttgagtcccgtgaattgtttacggccaaggatcgtaattgccttcacatggagatcggcattgctggcagtaacctcacttatcagactggtgaccacatcgctatctggcctaccaacgcgggtgttgaagtcgatcgtttcctcgaggtctttggcattgaaaagaagcgccatacagttattaacatcaaaggtcttgatgtcactgccaaggttcccattccgaccccaaccacatacgacgcggccgttcgcttctacatggaaatttgcgcacctgtttcgcgtcagttcgtgtcctctttggtgccattcgcccccgacgaagaaagcaaagccgagatcgtgcgccttggtaatgataaggattactttcacgagaagatcagcaaccaatgcttcaacatcgctcaggctcttcagaatatcacctcgaagccgttctctgctgtcccgttttcgctccttatcgaaggcctcaacaggcttcagcctcgttactactccatctcgtcttcctcccttgttcaaaaggataagattagtatcacagctgtcgttgaatctgtccgcttgcctggtgcgtcccatatcgtcaagggcgtgaccacgaattacctactcgccctcaagcagaagcagaacggtgatccctcacccgatccccatggtttgacatatgctattactggtcctcgcaacaagtacgatggaattcacgtcccagtccatgttcgccactccaatttcaagttgccttctgatccttccaaacccatcatcatggtcggacccggtactggcgttgcgcctttccgtggcttcatccaggaacgagctgctctggctgaaagtggcaaggacgttggacctacgattctgttctttggttgccgtaatagaaatgaggacttcttgtacaaggaggagtggaaggtatgtttgcagtcttcttatgagcacattcggagccgtttgtctgactcttaataggtctatcaagagaaacttggagacaagctcaagatcatcactgccttctctcgtgagaccgccaagaaagtatacgtccagcaccgactgcaagagcatgccgaccttgttagcgatctcctcaagcagaaggctaccttttacgtctgtggagatgcagccaacatggctcgggaagtcaatcttgtccttggtcaaatcattgccaagtctcgtggcttgcccgctgagaagggtgaggaaatggtgaagcacatgcggagcagcggcagctaccaggaggatgtctggtcgtga

SEQ ID NO: 38 - DNA, 1937 bp, Aspergillus fumigatus
  source 1..1937: mol_type = genomic DNA
ctagtagtcgtcgccacgactaatcacctctttgacggtgggtcgaagcagaatggtcttttcccaccattagtgcatattcttcttgaggaatcaaccggacttacgtgttcgaactgggccgtatacgtccctggcttctcgttcagagggggatagtcttccacaatgccggatttcaccaggtaattcagctatcgccggttagccgagagctctgattattgcccttggtaatggacctcatacaccgaggagatacttctcttggccaatccggtccaaatagcgccggcagaaggggatcgtgctaaagttcttcttgatggctgtcaagagggacctggccgaagacaacgtcaagtcttttcggtccgcgtccccgcgcagggcgtaatgggagacctcgcctccttcgacgtatcggccactgccggtactcccaaaggtttcgatggcaaagacgtccccttcctccatcttggtcatgtcgttcgacttgacaaaggggacattcttggtgccatggatggagtacggcaggatcgtgtggccacacaggttgcgaatcgccttgatcgggtacgtcttgccgcggatttcgcactcgtagctttccatcgcctcctggatgtagccgcctagttcgcccacacggacatcgatgcccgcttcgcgcacccccgtgttggtggcatccttgaccgccgcgagcaggttatcgtacatgggatcaaacgccatggtgaaggcactgtcgacaatgcgaccgccgacatgaatgccaatatcgaccttgaggacattgttctgcgccaggacggtcttgcagccggcattggggctgtagtgggcgacaatgttatcgatgttcaaccccgtgggaaaccccatgccggcgatcaaggagtccccctccgttaagccgtcatggcccaccaggcatcgcgcgctctcctcgatgccattcgcaatctccagcagcgtttgaccgggcttgatgtttctctgcgcccactgacggacctggcgatgcgcttccgctgcctgacggtagtccgagaggaagtcgctattcaggttgtcgaggtgacgtttctcctcgctcgtcgtgcgatagcgattctcgtccttgtactcgacctcttcacccttgggataggagttgttcgggaatagctgcgagaggggaatcgatggcggatcggtctggaccttgggttgcttcttcttgggcttcctctttttgttcttctttttcttcgcagtgctgtgctcggcagctactgcgggggggttttcagtgccatcgtcgtccgagccgtcgtcaacttcctttccagtcccgtttgctgcggccgaagtcgaacttgacatgtcggctccattggcaccagcatctgtgagttgcatccagtatgagctgggatcatcgtataggttgggaacctgatgctggactcttaccggtgattctcagcttctcaagaagctctggcgcgtcgacagtcatatctagcaagggaggacaccaggagaaaagggacggtcgcaagtctgtgggaaccaaatgatatgtaacttagccaagcacaccaataccaacgaaacgcgagagggcttcggagtgtgcagtcctggacctcggatgtgcggcgtactccgtagcgtggacaacgcagtgagtgagatccagcgcgaggcggggctggaggggcaataacacagaagcagcgcagtgccaggagacgacgactgcagttgcacggtgggcaccaagggtacgtgctaggcgctggccctggtccaccgtttgacagggaaagatttggaaacttgggtatccagcatgtagatgcaagtcgggtatacgctatccctctgctttcgacaacgagcaaaatccaatcgagtccacgtctttggctttgaagcat

SEQ ID NO: 39 - DNA, 1713 bp, Aspergillus nidulans
  source 1..1713: mol_type = genomic DNA
tcaaacatgctcgcgaggcctgacgggcgcagtatcgtgaaggtcccattcctcttccagctcatccgcaagagacgatggcccaacagtctgctcgacaagccgtggagggcatttgttcttcatctcccagctgccatgccgctcatgtcctttggctgaagcggtgggagcctttattgcatccccccattgcggattcttaaaggtcaggtccgaatcacttgccatcttgctatccctcttgaagcctccaagaccgggattccgcaaccgcttggtgcgcagacataaaaagaatccgacaacaccgatcagtgctaaaaccgcgatagtaacgacagatccgataactccagcaacagcagcactgattccaccgcggtcctttcgcgccttgctggcggccgactgtcgtggcagtggttttacaacccccgagcagaacacagcggacgagttacaacgacggcaccaatcttcggttgaccctaatgccattctttgcatctcagcttggaactcactgaatgggatggccgtattgctcgggccatgtccaaacagcggataaggcacgaattcagcccgtgttccattgtgcaggaggaatcgaacgtagagctgcgccgggtcggggtacgtagggtatctctcgctctccaggctgaacagttccaggacaagggacgatcctggacgcggcagggacgaagtaaagtttggacttgtggtggccagctgcaaaagtgaggtgaaggacacagcggtttggtagttgccgaactgaagtgtcatcttcccattagcgcctcggttctggatggttgtctcgaaagcgtttagaacacttgatgcgaggccttggcctgcgattgtccggataccgccgtcaacggttgtgccggatgccgacgtgtttccattcgtcgcaaagacatatcggccagcataccaacgtgcgcgcttgatgtcctctgcacttagactatgtaaaagggattcattatgcaccacctggtagtccagaaattcggcaatcgcggttgcgttcgcatagctggatgcctttgtatcaaagtgaccggacaaagatagagtatgaagacggttgtaaaatactgctgactcttcataggtccgccagaactctttgctggcaatgtactccagatttgcggcagcgtgccttgaacaaagcgcctggccggacaccatgagggactgagggtcttccgcgccgacggtaacaatcgaagggtactgatatccacccagcggaggagagataagagacccatcggccaattccagttggttgtcgaagaaagtggtgttgaacgactcattcaggggcgggtaaacaccctgcatgaatgcctgggcggatgcgagcacggccacatcaggcgtagaggtgattttgatgtcgtcgttgtccaccagataaggagagagattctcgatccttgcatcgggtctatcggcgccggcttttacagagacatatcgacctcggaatgccgagccggcttcatgaagttggtatgctccgtacggtgtcaatgcccttgaacgcgggaagactcgtggtatggtttctccgttgatagtatatgcatatactgcccagacccgcgccgtctggccgcttgctgcagctgcgacaactcccagaaggctcaggagtgcgaaagacagcccaaccatcat

SEQ ID NO: 40 - DNA, 653 bp, Aspergillus nidulans
  source 1..653: mol_type = genomic DNA
gttgccggtgcggtgggatgcattcttcacgtttcttccgctgggactggtcgacctaataagaataagaaggtcgatttactttcgcaaggatatcgcgacatgacgacatgatacggtcgtaaccatgttccaagattcaacttactttgccctattccggctggcggggtgaattttccgccgcaatcaacacgaattaggtcagagtgtagatagagccacatagattccgagcgtattactgttcggaaatcacgggcctgtatagaaaattctgctaatggacttcactttcgatttctaggattgtatgacgtgaagacagagcaaggttacattctaactctcagtagtggagttctacctagcccggcccggcgcgccctagataaccctaaatcaaagataattggcctgccttcgacgtttctcaacgagctatgtccgaaattttatctttaccaaggtcgaagtttcgtaggaactcaggcccattttgtgcgacatgagctgcttgttcggaactgtatccgctcgttccaaaccgttccatccgggcagttgcggaatcagtcttaggacctgatagatgcatgaaatagatggaccatcctgaacatctcacaaactcaaaaaaaaatttccaaccg

SEQ ID NO: 41 - DNA, 1534 bp, Aspergillus nidulans
  source 1..1534: mol_type = genomic DNA
tcacctgttataggcctggtaccgaatctccaacaaaactaccgtgctttctctggactgaatcttattcaccaccaccaaccggcccatactatcactgacattgctcagcagattaatccactccgccagctcaatctcacgctcatttgccaactgcatcagcgtcaagtcgcgcagtcgcgcgcttgcttcgacctcgctatgcacagctgagggttcaggcaagggccgcggggtgagaatcagggtcgccgatgggtttgagcggaggatgtcgagatgtgagcggagttctgcgaggatgtgcgttgcaagggaggcgaaaggaactgttggtgagggagaggggaggtgtaggatgtagagatttgctgaggtaatgggttgcggtgctgttggaagccggtgttggatcgttatgttggaggcctggggtatgctattggtggtatgcgtatgggtgtggttgtggctagaggccggtgttgtactggccgtgcttgcagtgagtgcgcgaaggtcgtcgtgtttgtggctaccgccgggagttggggggcggatgggattggggtgtgctggtgaccaggcagttgggcctgctggggatgcgatttggactgtgatgttgatggatgggtagaggtttgcaagggttgttgcgcggtcgagggagcgggcgctgacctataatttgggcaattggttagacaatcgtatggatgatctcaaaatgaagatggattggatgaagtacctcaacgacagatatacttcctcttcgaaaatggtccagcctcgacaacagatccgtcactcgatcgtcggtatcactggtcccatactgaagaaaagcaggccactggcgttgaagctttggccgttgctcagacgtactggcgaatgtcgctgggttatttaaagctaggttgtacgcggtctcgttcggacgcaaactcgcgccaaatcgctgcgttgcagtaggcatctgcaaagcagaaggggcaatggtgccagccaaaaacatcacagcgtcaagataagacggtttggtgacgaaaggagcggagagcgcgctgtgagcgacttgaccggggtctggctcatccaggaagccagcggtggctgtcatccggataatacgtgagagatgagtctctgggacaccggccagctcagcgacatcttttatgggaacggtgccggtgaggggaatgcaagcgaggacttggaactctccgagccattgtaggcaggcaagcagctggttctgaacggcgaggtggtggaggaagtcggttgggctggtgaggagcttctggaggccagaaatggttgataagatcgattgttgggctcgatgcgcttccttggaagcgctagaggtgatgaggggttgagttctgctgcgagaggcggcattttggcgagggcattgcgagatgatcgtcttgacagcgcttgtgagctcactggcgtgggtttcaaggtcggatagactagacatcatgtcctggaagtcccttgacaccat

SEQ ID NO: 42 - DNA, 314 bp, Aspergillus nidulans
  source 1..314: mol_type = genomic DNA
atattggtgggcagtatatattagtagaatcacatcaggaaaggttctgagctatataagcacaaccgatagagcctgaacctcactcgggatatttcaggcaacacagcagaagaatgcatatgcagccgaacatgaccgcgaacagtgaagcaacacgaataacggccttacacaaaccccgatggggagcaagaggcgattccgacgcagaaactacctttcctcagtaccaagatatatggaactaattacccgataggttgtaggcgatattatatagtttatggatataccagccgtctaacacatga

SEQ ID NO: 43 - DNA, 692 bp, Aspergillus nidulans
  source 1..692: mol_type = genomic DNA
tcagacggacctgacctcaaccgctttgttcaccacatgaccattcctctcctctacctcactcgaaccattcgagttcatcacttggtcgtctgcagcgactccgttctcttccttctcagggggtccaaagatcccctctccaccaaactcagtccatcgtatattcggttcaattccggcaaacttccactcgccattgatcttgcggtacgtcaccgtcgctgagccatgaccgtgaccttttgcaacgacttctttcatctgagaatcaaggtgtttctgatgggcgactctcatctgatgatacccaactattttcgagtcgtcgaccttctcccatttcattgttcccacaaagtgctgcgttttgaggagtgggttacccaagaagtggggatgagagaccatagccacgaattcttcggccggcatcttctcccagagcttgtccaagaaggctctgtaatcgatctggtaccgtcagcactgattatataggacgagactgggaagctgacgcgaaggaaaggggcgatgcattgttttaagcgatcccagtctttgctgtcgtagctctctgcccattcgaacagggcagcttgacagcccgtaatgtctggatggctatcagtatgcacattcaaacactgttcaggggtcctaccttcaaatgttggctgcagcgtcat

SEQ ID NO: 44 - DNA, 295 bp, Aspergillus nidulans
  source 1..295: mol_type = genomic DNA
tgtgccgtccctgtttctctacaagatgggacaaacggagaaaaggtagactcaaaagcaatattttaagtcgatcccaactcacaagacagtgtctaggacgggaagaccatgcaagggtacttcaggtcggtgacttgctaagtaccgtatgaaggcgggttttacttggtccccgaccttcggtgtccggtacctatatttgagtggaacccatttcaatgcagcctagatcatcaacgcaatgtgccattttattgttctggctacgacttagctactaaatctagcagaa

SEQ ID NO: 45 - DNA, 925 bp, Aspergillus nidulans
  source 1..925: mol_type = genomic DNA
ctaagcagcgcctccgtcgacggtaatgatctttccgttcacccactccgcttctctactcgccagaaaaccgacaacctttgcaatatccactggaaacccattccgcttcaacggcgataccgttgccgccattttttgcagctcttccgcgctgtgtttttcgccgtttggaatataatgctgcgccacgtcgtaaaacatgtccgtcaccgttcccccgggggcgacagcattgacggtaatctgcttgtcgccgcagtctttagccatcacacgcacaaaggactcaattgcgcccttggagccagagtacacggagtgccgggggacgctgaactctttagcagtgttggaggacatgaggattatgcggccgtgggtgttgaggtggcggtaagcttcacgcgcaacgaagaactgggcgcgggtgttcagactgaagacccggtcgaattcctcctatgaaaaatcgtcaatacctcaaccaggagtcgaatgaaaagcgggttcatacctctgtgacctcgcccagatgcccaaaactaacaacccccgcattactacaaacaatatccaggcccccaaaatgcgccactgcatcatccatgaccctcacaatctcgctcacgttgcggatgttggcttgcagcgcgatcgcatcggtacccagctctttaatctcctggactaatttctcagcgggttcacgggagttagcgtaattcaccacgacctttgcaccgagtcgtcctagttcaagggccattgctgcgccgattccccggccggagccagtcacaagggcaactttgccttcgaggcggtatggggcgtgcgtggttgcggtcatttttgggagcgcgctgacgttggaattgaggtgggaggacacgagggagagacgttggattgctggagacat

SEQ ID NO: 46 - DNA, 558 bp, Aspergillus nidulans
  source 1..558: mol_type = genomic DNA
tggtgctttcctacctaccttatgtatcttgcgctcaggtttcttagaaacggatgattagagccctaagttcgtaagcacatggtgtgcaagggtacggtgcccgagtctcgatcgggatatgtaacttgggcgcaggggataagagagaggtttcggtgacttagatgcattatgcgagtacggacagcgatgttttacctgcatataatactattacttctgccttgaggatgggcatgagcgtgttgcaacacgagctgtgaatatgtgatcaatttggcccgaccaagagaatataagagttaccattattgctgagtagcactcgttaagtatccatggttgagaagaatgactttgatatcagtagatcagaatcattgtctcttaatcaaggatgaactgctagctaggtcgccctacttagattttctgggaaatacgaatatcaaaccatttatgaatctagccttgagcgccagctttaagctcaatcacattgcgactgatgatatccaaatcaatatatattctaaatctttggagaaaaggtaa

SEQ ID NO: 47 - DNA, 1644 bp, Aspergillus nidulans
  source 1..1644: mol_type = genomic DNA
ctaagaccaatcaccatccaacaaatcctccactctcttcccatctgcaatattcctccaaacctcctccaccgtccaagccctaaactcatggcccggaggatagttcgtattcacaaactctctcccatcaagtaaatgcgcaaacgcctcgccgaacttttcatatgcatacgcctccggatcatgctgaaagatccacttaggaaaccttgtcctgatcttcgccggatccttccagatcgcatcccagtccgtgcccgtcttcagctgagaattcacgaacgacattttttgtgcacaagagacccgctcataccggagaagattgtagatcttggtcccaagatatgcacgctgcgagcttccggctaattggaggcatgttgcaagcgtgattgcgtcttccaaggcctgcgagcctccgtttcctgaggtaggaatgaagctgtgcgcgctgtcgccgacttgcactacccgtccggcaggtgaggtccactcgcggcgaaggtcccgccagaggagaggccaatgaacaattgcgcctttcggcgcgcttcgaatgagcgctagcacagcgggatcccagtctcctgcaccggagagcatagcctgcgccacagtctcgggatctgtatcaggctcccatgattcagtggctgtgccttcaacgatgtcatcacggggcgtgaatccgaaggagataatatcgtcgccgacgaagacaccaagatacatgcccggtccaagccagtattcccagatgggtggactatcgctccatcgcttccgtacgagctcattctgcattgctaaatctttcggaaatgcagtgcgatagatactcagcccgcttgatcttggaggaacatgctgaccggctatcaatatctctgaaggagatttgaggccgtccgctgcaacgacgatatcagccactctgacctctgcttctcctgttgttgcgattataacgccgcccttgccatccttttcatcttcaaaatagctcttcaccgtctttccatattcaacgcggagcccgcaccttgcgacctggcgcaggagcatgcggtagaatttccggcgaacctgagcgggggcaacaaatggacctttgcgtgtttccaggtgctcggggtcattgaacgaggggacggttgggccgtaaatgtgccgtccatcatgagtttcgtagctaacgacggcgtggacttgctccgctttcatatcatggagcatgtcgggccagtgccggattatagatacggcagaaggctgcatgacaatgatatctcctatctcgagaaagtatagagttaactactcggtttctcatgctctgggggaatacacgagacgtatcaacaaaatacctgaatacacaggtccctcactccgttctagaattcccgcaacatcatggccctttctccagcattctaacgccgtcatcagtccacccattccagcaccgacaatgaggacggagattccggtcgaggggtgccgagatggaagaccagaggtaggagcagtgccattctcgccgttaacactgctttcggtagtaggcgtctttgcccagcgctctggatcaaattcctgcttgtcactggcgatgttgacggggaaatgggtcat

SEQ ID NO: 48 - DNA, 526 bp, Aspergillus nidulans
  source 1..526: mol_type = genomic DNA
tgtgactctcagtgctggtggtgtttggggacctgggccgagtaggtagtgcgttgggtagggtcattgaagcaccgagccggtggtctagggctacctgtgttgattgagggagcactagatgatagaaactgtcactgaagcttggctattgtgctcgatactttctagtacaactagttaatatctagactagaagatcgcagcggatagagccattgaaagtcacagacgctgacataacacatttggattccaactaggagagctgatatgctcggggatataaatttagttcttgaacgggactgcccagtccaattgggaacttaatagccttaatccaaattacccctctatacgctggtcataatatggatactattacggcactgataagcacgggaaaaagactccgaccactcatatgctaggtcttattgtaacaactaagttgcaaatacaacgcgcgcacgaaacgcaatggaacagggtatatggattccggtacgataatgtttgacaa

SEQ ID NO: 49 - DNA, 1308 bp, Aspergillus nidulans
  source 1..1308: mol_type = genomic DNA
tcaacccctccgcaatcggtcgacaatctcacttgacaggctccgaagttgagctccgagatcaaccgccagcctttccagcagatgaaaagggagagagtaatgattgctgtcattgaccccagtggtactcaacctagcaggcttcccgcctgagacttggtctttcagtcgctgatagagattgcccactaatcgttgaacgcgatgcagttcgctgagaactagctgtgcggccatgcggccttgatcttcaccatcgatattgtagcccctgacaacagccggtgtcctgtcgatctcttctaatgcttggctgtcttcagatataggggagatatgcgctacggcgctataccaggctagtactttgaaggcagcaagagttatgattgtgatggtgtagccgtcttccgagcaggagcactctatgatctcagtaatatctcgtaaagtctgctcatttttagtgatgacttgttgaactgtaggaggacttgcactgccactttcacttgagggagtcacgcatgatagtgaagggtttggaaagagttcgcgcagcagtgtcagtgcacgtgggaaacagaaacattgccgtggtgtcccaacactggttacatctggtgtaggaggaactgaaggcgaatttgccggaacgggagaatccgcgaaactggtcttcaagatattctcttggagcgttggtatcggttctccagatgggaagaatgacggaggatcaggaaaaccatccatgacattcgcgctcatatcagctccagggaagtaatccatgtcaggcacatcgagaagcgatagagatataggcgaagcaagataaccgtcgtagtcagggggccccaaggtaagagggcttgtagctgaggttccggggccggttgatgagagaagacttggtatactctctggatagcttggtgtgcgctggtgatattgatttcttcggtatacttcgaggcttcggtcctgctgaagagcgtattgcatgagctctgtcgacacctccatgagctccctccgatcatcatctttgttgatagacgtggaatagtctgtcttcatattgtagaatgacttgaagctgcctgttttactgccttgtttgcgacctgcgcgcttgctggcgagatactgacatgctgtacctcttttgacgcatcgtgagcaagtaggtttatcttgactgcacttcaatttggacagggcacatgcgtgacagcttcctcgcagcttgactggcggagttttgatagcggggatacctggaccctctgaagatgtcat

SEQ ID NO: 50 - DNA, 1478 bp, Aspergillus nidulans
  source 1..1478: mol_type = genomic DNA
agtgggagtgaggcgatatcaatcgggggattacagcgtgggaaaatgagggggcccaggcttaaagtaagagagcatctgcaggaaggattcgactccatgctcgcatggccaccgcttggttcattggctttgatagcaccaggccagctgctggatgtcagcttacagttggataccattggagtctctaaactccatccggggcctgagctgatgcccagagtgggatccgggaaacagcccctggcaatgctcatgatccttttgtttcgggcgggtcaagtcttgctgtccccgacagtgatggtgatcagccagagtggcctgggagccgcaatccattcatatgcactatagtgctagcaacaaccgattttatcatgcatttgccggagtcaggtctcggatttaacggaggagaaggactttgctcatcgcagttaatcccattcgaccgataactccatctcaacgaaactataaatcaagcattaaccaagccaggcgccctactcgtacctacttcggagacgagtacagatgtacgcttacgggtaacggaatagatgtggagactttcggacccaggttaaccggcccccacgtcgttcccggtgaccgacatcaccgccgctgtccggtcattagcagttgtcatcgcaaaaggcgattcgaagatgaccgcttcatcaacgggaaaccggataggaaactttcaaaaagccaacgggaatgtttggaatccgcaaaagagagggtcggaaggtatctcgcgtggcttgctcagtgccgttgagctgatcggaaactatccatagtataacccaatcggctagtactgcactgcagatccacccgcaactatcggcacgctattcgcaaccggtcttagtccagcttagcgggcatgctaaattcgaccttattttgtcgtcactcgtcactttggcagagttcggggtggtatagcccgtcaagaatgggtttatggaatttgtctgttgcctcgtgtcgcagaaagcagttcccctgtcaacggcgcatatctgaagtagagacggcctagccatcgtcttatctacttcggctacaacgcgcaattggacgctcacggtctatctgttgacacgaaccgatcagcttggtcatcaatacagtgtatatggtgaatagtagagtcgagactgcgagcagttgacggttagatgtgtattaccgtacgtcgatgaatccacgccaaggacaaagacgcgcgtcaacagaggactgaagtagactgtaatctgcgtttagttgataatcttagagtgacaatctaggcagcagcaaaatcgtttgataaatctagtgaacaggttgtcggcaatcgtagaaatccgtttaatgtgttgttggagagcgaaggtggagtatgaaagaaagtgaaagcttcaggcttggcatcccaacctcactccatccaatgcctcgcttaa

SEQ ID NO: 51 - DNA, 926 bp, Aspergillus fumigatus
  source 1..926: mol_type = genomic DNA
ctaaaagtcgaggtgtttcctcataaaggcaagctgcctctgcaggacttcttcggctccctctccgttaattacatccatatgacctcggccagcgacgagatcgaactccttgtttggctccggaatcatgtcgaaaacagctctttgatccgccggcgacgagatggtgtcctcggcgggagtcaccatcatgactggcgtccctttgatgaggcgcatcgctccgtacggctgccacgccagcacatggtagtagctctgcacggtcgtccggttggtaaagaacgaggtccccaggaatgctcggaactgctccatgttgtactgattgccccaaccagcggggttgtagccgtcatccccgacaaacgggatgtataccgggtcgttgcccgcgagagtggagacacgatcctgcattgccagcgccatgacgttgttcttttccttctctcgaaagtcgtagttggcaattggggtcaccgagatggcggctccaacacggtggtcgagaccagccgcaacaagcgccgtcatggcactgaaggagtagccatagagaatgatcttgtcctcatcgaccatgggatgacgagccataaaggtcagagcatcgtgaaagtcctccaccagcttggccggcttgacatcattgcgcggttcgccatcactggcaccaatgcaacgattatcatacaagaggaccgttactccctgttgctggaaccagacggcaacatccggtaacaagatctccttgggggtgttgaactggaccaatcagcctacgaatacagagacaaatcaatcagacgtactccctgattcatgacaatggccggcccacgaattgtcccagggtacagccagcctcgcaatatcaacccatcacaggtcggaaactcgacatcctcgcggttcat

SEQ ID NO: 52 - DNA, 468 bp, Aspergillus nidulans
  source 1..468: mol_type = genomic DNA
tgtgtctggttagaaaatgcacaaccccaagtctagccgatgcttttgcaccttattgagagcagtggaaaaaagctggaatcatctgggacatatcaagctgaactgggcgaaataaacattacaacacttccatactatcggcattgctaataatagccccgtcagccgcaaatcgactggactccgaccggggatctagtattccgagtacgagtacgagtccagagtactcatcgccgaatgccgccccggtcaaattggccgatctgacgcttgtcacttggcagcctgatagcagtctttattgatcacaataaagctgacctggtgcaacaaaaatctgtcttgcacttgattccaattttgcagactgctctccttattatctcaggccgagtctgcattttcctgtcttttttttttttgttgttttccaccttctcttggtggttccatcgcctcaga

SEQ ID NO: 53 - DNA, 7603 bp, Aspergillus fumigatus
  source 1..7603: mol_type = genomic DNA
atgaccctcacatatggccataagcgcctccaggatgccccagagcctatcgcgatcgtttctgcagcatgtcgattaccggggcatgtgaatggcccgcacaaactatgggaactccttcagtcgggaggcactgccgtttccaatgaggtgccccaatctcgatttagttccgagggccatttcgacgggtcaggccggccgggcaccatgaaagcgctgagcggcatgttcatcgaggatatcgatcctgccgcctttgatgcggcctttttcaacctcacccgggctgacgcgattgccatggacccccagcagcgtcagcttcttgaagtggtatacgagtgctttgaaaacggcggcataccgattgagaaagtgagggggaaacaaatcggctgctacgttggcagtctcaacggcggtaagagcctctggatgtcgcggtggtccgttgcagacataattcggattctcattgatgcagattaccacgacatgcagatgcgagacccggagcaaagggtgtcgggtcatgcagttggcacgggtcgagccatactgagtaacagaattagccacttcttcgacctaagaggatcgaggtgagtttccaagacactcgatggtctcttcggcagtgactgagatcgactccatgcagtttcacaattgacacagcgtgctcgagcggccttgtgggagtagacgtcgcctgcaagaatctccgcgcgggaacactgaccggagcagtcgtggctggtgtcaatctgtggctatcaccagaacacaccgaagaaaggggcaccatgcgggcagcgtactcagcgagcggcaagtgtcacaccttcgatgctaaggctgacggatactgccgcgcggaggccgttaatgctgtgtacctgaagcgtctatcagatgctgtgagggacggcgatcctatccgcgcagtgattcggggaaccgcgagtaacagcgacgggtggacccccgggatcaacagccctagcgcccaagctcaagcggcgatgattcgcgaagcttatgcaaatgctggtatcgacagcagcgagtacgccgagacgggatacctcgagtgtcatggaacgggtaccccggcgggagaccctactgaagtcaaaggcgcggcgtcagtgcttgctcacatgcgcccaccggcgagccccttgatcatcggatcggtgaagagcaacattgggcactcggagccaggagcaggtctctctggcctcatcaaggcgatgctggtggtcgaggagggcgaaatccccggcaatcccacgtttctcaacccaaatccagccatcgatttcgataacctccgggtatatgccacccggataaggattccatggcccaaagaatcaagccactacagacgtgcaagcgtcaactcgtttggctttggaggctccaatgcacatgctgtactagacaatgcggagcactaccttgggaagtactgggcatccctcgagataccccgatctcacctcagctcatatatcaatctgtccgacatgctgtccttgtttgacggacggcgatcatccaaaacagtcactcggcggccccaagtactggttttctcggccaacgacatggattcgctcaaacgccagatatcgacgctttcagcccatctcctcaacccccgagtcaaagtcaagctttcagatctcagctatacactctcggagcggcgatcccgtcatttttgccgcgcattcctgctaagctaccccgcgaagagtggacatgccagtaagatcgccgtggaggaggctcagttttccaagatctcgcaagaggcaaccagaatcggctttgttttcaccggccaaggcgcgcagtggtcacaaatggggctggagctggtcagaacgttcccaggggtagtgaagcccattctggagcagctcgacaacgtgctacaggagctgccagcagacctcaagtcagagtggtcgctgctgcaagagcttacggaagctcgctcgtctgagcatctgagcaggccggaattctcgcaacctctcgtgaccgcgctccagctggcacaactagcggtattgcaatcctggggtgtgcgggcagaagccgtgataggtcattcttcaggtgaaatagcagccgcgtgcagcgcaggactccttacaccccggcaggctattctgaatgcgtatttcagaggactcgcagggaaaagtgctctggcaactagtccgaagggcatgatggctgtgggactcggtgcacaggatgtccagccgtacctcgagggcgtaagtgccgacgtggtaatcgcatgccacaacagcccagctagtgtcacgctgtccggttcggcctccacattagcggagctggaagggaccatcaaagccgctggacactttgcccgaatgttgcgagtggaggtcgcgtaccactcgcctcacatggccaagatagccaaccgttacgaagagctgctgaaggagcacggaaggctggacgatggcagtaaaaccaataagagatcgaatcgtatgatctccaccgtgaccgaagatgaggttactggagctcaagtctgtgacgcggcatattggaaagcgaacatgctgtcgcccgttcgattcgacggcgcatgcaacaagctgttaacgaacacgcaactcgctcccaatttcctcatagaactggggcccagcaacacgctcgcaggaccagtcactcagattgccagagcagccaaggtggacaacctcacgtatgctgccgcgaataagcgtggccccgacgagagctcccgcgcaatcttcgacgttgcaggccacctgttcctgcagaatgccgacatctcacttgacaaggtgaacctcggcgacaatacaccagataaggcgaagcccgcggtgatcgttgatctgcccaactaccagtggaagcattctacccactactggcacgagagtctggccagcaaggattggagattcaagaagttcccgtcccatgacttgcttgggagcaaggttatcggcacgctgtggcagagcccgtcctggcacaagatgctgcgtctgtccgacgtgccctggctgcgggaccaccggattggatcagagatactctttcccgctgctggctatctggccatggccatggaagctgttcgccaagccgctttgtcgactgcaacagctgaagctcgagagctcctgaagacgagacactaccgctactgcctccgggatgtacaatttccgcgaggactggtgctcgaggatgatgccgaagttcatattatgcttttactggtacccatggcaaagctcgggcagggatggtgggaatataagatcacctctctcgcggaatcggattcagtagcatcgtcatcatcgtcaaccttgtccccggagaagtggaacatcaactccaccggattggttcgactagagacaatcctagaggcatcatcgtctcgagcaccagagcacacctgcagcttgcctttggataacccgacacctggacagatgtggtacaagtctctcagggacgccggatactcttacggtccaagtttccagagactggtagccgtcgagagcacggagggaaagtcagccacgcgctctcttatctctttggaaccgccacgatccaagtgggagccgcagtcagaatacccactgcacccagctcctctggacagcgtcctccagagcatgttcccctcgcttcatcgtggaaatcgaactaaactagaccagctactcgtcccaagaggaatcggtgagctgaccgtctctggagacatctggaagtccggagaagcaatttctgtgaccacctggaacaaggtgtccggagacgcgtctttgtacgatcctgccagtcgatcgctaatcatgcagctcaacagcgtgtcgttctctcccatgctggatggtcgagacagtctttacatgtcccatgtctatactcaattgacgtggaagccagatttccaacttctggatactgatgagaagctccaacaggccctcagcggtggtgatggcgctgcgtcttcccttgtccaggatcttctcgacctcgccgctcacaaggcgcctaatttgagggttctcgagttcaatctcgttcccggaagctcggaatccctgtggcttgccggacatccaacaccgcgtgctgttcgcacggcccttactgaattccactttgctgccaacagcgctgatactgcgctcgccgcccaagaggaatatgcagagtggccggcggcacgaaccgcccgcttcagtgtgcttgatcctttcagcaaagcccttgctgtacccgcaggaagttcccagttcgatcttgtgataatcaggcggcctcagcatgcagacttgggcgagctcgacattctcgtcggcaacttgcgccgtctgacttccgacggcggcagtgtaatattctatgattccaaacagtccagtctgtcagggggtcgaggtttggcgaatgggcacaaccatttccccgctgcactgcaacgctttggtctcactaaggttcgccagacgagggatgggagctgcattgtggcagaggtcagcccagcacagaatctctctctccgcaatgatttcagagtcgttattgtgcggttctcaactgcgcggtccactattatcgatcacaccatttcgcagctgcgccaatttgggtggaccttgacggagatttgcatctacaatgaatccggcactgggcttccacaacttcctcccaaatcaacggtgctcgttctcgacgaattggaccggcctttgctggccaccgcgaccgaccatgaatggacggcgctccaggcgataatacagtcagaatgtaacttactttgggtgactgagggctcgcaagttaggcctactgcgccgctcaaggccgttgcgcatgggatctttcgtactgtccgcgccgaggtacccatgatgcgcatagtgactctggacgtcgagtcagccacaactgagagtttgggcacaaacgcgtcggccatcaatatggctctgagagagataactttagcggacagatcgtccctccccattgagtgcgagattgcggaacgaggtggtctgttgcatgtcagccggatatggccggatgctggcgtgaataaacgcaaggtggaagacaacgcaggaggcgcaccacctgtgctaaccaatctgcatgattcaaagtctaccattcgcttgatggcaagcagacctggtagtttggaggcgctgcatttcgccgagcaaggtcgagatgtgtgcagtaggcaagatatgggaccggatgatgttgaggtcgagatcttcgccgctggttgcaactccagagacattgatgtggctatgggcgatatctctggggatttggatggactcggcttggaaggtgctggcgtggtcgtccgcgtcggcgcctgtgtcagcgctcgctgtgttggccagcgggtggcagtgtttggcaaaggctgctttgcgaaccgagtcaccgtctcatgcaaagccacctttcctttgcctgatgccatgtcgtttgagcaggctgcgacgctgccaatcgccttgctcaccgctttatacgccgttggtcgtctcgcacatgtacagggagatgatcgtgttttagtccattcaccttgtactgatgttgggatcgcttgcatccgactctgccagcgctcggggtcgactcccttcgcgacggtggacaacctggagcagcgccattttctgactcacgagcttggactaccggaagatcatatcttcatgtcggagcctgcagcatttcctcgcgctctccgccacgcaaccaagggccatgggcttgacgtgattatcagtcagcctgcaaatcgcaatctcgacaatgaaaacatgcggctacttgcccctggtggacgacttatcgggatagcaaacggaggcgccgatgttggaaatttgctgcccacgggatctctcgctcccaactgttctttccagaggttggatgtaacagctttaccggagaaaaccattgaatcgtaagtaaacgttggagaaatattggcttatcttttatcgagagtggaaactcatttgacagtgtgttcttggagctttctcggctcgtcacagatggcagtgtgcagcccctgtcaccaagcacactcttgggttatgaagagatacccaaggccctgcagcttcttcgagaaggcacccacatcggaaagatcgttatttcagacccccgtggcacgaagcttgctgttctggtaagagtttgaacttgacgtgtctgaatcggattctaacctgtccagacccgacctgcaacaaccctggcacagagtatgattaaccctagccactgttatctcttggtgggtggtttgaaagggatctgcggtagtcttgccatccatttagcctcccacggggccaagaacattgccgtcatgtcccgcagtggtggtggagaccaggtgtctcagggcatcgctcgaaacatcagagcactggggtgttctcttgacctgcttcaaggcgatgtcacttctatcagcgacgtcaggcgggcctttagccagatctcggttcctctgggtggaatcatccaaggagccgccgtattccgagtaagacagcactcccgaagccattctctgctattcatttcgttctgacctagaaaccatcaggatcggacgtttgaatccatgtctcacgaagactaccacgccgctgtgtcgagcaaggtgacgggcacatgcaacctacatacggtctccctcgaaacaaatcaaccgatctcattcttcaccatgctgtcttccatttcaggcgtcataggccagaagggacaagccaactacgctggtggcaatgcattccaagacgcctttgcagagtatcgccgcgcattggggctgcccgccatcagtattgacctcggacccgtagaagacgtcggagtcattcacggtaacgaagacctccagaataggttcgacggtagcactctgctcagcatcaatgagggcctgctgcgccgaatctttgactactcaatccttcagcagcatccggatccacagcaccgtctgaacgtcacgagccaaggccagatgattaccagtatactcgttccccagcctgaagacagcgatctgctcagagattgccgctttcgaggcttgcgagcccttggagaacatagtccacgctcacggcgggaccctaccaaagataaagagatccagagcctcttgtttctggcccaatcccaggatcccgatcgtgcagccctgcgcgccgccgctatcacggtcgtgggtgcgcggctggcaaagcagcttcgcttaacggatgcagtcgacccggcacgtcccttgtcctactacgggttagactctctggcggctgtcgagctacggacctgggtgcgtatgacactggcgatagagctcaccactttggatgtgatgaatgcagccagcctgggagaattgtgtgagaaggtgattgggaaaatgggatttggcatgtag

SEQ ID NO: 54 - DNA, 305 bp, Aspergillus nidulans
  source 1..305: mol_type = genomic DNA
gcagtatgttaaccggtagtgaaagggctgcgctgttgctttcggttgttagagttatggtatataggtacagatgaaaacactggtctatgcatatttcactatccttgacgcgacgaagtaagcctcgatgtgatctatcgtcgtagataacagcttaatgacccgatctgtgcttaatttcccgccgctgtccggatctcgtctcgggtcattttgcattatatagggagcctccactcgcccatcctcactcatcaaccacatcgaccagctcagaattcacccgcatcaattcaaagaaa

SEQ ID NO: 55 - DNA, 895 bp, Aspergillus fumigatus
  source 1..895: mol_type = genomic DNA
atggatcagtcgatgaagccccttctctcacccacagaacgaccacgtcggcatctgacagcgtccgtcatctccgtaagcccctcctcaaccatgcagaagtaggatctaatgaagcaaccgctaacgccatggtaaaaagttcttcctcccaaatcaattccgtctcagcacgatcctttgcattggtgctctcctgcagaccatcctctgcgccgtcctccccctccgctacgccgccgtcccatgtgtaactgttctcctcatatccgttctcaccacaatccaagagtgcttccaaccgaacacgaattctttcatggccgatgtcattcgcggaagaactaccgcgcagatcccaggcaaagatggaacacacggccgggagccggggaagggctcggtggtagtgttccaccttggaatacaatacaatcaccccctcggagtttttgcaccgcacatgcgcgaaatctcgaaccggtttctcgccatgcagcaggacatactccgccgcaaggatgagctcggcctgctggcggttcagaactggcgagggagcgagcgcgactccggtaacaccacgctgatcaagtatttcttcaaagacgtggaaagtattcataaatttgcccacgaaccgctacataaggagacttggacgtactataaccagcatcaccctggtcatgtgggcatctttcatgagacatttatcaccaaggatggcggatatgagagcatgtatgtaaattgccatccaattctacttgggagaggcgaggtcaaggtcaataatcggaaagacggcacagaggagtgggtggggacactggtcagtgctgatacgcctgggttgaagtcttttaaagcaaggttgggtagagatgactga

SEQ ID NO: 56 - DNA, 374 bp, Aspergillus nidulans
  source 1..374: mol_type = genomic DNA
caatttttttatcattttctggctattcgttcaaataacagggtttctttggtctgggtaatggtttctgtcctaaggcttacggtcagggagcagttagttacctagagtcgcttcgggacatcaaccgtatctgtttgttgatatgacaactattacttgattacttttgtttttcttggtcgtcttctttatttatctgattactgagttccagatgcacaccggaccccgacagttccactgaaacccgagctcggatagcacgacgctgacgctgacgctgcatgtccagtcaccacggctcgtattttgaaacagtcaaagcagtgaccagagtctacagtggagtattcaagcacctatcaaacaga

SEQ ID NO: 57 - DNA, 1857 bp, Aspergillus nidulans
  source 1..1857: mol_type = genomic DNA
atgtcggtttcacgctcgtgcttcaggcctttcctcccagcagaaatcgatggtgggcacctacccgttgacccttcggtctttacacacattgagcgtggcctccatcagaatccacagggttttgctattcagagtacccatcaacaaccgtgtcatttctctgcgcttgttcagacaggaagtgggactgaaaatggcggtgcgccaaactatgatgcggtcgagagagaaccggggacatgcctcgcctggacatatacacaactccaccacgctgcgttacggattgcggcggggctgctggcgagaaatgcccagccaagcacgagaatgctcttgctcatccccaacggcgccgagttctgtcttctgctttggactgcggttgttctccgcgtgacgattgtctgtctcgatgaggaactgcttaacgttgagcagcatgatgagttacgcagaatgctaaagactatcaatccaagggttattgttgtgcaagacgtaaaaggcgcggatgtgatcgatgtcgcgttgcggaatctaccgcttgacccggatatcctcaagatcactctatccgagcttgcgggaagtcaaccagactcagcctggagatcccttctgtccctatctctgacaccagctctttcagcttctgaaaccgagtctcttctatcttctgctcgctgggactcttccaacgcagcccgtacatactccatcctctatacgtcaggaacatccggggtccctaaagggtgcccgttgcatatttcgggaatgagctacgttctccaatcccagtcgtggctggtcaacgcagagaactgcacgcgggcactgcaacaagcgcatccgtgtcggggcattgccattgcacagacactccagacatggagggaaggtgggacagtagtcatgacggggaatggcttcaatgcgggcgatttggtgcatgcggtaaaaaggcacgcggttagtttcgtggtgctcacgccggcgatggttcatccagttgcagacgagttgaagggtagaaatggcgcagctgattctgtcaggacagttcaaatcggtggcgatgcggtgacaagaggcgcacttgagatatgtacgcgattgtttccgaaagcgagagttgtcgtgaatcacgggatgacggagggtggaggggcgtttgtttggcctttcaacaggcccagagatattccgttctatggtgagatgagtcctgttggatccgttgcacgaggcgctgctgtcaggatccgtggcgcaaacgcgacagtggcaagaggagagctgggcgagctccatgtctcctgcccaagtattatcccggggtatctgggtggagtttcagcccagtcgtttcacgacgaggatgggcgaagatggttcaaaacaggtgatgtgggcttgatggacaagcagggcgttgtttttatccttggccggatgaaggatatgattaatgggaaagtgatgcctgccccgattgagagttgccttgagaaatatacttctgttcaggtatgttttctttctttattcttcccccatacctccaccacatttgcctcagatctgagatctaaacaagcataccagacatgtgtggtaaatgctggcggcccctttgctgtcctggcacgatataccggcaagaaagaagcccagatcagaagacatgttgtgcgggcacttgggaagagcaatgcgttgaacggagtaatttatctgcaccagttgggactggaaaggtttccggttaatgggacgcataagattgctcgtggggatgtggagggggctatgctggcctatttgcagactgagcctaccagtagatag

SEQ ID NO: 58 - DNA, 145 bp, Aspergillus nidulans
  source 1..145: mol_type = genomic DNA
aaccctacctatagatggattgtgtgctgagggcgtctcaatatgctattcttaacgccaccgaaatcgtacatcagatcactcaagacgtcaagacatggctccaactagccgactcgggttgtcccattagacattctaatca

SEQ ID NO: 59 - DNA, 799 bp, Aspergillus nidulans
  source 1..799: mol_type = genomic DNA
ttaccattttatatcctctggaatctctaactcaagtcccaaatccgggacacctcccgcaaccttcttaaaccagccaatctcaaggaccccatcataccagctgcacagtgctccaaacctctcctgcatggatctcctaaacgccgcaaacgctcccatgagcagactcgcggcgaaaaaatccgcaatcgttatactttcccccacaagatatctgctgcgcttcagatgctcatctaggtacttgcaccgctgcagcatcgcacgcagtgagtccccgtcatcttgctggattatttgccgttgcccaatgcgtgggaggaagacgccgccgactgctggaaagaggtcggagtttgcaaaagacatccattggaggatcctgagcgaggagcgttcgtcattgcctaggagggattttgttatcgggtcctgactctgggatgcaactgctcagcaggacttttcagtcctctttcattaaccagggagtgtcggggctgagtacagtaaagagtcaatggaatacattcactcagcacgaacccgtctgcgcctacaaaagtagggacttgcccgagtggattatatctgcaaagctcctcaaatgcctctttattcttcttttctgcgtgtatgattttgacgtcgaggttgtggagctttgctagagcgatgagggtcgtcgagcgaggcgttggctgcaagattcaaggttagctaaacccccaattctaattctgggccctgaggtgtaagaacatacgttatgggtgtagagtgttccgaatgacat

SEQ ID NO: 60 - DNA, 364 bp, Aspergillus nidulans
  source 1..364: mol_type = genomic DNA
ttgtgcggtctggtctgtttggaaatgataatgcgggtgggtatgggctgtcggtgattatatctactccgtcgaaccggaacccgggggtctgcgactgcgatacgctcgatgaactccgagatttcgggggccgggggttgaggttgcactgcagatcttgatatccagcatctagcacggtatagttcgtatcttgagatatttgagacattgaagtctgaaaacgacggtttaggctacggtacccgactgccatagctctctatacgagtgctttataaacacccaaccaccatcaaccataatcctcacggcaccgtattggttacgaaatactaaattctgaatatcatcaatcgaa

SEQ ID NO: 61 - DNA, 798 bp, Aspergillus nidulans
  source 1..798: mol_type = genomic DNA
atgcctctggccacttacgccgttctgggcgcaaccggcaatactggcacggctctgatccagaatctgctctcgccaccatcttcagaaatgcacataaacgcctactgtcgaaacaagcccaaactcttaaacctcttgcccgaactcaacgacacgaaaaatgtgactatctttgaaggctccatcaccgacttatccctcatcaccgcatgcatacgcaacacacgtgcggtcttcttgaccgtcacttcaaacgacaatattcccggttgccgactgagtcaggactcggtgcagacggttctcgaggcactcaagcagattcgtacagcggaaccgaatgcagttgtgccgaaactggtccttctctcctccgcgacgatagatccgcacctaagccgcaaaatgccctcgtggttcttaccgattatgaaaacagctgcgagtaacgtctacgccgacctgatcaaggcagaggagatgctgcgagcgaacgagtcctgggtcacaagcattttcatcaagcctgccggcttgagcgtcgacattcagcgtggtcacaaactcgactttgacgagcaggagtcgttcatctcgtacctggatctggcggctgccatgcttgaggcggcaaatgatacagatgggaggtatgatgggaggaacgtctctgtggttaatacggggggcaaggcgaggttcccgcctggaactccgaaatgtatcattgttggcttgctcaggcatttcttcccggggttgcatcgatttctgccaacaacggggccttcctaa

SEQ ID NO: 62 - DNA, 360 bp, Aspergillus nidulans
  source 1..360: mol_type = genomic DNA
tggcctgggattgtagcctggggtatgtaatattgggtctctaggaggacgttttggttattagatgggtcaattttatggattcccaacaccgcaaaacgtagccctgatcgaggttaaggcctcagtcactcattcgtactagtcacgctcggcgtacctttgccatttgctagatatagagaaccagtccagtcgacaatatgtgaatatggctgctcggtcatcgggcttcgaggtctcgttatccgaagctagctgtgcagtatatatctttgggctcaggacattaaaccagtcagcaaaacccaaccatctaccataccaagtcaacaagaaagcacgaatacggcgtcaaaa

SEQ ID NO: 63 - DNA, 1341 bp, Aspergillus nidulans
  source 1..1341: mol_type = genomic DNA
atgtcctcttcgatcaatattctctcaaccaaactcggccagaacatctacgcccaaactcccccctcccagactctcactctgacaaatcacctcctacaaaagaaccacgacacgctgcacatctttttccgcaatctaaacggccacaaccacctggtccataaccttctcactcggctagtgctgggtgcaaccccagagcaactccaaaccgcctacgacgatgacctccctactcagcgcgccatgccgcctctcgtcccttctatcgtggaaaggttatctgacaactcctacttcgagtcccaaattacacagattgaccagtatacaaacttcctacgtttcttcgaagcggagatcgaccgacgagactcatggaaggacgtcgtgatagagtacgtcttctcgcgctcgcccattgctgagaagatcctcccgcttatgtacgacggcgcctttcactcaattattcatctcgggcttggagtcgagttcgaacagccggggatcatcgctgaggcattggcgcaggcggccgcgcacgactcttttgggaccgattactttttcctcacggccgaaaagcgagctgctgggcgaaacgaagagggagagactctcgtgaaccttttacagaaaatcagggacacacccaaacttgtcgaagccggacgcgtccagggcctcattgggacgatgaagatgagaaagtctattctcgtcaatgcagctgatgaaataatagacattgcgtcgcggtttaaagtcaccgaggaaacgctcgcgagaaagactgccgagatgctaaacctctgtgcttacttggctggtgcgtcgcagaggacgaaggacgggtatgagccaaagattgactttttcttcatgcactgcgtaacaagcagtatcttcttctctattctcgggcgtcaggactggatttccatgcgggatagagtaaggttagtcgagtggaagggccggctggatctgatgtggtatgctctctgcggtgtacccgagcttgatttcgaatttgtgagaacctacaggggggagagaacggggactatgtcctggaaggaattgtttgcgattgttaatgagcagcatgatgatgggcatgtggcgaagtttgtgcgagcgctgaagaacgggcaggaggtttgcgggcagtttgaggatggagaggagtttatggtcaagggggatatgtggttgaggattgcgaggatggcgtatgagacgacgattgagacgaacatgcaaaatcggtgggtggttatggcaggcatggacggggcttggaaggacttcaaagtgcagtcgtctgattga

SEQ ID NO: 64 - DNA, 459 bp, Aspergillus nidulans
  source 1..459: mol_type = genomic DNA
ttagatatacgcagtgctgtatatgggtcttggccatctagtacgatcaacaagccaagagtgactctactctctactctttacaggtctatcgatagcagtcaatctatgcatcgacaagagttcaatttgacttcccgatttcgactcagagaatcctaggcccatgccaggacttataaatgcctatccatgattgcatgaagtcctttctccaaacacctcaaagaccattgcttgtgagcgtcagtttacctttttgactatgtcgggtcctcaggctggatcatagcgctattccatattcagcttggcgtagaatggtttacgctagcccactccggctagacggcctgaacgccgggatatttccacgtgacggcattcttttcaacttcaagccctacaagcgcgccctacccctaagccctcattgctgatcctggaagcatcatcttc

SEQ ID NO: 65 - DNA, 2778 bp, Aspergillus nidulans
  source 1..2778: mol_type = genomic DNA
atgtcagcgccaactcctcccgtcatggccgatgccagtgcatcaggaccctccgttgacacgcagggagcgtccgacctccctgcctcgccggtgcccaaggaggagggtcaccatggtaagccacctagccgcattcactgcctgactccggcagtaacaccaccccaagtctattcactcaacccaatgacttactcttgtcacactagaactccccaagctgtttcatcccatcgaggatgattctctttcgccgcgggcatccaaaaaacgtcggcttgatgaaccggaggactccgtagcggaaacgacaacgacaacaccaccgtcccagcaacctcaagagcaaacccgggaaccgtcgcagcaaacggagcagagccagttccagcaacaacacacgaatcttcttcctggtgctggagaccagattgaagaagaattggcatcggcccttgccgcgggggtcgtcgattcggtggaaactgcggatagcaagaatggtcagaccgagatcggagcaagtcctgtgcaagagcaaaacacgaatatcgactcggacgtagctactgtcatctcgaacatcatgaatcattccgagcgtgtcgaggagcagtgcgccatgggtccccagcagttgccggatttgtccggtcagggcgctcccaaggggatggtttttgtcaaggccaattcgcatctaaaaattcagagtttacccattcttgataatctggtgagttctctaattcaggctcagagttttggttaggaagctaatttgcagtccacgcaaattctgtcgctgctggccaagtccacgtaccaagatattacctccttcgtatctgagccggagtcggagaatggtcaggcgtacgctacgatgcggtcactgtttgaccacacaaaaaaggtctattcaaccaagaaatcgttcctctcgcccacggagctcgagctcactgaaccttcgcaagtcgacatcatccgcaaagcaaacctggcatcgtttgtctccagcatctttggtactcaggagatcagcttctctgagctcaatgataactttctcgacgtatttgtccccgaaggtggacggcttctcaaacagcaaggtgccctttttcttgagatgaagactcaagcgttcatcgcgtcgatgaacaacaccgaacgtacccgcaccgaattgctttatactttgttcccagataatcttgagcagcaactccttgacagacgacccgggacgcgtcagctggctccgagcgaaaccgactttgtcaaccgtgcacattcgcgccgtgagatattgcttaatgatatcaacaatgaggaggccatgaaagctttaccagacaaataccactgggaggactttctccgggacctcagctcgtatattacaaagaactttgataccatcaacaaccaacaggttagactctacatatggttttaaacaaatagatcgctaatgcggattagtcaaagaagatcacaaaaggacggcaaccatcttcatcaaatggtgattctgagccgcctagtgcgcctcttcagagccagtttcctgtcgccacgcaggcgccggaggtcccagtcgataaaaacatgcacggtgacctggttgcccgtgccgccagagctgcgcagattgcgctgcagggtcacgggctcagacgttctcagcagcaggcacagcaggcccagcagcaacaagcccagcagcaacaagcccagcagcaggcccagcagcaggcccagcagcagcaacaggctcggcagcaggctcagcaatatcagcagcagcagcaacagcaacagcaacagcaacaacagcaacaacaggctcagcagcaggcgccccagcagggcatccagattctacaaggatatacccccgcgcagcaaccctaccagagcagcccagctccttcaggatatcaacagtctcagacatataacttccaacagagcccaatgcagacaaacttccagcagtacaaccacccctcgccgtcgccaatacccggtcgacctaactcgtctactgccaaccacggctacatgcccggcattccccactactctcaatctcagccgacacaagttctctatgagcgggctcggatggccgcatccgccaaatcctcgcccagcagccgcaagtctggccttcccagtcaacgccgcccatggacgactgaagaagaaaacgccctcatggctggccttgaccgcgtcaagggaccccactggagtcagatcctggccatgttcggccccggcggtacgattagcgaagctctcaaggatcgcaaccaggtacaacttaaagataaagctcgaaacctgaagctcttctttcttaagagtgggattgaggtgccatactacctcaaattcgtcacgggtgagttgaaaacgcgtgctccagcacaagccgccaaacgtgaggcccgcgagcgccagaagaaacaaggggaggaggataaggcacatgtcgaggggatcaagggcatgatggccctggcgggggcgcatccgcagcaggtcggccatcctcatcatggagttcctggagttccgcaccacggccacgagagcatgtctgcgtcgccgatgccgccagatccaaactttgatcagacggcggagcaaaatctcatgcagacgctgggaaaggaagtccatggagagtcattcgggcagcctgggcagcctgggcacccggggcatcatcctgagaatatgcatatggggcaatga

SEQ ID NO: 66 - DNA, 20 bp, Synthetic construct
  source 1..20: mol_type = other DNA
ggagcgacagaaccaaagtc

SEQ ID NO: 67 - DNA, 20 bp, Synthetic construct
  source 1..20: mol_type = other DNA
tgggccatgggctatcttcc

SEQ ID NO: 68 - DNA, 41 bp, Synthetic construct
  source 1..41: mol_type = other DNA
ctatcacaatcagcttttcagttacgagcgagttacgaacg

SEQ ID NO: 69 - DNA, 21 bp, Synthetic construct
  source 1..21: mol_type = other DNA
ctgaaaagctgattgtgatag

SEQ ID NO: 70 - DNA, 20 bp, Synthetic construct
  source 1..20: mol_type = other DNA
tgctggggtatggctatctc

SEQ ID NO: 71 - DNA, 20 bp, Synthetic construct
  source 1..20: mol_type = other DNA
atggcagtgagcagacattg

SEQ ID NO: 72 - DNA, 20 bp, Synthetic construct
  source 1..20: mol_type = other DNA
aatgactggtccgtccgtac

SEQ ID NO: 73 - DNA, 43 bp, Synthetic construct
  source 1..43: mol_type = other DNA
cgaagagggtgaagagcattgggtgccttgtggatggggatta

SEQ ID NO: 74 - DNA, 21 bp, Synthetic construct
  source 1..21: mol_type = other DNA
caatgctcttcaccctcttcg

SEQ ID NO: 75 - DNA, 24 bp, Synthetic construct
  source 1..24: mol_type = other DNA
ctgtctgagaggaggcactgatgc

SEQ ID NO: 76 - DNA, 46 bp, Synthetic construct
  source 1..46: mol_type = other DNA
gcatcagtgcctcctctcagacagattcagcctattgagattacag

SEQ ID NO: 77 - DNA, 20 bp, Synthetic construct
  source 1..20: mol_type = other DNA
cctagtaggtgggatttgaa

SEQ ID NO: 78 - DNA, 22 bp, Synthetic construct
  source 1..22: mol_type = other DNA
atgtgctctacggacgaaaaat

SEQ ID NO: 79 - DNA, 20 bp, Synthetic construct
  source 1..20: mol_type = other DNA
atgaagagcgcctgtttctg

SEQ ID NO: 80 - DNA, 20 bp, Synthetic construct
  source 1..20: mol_type = other DNA
aatgactggtccgtccgtac

SEQ ID NO: 81 - DNA, 43 bp, Synthetic construct; identical to SEQ ID NO: 73
  source 1..43: mol_type = other DNA

SEQ ID NO: 82 - DNA, 21 bp, Synthetic construct
  source 1..21: mol_type = other DNA
caatgctcttcaccctcttcg

SEQ ID NO: 83 - DNA, 24 bp, Synthetic construct
  source 1..24: mol_type = other DNA
ctgtctgagaggaggcactgatgc

SEQ ID NO: 84 - DNA, 45 bp, Synthetic construct
  source 1..45: mol_type = other DNA
gcatcagtgcctcctctcagacagggcatcgtctacaagcagatg

SEQ ID NO: 85 - DNA, 22 bp, Synthetic construct
  source 1..22: mol_type = other DNA
tttggtctcttccacaaggact

SEQ ID NO: 86 - DNA, 22 bp, Synthetic construct
  source 1..22: mol_type = other DNA
atgtgctctacggacgaaaaat

SEQ ID NO: 87 - DNA, 22 bp, Synthetic construct
  source 1..22: mol_type = other DNA
gtctttgactaccggagcaagt

SEQ ID NO: 88 - DNA, 20 bp, Synthetic construct
  source 1..20: mol_type = other DNA
aatgactggtccgtccgtac

SEQ ID NO: 89 - DNA, 22 bp, Synthetic construct
  source 1..22: mol_type = other DNA
ggtgccttgtggatggggatta

SEQ ID NO: 90 - DNA, 21 bp, Synthetic construct
  source 1..21: mol_type = other DNA
gctgcatcggtcatgttgttc

SEQ ID NO: 91 - DNA, 22 bp, Synthetic construct
  source 1..22: mol_type = other DNA
ggtggatagccgtatctccctc

SEQ ID NO: 92 - DNA, 23 bp, Synthetic construct
  source 1..23: mol_type = other DNA
cctggtgtgattgggctgattag

SEQ ID NO: 93 - DNA, 29 bp, Synthetic construct
  source 1..29: mol_type = other DNA
agtactgctttcaaaagtatatcatctgc

SEQ ID NO: 94 - DNA, 18 bp, Synthetic construct
  source 1..18: mol_type = other DNA
tgcgggagggtaggaggg

SEQ ID NO: 95 - DNA, 22 bp, Synthetic construct
  source 1..22: mol_type = other DNA
tataaccacttgcctgaggatc

SEQ ID NO: 96 - DNA, 21 bp, Synthetic construct
  source 1..21: mol_type = other DNA
cctgtttagagtggccagaag

SEQ ID NO: 97 - DNA, 18 bp, Synthetic construct
  source 1..18: mol_type = other DNA
tatgcaactgggccggag

SEQ ID NO: 98 - DNA, 22 bp, Synthetic construct
  source 1..22: mol_type = other DNA
attcagcctattgagattacag

SEQ ID NO: 99 - DNA, 22 bp, Synthetic construct
  source 1..22: mol_type = other DNA
tgcgcctggattcgggatgtag

SEQ ID NO: 100 - DNA, 22 bp, Synthetic construct
  source 1..22: mol_type = other DNA
ggcatcgtctacaagcagatgc

SEQ ID NO: 101 - DNA, 22 bp, Synthetic construct
  source 1..22: mol_type = other DNA
ctggttactgtttattttgact

SEQ ID NO: 102 - DNA, 22 bp, Synthetic construct
  source 1..22: mol_type = other DNA
aacgaggtccaggtgacggtaa

SEQ ID NO: 103 - DNA, 22 bp, Synthetic construct
  source 1..22: mol_type = other DNA
gattgctggtctttgtagtctc

SEQ ID NO: 104 - DNA, 45 bp, Synthetic construct
  source 1..45: mol_type = other DNA
ccataatccccatccacaaggcaccatggcacacatggaaccgat

SEQ ID NO: 105 - DNA, 44 bp, Synthetic construct
  source 1..44: mol_type = other DNA
agaagaacaacatgaccgatgcagctcagtcatggtccccctcc

SEQ ID NO: 106 - DNA, 43 bp, Synthetic construct
  source 1..43: mol_type = other DNA
ctggagggagatacggctatccaccctagcgacgaggcttccg

SEQ ID NO: 107 - DNA, 46 bp, Synthetic construct
  source 1..46: mol_type = other DNA
tcctaatcagcccaatcacaccaggatgacctcctaccagctttcc

SEQ ID NO: 108 - DNA, 50 bp, Synthetic construct
  source 1..50: mol_type = other DNA
atgatatacttttgaaagcagtacttcatacttccttgacattgaacacc

SEQ ID NO: 109 - DNA, 44 bp, Synthetic construct
  source 1..44: mol_type = other DNA
cctcctaccctcctaccctcccgcaatggaaggaaagcaccctc

SEQ ID NO: 110 - DNA, 46 bp, Synthetic construct
  source 1..46: mol_type = other DNA
agcgatcctcaggcaagtggttatatcagaattgagattcctcccg

SEQ ID NO: 111 - DNA, 43 bp, Synthetic construct
  source 1..43: mol_type = other DNA
acaccttctggccactctaaacaggatggccctttcagcctac

SEQ ID NO: 112 - DNA, 46 bp, Synthetic construct
  source 1..46: mol_type = other DNA
tgcaattctccggcccagttgcatacaatgctcttcaccctcttcg

SEQ ID NO: 113 - DNA, 43 bp, Synthetic construct
  source 1..43: mol_type = other DNA
tggctgtaatctcaataggctgaatctgtctgagaggaggcac

SEQ ID NO: 114 - DNA, 22 bp, Synthetic construct
  source 1..22: mol_type = other DNA
attcagcctattgagattacag

SEQ ID NO: 115 - DNA, 20 bp, Synthetic construct
  source 1..20: mol_type = other DNA
cctagtaggtgggatttgaa

SEQ ID NO: 116 - DNA, 22 bp, Synthetic construct
  source 1..22: mol_type = other DNA
atgtgctctacggacgaaaaat

SEQ ID NO: 117 - DNA, 22 bp, Synthetic construct
  source 1..22: mol_type = other DNA
gggagaagatgaaccagttgtc

SEQ ID NO: 118 - DNA, 22 bp, Synthetic construct
  source 1..22: mol_type = other DNA
tcggtggcatagacactatcac

SEQ ID NO: 119 - DNA, 44 bp, Synthetic construct; identical to SEQ ID NO: 109
  source 1..44: mol_type = other DNA

SEQ ID NO: 120 - DNA, 22 bp, Synthetic construct
  source 1..22: mol_type = other DNA
gcagtacctcaccgttgtatga

SEQ ID NO: 121 - DNA, 20 bp, Synthetic construct
  source 1..20: mol_type = other DNA
atgaagagcgcctgtttctg

SEQ ID NO: 122 - DNA, 20 bp, Synthetic construct
  source 1..20: mol_type = other DNA
aatgactggtccgtccgtac

SEQ ID NO: 123 - DNA, 22 bp, Synthetic construct
  source 1..22: mol_type = other DNA
gggatcacgtctactggaactc

SEQ ID NO: 124 - DNA, 22 bp, Synthetic construct
  source 1..22: mol_type = other DNA
gccatgttagaagggtatgagc

SEQ ID NO: 125 - DNA, 22 bp, Synthetic construct
  source 1..22: mol_type = other DNA
tctgggtatacagcagggtctt

SEQ ID NO: 126 - DNA, 22 bp, Synthetic construct
  source 1..22: mol_type = other DNA
gagctggttaggatcaactgct

SEQ ID NO: 127 - DNA, 22 bp, Synthetic construct
  source 1..22: mol_type = other DNA
atggagtcctgtagtccgaaaa

SEQ ID NO: 128 - DNA, 22 bp, Synthetic construct
  source 1..22: mol_type = other DNA
atatgccgtctagcaatggact

SEQ ID NO: 129 - DNA, 20 bp, Synthetic construct
  source 1..20: mol_type = other DNA
cctagtaggtgggatttgaa

SEQ ID NO: 130 - DNA, 50 bp, Synthetic construct
  source 1..50: mol_type = other DNA
ccataatccccatccacaaggcaccatgagaatacctaacgtctttctct

SEQ ID NO: 131 - DNA, 50 bp, Synthetic construct
  source 1..50: mol_type = other DNA
agaagaacaacatgaccgatgcagcctactctgcgatgtacaacttttcc

SEQ ID NO: 132 - DNA, 50 bp, Synthetic construct
  source 1..50: mol_type = other DNA
ctggagggagatacggctatccacctcaatggtggattccattgctcccg

SEQ ID NO: 133 - DNA, 50 bp, Synthetic construct
  source 1..50: mol_type = other DNA
tcctaatcagcccaatcacaccaggatgggtctatctgaagatcttcatg

SEQ ID NO: 134 - DNA, 50 bp, Synthetic construct
  source 1..50: mol_type = other DNA
atgatatacttttgaaagcagtactctacaacgcagcgaacgcttcctta

SEQ ID NO: 135 - DNA, 50 bp, Synthetic construct
  source 1..50: mol_type = other DNA
cctcctaccctcctaccctcccgcaatgctgtccgtcgacctcccgtctg

SEQ ID NO: 136 - DNA, 50 bp, Synthetic construct
  source 1..50: mol_type = other DNA
agcgatcctcaggcaagtggttatactaatagtctgcaacatcgtggatc

SEQ ID NO: 137 - DNA, 50 bp, Synthetic construct
  source 1..50: mol_type = other DNA
acaccttctggccactctaaacaggatgaatctttctgctctgaaggctg

SEQ ID NO: 138 - DNA, 50 bp, Synthetic construct
  source 1..50: mol_type = other DNA
tgcaattctccggcccagttgcataatggaaggcaaggtcgcaatcgtca

SEQ ID NO: 139 - DNA, 50 bp, Synthetic construct
  source 1..50: mol_type = other DNA
tggctgtaatctcaataggctgaatctaaatgacactccacccgttatcg

SEQ ID NO: 140 - DNA, 46 bp, Synthetic construct
  source 1..46: mol_type = other DNA
tgtctacatcccgaatccaggcgcacaatgctcttcaccctcttcg

SEQ ID NO: 141 - DNA, 49 bp, Synthetic construct
  source 1..49: mol_type = other DNA
ctagcatctgcttgtagacgatgccctgtctgagaggaggcactgatgc

SEQ ID NO: 142 - DNA, 45 bp, Synthetic construct; identical to SEQ ID NO: 84
  source 1..45: mol_type = other DNA

SEQ ID NO: 143 - DNA, 22 bp, Synthetic construct
  source 1..22: mol_type = other DNA
tttggtctcttccacaaggact

SEQ ID NO: 144 - DNA, 22 bp, Synthetic construct
  source 1..22: mol_type = other DNA
atgtgctctacggacgaaaaat

SEQ ID NO: 145 - DNA, 22 bp, Synthetic construct
  source 1..22: mol_type = other DNA
atggagtcctgtagtccgaaaa

SEQ ID NO: 146 - DNA, 22 bp, Synthetic construct
  source 1..22: mol_type = other DNA
aactcaatccagctacgaccat

SEQ ID NO: 147 - DNA, 22 bp, Synthetic construct
  source 1..22: mol_type = other DNA
gtctttgactaccggagcaagt

SEQ ID NO: 148 - DNA, 20 bp, Synthetic construct
  source 1..20: mol_type = other DNA
aatgactggtccgtccgtac

SEQ ID NO: 149 - DNA, 22 bp, Synthetic construct
  source 1..22: mol_type = other DNA
tataaccacttgcctgaggatc

SEQ ID NO: 150 - DNA, 22 bp, Synthetic construct
  source 1..22: mol_type = other DNA
gagctggttaggatcaactgct

SEQ ID NO: 151 - DNA, 22 bp, Synthetic construct
  source 1..22: mol_type = other DNA
tttggtctcttccacaaggact

SEQ ID NO: 152 - DNA, 25 bp, Synthetic construct
  source 1..25: mol_type = other DNA
atggaaggcaaggtcgcaatcgtca

SEQ ID NO: 153 - DNA, 22 bp, Synthetic construct
  source 1..22: mol_type = other DNA
tgcgcctggattcgggatgtag

SEQ ID NO: 154 - DNA, 50 bp, Synthetic construct
  source 1..50: mol_type = other DNA
tgtctacatcccgaatccaggcgcaatgaagcccttctcaccagaacttc

SEQ ID NO: 155 - DNA, 50 bp, Synthetic construct
  source 1..50: mol_type = other DNA
ctagcatctgcttgtagacgatgccctactgtgctacacgagggggattc

SEQ ID NO: 156 - DNA, 50 bp, Synthetic construct
  source 1..50: mol_type = other DNA
gccagtcaaaataaacagtaaccagctagccactagcaggcttcgtgaac

SEQ ID NO: 157 - DNA, 50 bp, Synthetic construct
  source 1..50: mol_type = other DNA
acgttaccgtcacctggacctcgttatggctccgtcaacggaacgtgctc

SEQ ID NO: 158 - DNA, 46 bp, Synthetic construct
  source 1..46: mol_type = other DNA
ccagagactacaaagaccagcaatccaatgctcttcaccctcttcg

SEQ ID NO: 159 - DNA, 20 bp, Synthetic construct
  source 1..20: mol_type = other DNA
atggcagtgagcagacattg

SEQ ID NO: 160 - DNA, 22 bp, Synthetic construct
  source 1..22: mol_type = other DNA
cgctggtatttcggactacttc

SEQ ID NO: 161 - DNA, 20 bp, Synthetic construct
  source 1..20: mol_type = other DNA
tgctggggtatggctatctc

SEQ ID NO: 162 - DNA, 25 bp, Synthetic construct
  source 1..25: mol_type = other DNA
atggaaggcaaggtcgcaatcgtca

SEQ ID NO: 163 - DNA, 20 bp, Synthetic construct
  source 1..20: mol_type = other DNA
atggcagtgagcagacattg

(19) United States
(12) Patent Application Publication
(10) Pub. No.: US 2023/0242866 A1
(43) Pub. Date: Aug. 3, 2023

(54) PLATFORM FOR TOTAL BIOSYNTHESIS OF NATURAL PRODUCTS

(71) Applicant: University of Southern California, Los Angeles, CA (US)

(72) Inventors: Clay C. WANG, Los Angeles, CA (US); Yi-Ming CHIANG, Los Angeles, CA (US)

(21) Appl. No.: 18/065,449

(22) Filed: Dec. 13, 2022

Related U.S. Application Data
(60) Provisional application No. 63/289,390, filed on Dec. 14, 2021.

(51) Int. Cl. C12N 1/14

(52) U.S. Cl. CPC C12N 1/145

(57) ABSTRACT

The present disclosure relates to transgenic fungal cells and methods of making the same such that the transgenic fungal cells include one or more exogenous biosynthetic gene clusters integrated into the host genome. The genes of the exogenous biosynthetic gene cluster may be operably linked to a transgenic region of an endogenous biosynthetic gene cluster that includes a native promoter to control expression of the exogenous genes.

### RELATED APPLICATIONS

This application claims priority under 35 U.S.C. § 119(e) to U.S. Provisional Patent Application No. 63/289,390, filed Dec. 14, 2021, which is incorporated herein by reference.

### SEQUENCE LISTING

The instant application contains a Sequence Listing which has been submitted electronically in ST.26 format and is hereby incorporated by reference in its entirety. The ST.26 copy, created on Apr. 14, 2023, is named 530-020US1 SL, and is 244,000 bytes in size.

### BACKGROUND

Fungal natural products (NPs) are invaluable sources of new leads for the pharmaceutical and agricultural industries. Genome sequencing projects have revealed that biosynthetic genes of individual NP pathways are usually clustered together in the genome and that these biosynthetic gene clusters (BGCs) vastly outnumber known NPs. The latter observation indicates that firstly, the chemical diversity of fungi is largely untapped. Secondly, most BGCs remain silent or expressed at levels below detection limits under laboratory cultivation conditions. Although most fungal NPs exhibit bioactivities, many of them are natively produced at very low titers such that commercialization is hindered by the cost of the production. The stereocenters often found in complex NPs, moreover, render total synthesis challenging. Consequently, reconstitution of fungal BGCs in genetically tractable hosts offers an alternative route for scalable and economical production.

Various hosts have been explored as heterologous expression platforms for fungal BGCs. While E. coli is a well-established prokaryotic host, its application for heterologous expression of fungal genes is limited by its inability to perform RNA splicing and post-translational modification as well as the codon bias between E. coli and fungi. Yeast, Saccharomyces cerevisiae, has been proven to be a successful platform. However, yeast lacks the ability to splice fungal mRNA accurately and might be deficient in specialized compartments to produce certain fungal NPs. For these reasons, genetically tractable filamentous fungi may be better heterologous expression hosts for fungal BGCs. The whole penicillin, citrinin, fusatins, and W493 BGCs were transferred from their native producers and successfully expressed. Bok and Clevenger et al. used fungal artificial chromosomes to introduce large intact BGCs from three Aspergillus species into A. nidulans, and about 27% of the transferred BGCs produced detectable products. Despite these examples of success, the production of heterologous compounds is often low. In some cases, titers could be increased by overexpression of the BGC; however, this can lead to unwanted side effects such as cell toxicity.

Accordingly, there is a need for an easily adaptable expression system that produces strong expression of a desired gene or genes and subsequent target compound without being toxic to the host cell. The present invention satisfies these needs.

### SUMMARY

The present disclosure reports the development of a robust fungal NP heterologous expression platform in the fungal model organism A. nidulans. The chassis strains used are nKuA and stc BCG null mutants and engineered so that afoA, the positive activator of the afo gene cluster, is under the control of the inducible promoter PalcA. It is shown that the refactored BGCs under the regulation of afo transcriptional regulatory sequences produced the target compounds in good to high yield and purity under PalcA inducing condition.

Compared to the existing fungal expression systems developed in A. oryzae and A. nidulans, there are several advantages of the present platform. The DNA fragments used for transformation were made by Gibson assembly and PCR, bypassing bacterial DNA cloning and yeast assembly. DNA fragments were generated as large as 9.2 kb (as in the case of plu-F1) in this way. The large DNA fragments were then assembled in vivo via HR with high efficiency in the A. nidulans nKuAΔ strains, allowing the simultaneous integration of multiple genes in one transformation, in contrast to the sequential addition of genes through iterative gene targeting. Applicants demonstrated the assembly of three large DNA fragments by HR, but this strategy will work with even more fragments such that a heterologous BGC of <35 kb could be assembled in vivo with four large DNA fragments (FIG. 2) in one transformation, and introduction of even larger BGCs could be possible with optimization of the transformation process. Thus, the Gibson-assembly-HR approach has the potential to greatly expedite pathway refactoring compared to conventional methods.

Since the afo promoters are co-regulated by afoA, concerted expression of all the GOIs can be elicited by one inducer in one step. While multiple copies of the same inducible promoter can be integrated into the genome, the chances of unwanted deletions caused by HR increases with the number of identical copies. The disclosed system also bypasses the process of screening for sequence-divergent promoters with sufficient expression levels by using a set of promoters fine-tuned for metabolite expression by nature. Additionally, since high expression levels do not always translate into high compound yield, the employment of a robust secondary metabolism transcriptional machinery may provide the optimum environment for the biosynthesis of our target molecules. Also, targeted GOIs are inserted into a defined locus, which circumvents the positional effects of genes integrated into different chromosomal loci and allows further strain engineering to be designed more rationally. Lastly, the well-established efficient gene targeting system and well-understood metabolite background in A. nidulans render subsequent strain engineering for titer improvement or combinatorial biosynthesis relatively simple. The goal is to engineer “microbial factory” strains that produce high-value fungal NPs with high yield and high purity. This “one strain one compound” approach will greatly simplify downstream purification and, therefore, lower the cost of production.

Another application of the disclosure is the elucidation of cryptic biosynthesis pathways. Given that most fungi lack genetic tools for cluster manipulation, heterologous expression is perhaps the most universal solution to accessing molecules from silent or cryptic BGCs. Although the afo regulon only accommodates seven genes, two other BGCs in A. nidulans, mdp (8 non-regulatory genes) and apd (6 non-regulatory genes), also contain a positive activator and produce good yields upon activation. Therefore, biosynthetic pathways with more than seven genes can be additionally refactored with the mpd or apd activator elements with the same approach as with afo. Given the relative ease of refactoring and constructing a biosynthetic pathway in A. nidulans with our platform, the question now becomes how to prioritize the vast number of fungal BGCs so that the most valuable biosynthetic dark matter can be brought to light.

Accordingly, the present disclosure generally provides for methods of producing a target compound in a host cell comprising: a) amplifying i) one or more polynucleotide sequences from a first target sequence, the first target sequence comprising one or more genes of an exogenous biosynthetic gene cluster for producing the target compound, and ii) amplifying one or more polynucleotide sequences from a second target sequence, the second target sequence comprising one or more intergenic regions of an endogenous biosynthetic gene cluster of the host cell, wherein the one or more intergenic regions comprise a promoter sequence for at least one gene of the endogenous biosynthetic gene cluster, and wherein the promoter sequence is controlled by a positive activator protein; b) assembling the amplified one or more polynucleotide sequences of the first target sequence and the amplified one or more polynucleotide sequences of the second target sequence in vitro to provide assembled sequences; c) using the assembled sequences as a template for a second amplification step to produce one or more final polynucleotide sequences; and d) transforming the one or more final polynucleotide sequences into the host cell wherein the one or more final polynucleotide sequences induce one or more homologous recombination events at an integration site of the host cell, wherein expression of one or more genes of the one or more final polynucleotide sequences causes production of the target compound.

In some embodiments, the host cell is a species of Aspergillus fungi selected from the group consisting of Aspergillus nidulans, Aspergillus fumigatus, Aspergillus oryzae, Aspergillus clavatus, Aspergillus flavus, Aspergillus niger, Aspergillus terreus, and Aspergillus sojae.

In some embodiments, the one or more intergenic regions of the endogenous biosynthetic gene cluster comprise intergenic regions of the afo biosynthetic gene cluster or the mdp biosynthetic gene cluster of Aspergillus nidulans. In some embodiments, the one or more intergenic regions of the afo biosynthetic gene cluster is at least about 85% identical to one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15 and/or the one or more intergenic regions of the mdp biosynthetic gene cluster is at least about 85% identical to one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64.

In some embodiments, a polynucleotide sequence of the positive activator protein is operably linked to an inducible or a constitutive promoter. Preferably, the inducible promoter comprises the PalcA promoter sequence, and the polynucleotide sequence of the positive activator protein comprises the polynucleotide sequence of afoA, the polynucleotide sequence of mdpE, or a combination thereof.

In some embodiments, the assembling step comprises Gibson assembly of the amplified one or more polynucleotide sequences of the first target sequence and the amplified one or more polynucleotide sequences of the second target sequence.

In some embodiments, the exogenous biosynthetic gene cluster comprises citreoviridin, mutilin, pleuromutilin, or fumagillin.

In some embodiments, the integration site is one or more of an afo biosynthetic gene cluster and an mdp biosynthetic gene cluster of Aspergillus nidulans.

The disclosure also provides for a transgenic Aspergillus nidulans cell for producing a target compound comprising: a recombinant biosynthetic pathway comprising: one or more genes of an exogenous biosynthetic gene cluster operably linked to a polynucleotide sequence of an intergenic region of a gene of an endogenous asperfuranone (afo) gene cluster and/or a gene of an endogenous monodictyphenone (mdp) gene cluster, wherein the intergenic region comprise a promoter sequence of the gene of the endogenous afo gene cluster and/or the endogenous mdp gene cluster; and a gene encoding a positive activator protein operably linked to an inducible promoter sequence wherein the positive activator protein is configured to bind to the promoter sequence of the gene of the endogenous afo gene cluster and/or the endogenous mdp gene cluster, thereby enabling expression of the one or more genes of the exogenous biosynthetic gene cluster and production of a target compound.

In some embodiments of a transgenic Aspergillus nidulans cell, the gene encoding the positive activator protein is afoA, mdpE, or a combination thereof.

In some embodiments, the polynucleotide sequence of the intergenic region of a gene of the endogenous afo gene cluster comprises one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15.

In other embodiments, the polynucleotide sequence of the intergenic region of a gene of the endogenous the mdp gene cluster comprises one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64

In some embodiments, the exogenous biosynthetic gene cluster comprises a citreoviridin biosynthetic gene cluster, a mutilin biosynthetic gene cluster, pleuromutilin gene cluster, or a fumagillin biosynthetic gene cluster.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Biosynthesis of asperfuranone in A. nidulans. (a) Gene organization of the afo regulon in chromosome VIII. AN1029 (afoA) is the positive activator of the afo regulon. All afo genes are transcribed by their own promoters, which are under the regulation of afoA. The insertion of the inducible alcA promoter (PalcA) into the 5′ region of afoA generated the strain YM47. Induction of PalcA drives the expression of AfoA, which then activates the afo cluster (AN1036-AN1030), leading to the production of asperfuranone. pyrG is an auxotrophic selection cassette. (b) The biosynthesis of asperfuranone and its intermediates.

FIG. 2. Homologous recombination (HR) among the large foreign DNA fragments (gray) and the chromosome (black) during a transformation in an A. nidulans nkuAΔ strain. Assuming that DNA fragments are 10 kb in size and flanking regions for HR are 1 kb, (a) two DNA fragments with 3 HR events will insert 17 kb of foreign DNA, (b) three DNA fragments with 4 HR events will insert 26 kb of foreign DNA, and (c) four DNA fragments with 5 HR events will insert 35 kb of foreign DNA.

FIG. 3. Reconstitution of the citreoviridin biosynthetic pathway in the afo regulon. (a) The biosynthesis of citreoviridin (1). (b) HR among three large DNA fragments (ctvF1-F3) and the afo locus of the recipient strain (YM87) reconstitutes the ctv genes in the afo regulon (YM192) so that the coding sequences of AN1036-AN1032 were replaced by ctvA-D, and the pyrG cassette, respectively. Schematic representation of the comparison between YM192 and YM81 (asperfuranone producing strain, FIG. 6). Gray boxes in between indicated the location of identical DNA sequences. (c) HPLC profiles (400 nm) of the culture media from strains YM87 and YM192.

FIG. 4. Reconstitution of the pleuromutilin biosynthetic pathway in the afo regulon. (a) The biosynthesis of mutilin (2) and pleuromutilin (3). (b) HR among two large DNA fragments (pluF1 and pluF2) and the afo locus of the recipient strain (YM137) reconstitutes the five pl genes in the afo regulon (YM283) so that the coding sequences of AN1036-AN1031 were replaced by the cDNA sequences of Pl-ggs, cyc, p450-1, p450-2, sdr, and the pyroA cassette, respectively. Schematic representation of the comparison between YM283 and YM81 (asperfuranone producing strain, FIG. 6). Gray boxes in between indicated the location of identical DNA sequences. The pyroA cassette is placed at pluF2. (c) HR between pluF3 and the afo locus of the recipient strain (YM283) reconstitutes the additional two pl genes in the afo regulon (YM343) so that the coding sequences of AN1036-AN1030 were replaced by the cDNA sequences of Pl-ggs, cyc, p450-1, p450-2, sdr, atf, and p450-3, respectively. Schematic representation of the comparison between YM343 and YM81. The pyrG cassette is located at 5′ of the PalcA. (d) MS total ion current (TIC) profiles of culture media from strains YM283 and YM343.

FIG. 5. Four DNA regions that have identical sequences between the DNA fragment pluF3 and the afo locus of the recipient strain (YM283).

FIG. 6. The procedure of creating the recipient strains YM87 and YM137 used for reconstituting the citreoviridin (1) and mutilin (2) biosynthesis pathways, respectively. Replacing the native promoter of AN1029 in L04389 with PalcA and the pyrG auxotrophic marker generated YM47. Marker recycling of pyrG in YM47 with 5-FOA generated YM81. Deletion of AN1036-AN1032 in YM81 with riboB auxotrophic marker generated YM87. Deletion of AN1036-AN1031 in YM81 with riboB auxotrophic marker generated YM137. Genotypes of the strains created in this study are listed in Table 5. Primer sets for generating transformation DNA cassettes are listed in Table 6.

FIG. 7. Gel images of PCR products used in the construction of the citreoviridin pathway in the afo locus. (a) The gel image of DNA marker used and the gene organization of the afo locus in the strain YM192. (b) Intergenic regions of the afo locus were amplified from gDNA of strain LO4389. Coding regions of ctvA-ctvD were amplified from gDNA of A. terrus var. aureus. M: marker, Lanes 1: 1036P (1487 bp), 2: ctvA (7527+50 bp), 3: 1036T (1768 bp), 4: ctvB (687+50 bp), 5: 1035P (527 bp), 6: ctvC (1611+50 bp), 7: 1034P (849 bp), 8: ctvD (1132+50 bp), 9: 1033P (605 bp), 10: pyrG cassette (1885+50 bp), and 11: 1031P-partialAN1031 (1145 bp). (c) PCR products of large fragments amplified from Gibson assembly. M: marker, Lanes 1: ctvF1 (6935 bp, amplified from 1036P and ctvA assembly), 2: ctvF2 (7479 bp, amplified from ctvA, 1036T, ctvB, 1035P, ctvC, and 1034P assembly), and 3: ctvF3 (6926 bp, amplified from ctvC, 1034P, ctvD, 1033P, pyrG cassette, and 1031P-partialAN1031 assembly). (d) Diagnostic PCR of strains YM186-YM195 (lanes 1 to 10). The locations of primer sets used are shown at the top of the figure. From top to bottom, PCR products from primer set 1 (2701 bp), set 2 (3242 bp), set 3, (2345 bp), and set 4 (2199 bp). Primers used are listed in Table 6.

FIG. 8. Gel images of PCR products used in the construction of the mutilin pathway in the afo locus. (a) The gel image of DNA marker used and the gene organization of the afo locus in the strain YM283. (b) Intergenic regions of afo locus were amplified from gDNA of strain LO4389. Coding regions of pl-ggs, pl-cyc, pl-p450-1, pl-450-2, and pl-sdr were amplified from cDNA of C. passeckerianus. M: marker, Lanes 1: pl-ggs (1053+50 bp), 2: pl-cyc (2880+50 bp), 3: pl-p450-1 (1572+50 bp), 4: pl-450-2 (1578+50 bp), 5: pl-sdr (762+50 bp), 6: pyroA cassette (2088+50 bp), and 7: 1031T-partial AN1030 (1341 bp). (c) PCR products of large fragments amplified from Gibson assembly. M: marker, Lanes 1: pluF1 (9224 bp, amplified form 1036P, pl-ggs, 1036T, pl-cyc, 1035P, pl-p450-1 and 1034P assembly) and 2: pluF2 (8227 bp, amplified from pl-p450-1, 1034P, pl-p450-2, 1033P, pl-sdr, 1031P, pyroA cassette, and 1031T-partialAN1030 assembly) (d) Diagnostic PCR of strains YM283-YM287 (lanes 2 to 6) and the recipient strain (YM137, lane 1) as negative control. The location of primer sets used are shown at the top of the figure. From top to bottom, PCR products from primer set 1 (10136 bp) and set 2 (9500 bp). Primers used are listed in Table 6.

FIG. 9. Gel images of PCR products used in the construction of the pleuromutilin pathway in the afo locus. (a) The gel image of DNA marker used and the gene organization of the afo locus in the strain YM343. (b) Intergenic regions of afo locus were amplified from gDNA of strain L04389. Coding regions of pl-atf and pl-p450-3 were amplified from cDNA of C. passeckerianus. The sdr-1031P fragment was amplified from the recipient strain YM283. M: marker, Lanes 1: sdr-1031P fragment (1146 bp), 2: pl-atf (1134+50 bp), 3: 1031T (591 bp), 4: pl-450-3 (1569+50 bp), 5: 1029P (1370 bp), and 6: pyrG cassette-PalcA-partial AN1029 (3395+25 bp). (c) PCR products of large fragments amplified from Gibson assembly. M: marker, Lanes 1: pluF3 (8900 bp, amplified from sdr-1031P fragment, pl-atf, 1031T, pl-450-3, 1029P, and pyrG cassette-PalcA-partial AN1029 assembly). (d) Two other possible HR transformations (see FIG. 5). HR between DNA regions 2 and 4, or 3 and 4 will create strains without recycling of the pyroA cassette which can grow on an agar plate without pyridoxine. (e) Diagnostic PCR of strains YM343-YM357 (lanes 1 to 15) and the recipient strain (YM283, lane R). The sizes of PCR products from the recipient strain YM283, HR between DNA regions 1 and 4, 2 and 4, and 3 and 4 are 7774, 9205, 10109, and 9808 bp, respectively. Strains YM343 (lane 1), YM344 (lane 2), YM346 (lane 4), YM347 (lane 5), YM350 (lane 8), YM352 (lane 10), YM355 (lane 13), and YM357 (lane 15) require pyridoxine to grow and to have the correct size of diagnostic PCR products.

FIG. 10. Biosynthesis of fumagillin in A. fumigatus. (a) Gene organization of the fma gene cluster in chromosome VIII of A. fumigatus. (b) The biosynthetic pathway of fumagillin.

FIG. 11. Replacing the coding sequences of the afo and mdp clusters with the coding sequences of genes involved in the fumagillin biosynthesis creates an A. nidulans strain YM727 that produces fumagillin. (a) Seven genes from A. fumigatus (fma-TC, P450, C6H, MT, KR, afCPR, and fix/II) were incorporated into the afo regulon. (b) Three genes (fma-AT, PKS, and ABM) were incorporated into the mdp regulon. PyrG is a nutritional marker used for selecting the correct transformants. The pyrG marker has been recycled in the fma-AT, PKS, and ABM heterologous expression stain.

FIG. 12. Biosynthesis of monodictyphenone in A. nidulans. (a) Gene organization of the mdp gene cluster in chromosome VIII of A. nidulans. After replacing the native promoter of AN0148 (mdpE) with the inducible promoter PalcA, the expression of mdpE is under the control of PalcA. PyrG encodes orotidine-5′-phosphate decarboxylase and is a nutritional marker used for selecting the correct transformants. Induction of mdpE expression resulted in the expression of genes in the mdp cluster and the production of monodictyphenone. (b) The biosynthetic pathway of monodictyphenone.

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, PCR amplification, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of or “consisting essentially of are used instead. As used herein, “comprising” is synonymous with “including,” “containing,” or “characterized by,” and is inclusive or open-ended and does not exclude additional, unrecited elements or method steps. As used herein, “consisting of excludes any element, step, or ingredient not specified in the aspect element. As used herein, “consisting essentially of does not exclude materials or steps that do not materially affect the basic and novel characteristics of the aspect. In each instance herein any of the terms “comprising”, “consisting essentially of and “consisting of may be replaced with either of the other two terms. The disclosure illustratively described herein may be suitably practiced in the absence of any element or elements, limitation, or limitations not specifically disclosed herein.

The term “genome” or “genomic DNA” is referring to the heritable genetic information of a host organism. Said genomic DNA comprises the entire genetic material of a cell or an organism, including the DNA of the bacterial chromosome and plasmids for prokaryotic organisms and includes for eukaryotic organisms the DNA of the nucleus (chromosomal DNA), extrachromosomal DNA, and organellar DNA (e.g., of mitochondria). Preferably, the terms genome or genomic DNA is referring to the chromosomal DNA of the nucleus.

The term “chromosomal DNA” or “chromosomal DNA sequence” in the context of eukaryotic cells is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), in situ PCR and next generation sequencing (NGS).

The term “promoter” refers to a polynucleotide which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent, if the promoter is a constitutive promoter. The term “enhancer” refers to a polynucleotide. An enhancer can increase the efficiency with which a particular gene is transcribed into mRNA irrespective of the distance or orientation of the enhancer relative to the start site of transcription. Usually, an enhancer is located close to a promoter, a 5′-untranslated sequence or in an intron.

“Transgene”, “transgenic” or “recombinant” refers to a polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill will recognize that polynucleotides can be manipulated in many ways and are not limited to the examples above.

In case the term “recombinant” is used to specify an organism or cell, e.g., a microorganism, it is used to express that the organism or cell comprises at least one “transgene”, “transgenic” or “recombinant” polynucleotide, which is usually specified later on.

The terms “heterologous” or “exogenous” refer to a polynucleotide or amino acid sequence that originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e. g. a genetically engineered coding sequence or an allele from a different ecotype or variety).

Reference herein to an “endogenous” gene not only refers to the gene in question as found in an organism in its natural form (i.e., without there being any human intervention), but also refers to that same gene (or a substantially homologous nucleic acid/gene) in an isolated form subsequently (re)introduced into a microorganism (a transgene). For example, a transgenic microorganism containing such a transgene may encounter a substantial reduction of the transgene expression and/or substantial reduction of expression of the endogenous gene. The isolated gene may be isolated from an organism or may be manmade, for example by chemical synthesis.

The terms “orthologues” and “paralogues” encompass evolutionary concepts used to describe the ancestral relationships of genes. Paralogues are genes within the same species that have originated through duplication of an ancestral gene; orthologues are genes from different organisms that have originated through speciation and are also derived from a common ancestral gene.

The terms “operable linkage” or “operably linked” are generally understood as meaning an arrangement in which a genetic control sequence, e.g., a promoter, enhancer or terminator, is capable of exerting its function with regard to a polynucleotide being operably linked to it, for example a polynucleotide encoding a polypeptide. Function, in this context, may mean for example control of the expression, i.e., transcription and/or translation, of the nucleic acid sequence. Control, in this context, encompasses for example initiating, increasing, governing or suppressing the expression, i.e., transcription and, if appropriate, translation. Controlling, in turn, may be, for example, tissue- and/or time-specific. It may also be inducible, for example by certain chemicals, stress, pathogens and the like. Preferably, operable linkage is understood as meaning for example the sequential arrangement of a promoter, of the nucleic acid sequence to be expressed and, if appropriate, further regulatory elements such as, for example, a terminator, in such a way that each of the regulatory elements can fulfill its function when the nucleic acid sequence is expressed. An operably linkage does not necessarily require a direct linkage in the chemical sense. For example, genetic control sequences like enhancer sequences are also capable of exerting their function on the target sequence from positions located at a distance to the polynucleotide, which is operably linked. Preferred arrangements are those in which the nucleic acid sequence to be expressed is positioned after a sequence acting as promoter so that the two sequences are linked covalently to one another. The distance between the promoter and the amino acid sequence encoding polynucleotide in an expression cassette, is preferably less than 200 base pairs, especially preferably less than 100 base pairs, very especially preferably less than 50 base pairs. The skilled worker is familiar with a variety of ways in order to obtain such an expression cassette. However, an expression cassette may also be constructed in such a way that the nucleic acid sequence to be expressed is brought under the control of an endogenous genetic control element, for example an endogenous promoter, for example by means of homologous recombination or else by random insertion. Such constructs are likewise understood as being expression cassettes for the purposes of the invention.

The term “expression cassette” means those constructs in which the nucleic acid sequence encoding an amino acid sequence to be expressed is linked operably to at least one genetic control element which enables or regulates its expression (i.e., transcription and/or translation). The expression may be, for example, stable or transient, constitutive or inducible.

The terms “express,” “expressing,” “expressed” and “expression” refer to expression of a gene product (e.g., a biosynthetic enzyme of a gene of a pathway or reaction defined and described in this application) at a level that the resulting enzyme activity of this protein encoded for or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

In some embodiments, a microorganism can be physically or environmentally altered to express a gene product at an increased or lower level relative to level of expression of the gene product unaltered microorganism. For example, a microorganism can be treated with, or cultured in the presence of an agent known, or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

The term “motif or “consensus sequence” or “signature” refers to a short, conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res30, 242-244), InterPro (Mulder et al., (2003) Nucl. Acids. Res. 31, 315-318), Prosite (Bucher and Bairoch (1994) (In) ISMB-94; Proceedings 2nd International Conference on Intelligent Systems for Molecular Biology. Altman et al., Eds., pp53-61, AAAI Press, Menlo Park; Hulo et al., Nucl. Acids. Res. 32:D134-D137, (2004)), or Pfam (Bateman et al., Nucleic Acids Research 30(1): 276-280 (2002); Finn et al., Nucleic Acids Research (2010) Database Issue 38:D21 1-222). A set of tools for in silico analysis of protein sequences is available on the ExPASy proteomics server (Swiss Institute of Bioinformatics (Gasteiger et al., Nucleic Acids Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e., spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimize alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

Typically, this involves a first BLAST involving BLASTing a query sequence against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTS are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits. High-ranking hits are those having a low E-value. The lower the E-value, the more significant the score (or in other words the lower the chance that the hit was found by chance).

Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbor joining tree, to help visualize clustering of related genes and to identify orthologues and paralogues.

The term “sequence identity” between two nucleic acid sequences is understood as meaning the percent identity of the nucleic acid sequence over in each case the entire sequence length which is calculated by alignment with the aid of the program algorithm GAP (Wisconsin Package Version 10.0, University of Wisconsin, Genetics Computer Group (GCG), Madison, USA), setting, for example, the following parameters: Gap Weight: 12 Length Weight: 4; Average Match: 2,912 Average Mismatch: −2,003.

The term “sequence identity” between two amino acid sequences is understood as meaning the percent identity of the amino acids sequence over in each case the entire sequence length which is calculated by alignment with the aid of the program algorithm GAP (Wisconsin Package Version 10.0, University of Wisconsin, Genetics Computer Group (GCG), Madison, USA), setting, for example, the following parameters: Gap Weight: 8; Length Weight: 2; Average Match: 2,912; Average Mismatch: −2,003.

The term “hybridization” as defined herein is a process wherein substantially homologous complementary nucleotide sequences anneal to each other. The hybridization process can occur entirely in solution, i.e., both complementary nucleic acids are in solution. The hybridization process can also occur with one of the complementary nucleic acids immobilized to a matrix such as magnetic beads, Sepharose beads or any other resin. The hybridization process can furthermore occur with one of the complementary nucleic acids immobilized to a solid support such as a nitro-cellulose or nylon membrane or immobilized by e.g., photolithography to, for example, a siliceous glass support (the latter known as nucleic acid arrays or microarrays or as nucleic acid chips). In order to allow hybridization to occur, the nucleic acid molecules are generally thermally or chemically denatured to melt a double strand into two single strands and/or to remove hairpins or other secondary structures from single stranded nucleic acids.

The term “stringency” refers to the conditions under which a hybridization takes place. The stringency of hybridization is influenced by conditions such as temperature, salt concentration, ionic strength and hybridization buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridization conditions are typically used for isolating hybridizing sequences that have high sequence similarity to the target nucleic acid sequence. However, nucleic acids may deviate in sequence and still encode a substantially identical polypeptide, due to the degeneracy of the genetic code. Therefore, medium stringency hybridization conditions may sometimes be needed to identify such nucleic acid molecules.

The Tₘ is the temperature under defined ionic strength and pH, at which 50% of the target sequence hybridizes to a perfectly matched probe. The Tₘ is dependent upon the solution conditions and the base composition and length of the probe. For example, longer sequences hybridize specifically at higher temperatures. The maximum rate of hybridization is obtained from about 16° C. up to 32° C. below Tₘ. The presence of monovalent cations in the hybridization solution reduces the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridization to be performed at 30 to 45° C., though the rate of hybridization will be lowered. Base pair mismatches reduce the hybridization rate and the thermal stability of the duplexes. On average and for large probes, the Tₘ decreases about 1° C. per % base mismatch. The Tₘ may be calculated using the following equations, depending on the types of hybrids:
1) DNA-DNA hybrids (Meinkoth and Wahl, Anal. Biochem., 138: 267-284, 1984):
   Tₘ=81.5° C. +16.6xlog io[Na⁺]^{a}+0.41x %[G/C^{b}]−500x[L^{c}]−¹−0.61x % formamide
2) DNA-RNA or RNA-RNA hybrids:
   Tₘ=79.8° C.+18.5 (log io[Na⁺]^{a})+0.58 (% G/C^{b})+11.8 (% G/C^{b})²−820/L^{c}
3) oligo-DNA or oligo-RNA^{d} hybrids:
   For <20 nucleotides: Tₘ=2 (lₙ)
   For 20-35 nucleotides: Tₘ=22+1 0.46 (lₙ)
a or for other monovalent cation, but only accurate in the 0.01-0.4 M range.
b only accurate for % GC in the 30% to 75% range.
c L=length of duplex in base pairs.
d oligo, oligonucleotide; in, =effective length of primer=2x(no. of G/C)+(no. of A/T).

Non-specific binding may be controlled using any one of a number of known techniques such as, for example, blocking the membrane with protein containing solutions, additions of heterologous RNA, DNA, and SDS to the hybridization buffer, and treatment with RNAse. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridization and which will either maintain or change the stringency conditions.

Besides the hybridization conditions, specificity of hybridization typically also depends on the function of post-hybridization washes. To remove background resulting from non-specific hybridization, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridization stringency. A positive hybridization gives a signal that is at least twice of that of the background. Generally, suitable stringent conditions for nucleic acid hybridization assays or gene amplification detection procedures are as set forth above. More or less stringent conditions may also be selected. The skilled artisan is aware of various parameters which may be altered during washing and which will either maintain or change the stringency conditions.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. The length of the hybrid is the anticipated length for the hybridizing nucleic acid. When nucleic acids of known sequence are hybridized, the hybrid length may be determined by aligning the sequences and identifying the conserved regions described herein. 1×SSC is 0.15M NaCl and 15 mM sodium citrate; the hybridization solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A “deletion” refers to removal of one or more amino acids from a protein.

An “insertion” refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag^{«}100 epitope, c-myc epitope, FLAG®-epitope, lacZ, CMP (calmodulin-binding peptide), HA epitope, protein C epitope and VSV epitope.

A “substitution” refers to replacement of amino acids of the protein with other amino acids having similar properties (such as similar hydrophobicity, hydrophilicity, antigenicity, propensity to form or break a-helical structures or 3-sheet structures). Amino acid substitutions are typically of single residues but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds).

The term “vector”, preferably, encompasses phage, plasmid, fosmid, viral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a recombinant microorganism. The vector may be incorporated into a recombinant microorganism by various techniques well known in the art. If introduced into a recombinant microorganism, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques.

The terms “transformation” and “transfection”, conjugation and transduction, as used in the present context, are intended to comprise a multiplicity of prior-art processes for introducing foreign nucleic acid (for example DNA) into a recombinant microorganism, including calcium phosphate, rubidium chloride or calcium chloride co-precipitation, DEAE-dextran-mediated transfection, lipofection, natural competence, carbon-based clusters, chemically mediated transfer, electroporation or particle bombardment. Methods for many species of microorganisms are readily available in the literature.

A “gene cluster” or “regulon” may commonly refer to a group of genes building a functional unit. As used herein, a “gene cluster” is a nucleic acid comprising sequences encoding for polypeptides that are involved together in at least one biosynthetic pathway, preferably in one biosynthetic pathway. Particularly, said sequences are adjacent. Preferably, said sequences directly follow each other, wherein they are separated by varying amounts of non-coding DNA. Preferably, a gene cluster of the invention has a size from 10 kb to 50 kb, more preferably from 14 kb to 40 kb, even more preferably from 15 kb to 35 kb, even more preferably from 20 kb to 30 kb, particularly from 23 kb to 28 kb.

### Embodiments of the Invention

The present disclosure describes a complete biosynthetic gene cluster (BCG) refactoring strategy and heterologous expression platform in A. nidulans based on the replacement of endogenous inducible biosynthetic pathway regulons, and in particular, the asperfuranone (afo) and monodictyphenone (mdp) regulons, with a biosynthetic gene cluster of interest. Although the afo and mdp regulons are discussed in detail, other transcriptionally regulated biosynthetic gene clusters may be used if transcription of the BCG is controlled by a positive regulator (such as AfoA and MdpE for the afo and mdp regulons, respectively).

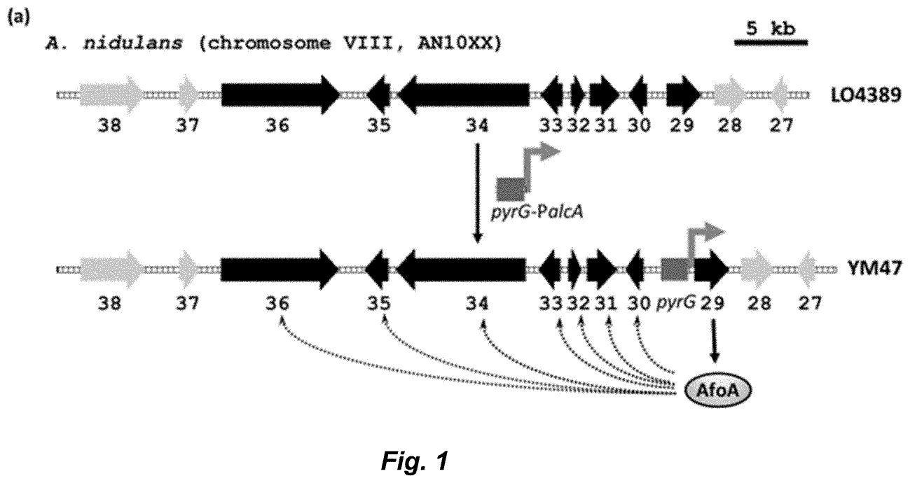

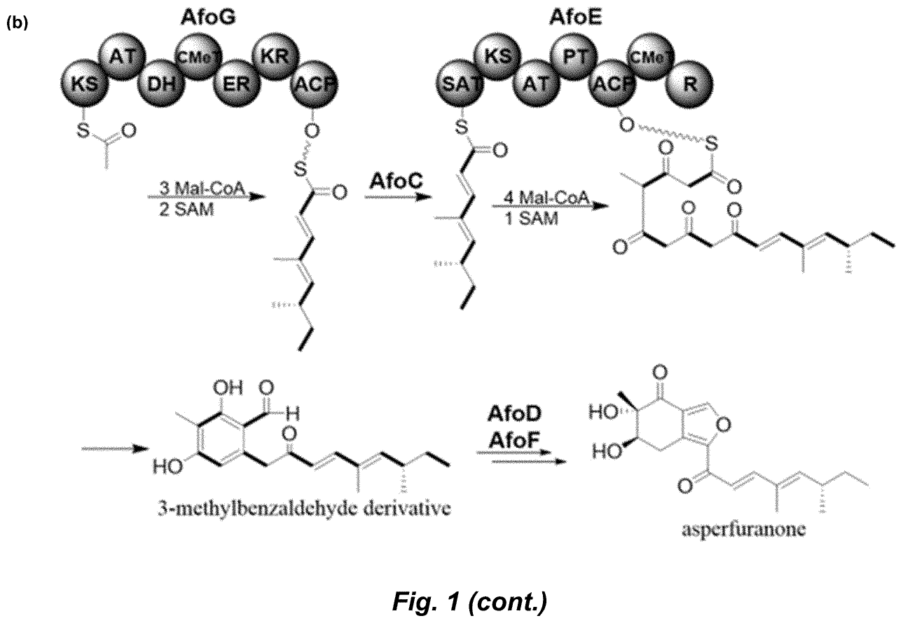

In the afo regulon, induction of AfoA, the pathway-specific transcription activator, led to the concerted expression of all the afo genes and the robust production of asperfuranone and its intermediate (FIG. 1, Table 1). Taking advantage of the transcriptional regulatory elements of afo, afo genes were replaced with genes of interest (GOIs) from a target BGC. Induction of afoA would thus result in the specific activation of our refactored BGC and production of the encoded molecule, which, is hypothesized, would be in similar abundance as asperfuranone and its intermediate. Advantageously, embodiments of the disclosure provide cloning-free and generates compound-producing strains rapidly. The host is easily amendable to subsequent titer optimization or genetic dereplication.

TABLE 1
Sizes and putative functions of genes identified in the afo cluster.

| | Gene Size | Putative |
|---|---|---|
| Gene Name | (base pairs) | Function |
| AN1029 (afoA) | 2345 | Positive regulator |
| AN1030 | 1218 | Dehydrogenase |
| AN1031 (afoB) | 2033 | Efflux pump |
| AN1032 (afoC) | 894 | Esterase/lipase |
| AN1033 (afoD) | 1452 | Salicylate monooxygenase |
| AN1034 (afoE) | 8931 | NR-PKS |
| AN1035 (afoF) | 1593 | FAD-dependent oxygenase |
| AN1036 (afoG) | 8049 | HR-PKS |

Accordingly, the disclosure provides for, inter alia, methods of producing a recombinant host cell expression system. In particular, the disclosure provides for methods of expressing a exogenous biosynthetic gene cluster or portions thereof in a non-native host to produce a target compound comprising a) amplifying i) one or more polynucleotide sequences from a first target sequence, the first target sequence comprising a coding sequence of one or more genes of an exogenous biosynthetic gene cluster for producing the target compound, and ii) amplifying one or more polynucleotide sequences from a second target sequence, the second target sequence comprising one or more intergenic regions of an endogenous biosynthetic gene cluster of the host cell, wherein the one or more intergenic regions comprise a promoter sequence for at least one gene of the endogenous biosynthetic gene cluster, and wherein the promoter sequence is controlled by a positive activator protein; b) assembling the amplified one or more polynucleotide sequences of the first target sequence and the amplified one or more polynucleotide sequences of the second target sequence in vitro to provide assembled sequences; c) using the assembled sequences as a template for a second amplification step to produce one or more final polynucleotide sequences; and d) transforming the one or more final polynucleotide sequences into the host cell wherein the one or more final polynucleotide sequences induce one or more homologous recombination events at an integration site of the host cell, wherein expression of one or more genes of the one or more final polynucleotide sequences causes production of the target compound.

In another embodiment, a method of expressing a exogenous biosynthetic gene cluster or portions thereof in a non-native host cell to produce a target compound comprises the steps of a) amplifying i) one or more polynucleotide sequences from a first target sequence, the first target sequence comprising one or more genes of an exogenous biosynthetic gene cluster for producing the target compound, and ii) amplifying one or more polynucleotide sequences from a second target sequence, the second target sequence comprising one or more intergenic regions of an endogenous biosynthetic gene cluster of the host cell, wherein the one or more intergenic regions comprise a promoter sequence for at least one gene of the endogenous biosynthetic gene cluster, and wherein the promoter sequence is controlled by a positive activator protein; b) purifying the amplified polynucleotide sequences of the first target sequence and the amplified polynucleotide sequences of the second target sequence; c) assembling the amplified polynucleotide sequences of the first target sequence and the amplified polynucleotide sequences of the second target sequence in vitro to provide assembled sequences; d) isolating the assembled sequences; e) using the assembled sequences as a template for a second amplification step to produce one or more final polynucleotide sequences; and f) transforming the one or more final polynucleotide sequences into the host cell wherein the one or more final polynucleotide sequences induce one or more homologous recombination events at an integration site of the host cell, wherein expression of one or more genes of the one or more final polynucleotide sequences causes production of the target compound. The biosynthetic gene clusters comprise nucleic acid sequences that encode enzymatic pathways that enable the production of the target compound.

In some embodiments, the host cell is a species of Aspergillus. Species of Aspergillus include Aspergillus nidulans, Aspergillus fumigatus, Aspergillus oryzae, Aspergillus clavatus, Aspergillus flavus, Aspergillus niger, Aspergillus terreus, or Aspergillus sojae. In preferred embodiments, the host cell is Aspergillus nidulans.

In some embodiments, the first target sequences comprise one or more genes of an exogenous biosynthetic gene cluster. In some embodiments, the exogenous biosynthetic gene clusters originate from a mammal, a plant, a fungus, or a bacterium.

In some embodiments, the first target sequences comprise the coding sequences of all the genes of the exogenous biosynthetic gene cluster necessary to produce a target compound. In some embodiments, the exogenous biosynthetic gene cluster inserted into the host cell comprises the citreoviridin pathway (comprising at least the genes ctvA, ctvB, ctvC, and ctvD), the mutilin pathway (comprising at least the genes of Pl-ggs, cyc, p450-1, p450-2, and sdr), the pleuromutilin pathway (comprising at least the genes of Pl-ggs, cyc, p450-1, p450-2, sdr, atf, and p450-3), or the fumagillin pathway (comprising at least the genes of fma-TC, P450, C6H, MT, KR, afCPR, fpaII, fma-AT, PKS, and ABM).

Other biosynthetic pathways include, but are not limited to, the ergothioneine pathway for making ergothioneine comprising egt1 and egt2 genes from, for example, Neurospora crassa (Van der Hoek et al., Front Bioeng Biotechnol 2019, 7, 262); the atpenin pathway for making atpenin B comprising apnA, apnB, apnC, apnD, apnE, and apnG genes from, for example, Penicillium oxalicum (Bat-Erdene et al., J Am Chem Soc 2020, 142 (19), 8550-8554.); the beauveriolide pathway for making beauveriolides comprising cm3A, cm3B, cm3C, and cm3D genes from, for example, Cordyceps militaris (Wang et al., J Biotechnol 2020, 309, 85-91.); and the mycophenolic acid pathway for making mycophenolic acid comprising mpaA, mpaB, mpaC, mpaDE, and mpaG genes, from, for example, Penicillium brevicompactum (Regueira et al., Appl Environ Microbiol 2011, 77 (9), 3035-3043.) or Penicillium griseofulvum (Chen et al., Acta Pharm Sin B 2019, 9 (6), 1253-1258.). The nucleic acid sequences of the genes of the ergothioneine pathway, atpenin pathway, beauveriolide pathway, mycophenolic acid pathway may be found in known and publicly available databases such as, for example, the National Center for Bioinformatics Information database (www.ncbi.nlm.nih.gov/), the Fungal and Oomycete Informatics Resources database (www.fungidb.org), the Joint Genome Institute MycoCosm database (www.mycocosm.jgi.doe.gov). Also see Chiang et al., Journal of Natural Products 2022 85 (10), 2484-2518) and Klejnstrup et al., Metabolites 2012 March; 2(1): 100-133.

In some embodiments, the second target sequences comprise one or more intergenic regions of an endogenous biosynthetic gene cluster. Preferably, the intergenic regions include a promoter sequence that controls a gene of the endogenous biosynthetic pathway. Preferably the endogenous gene cluster includes 2, 3, 4, 5, 6, 7, 8, 9, 10, 11, 12, 13, 14, or 15 genes, wherein each gene is controlled by a promoter sequence positioned in the intergenic regions of the biosynthetic gene cluster. For example, the afo biosynthetic gene cluster comprises seven non-regulatory genes, each under transcriptional control of specific promoter sequence (i.e., seven unique promoter sequences). Thus, each of the seven intergenic regions comprising the seven unique promoter sequences may be operably linked to different gene from an exogenous biosynthetic gene cluster and inserted into the afo locus. Activation of the afo promoter sequences cause transcription of the exogenous genes and production of the target compound of interest. The mdp biosynthetic gene cluster comprises eight non-regulatory genes, each under transcriptional control of specific promoter sequence (i.e., eight unique promoter sequences). Thus, each of the eight intergenic regions comprising the eight unique promoter sequences may be operably linked to different gene from an exogenous biosynthetic gene cluster and inserted into the mdp locus. Activation of the mdp promoter sequences cause transcription of the exogenous genes and production of the target compound of interest.

As a simple example using the afo gene cluster, gene 1 and gene 2 of a gene cluster of interest is to be inserted into the host cell having the formula IR1-G1-IR2-G2 wherein IR-1 is a first intergenic region comprising a promoter sequence of a first gene of the afo gene cluster, G1 is gene 1, IR-2 is a second intergenic region comprising a promoter sequence of a second gene of the afo gene cluster, and G2 is gene 2.

Accordingly, in some embodiments, an exogenous biosynthetic gene cluster may be inserted into more than one endogenous gene clusters. For example, an exogenous gene cluster comprising eight or more genes may be divided, and part of the gene cluster (e.g., up to seven of the genes) inserted into the afo locus and the remaining genes inserted into the mdp locus. In this way, larger biosynthetic gene clusters may be inserted into the host cell. Thus, through the use of the afo and mdp gene clusters, an exogenous biosynthetic gene cluster of up to 15 genes may be inserted into the host cell. Alternately, the genes of an exogenous biosynthetic gene cluster may be divided equally between two or more endogenous loci. Other endogenous biosynthetic gene clusters may be used to increase the number of exogenous genes that may be inserted into the host cell. In one embodiment, the endogenous biosynthetic gene cluster is the aspyridone (apd) biosynthetic gene cluster (Bergmann et al., Nat Chem Biol 3, 213-217 (2007) comprising apdA (AN8412), apdB (AN8404), apdC (AN8409), apdD (AN8410), apdE (AN8411), apdF (AN8413), adpG (AN8415), and apdR (AN8414). The gene sequences and intergenic regions of the apd gene cluster can be found at www.fungidb.org/.

In some embodiments, the one or more intergenic regions of the afo biosynthetic gene cluster is about 80% identical, 85% identical, about 90% identical, about 95% identical, about 96% identical, about 97% identical, about 98% identical, about 99% identical, or identical to one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15.

In some embodiments, the one or more intergenic regions of the mdp biosynthetic gene cluster is about 80% identical, 85% identical, about 90% identical, about 95% identical, about 96% identical, about 97% identical, about 98% identical, about 99% identical, or identical to one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64.

In some embodiments, the host cell further comprises a gene encoding a positive activator protein that is operably linked to an inducible or a constitutive promoter. Contacting the host cell with an inducing agent causes induction of the inducible promoter and activates transcription of the operably linked gene. The positive activator protein is then produced and able to bind to an endogenous promoter to cause activation of said promoters. Inducible promoters for use with the invention are well known in the art and include, for example, the alcohol dehydrogenase I promoter (PalcA) % (Caddick et al., (1998) Nat. Biotechnol 16:177-180), the alcohol dehydrogenase III promoter (PalcC), the acetamidase promoter (PamdS), the α-amylase promoter (PamyB), the glucoamylase promoter (PglaA), the thiamine-dependent promoter (PthiA), the xylose-inducible promoter (PexlA), and the superoxide dismutase promoter (PsodM). Exemplary constitutive promoters include, for example, the alcohol dehydrogenase promoter (PadhA), the glyceraldehyde-3-phosphate dehydrogenase promoter (PgpdA), the ATP synthase promoter (PoliC), and the triosephosphate isomerase promoter (PtpiA) (see, for example, Kluge et al., Appl Microbiol Biotechnol. 2018; 102(15): 6357-6372; Waring et al., Gene. 1989 Jun. 30; 79(1):119-30). Preferred positive activator proteins may be determined by which target sequence the exogenous biosynthetic pathway genes are inserted. For example, if the exogenous biosynthetic pathway genes are inserted into the afo locus, then the preferred positive activator protein is AfoA, which is the positive activator protein of the afo locus. Other positive activator proteins include MdpE (encoded by the mdpE gene), which is the positive activator protein of the mdp locus, and ApdR (encoded by the apdR gene), which is the positive activator protein of the apd pathway.

In some embodiments, the inducible promoter is a PalcA promoter sequence operably linked to the afoA gene encoding the activator protein AfoA. In some embodiments, the inducible promoter is a PalcA promoter sequence operably linked to the mdpE gene encoding the positive activator protein MdpE. In another embodiment, the inducible promoter is a PalcA promoter sequence operably linked to one or more of the afoA gene encoding the positive activator protein AfoA and the mdpE gene encoding the positive activator protein MdpE. In other embodiments, the inducible promoter may be the same or different for each positive activator protein.

In some embodiments, the assembling step comprises the use of the technique known as Gibson assembly of the amplified target sequences or of the purified amplified target sequences as described in Gibson et al., Nat. Methods (2009) 6(5), 343-345.

Other cloning methods are known in the art and include, by way of non-limiting example, fusion PCR and assembly PCR (see, e.g. Stemmer et al. Gene 164(1): 49-53 (1995)), inverse fusion PCR (see, e.g. Spiliotis et al, PLoS ONE 7(4): 35407 (2012)), site directed mutagenesis (see, e.g. Ruvkun et al. Nature 289(5793): 85-88 (1981)), Quickchange (see, e.g. Kalnins et al. EMBO 2(4): 593-7 (1983)), Gateway (see, e.g. Hartley et al. Genome Res. 10(11):1788-95 (2000)), Golden Gate (see, e.g. Engler et al. Methods Mol Biol. 1116:119-31 (2014)), restriction digest and ligation including but not invited to blunt end, sticky end, and TA methods (see, e.g. Cohen et al. PNAS 70 (11): 3240-4 (1973)). Methods for integrating heterologous nucleic acid molecules into a host cell genome by techniques such as single- and double-crossover homologous recombination and the like are well known in the art (See for example, U.S. Pub. No. 2009/0124000 and International Pub. No. WO2009085135).

In some embodiments, the amplified target sequences may be purified and/or isolated using techniques known in the art. For example, in some embodiments, the purification step comprises gel purification of the amplified target sequences. Other methods, such as column purification of the use of commercially available purification kits are available and known in the art.

Transformation of the host cell may be conducted by any suitable known methods, including e.g., electroporation methods, particle bombardment or microprojectile bombardment, protoplast methods and Agrobacterium mediated transformation (AMT). In some embodiments, the protoplast method is used. Procedures for transformation are described, for example, by J. R. S. Fincham, Transformation in fungi. 1989, Microbiological reviews. 53, 148-170.

Transformation may involve a process consisting of protoplast formation, transformation of the protoplasts, and regeneration of the cell wall in a manner knownper se. Suitable procedures for transformation of Aspergillus cells are described in Boel et al., European patent App. No. EP 238023 and Yelton et al., 1984, Proceedings of the National Academy of Sciences USA 81:1470-1474. Suitable procedures for transformation of Aspergillus and other filamentous fungal host cells using Agrobacterium tumefaciens are described in e.g., De Groot et al., Nat Biotechnol. 1998, 16:839-842. Erratum in: Nat Biotechnol 1998 16:1074.

Typically, the cells transformed with the selectable marker can be selected based on the presence of the selectable marker. In case of transformation of (Aspergillus) cells, usually when the cell is transformed with all nucleic acid material at the same time, when the selectable marker is present also the polynucleotide(s) encoding the desired polypeptide(s) are present.

Selectable marker genes that can be used for transformation of most filamentous fungi and yeasts such as acetamidase genes or cDNAs (the amdS, niaD, facA genes or cDNAs from A. nidulans, A. oryzae or A. niger), or genes providing resistance to antibiotics like G418, hygromycin, bleomycin, kanamycin, methotrexate, phleomycin orbenomyl resistance (benA).

Alternatively, specific selection markers can be used such as auxotrophic markers which require corresponding mutant host strains: e.g., URA3 (from S. cerevisiae or analogous genes from other yeasts), pyrG or pyrA (from A. nidulans or A. niger), argB (from A. nidulans or A. niger) or trpC. Preferred for use in Aspergillus are the amdS (see for example Swinkels et al., U.S. Pub. Nos. 2004/0005692, 2003/0124707; Sagt et al., U.S. Pat. No. 2008/0070277, Swinkels et al., Int. Pub. No. WO1997/0006261; and Selten et al., U.S. Pat. No. 6,955,909) and the pyrG genes of A. oryzae and the bar gene of Streptomyces hygroscopicus. In some embodiments, the selection marker is deleted from the transformed host cell after introduction of the expression construct so as to obtain transformed host cells capable of producing the polypeptide which are free of selection marker genes.

Other markers include ATP synthetase, subunit 9 (oliC), orotidine-5′-phosphate decarboxylase (pvrA), the bacterial G418 resistance gene (this may also be used in yeast, but not in fungi), the ampicillin resistance gene (E. coli), the neomycin resistance gene (Bacillus) and the E. coli uidA gene, coding for β-glucuronidase (GUS). Vectors may be used in vitro, for example for the production of RNA or used to transfect or transform a host cell.

In some embodiments, the integration site of a host cell into which the exogenous biosynthetic gene cluster is inserted comprises one or more of the afo gene cluster and the mdp gene cluster. Preferably, insertion of the exogenous biosynthetic gene cluster into the host cell replaces or deletes some or all of the genes of the endogenous biosynthetic gene cluster. In some embodiments, some or all of the genes of the endogenous biosynthetic gene cluster are deleted prior to transformation to prevent unwanted homologous recombination.

In one embodiment, a method of producing a target compound in a recombinant Aspergillus nidulans host cell comprises the steps of: a) amplifying i) one or more polynucleotide sequences from a first target sequence, the first target sequence comprising one or more genes of an exogenous biosynthetic gene cluster for producing the target compound, and ii) amplifying one or more intergenic regions of an endogenous biosynthetic gene cluster of the host cell, wherein the one or more intergenic regions comprise a promoter sequence for at least one gene of the endogenous biosynthetic gene cluster, the one or more intergenic regions comprising one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15, one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64, or combinations thereof, and wherein the promoter sequence is controlled by a positive activator protein; b) assembling the amplified one or more polynucleotide sequences of the first target sequence and the amplified one or more polynucleotide sequences of the second target sequence in vitro using Gibson assembly to provide assembled sequences; c) using the assembled sequences as a template for a second amplification step to produce one or more final polynucleotide sequences; and d) transforming the one or more final polynucleotide sequences into the host cell wherein the one or more final polynucleotide sequences induce one or more homologous recombination events at an integration site of the host cell, wherein expression of one or more genes of the one or more final polynucleotide sequences causes production of the target compound.

Also provided are transgenic or engineered Aspergillus nidulans host cells for exogenous gene expression and, in particular, production of a target compound comprising an exogenous biosynthetic pathway gene cluster inserted into one or more endogenous biosynthetic gene clusters of the host cell.

In some embodiments, a transgenic strain of Aspergillus nidulans cells for producing a target compound comprises a recombinant biosynthetic pathway comprising: one or more genes of an exogenous biosynthetic gene cluster operably linked to a polynucleotide sequence of an intergenic region of a gene of an endogenous asperfuranone (afo) gene cluster and/or a gene of an endogenous monodictyphenone (mdp) gene cluster, wherein the intergenic region comprise a promoter sequence of the gene of the endogenous afo gene cluster and/or the endogenous mdp gene cluster; and a gene encoding a positive activator protein operably linked to an inducible promoter sequence wherein the positive activator protein binds to the promoter sequence of the gene of the endogenous afo gene cluster and/or the endogenous mdp gene cluster, thereby causing expression of the one or more genes of the exogenous biosynthetic gene cluster to produce the target compound.

In some embodiments, the promoter sequence of the one or more genes of the afo locus is at least 80% identical, at least 85% identical, at least 90% identical, at least 95% identical, at least 96% identical, at least 97% identical, at least 98% identical, at least 99% identical, or identical to one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15. In some embodiments, the promoter sequence of the one or more genes of the mdp locus is at least 80% identical, at least 85% identical, at least 90% identical, at least 95% identical, at least 96% identical, at least 97% identical, at least 98% identical, at least 99% identical, or identical to one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64 In some embodiments, an engineered strain of A. nidulans comprises a deletion of the native afoA gene and replaced with an afoA gene operably linked to an inducible promoter. In some embodiments, the inducible promoter is PalcA. In some embodiments, an engineered strain of A. nidulans comprises a deletion of the native mdpE gene and replaced with an mdpE gene operably linked to an inducible promoter. In some embodiments, the inducible promoter is PalcA.

In some embodiments, a transgenic strain of A. nidulans comprises one or more exogenous biosynthetic pathway genes inserted within the endogenous afo gene cluster. In other embodiments, a transgenic strain of A. nidulans comprises one or more exogenous biosynthetic pathway genes inserted within the endogenous afo and/or mdp gene clusters. In some embodiments, the afoA gene and/or the mdpE gene is operably linked to the PalcA inducible promoter.

In some embodiments, a transgenic strain of A. nidulans (e.g., strain YM192) for producing citreoviridin comprises one or more exogenous biosynthetic pathway genes within the endogenous afo and/or mdp regulon wherein the one or more exogenous biosynthetic pathway genes comprise the genes ctvA, ctvB, ctvC, and ctvD within the afo regulon or within the mdp regulon, wherein each of the exogenous genes is operably linked to an afo promoter or mdp promoter, and the afoA gene and/or the mdpE gene is operably linked to an inducible promoter. In some embodiments, the transgenic strains of A. nidulans further comprise a selectable marker such as pyrG. In some embodiments, the afoA gene and/or the mdpE gene is operably linked to the PalcA inducible promoter. In some embodiments, the exogenous biosynthetic pathway genes ctvA, ctvB, ctvC, and ctvD are from Aspergillus terreus var. aureus.

In some embodiments, a transgenic strain of A. nidulans (e.g., strain YM137) for producing mutilin comprises one or more exogenous biosynthetic pathway genes within the endogenous afo and/or mdp regulon wherein the one or more exogenous biosynthetic pathway genes comprise the genes Pl-ggs, cyc, p450-1, p450-2, sdr, within the afo regulon or within the mdp regulon, wherein each of the exogenous genes is operably linked to an afo promoter or mdp promoter, and the afoA gene and/or the mdpE gene is operably linked to an inducible promoter. In some embodiments, the transgenic strains of A. nidulans further comprise a selectable marker such as pyrG. In some embodiments, the afoA gene and/or the mdpE gene is operably linked to the PalcA inducible promoter.

In some embodiments, a transgenic strain of A. nidulans (e.g., strain YM343) for producing pleuromutilin comprises one or more exogenous biosynthetic pathway genes within the endogenous afo and/or mdp regulon wherein the one or more exogenous biosynthetic pathway genes comprise the genes Pl-ggs, cyc, p450-1, p450-2, sdr, atf, and p450-3, within the afo regulon or within the mdp regulon, wherein each of the exogenous genes is operably linked to an afo promoter or mdp promoter, and the afoA gene and/or the mdpE gene is operably linked to an inducible promoter. In some embodiments, the transgenic strains of A. nidulans further comprise a selectable marker such as pyrG. In some embodiments, the afoA gene and/or the mdpE gene is operably linked to the PalcA inducible promoter. In some embodiments, the exogenous biosynthetic pathway genes Pl-ggs, cyc, p450-1, p450-2, sdr, atf, and p450-3 are from C. passeckerianus.

In some embodiments, a transgenic strain of A. nidulans for producing fumagillin comprises one or more exogenous biosynthetic pathway genes within the endogenous afo and/or mdp regulon wherein the one or more exogenous biosynthetic pathway genes comprise the genes fma-TC, P450, C6H, MT, KR, afCPR, and fpaII, wherein each of the exogenous genes is operably linked to an afo promoter, and the afoA gene and/or the mdpE gene is operably linked to an inducible promoter. In some embodiments, the transgenic strains of A. nidulans further comprise a selectable marker such as pyrG. In some embodiments, the afoA gene and/or the mdpE gene is operably linked to the PalcA inducible promoter.

In some embodiments, a transgenic strain of A. nidulans for producing fumagillin comprises one or more exogenous biosynthetic pathway genes within the endogenous afo and/or mdp regulon wherein the one or more exogenous biosynthetic pathway genes comprise the genes fma-TC, P450, C6H, MT, KR, afCPR, and fpaII within the afo regulon and fma-A T, PKS, and ABM within the mdp regulon, wherein each of the exogenous genes is operably linked to an afo promoter or an mdp promoter, and the afoA gene and/or the mdpE gene is operably linked to an inducible promoter. In some embodiments, the transgenic strains of A. nidulans further comprise a selectable marker such as pyrG. In some embodiments, the afoA gene and/or the mdpE gene is operably linked to the PalcA inducible promoter. In some embodiments, the exogenous biosynthetic pathway genes fma-TC, P450, C6H, MT, KR, afCPR, fpaII, fma-AT, PKS, and ABM are from A. fumigatus.

In some embodiments, a transgenic strain of Aspergillus nidulans comprises SEQ ID NO: 1, 3, 5, 7, 9, 11, 13, 15, 16, and 17. In some embodiments, a transgenic strain of Aspergillus nidulans comprises SEQ ID NO: 16, 39, 40, 42, 44, 46, 48, 50, 52, 54, 56, 58, 60, 62, and 64.

In other embodiments, a transgenic strain of Aspergillus nidulans comprises SEQ ID NO: 1, 3, 5, 7, 9, 11, 12, 15, 16, 17, 18, 19, 20, 21, and 22. In other embodiments, a transgenic strain of Aspergillus nidulans comprises SEQ ID NO: 1, 3, 5, 7, 9, 11, 13, 14, 15, 16, 17, 23, 24, 25, 26, 27, and 28. In other embodiments, a transgenic strain of Aspergillus nidulans comprises SEQ ID NO: 1, 3, 5, 7, 9, 11, 13, 14, 15, 16, 17, 23, 24, 25, 26, 27, 28, 29, 30, and 31. In other embodiments, a transgenic strain of Aspergillus nidulans comprises SEQ ID NO: 1, 3, 5, 7, 9, 11, 13, 14, 15, 16, 17, 32, 33, 34, 35, 36, 37, and 38. In other embodiments, a transgenic strain of Aspergillus nidulans comprises SEQ ID NO: 1, 3, 5, 7, 9, 11, 13, 14, 15, 16, 17, 32, 33, 34, 35, 36, 37, and 38. In other embodiments, a transgenic strain of Aspergillus nidulans comprises SEQ ID NO: 16, 39, 40, 41, 42, 43, 44, 45, 46, 47, 48, 49, 50, 51, 52, 53, 54, 55, 56, 57, 58, 59, 60, 61, 62, 63, 64, and 65.

In some embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 1, 3, 5, 7, 9, 11, 13, 15, 16, and 17.

In some embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 16, 39, 40, 42, 44, 46, 48, 50, 52, 54, 56, 58, 60, 62, and 64.

In other embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 1, 3, 5, 7, 9, 11, 12, 15, 16, 17, 18, 19, 20, 21, and 22.

In other embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 1, 3, 5, 7, 9, 11, 13, 14, 15, 16, 17, 23, 24, 25, 26, 27, and 28.

In other embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 1, 3, 5, 7, 9, 11, 13, 14, 15, 16, 17, 23, 24, 25, 26, 27, 28, 29, 30, and 31.

In other embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 1, 3, 5, 7, 9, 11, 13, 14, 15, 16, 17, 32, 33, 34, 35, 36, 37, and 38.

In other embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 1, 3, 5, 7, 9, 11, 13, 14, 15, 16, 17, 32, 33, 34, 35, 36, 37, and 38.

In other embodiments, a transgenic strain of Aspergillus nidulans comprises polynucleotide sequences least 80% identical, at least 85% identical, at least 90% identical, at least 95% identical, at least 96% identical, at least 97% identical, at least 98% identical, or at least 99% identical to SEQ ID NO: 16, 39, 40, 41, 42, 43, 44, 45, 46, 47, 48, 49, 50, 51, 52, 53, 54, 55, 56, 57, 58, 59, 60, 61, 62, 63, 64, and 65.

In some embodiments, a transgenic strain of Aspergillus nidulans comprises any one of the strains listed in Tables 8-12.

In some embodiments, the target compound is a natural product or secondary metabolite comprising a violacein, a butadiene, a propylene, a 1,4-butanediol, an isopropanol, an ethylene glycol, a terephthalic acid, an adipic acid, a hexamethylenediamine (H/IDA), a caprolactam, a cyclohexanone, a aniline, a Methyl Ethyl Ketone (MEK), a fatty alcohol, an acrylic acid, an acrylate ester, a methyl methacrylate, a lipid, a carbohydrate, or an antibiotic, a butadiene, a propylene, a 1,4-butanediol, a 1,3-butanediol, a crotyl alcohol, a methyl vinyl carbinol, an isopropanol, an ethylene glycol, a terephthalic acid, an adipic acid, a hexamethylenediamine (HMDA), a caprolactam, a caprolactone, a hexanediol, a cyclohexanone, an aniline, a Methyl Ethyl Ketone (MEK), a fatty alcohol, an acrylic acid, an acrylate ester, a methyl methacrylate, a lipid, a carbohydrate, a beta-lactam, a polyketide, a macrolide, a macrolide having a 14-, 15- or 16-membered macrocyclic lactone ring, a ketolide, a taxane, a trans-AT type I PKS, a Type II PKS, or a Type III PKS, a heterocyst glycolipid PKS-like, a cyclic peptide, or a bottromycin, a terpenoid, a steroid, an alkaloid, a fatty acid, a nonribosomal polypeptide, an enzyme cofactor, an aminocoumarin, a melanin, an aminoglycosides/aminocyclitol, a microcin, an aryl polyene, a microviridin, a bacteriocin, a nucleoside, an oligosaccharide, a butyrolactone, a phenazine, a phosphoglycolipid, a cyanobactin, a phosphonate, a (dialkyl)resorcinol, a polyunsaturated fatty acid, an ectoine, a furan, a lycocin, a Head-to-tail cyclized peptide, a proteusin, a homoserine lactone, a sactipeptide, an indole, a siderophore, a ladderane lipid, a terpene, a lantipeptide, a thiopeptide, a linear azol(in)e-containing peptides (LAPs), a lasso peptide, or a linaridin,

In some embodiments, the target compound comprises antibacterial agents, antifungal agents, cytotoxins, anticancer and antitumor agents, immunomodulators, anti-inflammatory, anti-arthritic, anthelminthic, insecticides, coccidiostats and anti-diarrhea agents. In other embodiments, the target compound comprises a cytotoxin, an aminoglycoside antibiotic, a macrolide polyketide (Type I PKS), an oligopyrrole, a nonribosomal peptide, an aromatic polyketide (optionally an aromatic polyketide of a Type III PKS, an aromatic polyketide of Type II PKS), a complex isoprenoid, a beta-lactam, a terpenoid, a hybrid peptide-polyketide (from Type I PKS and NRPS), and/or a taxane, and also optionally comprising an antibacterial compound, optionally a vancomycin, erythromycin, daptomycin; antifungal agents (optionally amphotericin, nystatin); anticancer and antitumor agents for example doxorubicin, bleomycin; immunomodulators or immunosuppressants for example rapamycin, tacrolimus; anthelminthics for example avermectins; insecticides for example spinosyns; coccidiostats for example monensin, narasin; animal health compounds for example avilamycin, tilmicosin; optionally comprising acetogenins, actinorhodine, aflatoxin, albaflavenone, amphotericin, amphotericin b, annonacin, ansamycins, anthramycin, antihelminthics, avermectin, avilamycin, azithromycin, bleomycin, bullatacin, caprazamycins, carbomycin a, cephamycin c, cethromycin, chartreusin, calicheamicin, chloramphenicol, clarithromycin, clavulanate, coelchelin, cytotoxins, daptomycin, discodermolide, doxycycline, daunomycin, docetaxel, dolastatin, doxorubicin, echinomycin, endophenazine, epithienamycin, erythromycin, erythromycin a, fidaxomicin, FK506, flaviolin, fredericamycin, geldanamycin, ginsenoside compound K, Rh2, Rh1, Rg5, Rkl, Rg2, Rg3, Rg1, Rf, Re, Road, Rb2, Rc and Rb, geosmin, glucosyl-a47934, iso-migrastatin, ivermectin, josamycin, ketolides, kitasamycin, lovastatin, macbecin, macrolides, macrotetrolide, midecamycin, molvizarin, monensin, napyradiomycin, narasin, novobiocin, nystatin, oleandomycin, oxytetracycline, paclitaxel, pentalenolactone, phenalinolactione, pikromycin, pimaricin, pimecrolimus, polyene antimycotics, polyenes, polyketide macrolides, polyketides, radicicol, rapamycin, rifamycin, roxithromycin, sirolimus, solithromycin, spinosad, spinosyns, spiramycin, squamocin, staurosporine, streptomycin, tacrolimus, telithromycin, tetracenomycin, tetracyclines, teixobactin, thiocoraline, tilmicosin, troleandomycin, tylocine, tylosin, undecylprodigiosin, usnic acid, uvaricin, vancomycin and analogs thereof, and other target compound such as is described in Culler et al., U.S. Pat. Pub. No. 20180237847 and Konieczka et al., U.S. Pat. No. 11,421,223.

In certain embodiments, the target compound is an antifungal agent, antibacterial agent, bacteriostatic agent, anti-parasitic agent. In some embodiments, the target compound is citreoviridin, mutilin, pleuromutilin, or fumagillin.

In some embodiments, the target compounds can be an organic small molecule, for example, an organic compound having a molecular weight of less than 950 Da and greater than 90 Da. In various embodiments, the target compound has a molecular weight of less than about 900 Da, less than about 800 Da, less than about 700 Da, less than about 600 Da, less than about 500 Da, less than about 450 Da, less than about 400 Da, or less than about 300 Da, and the target compound can have a molecular weight of at least 100 Da, at least 150 Da, at least 200 Da, at least 250 Da, at least 300 Da, or at least 500 Da, or a range in between any of the aforementioned values, provided that the upper limit is greater than the lower limit of the combination of values that make up the range. For example, in some embodiments, the target compound has a molecular weight of less than about 500 Da and greater than about 350 Da. In some embodiments, the target compound is an antibacterial compound, an anti-parasitic compound, or a mycotoxin. As would be readily recognized by one of skill in the art, the target compound can be a terpene, a cycloalkyl compound, a heterocyclic compound, a polycyclic compound, or a combination thereof, each optionally substituted, for example, with one or more hydroxyl, oxo, alkyl, alkoxy, carboxylic acid, or oxycarbonyl substituents, wherein a carbon chain (any moiety of two or more carbon atoms) of the compound is saturated, unsaturated, unbranched, branched, or epoxidized, or a combination thereof, such as is present in the structures of the compounds citreoviridin, mutilin, pleuromutilin, or fumagillin.

### Results and Discussion

Design of Cluster Reconstitution and Refactoring; Obtaining Transforming DNA Fragments

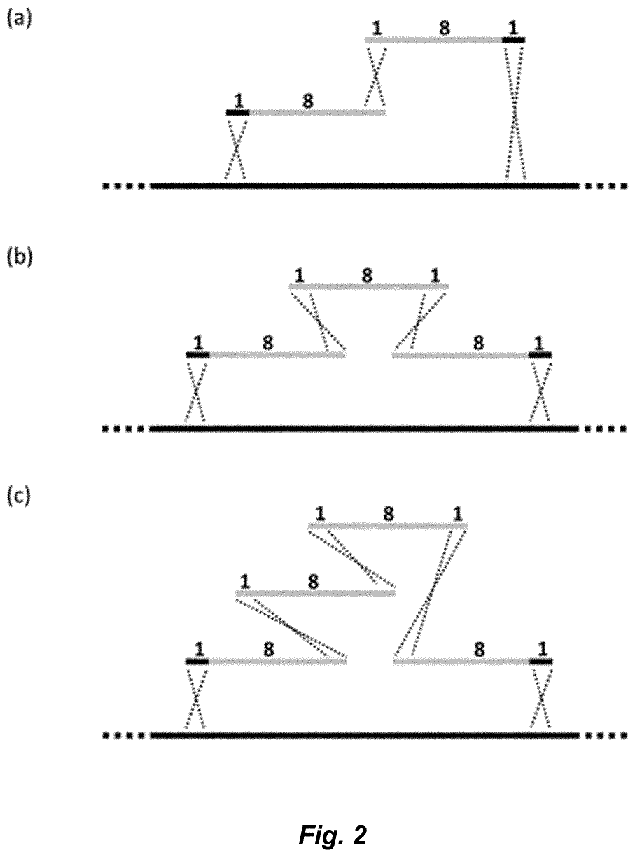

In order to efficiently replace the coding sequences of the afo genes with our GOIs, Applicants need to integrate large sequences of foreign DNA into the afo regulon in as few transformations as possible. It has been shown in the A. nidulans nkuAΔ strain that high efficiency gene targeting can be achieved by HR with 1 kb of flanking regions and that two DNA fragments can be fused by HR in vivo. In a previous study, Applicants successfully integrated three genes at three different loci in one single transformation, which required six HR events to occur concurrently. Therefore, Applicants envisioned the assembly of multiple large DNA fragments containing our GOIs and the transcriptional regulatory elements of afo (i.e., the intergenic regions of the afo regulon) in vivo through HR in one transformation. In theory, three HR events among the chromosome and two 10 kb DNA fragments each containing 1 kb of flanking regions on both the 3′ and 5′ ends would allow integration of 17 kb of foreign DNA in one transformation (FIG. 2a). Four HR events among three DNA fragments and the chromosome in vivo would allow integration of 26 kb of foreign DNA (FIG. 2b) and five HR events would allow 35 kb (FIG. 2c).

Applicants used isothermal Gibson assembly to generate our transforming fragments. In contrast to time-consuming yeast assembly and bacterial cloning, Gibson assembly can be done within 1 hour and the assembled DNA can be used immediately as a template for PCR. Therefore, sub-picomolar levels of large DNA fragments for transformation can be obtained within one day from amplifying GOIs.

Reconstitution of the Citreoviridin Biosynthetic Pathway in the Afo Regulon

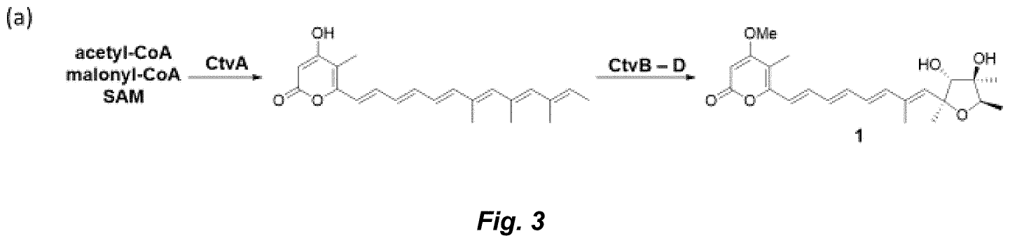

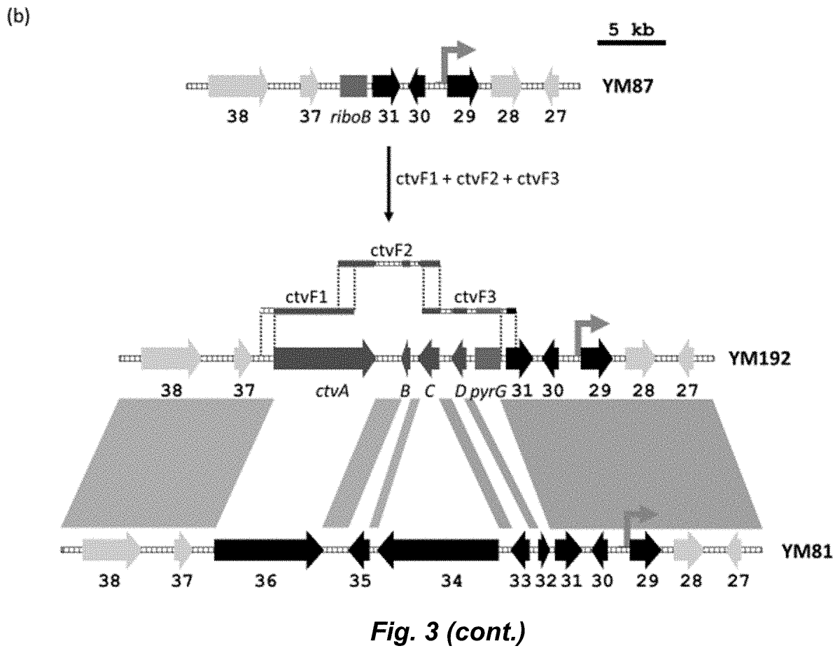

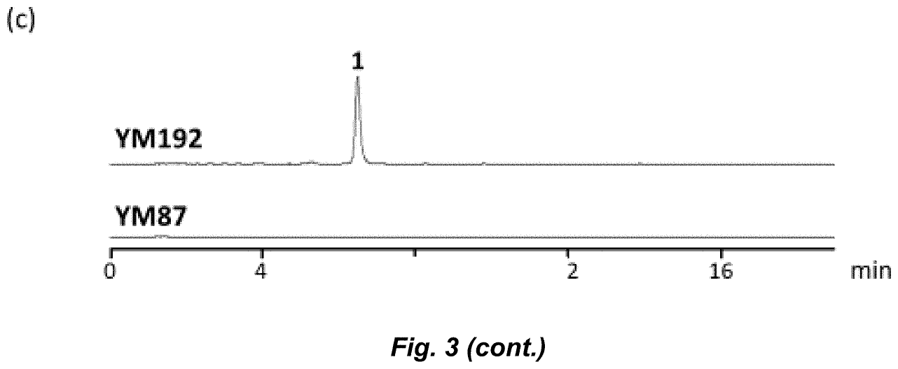

As a proof of principle, Applicants selected the citreoviridin biosynthetic pathway to be reconstructed in the afo regulon. Citreoviridin (1) is a mycotoxin that belongs to a class of F1-ATPase inhibitors. Applicants have shown that it is biosynthesized by a highly-reducing polyketide synthase (CtvA) and three auxiliary enzymes (CtvB-D) (FIG. 3a). By placing the four genes under the control of PalcA in A. nidulans, 1 was produced at a moderate yield (˜10.5 mg/L).

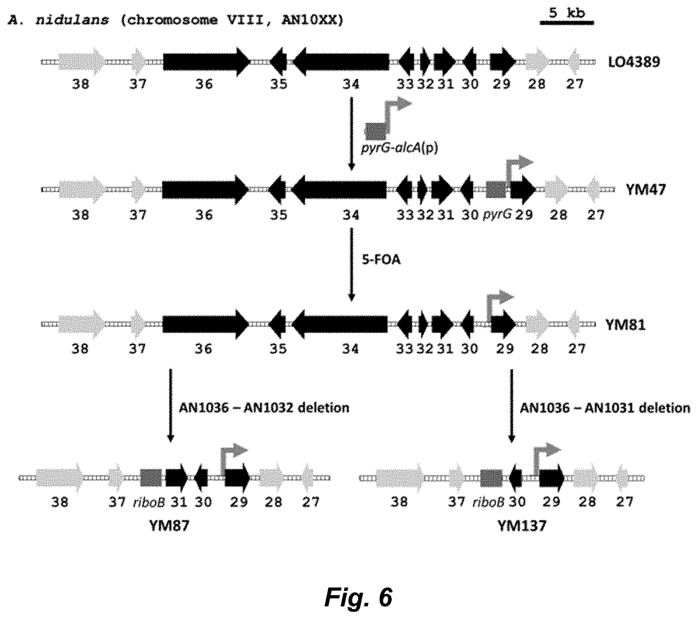

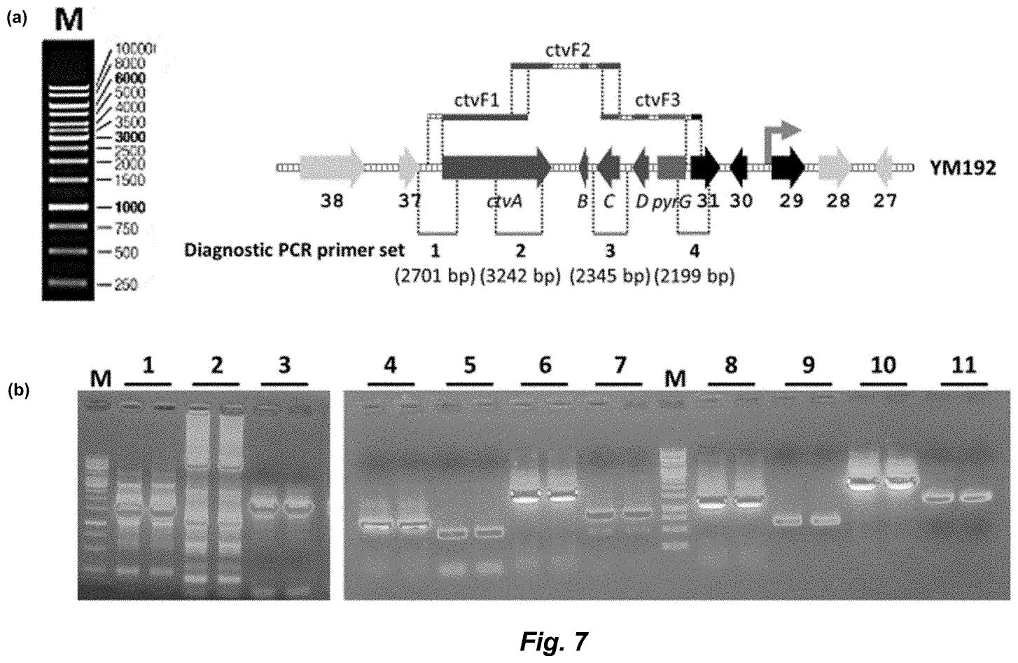

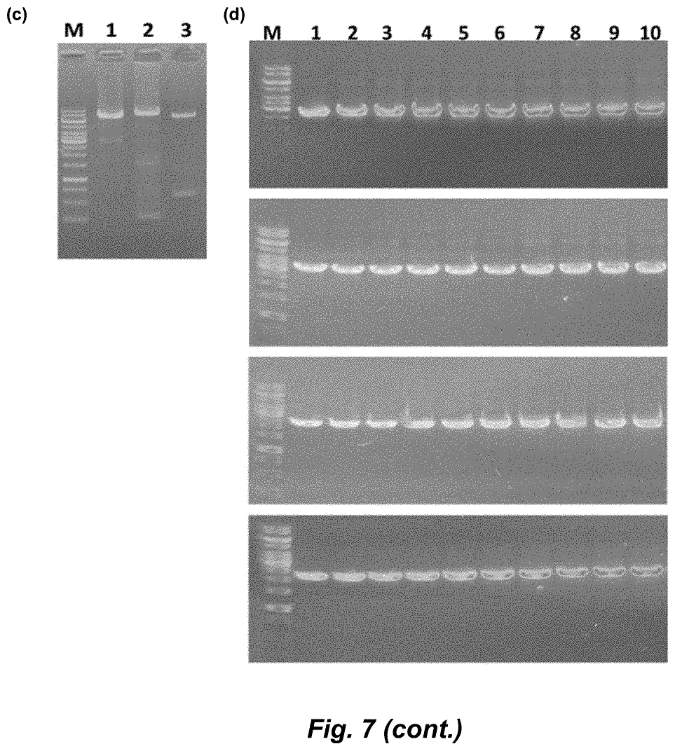

Intergenic regions of the afo regulon and the four ctv genes were amplified by PCR from the gDNA of A. nidulans and A. terreus var. aureus, respectively (FIGS. 7a and 7b). PCR fragments were gel-purified and assembled by Gibson assembly. The assembled DNA were then used as templates for PCR to generate large transforming fragments (ctvF1-F3) ranging from 6.9 kb to 7.5 kb in sub-picomolar quantities (FIG. 7c). Applicants used the recipient strain YM87 (FIG. 6), in which the stc BGC has been deleted to eliminate the production of sterigmatocystin, the major metabolite detected under the PalcA induction condition, in order to obtain a cleaner metabolite background and free up polyketide precursors. Furthermore, AN1029 (afoA) was placed under the control of PalcA in order to create an inducible system, which would be useful for metabolites toxic to the host. Lastly, Applicants deleted the DNA region from AN1036 to AN1032 to prevent unwanted HR with the intergenic regions on the transforming fragments (FIGS. 3b and 6).

The three transforming fragments, ctvF1-F3, would constitute an 18.7 kb region of ctvA-D genes under the control of the afo regulon if the four HR events outlined in FIG. 3b occur. Transformation with ctvF1-F3 yielded 86 prototrophic colonies. In contrast, the negative control transformation with only the fragment ctvF3 (where the selectable marker pyrG was placed) yielded only one colony. Applicants were able to acquire two correct transformants from six prototrophic colonies in a co-transformation of three fragments with six HR events. Therefore, Applicants reasoned that Applicants could acquire correct transformants from a co-transformation with four HR events from as little as ten prototrophic colonies. Gratifyingly, when Applicants randomly picked ten of the 86 colonies (YM186-YM195) and screened them by diagnostic PCR, Applicants found that all 10 were correct transformants (FIG. 7d).

After cultivation, all ten transformants were found to produce high levels of citreoviridin (352.3-615.7 mg/L) under the PalcA inducing condition (Table 2). Since citreoviridin was the major peak detected when Applicants ran the culture medium on high-performance liquid chromatography (HPLC), Applicants wanted to examine the purity of citreoviridin that could obtain after extraction with organic solvent. Applicants selected one transformant, YM192, for cultivation and extraction as described in Material and Methods. In the ¹H NMR spectrum of the extracted sample, Applicants found that all the proton signals, except for those of organic solvent dichloromethane (DCM) and inducer methyl ethyl ketone (MEK), were attributed to citreoviridin. Our results demonstrated that large DNA fragments can be assembled in vivo with high efficiency in A. nidulans and that a 4-gene citreoviridin biosynthesis pathway can be reconstituted and refactored in the afo regulon in one transformation to give strains with high production yield and high purity.

TABLE 2
Quantification of citreoviridin production:
culture media of strains YM186-YM195.

| | Concentration |
|---|---|
| Strain | (mg/L) |
| YM186 | 561.3 |
| YM187 | 597.2 |
| YM188 | 560.9 |
| YM189 | 382.2 |
| YM190 | 521.0 |
| YM191 | 352.3 |
| YM192 | 615.7 |
| YM193 | 362.6 |
| YM194 | 497.2 |
| YM195 | 434.2 |
| Average | 488.4 |

Reconstitution of the Pleuromutilin Biosynthetic Pathway in the Afo Regulon

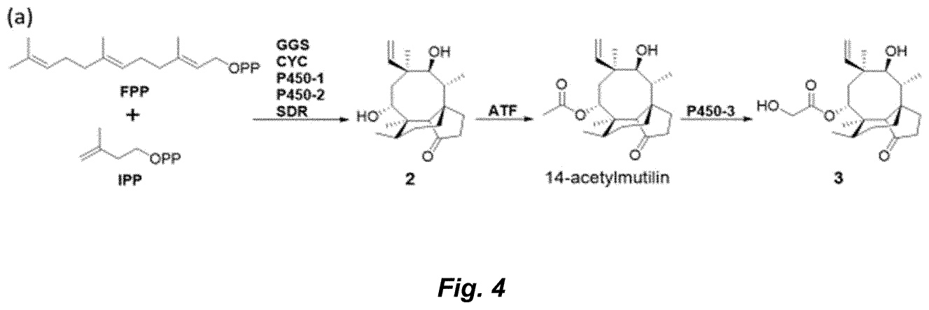

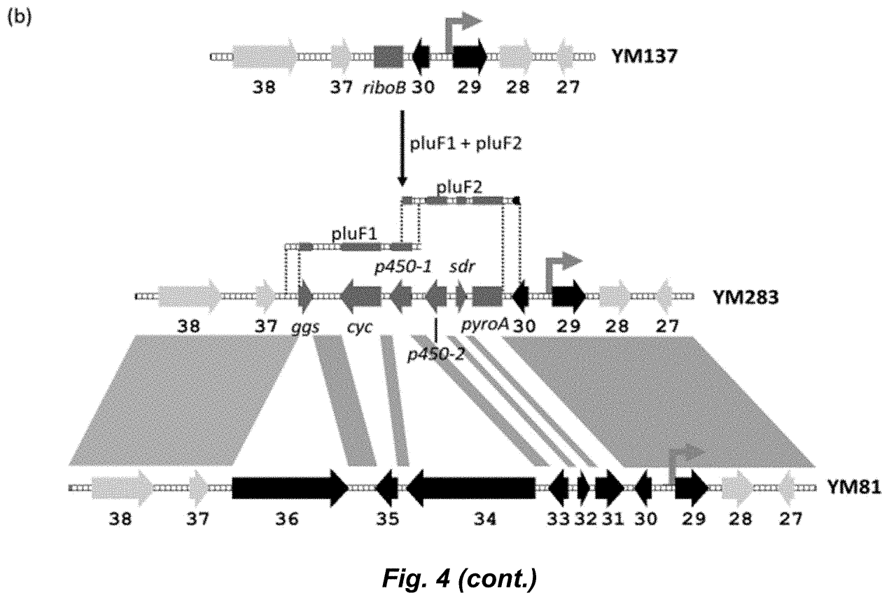

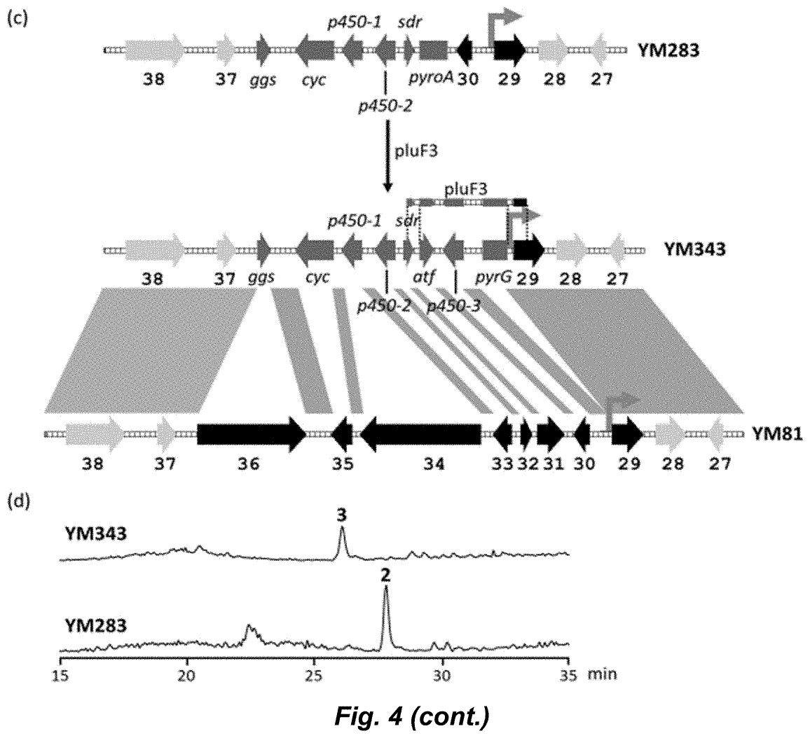

Encouraged by our success with the citreoviridin cluster, Applicants wanted to test our system on a seven-gene pathway, i.e., exchanging the coding regions of AN1030-AN1036 with seven heterologous genes. Applicants selected pleuromutilin, a diterpene antibiotic produced by basidiomycete fungi Clitopilus passeckerianus. Its biosynthesis involving seven genes (Pl-ggs, cyc, atf, sdr, p450-1, p450-2, and p450-3) was elucidated by heterologous expression in the A. oryzae NSAR1 strain (FIG. 4a). In their study, three expression vectors each with a different selectable marker were used to reconstitute the pleuromutilin pathway. The highest producing strain with a yield of ˜84 mg/L was obtained after screening 12 transformants. It should be noted that multiple copies of two genes, Pl-atf and Pl-sdr, were found in the highest producing strain. Since A. oryzae is the most popular heterologous expression system used to study fungal NP biosynthesis, our study would provide an opportunity to compare the two systems.

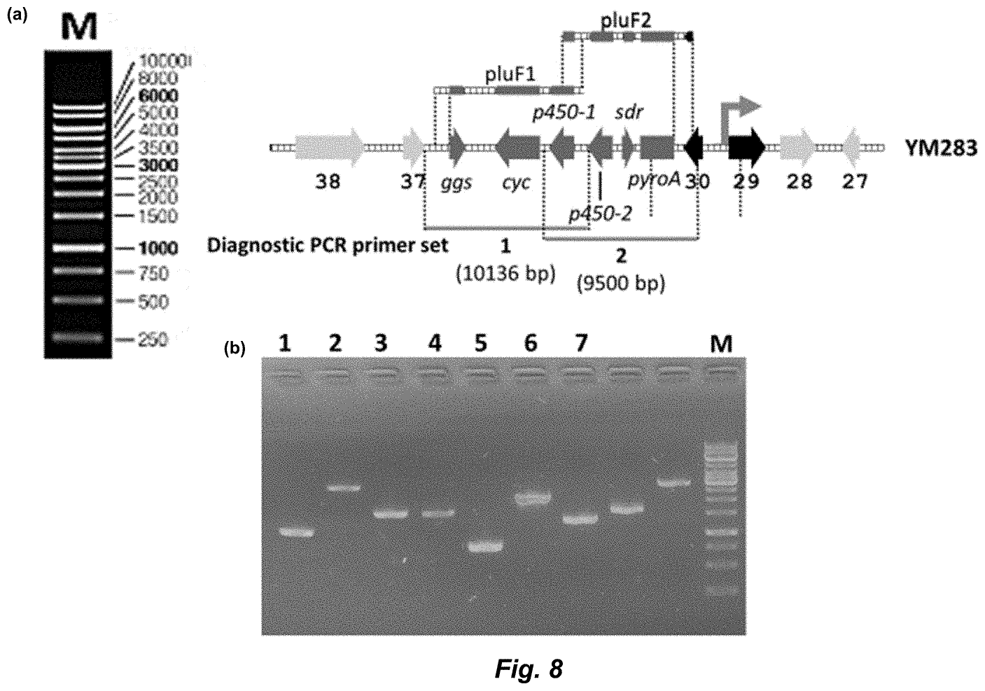

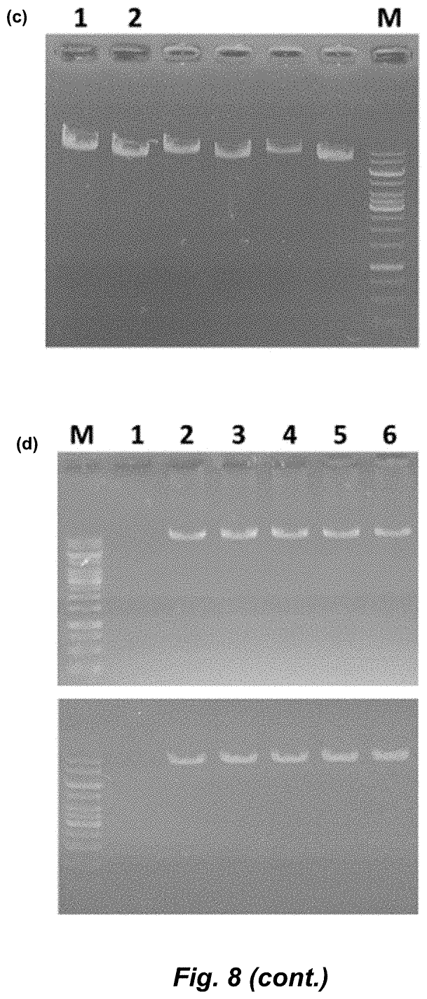

Applicants first aimed to create a strain that can produce mutilin (2), a key intermediate in the pleuromutilin biosynthetic pathway (FIG. 4a). Five pl genes (pl-ggs, pl-cyc, pl-p450-1, pl-p450-2, and pl-sdr) were amplified from the cDNA of Clitopilus passeckerianus (FIGS. 8a and 8b), gel-purified, and assembled with intergenic regions of the afo regulon by Gibson assembly. The assembled DNA were then used as templates for PCR to generate two large PCR fragments, pluF1 (9.2 kb) and pluF2 (8.2 kb) (FIG. 8c). Applicants used the recipient strain YM137 (FIG. 6), in which the DNA region from AN1036 to AN1031 has been deleted and AN1029 (afoA) has been placed under the control of PalcA. Since Applicants expected that most of the prototrophic colonies would be correct transformants, five (YM283-YM287, FIG. 4b) were randomly picked from >60 colonies and examined by diagnostic PCR. Again, all picked colonies were correct transformants as expected (FIG. 8d). Under inducing conditions, all five produced a major new peak in total ion chromatogram (TIC) and extracted ion chromatogram (EIC) at m z 303 detected by LC-MS. The mass spectrum of the new peak has a parent ion of m/z 321 ([M+H]⁺) and a base peak of m/z 303 ([M+H−H₂O]⁺), which corresponded to mutilin (MW=320). After extraction of the culture medium of YM283 (30 mL) with organic solvent, ¹H NMR analysis of the extract (3.8 mg) revealed largely pure mutilin (93%, estimated from ¹H NMR spectrum).

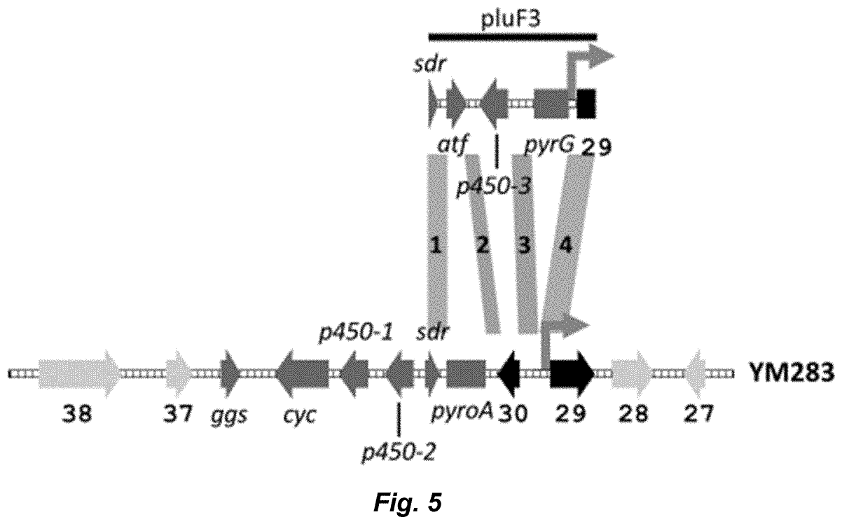

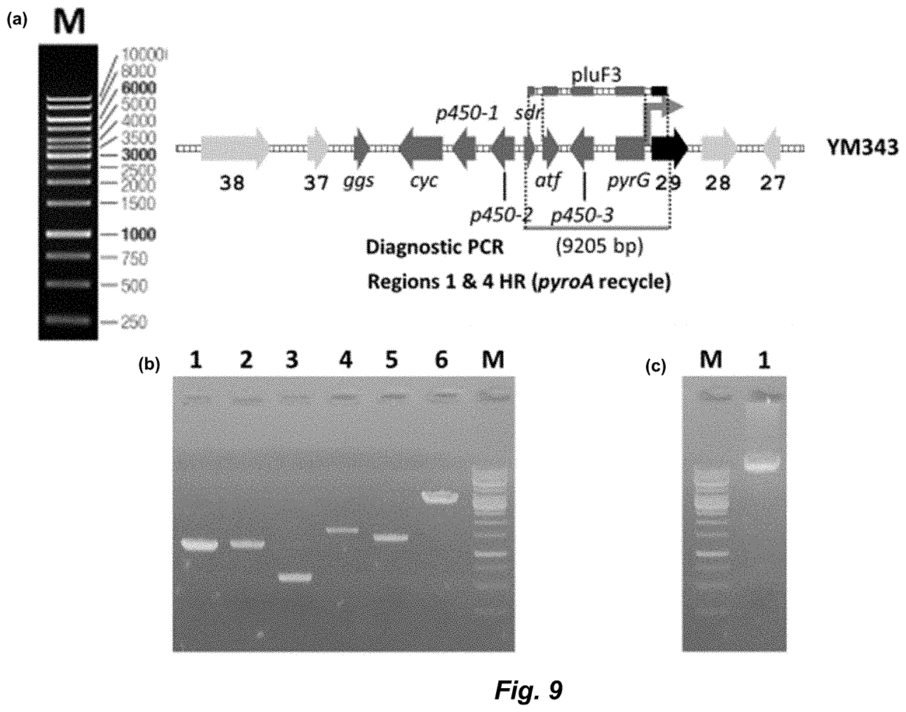

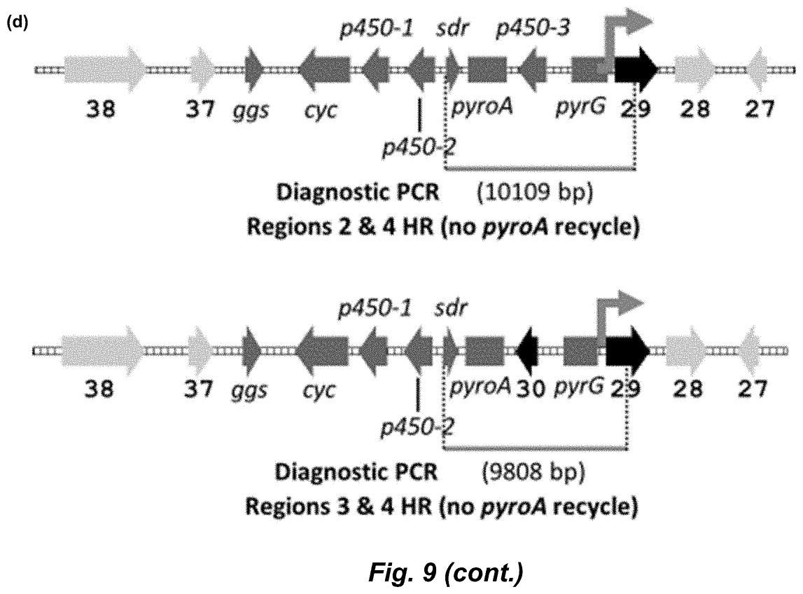

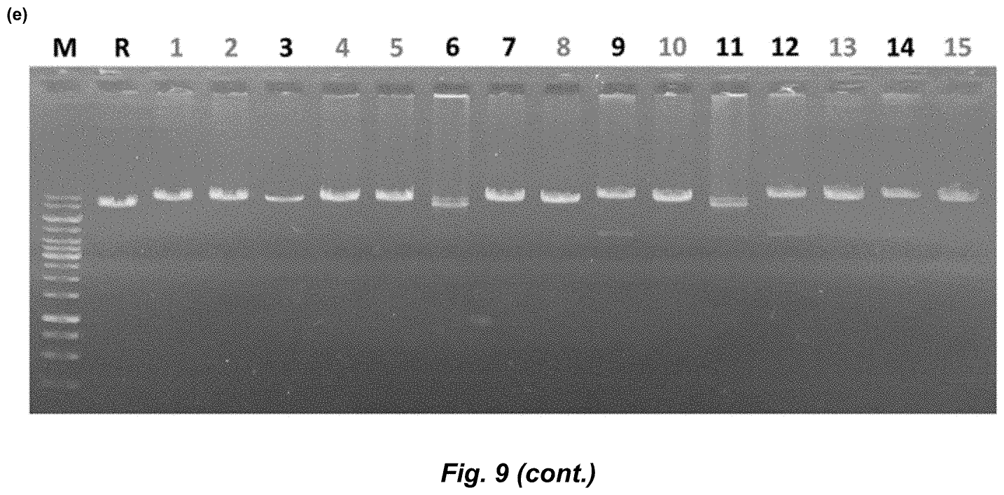

To reconstitute the entire pleuromutilin pathway, pl-atf and pl-p450-3 were inserted into the coding regions of AN1031 and AN1030 in the mutilin-producing strain YM283. The transforming fragment pluF3 (8.9 kb) containing pl-atf and pl-p450-3 was PCR amplified from the assembly of six DNA segments (FIGS. 9a, 9b and 9c). Notably, there are four regions in pluF3 that have identical sequences with the afo locus (FIG. 5). HR between regions 1 and 4 would result in the desired insertion of pl-atf and pl-p450-3 along with the pyrG cassette and recycling of the pyroA cassette (FIG. 4c), creating strains that would be uracil prototrophic but pyridoxin auxotrophic. However, HR between regions 2 and 4, or regions 3 and 4 would result in the insertion of the pyrG cassette but no recycling of pyroA (FIG. 9d), creating strains that would be both uracil and pyridoxin prototrophic. While the odds of HR between DNA regions 1 and 4 could be greatly enhanced by removing regions 2 and 3 from the recipient strain YM283, Applicants wanted to test if Applicants could bypass that step to acquire the desired transformants with one single transformation.

Since Applicants expected a mixed population of desired and undesired transformants, fifteen uracil prototrophic colonies were randomly picked from >60 colonies obtained. After screening, eight of them were found to be pyridoxin auxotrophs and showed correct diagnostic PCR patterns (FIG. 9e). Those strains were cultured under inducing condition and the culture media were screened by liquid chromatography-mass spectrometry (LC-MS). Four of them (YM343, 347, 355, and 357) produced a new peak (3) that eluted before mutilin and two (YM346 and 350) produced a new peak (4) that eluted after mutilin. Both peaks had almost identical mass spectrum with mutilin, indicating that both were mutilin derivatives. The organic extract of YM343 (4.6 mg from a 30 mL culture) was analyzed by ¹H NMR, which showed that pleuromutilin (3) was indeed obtained in high purity. Notably, the yield of YM343 (˜150 mg/L) is higher than the highest producing strain derived from A. oryzae NSAR1 strain (˜84 mg/L). Peak 4 was likely 14-acetylmutilin (FIG. 4a), an intermediate upstream of pleuromutilin (3), expected to have less polarity, given that 4 eluted after 2 on a reversed-phase column. Thus, although HR between the intergenic regions complicated the analysis of the prototrophic colonies, Applicants still successfully acquired pleuromutilin-producing strains.

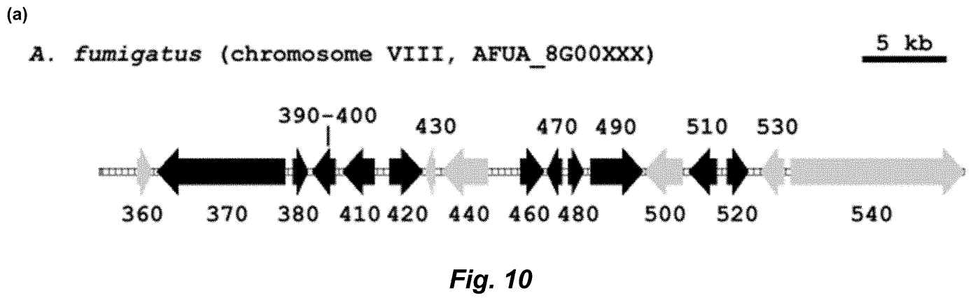

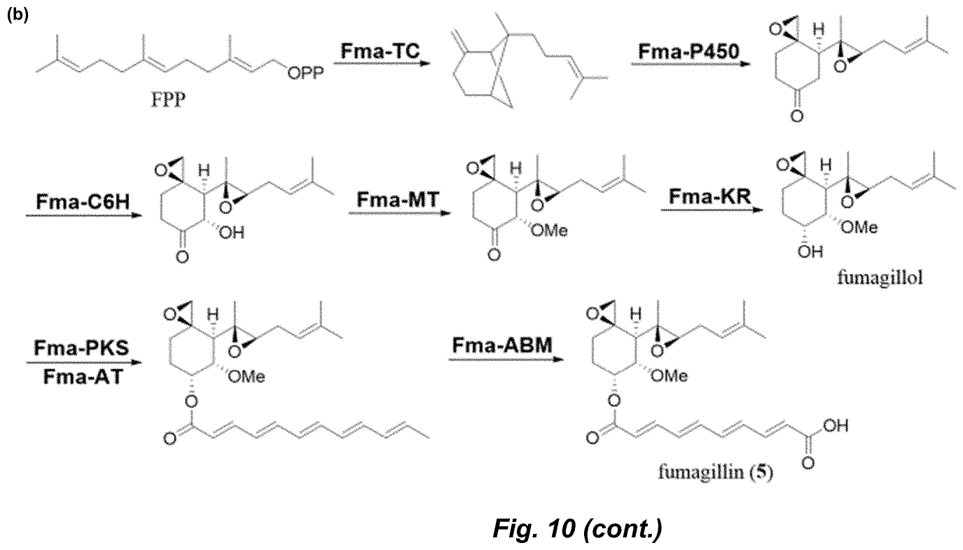

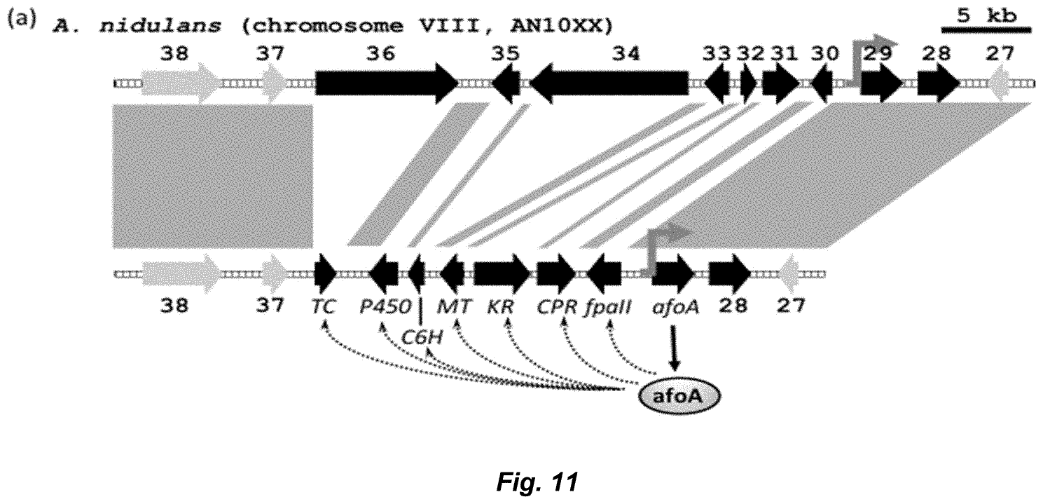

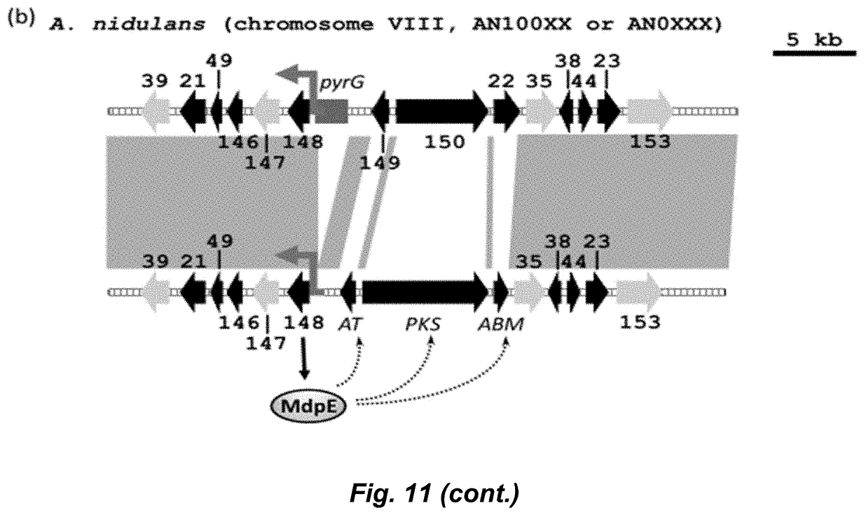

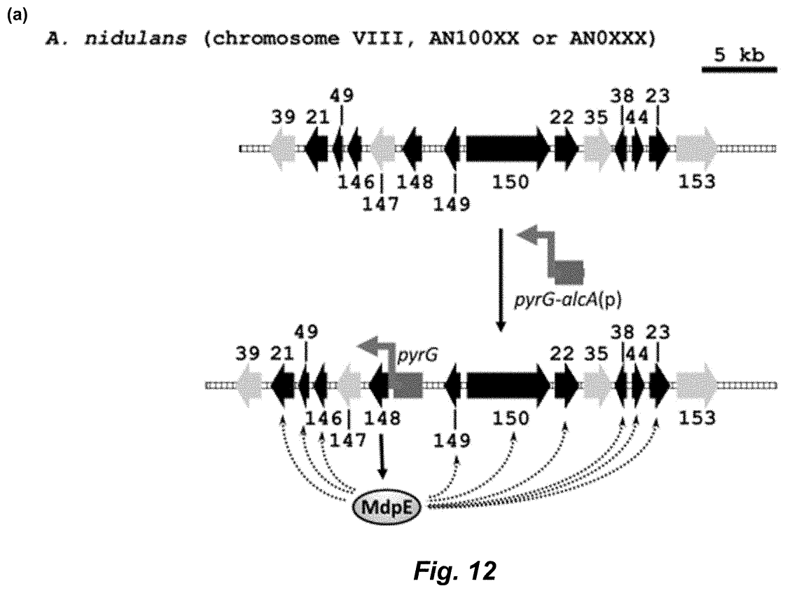

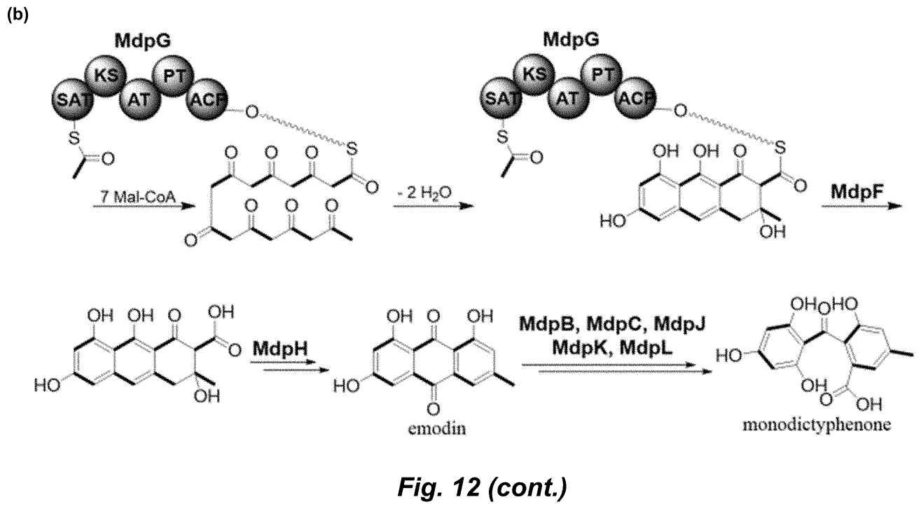

Using a similar approach, Applicants also generated a strain that produces fumagillin (5). Fumagillin is a methionine aminopeptidase 2 (MetAP2) inhibitor, and currently, it is the only commercialized NP used to treat Nosema infection in honeybees. The biosynthesis gene cluster of fumagillin has been identified from A. fumigatus (FIG. 10, Table 3). There are five enzymes (Fma-TC, P450, C6H, MT, and KR) that convert farnesyl pyrophosphate (FPP) to fumagillol which then transforms to fumagillin by three other enzymes (Fma-PKS, AT, and ABM). Besides the eight genes that involved in the enzymatic steps of the fumagillin biosynthesis, two addition genes, afCPR (Afu6g10990) and fpaII (Afu8g00410) were also inserted into the genome of the A. nidulans host for the optimized production of fumagillin. AfCPR (AFUA_6G10990) is a cytochrome P450 oxidoreductase that equips Fma-P450 with the optimal redox partner and FpaII (AFUA_8G00410) is a MetAP2 that confers the resistance of fumagillin. Expression of AfCPR and FpaII were expected to facilitate the biosynthesis of fumagillin and abolish the toxicity of fumagillin to the producing strain, respectively. The created strain YM727 incorporated fma-TC, P450, C6H, MT, KR, afCPR, and fpaII in the afo regulon (FIG. 11a); and fma-PKS, AT, and ABM in the mdp regulon (FIG. 12b). Similar to afo regulon, induction of the expression of mdpE gene elicits the expression of genes in the mdp cluster which led to the production of monodictyphenone (FIG. 12, Table 4). The resulting strain contains 10 heterologous genes from A. fumigutaus (FIG. 11), which produces ˜55 mg/L of fumagillin (5) after induction of afoA and mdpE.

TABLE 3
Sizes and putative functions of genes identified in the fma cluster.

| | | |
|---|---|---|
| | Gene Size | Putative |
| Gene Name | (base pairs) | Function |

| | | | |
|---|---|---|---|
| 370 | (fma-PKS) | 7603 | HR-PKS |
| 380 | (fma-AT) | 926 | Alpha, beta-hydrolase |
| 390-400 | (fma-MT) | 1379 | O-methyltransferase |
| 410 | (fpaII) | 1937 | MetAP type II |
| 420 | (fapR) | 1989 | Positive regulator |
| 460 | (fpaI) | 1425 | MetAP type I |
| 470 | (fma-ABM) | 895 | Monooxygenase |
| 480 | (fma-C6H) | 930 | Dioxygenase |
| 490 | (fma-KR) | 3155 | Partial PKS |
| 510 | (fma-P450) | 1665 | P450 oxidoreductase |

TABLE 4
Sizes and putative functions of genes identified in the mdp cluster.

| | Gene size | Putative |
|---|---|---|
| Gene name | (base pairs) | function |
| AN10021 (mdpA) | 1534 | Co-regulator |
| AN10049 (mdpB) | 692 | Scytalone dehydratase |
| AN10046 (mdpC) | 925 | Versicolorin |
| | | ketoreductase |
| AN10047 (mdpD) | 1644 | Monoxygenase |
| AN10048 (mdpE) | 1308 | Positive regulator |
| AN10049 (mdpF) | 1018 | Metallo-beta-lactamase |
| AN10050 (mdpG) | 5562 | NR-PKS |
| AN10022 (mdpH) | 1586 | DUF 1772 superfamily |
| AN10035 (mdpI) | 1857 | Acyl-CoA synthase |
| AN10038 (mdpJ) | 799 | Glutathione S-transferase |
| AN10044 (mdpK) | 798 | Oxidoreductase |
| AN10023 (mdpL) | 1341 | Baeyer-Villiger oxidase |

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Material and Methods

Reagents and General Experimental Procedures

Citreoviridin was purchased from Enzo Life Sciences (Farmingdale, N.Y., USA). DNA concentrations were determined by NanoDrop (ThermoFisher Scientific). NMR spectra were collected on a Varian Mercury Plus 400 spectrometer. Strains used in this study were listed in Table 5. Primers used for PCR amplification and diagnostic PCR were listed in Table 6.

DNA Fragment Preparation and Molecular Genetic Manipulations

DNA of intergenic regions of the afo regulon were PCR amplified from the strain LO4389. DNA of GOIs were PCR amplified from gDNA of A. terreus var. aureus (ctvA-D) and from cDNA of Clitopilus passeckerianus (Pl-ggs, cyc, atf, sdr, p450-1, p450-2, and p450-3) as described. DNA amplified were gel-purified and quantified by NanoDrop. Gibson assembly was performed using NEBuilder HiFi DNA Assembly Master Mix (NEB, #E2621) according to the manufacturer's protocol. Briefly, 0.05 picomole of each DNA fragment with 25 bp overlap regions were added to ddH₂O to make 10 μL, to which 10 μL of NEBuilder HiFi DNA Assembly Master Mix was added. The assembly mixture was incubated at 50° C. for 1 hour. Following incubation, the reaction mixtures were stored on ice for subsequent PCR amplification. Large DNA fragments were gel-purified and quantified by NanoDrop after PCR. Sub-picomole of large DNA fragments can be obtained from 200 μL of PCR.

Protoplast production and transformation were carried out according to techniques known in the art. Prototrophic colonies were randomly picked and examined by diagnostic PCR.

Fermentation, Induction, and HPLC Analysis

For fermentation, 3×10⁷ spores were grown in 30 mL of liquid LMM medium (15 g/L lactose, 6 g/L NaNO₃, 0.52 g/L KCl, 0.52 g/L MgSO₄·7H2O, 1.52 g/L KH₂PO₄, 1 ml/L Hutner's trace elements solution) in 125-mL flasks supplemented as necessary with riboflavin (2.5 mg/L), pyridoxine (0.5 mg/L), uracil (1 g/L), or uridine (10 mM). Flasks were incubated at 37° C. with shaking at 180 rpm. For PalcA induction, methyl ethyl ketone (MEK) at a final concentration of 50 mM was added to the medium after 18 h of incubation. The culture medium was collected 72 hours after MEK induction. For citreoviridin producing strains (YM186-YM195), 10 μL of the culture medium was diluted 10-fold and injected for IPLC analysis. IPLC (Agilent 1200 Series) analysis was performed using an RP-18 column (Agilent Eclise XDB-C18 5 pm, 4.6×150 mm) at a flow rate of 1.0 mL/min and detected by a DAD detector. The solvents used were 100% acetonitrile (solvent B) and 5% acetonitrile in H₂O (solvent A), both containing 0.05% formic acid. The gradient was 30-46% B from 0 to 8 min, 46-100% B from 8 to 11 min, maintained at 100% B from 11 to 14 min, 100-30% B from 14 to 15 min, and re-equilibration with 30% B from 15 to 19 min.

For mutilin (YM283-YM287), pleuromutilin (YM343, 344, 346, 347, 350, 352, 355, and 357), and fumagillin (YM727) producing strains, 10 μL of the culture medium was injected for LC-DAD-MS analysis.

NMR Analysis

For NMR analysis of citreoviridin (1), strain YM192 was cultured and induced as described above. After induction, about 25 ml of the cultural medium was collected. The medium was extracted with 25 ml of dichloromethane (DCM) and 13.2 mg of extracted material was obtained after evaporating the DCM in vacuo. Since citreoviridin is unstable under light, all procedures including culturing and extraction were protected from light. NMR was taken immediately after evaporating the DCM in vacuo.

For NMR analysis of mutilin (2), strain YM283 was cultured and induced as described above. After induction, about 25 ml of the culture media was collected. The media was then extracted with 25 ml of ethyl acetate (EA). After evaporating the EA in vacuo, the extract was resuspended in DCM followed by centrifugation to remove uridine and uracil. Supernatant containing 2 dissolved in DCM was carefully collected, and 3.8 mg of extracted material was obtained after evaporating the DCM in vacuo. The ¹H NMR of extracted material was taken without further purification.

For NMR analysis of pleuromutilin (3), strain YM343 was cultured, induced, and extracted as described above. After evaporating EA in vacuo, 4.6 mg of extracted material was obtained. The ¹H NMR of extracted material was taken without further purification.

### Example 2. Strains and Polynucleotide Sequences

TABLE 5
A. nidulans strains used in this study.

| Fungal strains | Genotypes |
|---|---|
| LO4389¹ | pyrG89; pyroA4; nkuA::argB; riboB2; stcA-stcWΔ |
| YM47² | pyrG89; pyroA4; nkuA::argB; riboB2; stcA-stcWΔ; |
| | AN1029::AfpyrG-PalcA-AN1029 |
| YM81 | pyrG89; pyroA4; nkuA::argB; riboB2; stcA-stcWΔ; |
| | AN1029::PalcA-AN1029 |
| YM87 | pyrG89; pyroA4; nkuA::argB; riboB2; stcA-stcWΔ; |
| | AN1029::PalcA-AN1029; AN1036-AN1032::AfriboB |
| YM137 | pyrG89; pyroA4; nkuA::argB; riboB2; stcA-stcWΔ; |
| | AN1029::PalcA-AN1029; AN1036-AN1031::AfriboB |
| YM186-YM195 | pyrG89; pyroA4; nkuA::argB; riboB2; stcA-stcWΔ; |
| | AN1029::PalcA-AN1029; AN1036-AN1032::ctvA-ctvB- |
| | ctvC-ctvD-AfpyrG |
| YM283-YM287 | pyrG89; pyroA4; nkuA::argB; riboB2; stcA-stcWΔ; |
| | AN1029::PalcA-AN1029; AN1036-AN1031Δ::pl_ggs- |
| | cyc-p450_1-p450_2-sdr-AfpyroA |
| YM343, 347, | pyrG89; pyroA4; nkuA::argB; riboB2; stcA-stcWΔ; |
| 355, and 357 | AN1029::PalcA-AN1029; AN1036-1029PΔ::pl_ggs- |
| | cyc-p450_1-p450_2-sdr-atf-p450_3-AfpyrG-1029P |
| YM727 | pyrG89; pyroA4; nkuA::argB; riboB2; stcA-stcWΔ; |
| | AN1029::PalcA-AN1029; AN1036-1029PΔ::fma_TC- |
| | P450-C6H-MT-KR-CPR-fpall-1029P; 0148P- |
| | AN10022Δ::PalcA-AN0148-fma_AT-PKS-ABM |

¹LO4389 has been reported previously (Chiang et al., 2013, J Am Chem Soc. 135, 7720-31).
²Primers used for replacing the promoter of AN1029 (afoA) with PalcA have been published previously (Chiang et al., 2009, J Am Chem Soc. 131, 2965-2970).

TABLE 6
Primers used in this study.

| Primers used for generating YM81 | | |
|---|---|---|
| (recycling the AfpyrG cassette) | | |
| alcA_AN1029_P1 | ggagcgacagaaccaaagtc | SEQ ID NO: 66 |
| alcA_AN1029_P2 | tgggccatgggctatcttcc | SEQ ID NO: 67 |
| alcAF- | ctatcacaatcagcttttcag | SEQ ID NO: 68 |
| alcA_AN1029_P3 | ttacgagcgagttacgaacg | |
| alcA_F | ctgaaaagctgattgtgatag | SEQ ID NO: 69 |
| alcA_AN1029_P5 | tgctggggtatggctatctc | SEQ ID NO: 70 |
| alcA_AN1029_P6 | atggcagtgagcagacattg | SEQ ID NO: 71 |
| Primers used for generating YM87 (AN1036-AN1032Δ) | | |
| 1. 1036P fragment (1487 + 21 bp) | | |
| 1036P_F | aatgactggtccgtccgtac | SEQ ID NO: 72 |
| pyrGF2-1036P_R | cgaagagggtgaagagcattg | SEQ ID NO: 73 |
| | ggtgccttgtggatggggatta | |
| 2. Afribo cassette fragment (2013 bp) | | |
| PyrGF2 | caatgctcttcaccctcttcg | SEQ ID NO: 74 |
| PyrGR | ctgtctgagaggaggcactgatgc | SEQ ID NO: 75 |
| 3. 1031P-partial AN1031 fragment (1145 + 24 bp) | | |
| pyrGR-1031P_F | gcatcagtgcctcctctcagacag | SEQ ID NO: 76 |
| | attcagcctattgagattacag | |
| 1031P_R1 | cctagtaggtgggatttgaa | SEQ ID NO: 77 |
| Fusion PCR primers (4062 bp) | | |
| 1036P_F3 | atgtgctctacggacgaaaaat | SEQ ID NO: 78 |
| 1031P_R2 | atgaagagcgcctgtttctg | SEQ ID NO: 79 |
| Primers used for generating YM137 (AN1036-AN1031Δ) | | |
| 1. 1036P fragment (1487 + 21 bp) | | |
| 1036P_F | aatgactggtccgtccgtac | SEQ ID NO: 80 |
| pyrGF2-1036P_R | cgaagagggtgaagagcattg | SEQ ID NO: 81 |
| | ggtgccttgtggatggggatta | |
| 2. Afribo cassette fragment (2013 bp) | | |
| PyrG_F2 | caatgctcttcaccctcttcg | SEQ ID NO: 82 |
| PyrG_R | ctgtctgagaggaggcactgatgc | SEQ ID NO: 83 |
| 3. 1031T-partial AN1030 fragment (1317 + 24 bp) | | |
| pyrGR-1031T_F | gcatcagtgcctcctctcagacag | SEQ ID NO: 84 |
| | ggcatcgtctacaagcagatg | |
| AN1030_R1 | tttggtctcttccacaaggact | SEQ ID NO: 85 |
| Fusion PCR primers (4131 bp) | | |
| 1036P_F3 | atgtgctctacggacgaaaaat | SEQ ID NO: 86 |
| AN1030_R2 | gtctttgactaccggagcaagt | SEQ ID NO: 87 |
| Primers used for amplifying intergenic regions | | |
| of the afo regulon | | |
| 1. Intergenic region between AN1037 and AN1036 | | |
| (named 1036P, 1487 bp) | | |
| 1036P_F | aatgactggtccgtccgtac | SEQ ID NO: 88 |
| 1036P_R | ggtgccttgtggatggggatta | SEQ ID NO: 89 |
| 2. Intergenic region between AN1036 and AN1035 | | |
| (named 1036, 1768 bp) | | |
| 1036T_F | gctgcatcggtcatgttgttc | SEQ.ID NO: 90 |
| 1036T_R | ggtggatagccgtatctccctc | SEQ. ID NO: 91 |
| 3. Intergenic region between AN1035 and AN1034 | | |
| (named 1035P, 527 bp) | | |
| 1035P_F | cctggtgtgattgggctgattag | SEQ ID NO: 92 |
| 1035P_R | agtactgctttcaaaagtatatcatctgc | SEQ ID NO: 93 |
| 4. Intergenic region between AN1034 and AN1033 | | |
| (named 1034P, 849 bp) | | |
| 1034P_F | tgcgggagggtaggaggg | SEQ ID NO: 94 |
| 1034P_R | tataaccacttgcctgaggatc | SEQ ID NO: 95 |
| 5. Intergenic region between AN1033 and AN1032 | | |
| (named 1033P, 605 bp) | | |
| 1033P_F | cctgtttagagtggccagaag | SEQ ID NO: 96 |
| 1033P_R | tatgcaactgggccggag | SEQ ID NO: 97 |
| 6. Intergenic region between AN1032 and AN1031 | | |
| (named 1031P, 384 bp) | | |
| 1031P_F | attcagcctattgagattacag | SEQ ID NO: 98 |
| 1031P_R | tgcgcctggattcgggatgtag | SEQ ID NO: 99 |
| 7. Intergenic region between AN1031 and AN1030 | | |
| (named 10317, 591 bp) | | |
| 1031T_F | ggcatcgtctacaagcagatgc | SEQ ID NO: 100 |
| 1031T_R | ctggttactgtttattttgact | SEQ ID NO: 101 |
| 8. Intergenic region between AN1030 and AN1029 | | |
| (named 1029P, 1370 bp) | | |
| 1029P_F | aacgaggtccaggtgacggtaa | SEQ ID NO: 102 |
| 1029P_R | gattgctggtctttgtagtctc | SEQ ID NO: 103 |
| Primers used for generating YM186-YM195 | | |
| (ctv in the afo regulon) | | |
| 1. ctvA gene fragment (7527 + 50 bp) | | |
| 1036P_R+ctvA_F | ccataatccccatccacaaggcacc | SEQ ID NO: 104 |
| | atggcacacatggaaccgat | |
| 1036T_F+ctvA_R | agaagaacaacatgaccgatgcagc | SEQ ID NO: 105 |
| | tcagtcatggtccccctcc | |
| 2. ctvB gene fragment (687 + 50 bp) | | |
| 1036T_R-ctvB_F | ctggagggagatacggctatccacc | SEQ ID NO: 106 |
| | ctagcgacgaggcttccg | |
| 1035P_F-ctvB_R | tcctaatcagcccaatcacaccagg | SEQ ID NO: 107 |
| | atgacctcctaccagctttcc | |
| 3. ctvC gene fragment (1611 + 50 bp) | | |
| 1035P_R-ctvC_F | atgatatacttttgaaagcagtact | SEQ ID NO: 108 |
| | tcatacttccttgacattgaacacc | |
| 1034P_F-ctvC_R | cctcctaccctcctaccctcccgca | SEQ ID NO: 109 |
| | atggaaggaaagcaccctc | |
| 4. ctvD gene fragment (1132 + 50 bp) | | |
| 1034P_R-ctvD_F | agcgatcctcaggcaagtggttata | SEQ ID NO: 110 |
| | tcagaattgagattcctcccg | |
| 1033P_F-ctvD_R | acaccttctggccactctaaacagg | SEQ ID NO: 111 |
| | atggccctttcagcctac | |
| 5. AfpyrG cassette fragment (1885 + 50 bp) | | |
| 1033P_R-pyrGF2 | tgcaattctccggcccagttgcata | SEQ ID NO: 112 |
| | caatgctcttcaccctcttcg | |
| 1031P_F-pyrGR | tggctgtaatctcaataggctgaat | SEQ ID NO: 113 |
| | ctgtctgagaggaggcac | |
| 6. 1031P-partial AN1031 fragment (1145 bp) | | |
| 1031P_F | attcagcctattgagattacag | SEQ ID NO: 114 |
| 1031P_R1 | cctagtaggtgggatttgaa | SEQ ID NO: 115 |
| PCR primers for large fragment ctvF1 (6935 bp) | | |
| 1036P_F3 | atgtgctctacggacgaaaaat | SEQ ID NO: 116 |
| ctvA_R1 | gggagaagatgaaccagttgtc | SEQ ID NO: 117 |
| PCR primers for large fragment ctvF2 (7454 + 25 bp) | | |
| ctvA_F1 | tcggtggcatagacactatcac | SEQ ID NO: 118 |
| 1034P_F-ctvC_R | cctcctaccctcctaccctcccgca | SEQ ID NO: 119 |
| | atggaaggaaagcaccctc | |
| PCR primers for large fragment ctvF3 (6926 bp) | | |
| ctvC_F1 | gcagtacctcaccgttgtatga | SEQ ID NO: 120 |
| 1031P_R2 | atgaagagcgcctgtttctg | SEQ ID NO: 121 |
| Diagnostic PCR primer set 1 (2701 bp) | | |
| 1036P_F | aatgactggtccgtccgtac | SEQ ID NO: 122 |
| ctvA_R2 | gggatcacgtctactggaactc | SEQ ID NO: 123 |
| Diagnostic PCR primer set 2 (3242 bp) | | |
| ctvA_F2 | gccatgttagaagggtatgagc | SEQ ID NO: 124 |
| ctvA_R3 | tctgggtatacagcagggtctt | SEQ ID NO: 125 |
| Diagnostic PCR primer set 3 (2345 bp) | | |
| 1035P_F1 | gagctggttaggatcaactgct | SEQ ID NO: 126 |
| 1034P_R1 | atggagtcctgtagtccgaaaa | SEQ ID NO: 127 |
| Diagnostic PCR primer set 4 (2199 bp) | | |
| pyrG_F3 | atatgccgtctagcaatggact | SEQ ID NO: 128 |
| 1031P_R1 | cctagtaggtgggatttgaa | SEQ ID NO: 129 |
| Primers used for generating YM283-YM287 | | |
| (5 plu genes in the afo regulon) | | |
| 1. pl-ggs gene fragment (1053 + 50 bp) | | |
| 1036P_R- | ccataatccccatccaccaggcacc | SEQ ID NO: 130 |
| GSS_START | atgagaatacctaacgtctttctct | |
| 1036T_F- | agaagaacaacatgaccgatgcagc | SEQ ID NO: 131 |
| GSS_STOP | ctactctgcgatgtacaacttttcc | |
| 2. pl-cyc gene frag ment (2880 + 50 bp) | | |
| 1036T_R- | ctggagggagatacggctatccacc | SEQ ID NO: 132 |
| Cyclase_STOP | tcaatggtggattccattgctcccg | |
| 1035P_F- | tcctaatcagcccaatcacaccagg | SEQ ID NO: 133 |
| Cyclase_START | atgggtctatctgaagatcttcatg | |
| 3. pl-p450-1 gene fragment (1572 + 50 bp) | | |
| 1035P_R-P450- | atgatatacttttgaaagcagtact | SEQ ID NO: 134 |
| 1_STOP | ctacaacgcagcgaacgcttcctta | |
| 1034P_F-P450- | cctcctaccctcctaccctcccgca | SEQ ID NO: 135 |
| 1_START | atgctgtccgtcgacctcccgtctg | |
| 4. pl-p450-2 gene fragment (1578 + 50 bp) | | |
| 1034P_R-P450-2- | agcgatcctcaggcaagtggttata | SEQ ID NO: 136 |
| STOP | ctaatagtctgcaacatcgtggatc | |
| 1033P_F-P450- | acaccttctggccactctaaacagg | SEQ ID NO: 137 |
| 2_START | atgaatctttctgctctgaaggctg | |
| 5. pl-sdr gene fragment (762 + 50 bp) | | |
| 1033P_R-SDR- | tgcaattctccggcccagttgcata | SEQ ID NO: 138 |
| START | atggaaggcaaggtcgcaatcgtca | |
| 1031P_F-SDR- | tggctgtaatctcaataggctgaat | SEQ ID NO: 139 |
| STOP | ctaaatgacactccacccgttatcg | |
| 6. AfpyrG cassette fragment (1885 + 50 bp) | | |
| 1031P_R-pyrG_F2 | tgtctacatcccgaatccaggcgca | SEQ ID NO: 140 |
| | caatgctcttcaccctcttcg | |
| 1031T_F-pyrG_R | ctagcatctgcttgtagacgatgcc | SEQ ID NO: 141 |
| | ctgtctgagaggaggcactgatgc | |
| 7. 1031T-partial AN1030 fragment (1317 + 24 bp) | | |
| pyrGR-1031T_F | gcatcagtgcctcctctcagacag | SEQ ID NO: 142 |
| | ggcatcgtctacaagcagatg | |
| AN1030_R1 | tttggtctcttccacaaggact | SEQ ID NO: 143 |
| PCR primers for large fragment pluF1 (9224 bp) | | |
| 1036P_F3 | atgtgctctacggacgaaaaat | SEQ ID NO: 144 |
| 1034P_R1 | atggagtcctgtagtccgaaaa | SEQ ID NO: 145 |
| PCR primers for large fragment pluF2 (8227 bp) | | |
| P450-1_F1 | aactcaatccagctacgaccat | SEQ ID NO: 146 |
| AN1030_R2 | gtctttgactaccggagcaagt | SEQ ID NO: 147 |
| Diagnostic PCR primer set 1 (10136 bp) | | |
| 1036P_F | aatgactggtccgtccgtac | SEQ ID NO: 148 |
| 1034P_R | tataaccacttgcctgaggatc | SEQ ID NO: 149 |
| Diagnostic PCR primer set 2 (9500 bp) | | |
| 1035P_F1 | gagctggttaggatcaactgct | SEQ ID NO: 150 |
| AN1030_R1 | tttggtctcttccacaaggact | SEQ ID NO: 151 |
| Primers used for generating YM343 | | |
| (7 plu genes in the afo regulon) | | |
| 1. pl-sdr-1031P fragment (1146 bp) | | |
| SDR_START_FF | atggaaggcaaggtcgcaatcgtca | SEQ ID NO: 152 |
| 1031P_R | tgcgcctggattcgggatgtag | SEQ ID NO: 153 |
| 2. pl-atf gene fragment (1134 + 50 bp) | | |
| 1031P_R-ATF- | tgtctacatcccgaatccaggcgca | SEQ ID NO: 154 |
| START | atgaagcccttctcaccagaacttc | |
| 1031T_F-ATF- | ctagcatctgcttgtagacgatgcc | SEQ ID NO: 155 |
| STOP | ctactgtgctacacgagggggattc | |
| 3. pl-p450-3 gene fragment (1569 + 50 bp) | | |
| 1031T_R-P450- | gccagtcaaaataaacagtaaccag | SEQ ID NO: 156 |
| 3_STOP | ctagccactagcaggcttcgtgaac | |
| 1029P_F-P450- | acgttaccgtcacctggacctcgtt | SEQ ID NO: 157 |
| 3_START | atggctccgtcaacggaacgtgctc | |
| 4. AfpyrG cassette-PalcA-partial AN1029 (3395 + 25 bp) | | |
| 1029P_R-PyrGF | ccagagactacaaagaccagcaatc | |
| | caatgctcttcaccctcttcg | SEQ ID NO: 158 |
| alcA_AN1029_P6 | atggcagtgagcagacattg | SEQ ID NO: 159 |
| PCR primers for large fragment pluF3 (8900 bp) | | |
| SDR_F1 | cgctggtatttcggactacttc | SEQ ID NO: 160 |
| alcA_AN1029_P5 | tgctggggtatggctatctc | SEQ ID NO: 161 |
| Diagnostic PCR primer set (9205 bp) | | |
| SDR_START_FF | atggaaggcaaggtcgcaatcgtca | SEQ ID NO: 162 |
| alcA_AN1029_P6 | atggcagtgagcagacattg | SEQ ID NO: 163 |

TABLE 7
Genomic DNA sequence of the afo locus in strain YM81.

| Region | DNA sequence |
|---|---|
| intergenic | aatgactggtccgtccgtacttagaaagggtgtttctgtccggcagttatttaatgtcggctgtctgctcttgcaatttctctt |
| region | ttgatttatctttcgtggtgtatctcgccggaacgaatggccacggttcgcgtttgcgttcatgttcatgttcatagagcagc |
| between AN1037 | tgcgaagtttcaaatgttcgttcgttcggctcggcttggctaggcgtatgatggtgttatgtttaggttgagaaggtattctt |
| and AN1036 | agttgggagctagagaaaagattatttgttccctgcaattttgctgtaccccggaaacatagaactgttactgtaccaata |
| (named 1036P, | ctctgcgttccctccccaatgcaccccatacatatggagttggagcctgtacctttgtcgataagcttattctccaatcaactc |
| 1487 bp) | tgctattgcagcttttcacttgagctttcttattcgtatgtgctctacggacgaaaaataagctttgttgcctgcagatcacctt |
| (SEQ ID NO: 1) | ggcagctgtgctgcgcctagacttataatgcaacgtttttaactttttgtttttcttttttctttcttttttaaactagttttca |
| | catgagctacccgttcattataaccatcagctctagctaggacaggatcgcatgagtatatacctatttatattccttccctccc |
| | aactcggactcacgctttatatatatgtctactattactcgtgggtgaagagaagtttacgactatttagcctagatgaagg |
| | ataggttgtgcaatgctcgatagcgtagcatttaaccctacctagtaatgagctacttgggctgctagaataaatctccca |
| | atccaagctaatgtagtcagagctgaacgcaagtctcgtacatggccctacgaggcatcacaatagccctaaagagta |
| | tcacgtgaccatactagcaccgcaatgagttcaggatccgacaatagcgaggctgtatccaagtgcgccgaataatgt |
| | ctatcactgtagaaatatatctgattcgctcagctggtcgataggcgaagcatcggagttggcggagttggcggagttg |
| | caggacttgctggattagggctgaggtcagacggactctcactctccgctatagacactgggcgatgttgtaggcagc |
| | gatgggagaatgtgcattgcacatggtccggagatttctggagtcaggtcatgcagtctagatcctgactgcagtagaa |
| | tgtgcagattccggagcttggggagttaacctgcagtaagctcagctcaagcaatgatcggtaggtaggcctggtggc |
| | catatcagctatagatgcgatccgcgcctcaagcgcatttcaagccctccctcttcaatacgtttgcgataccttagagaa |
| | acaaatcaacatccatcaactggcacagattcatctaccaactcaacgtgattacccgtccagctttgacctaaacctcc |
| | ataatccccatccacaaggcacc |
| AN1036 | atgggcagcacatcttccgagcccacatacgacagtgagcccatcgcgattattggcctttcgtgcaagttcgctgggt |
| (8049 bp) | ccgcagacagccccgagaaactatgggagatgcttgcggaagggcggaatgcatggtcagagatccctgagtcgc |
| (SEQ ID NO: 2) | ggtttaaccacaaggccgtgtatcatcctgatagtgagaagctggggacggtacgtctttccttctagacttgagtttcag |
| | tggtgaagtggatgggaagcaagaacctggccagactaacgcggaatcttcgcagacgcatgtcaaaggggcacat |
| | tttctcgagcaagatgtcgggctcttcgacgcggcattcttcaattattcggcggagacagctgctgtacggtccctatg |
| | aacgatttcaggatgaatggccaggctaactgagcatgatgtacggatagaccctcgatccgcaattccgcttccagct |
| | cgagtccgtctatgaggctcttgaaaatggtaccaccctccccccaacagcccttgcgcaaggctgaacagagagtac |
| | agctggcctgacgattccatccatcgccggcaccaacacctccgtctacgccggcgtcttcacgcatgactaccacga |
| | aggtctgattcgcgacgaagacaaactgccccggttcctccccatcggaaccctctccgccatgtcctcgaaccgcat |
| | cagccacttcttcgacctcaaaggagcaagcgtgactgtagacaccggctgctcgacggccctggtggccctgcacc |
| | aggccgtcctcggcctgcgcacgcgcgaagcagacatgagcatcgtctctggatgcaacatcatgctgtcgccggat |
| | atgttcaaggtgttttcaagtttgggaatgctaagccctgatgggaagagctacgcctttgactcaagggcgaatggata |
| | cggacggggcgagggcgtagcgacgattatcgtgaagcgactcgcggatgcgctgagggacggggatcccgtgc |
| | gcggcgtgatccgcgagagctatctgaatcaggatggaaaaacagagactatcacctcgccgtcacaggaagcgca |
| | ggaggcactgatcaaagaatgttatcggcgcgcggggctgtcgccgtcggatacacagtacttcgaagcgcatggg |
| | acaggcacccccactggagatccgattgaggcgcgctcaatcgcgtcagtatttggaaagaatcgagagcagccgtt |
| | gcggattggctctgtcaagacgaatatcgggcatactgaggcggccagtggtcttgccgggctgatcaaggtcgtgct |
| | ggccatggagaaggggttcatcccgcccagcgtaaactttgagaagccgaatccgaagctgaagctggatgaatgg |
| | aggctaaaggtggcagatactttggaaaagtggcctgcaccggcggagcggccatggagggcgagcgtgaacaac |
| | tttgggtatgggggtacgaacagccatgtcattgtggaaggggtgccgaagagattatacacaccggcaaatggaaat |
| | gagaccggccagataaagcatgagacagagagcaaagtgctcctcttctctggccgcgacgaacaagcctgccagc |
| | gcatggttgccagcacgaaggagtacctgaagaagcgcagggagcaggatcctcccatgacacctgaacaagtcaa |
| | gaccctcatgcaaaatctcgcctggacattaacgcagcaccgcactcgcttctcctgggtctccgcacacgcggtcaa |
| | gtactcgacctccctggacaccgtcattgacgccctcgagtctccgccgccggcctcaagacccgttcgcatccctga |
| | ctctccattccgtattggcatggtcttcacggggcaaggtgcgcagtggcacgccatgggccgcgagctgatcgccg |
| | cgtacccggtattcaaggcaaccctagacgaagcggaacagtatttgcgccaactgggggccggctggtccctcatc |
| | gaagagctgatgaaggatgcagccacgacaagagtcaacgacaccggcctcagcatccctatctgtgtcgccgtgc |
| | agatcgctctcgtccgcctgctcaaggcatgggggatcactgcctcggccgtgacatcccactcgtccggtgagatcg |
| | ccgccgcgtatacggttggcgctctctcgctgcgccaggccatggccgccgcctactaccgcgctgccatggcagca |
| | gacaagacgctgaagagcgcagaggggccccaaggcgcaatggttgccgtgggtgttgacaaggctgccgcgca |
| | ggcatacctggaccgcgttgagaaatcggcaggccgcgctgtggtggcatgcatcaacagccccagcagcatcacc |
| | attgccggcgacgaggcagccgtcgtcgcggtcgagaagttggccactgaggagggcgtctttgcgcgccgactca |
| | gggtcgagacgggatatcactcgcaccatatggagccaattgcgagcccgtaccgggaggcgcttcgcgccgcatt |
| | ggcccaggaagatgctgagtctggtaccaaggaccagactgatgtcccgggctttgcggatgccactaaaccgggc |
| | agcctagaccacaccgtcttctcctcccccgtcacgggcggccgtgtcacagatgccaaagtcctctctgacccggag |
| | cactgggtccgcagtctgctccagccagtgcggttcgtcgaggccttcactgatatggtgcttggctccacagatagca |
| | gcaatattgacctgatcctcgaggtcgggccgcatacagcccttggcggaccgatcaaggagatccttgccctgcctg |
| | acttcagcagcaggaatgtcagcctcccctacatgggctgcctcgttcgtaaagaagatgcgcgcgactgcatgctca |
| | ctgctgccttaaaccttttctccaagggccacagtatcgacctgctcagactcagcttctcgtctggcatcccagagttgc |
| | aagtcctgaccgacctcccctcatacccgtggaaccacagcatcagacactggtctgagtctcgccgcaatgccgcgt |
| | accgtaagcgcagccaggagccgcatgagctgctgggcgtgctggaaccgggcacgaacccggacgctgcctcgt |
| | ggaggcatatcatcaagctctccgaggcgccgtggctgcgcgaccacgttgtccaggggaacatcctctaccccggt |
| | gcaggattcgtgtgtctcgccattgaggcaatcaagatgcagtctgccatgagcgggacgaatgatgtgaccggtttca |
| | ggctgcgcgatgtcgagatccatcaggcgctcgtgattgcggacagtgcagacggcgtcgaagtgcagacgaccct |
| | ccggtccgtaggaggcaaggtcatcggcgccagaggctggaagcagtttgagatctggtcggtcagcgcagacag |
| | cgagtggacagagcacgcgaggggtctaatcaccgtcgacactgagaccaaggcatccacgctcgtggcaagcac |
| | tctcgatgaatccggctacacgcgccgcatcgacccgcaagacatgtttgctagcctgcgcgcaaaggggctcaacc |
| | acgggcccatgttccagaatacgctgagaatcctgcaggacggaagggccaaggagccgcagtgcgtcgtcgatat |
| | caagatcgccgacgtatcgagcagcaaggacagcggccggatgagtcttctgcacccgacgacgctcgactcaatc |
| | gttctctcctcatacgccgcagtacccagctcggatccgtccaacgacgacagcgcgcgcgttccccggtccatccgc |
| | agcctgtgggtgtcgagcatgatcagcagcgccccgggccatacgttcacctgtaatgtgaagatgccgcatcacgat |
| | gcgcagagttacgaagcgaacgtgacagtcgtggacgaggccggagccagagctgagagcatggtcgagatgca |
| | gggtcttgtctgccagtctctcggccgcagcgcaccagcagaggaccgagaaccctggacgaaggagctatgcgc |
| | gaacgtcgaatgggcgcctgatctctccctctctctcggccttccgggctcgtcagacgccatcgacaggcgcctcaa |
| | caccctccgcgaccagaatccagacgagaggagcatcgaagtgcagacggtcctgcgccgcgtctgcgtctacttc |
| | agccacgatgccctttcctccctgacagaaaacgacgtggcaaatctcgcattccaccatgtcaagttctacaagtgga |
| | tgcaggataccgtcaacctggcactcgcgcgccgctggagtgccgacagcgacacctggattcatgacagtcccgc |
| | cgtacgggaaaagtacatttcccttgctgggtcgcagacggtggacggagagctgatctgccagctaggcccattgct |
| | gctgccggtccttcgcggggaacgagcgccgctggaggttatgatggagggacgcctgctgtacaagtactacgcc |
| | aacgcataccggctggagcccgccttcgagcagctcaagtcattgctgggcgcgatcctgcataagaaccctcgtgc |
| | cagggttctcgagatcggagccggcaccggcgctgccacacgacacgcgctcaagaccctagggactgatgagga |
| | tggcggtcctcgctgcgagagctggcactttactgacatctcctccgggttcttcgaggcagcccgcgctgaattcgcc |
| | acctggggcggcctgctggagtttaataagctggatatcgagcaggaccccgaagcgcaggggttcaagctcggttc |
| | ttacgatgtcgtggtcgcctgccaggttctgcacgccacgaagagcatgcaccggactatgaccaatgtccggtccct |
| | gatgaaacccggcggcacgctgctccttatggagacgacacaggaccagattgacttgcagttcatctttggtctcctg |
| | ccgggttggtggctgagcgaagagcctgagcgccacgcgagccccagcctgagcattgacatgtgggatcgggtg |
| | ctcaagggggccggctttacgggagtcgagattgacctgagagatgtgaacgttgatgctgagagtgatctgtacggc |
| | atcagcaatatcatgagcacggctgtcggcacggcgggttcgagccctgagaaggtggatgccgcccaggtggtga |
| | tcgtgacgggcaacaagacgggctttcaggacgattgggtcaggggactgcaggcagccattgctcaggactccgg |
| | tagcgatgcccttccagagattatatccctcgagtctccctcgctcggggcagaggccttccagtcccggctggtcgtc |
| | ttcgtcggcgagcttgacagacccgttctggcgtctcttgactccacagagctcgagggaatcaagaccatggccctc |
| | gcctgcaaaggtcttctctgggtcacccgcggcggcgcggttgagtgtacggaccccgactctgcgcttgcatctggg |
| | ttcgtccgcgttctgcgcaccgagtatctcggccggcgcttcttgactctcgacctggacccagcagcccattcgcctg |
| | cgtctgatatctcagtcattgtgcacctcctctcctcgcgcctacagccggccgttgagacagcggccccggccgaca |
| | gcgagttcgctctgcgagacggcctcctccttgtgccgcgcctttacaaagacgttgtctggaatgcactgctggagcc |
| | tgaggtccccgactgggcctctccagagagtattcccgaaggcccccttcttccaagccaagcggccgcttaaactcg |
| | aggttgggatccctggtctgctcgatacactcgccttcggcgacgaccccgacgcgctggacgccgccgggcccat |
| | gcccgacgagatggtcgagatagagcctcgcgcttatggcctcaacttccgcgacgtcatggtggccatgggccagc |
| | tcaaagagcgcgtcatgggtctagagtgcgcaggcgtcatcacgcgcgtcggcgctgaagctgcggcgcaaggctt |
| | cgccgtgggtgaccgggtcatggccctgctgctgggcccgttcagctctcgtgcacgggtgagctggcacggagtc |
| | gccagtatgcccgcggggatggggtttgcagatgctgcctctatcccgatgatcttcaccacggcgtacgtcgctctcg |
| | tgcaagcagcgcgactgtcgcaggggcagacagtgcttattcacgccgctgcaggaggtgtagggcaagcagccgt |
| | gatactggccaaggaatatctcggagcagaagtctttgcaaccgtgggctcgcaggagaagcgagacctactgatca |
| | aggagtacggaatccccgacgaccacatcttcaactctcgcgacagttcctttgcaccggctgccctggccgcaacag |
| | ccggacggggcgtggactgcgtccttaactcgctaggtggcgccctcctccaagccagctaatcgaggttctcgcgc |
| | cctttggccactttgtcgagatcggcaagcgcgatctcgagcagaacagcctgctcgagatggccaccttcacgcgc |
| | gctgtctccttcacttcgctcgacatgatgaccctcctccgccagcgcggcgacgaggcgcaccgcgtcctgagcga |
| | gctcgcccggctggccggccaggggatcgtcaagcccgtccaccctgtgtccgtatacccaatgcgccaggttgaca |
| | aggccttccgtctgctgcagacggggaagcatctcggcaagctggtactgtccaccgagcctgacgaagaggttaga |
| | gttcttccccggccggccacgcccaaattgcgcgccgatgcatcttacctccttgtcggcggcgtgggaggtctcggc |
| | cgctccctcgccagctggatggtcgaacacggcgcaaaacaccttatcctcctctcgcggagtgcaggcaagcagg |
| | acagcagcgcattcgttaatggcctacgggacgcaggatgccgcgtcgccgcaatctcctgcgacgtcgccgacag |
| | ggccgacctcgaccgcgcgatcgcggccgcctcagagttggggttcccgcatgtccgcggcgtcatccagggcgc |
| | gatggtcttgcaagactcgatcattgagcagatgagcattgcagactggaatgcggcaatcaagcccaaggttgccgg |
| | gacacgcaacctccatgaccgcttctcccagcgcaacagcctcgacttcttcgtcatgctctcttccctatccgcgatcct |
| | gggttgggccagtcaggcctcctacgcggctggcggaacgtaccaggatgcgctggcgcgctggcgctgctccaa |
| | gggtctgcctgccgtatccctcgatatgggcgtaatcaaagatgtcggctacgtcgccgagtcgcggtcagtctcaga |
| | ccggctgcgcaaagttggccagtccctccgcctctctgaagagtcgatcctccagaccctggcaacggcggtcttgca |
| | cccattcggccggccccagctcctcctgggcctgaactccggcccaggcagccactgggacccttccagcgacagc |
| | cagatggggcgtgacgcccgcttcgcacctctccgctaccgtaagcccgcatctacgaagtccgctcagacatcttcc |
| | agcggcgacggcgaagagcccctttcatccaagctcaagtcagccgattcccccgatgcggcggcgaactatgtcg |
| | ggggtgcaattgccaccaagctcgcagacatcttcatggtccctgtggccgatatcgatctgaccaagccgccaagtg |
| | cgtacggggtcgactcgttggttgctgtcgagctgaggaatatgctggtgctccaggcggcgtgtgatgtgagtatcttt |
| | agtatcctgcagagtgtgagccttgcggcgctggcggggatggtggtcgaaaagagtgcgcatttcgagggaagtgc |
| | cacgggaactgtcgttgttgcttga |
| intergenic | gctgcatcggtcatgttgttcttctatagagttgaagcaaggtttgtagtttgctctgggtgtctggagttgtctggagttgtc |
| region | tggagttttgttatgatgttgatgggtacttcttcatactagcattttggcatgttataagaacatattatcagttaaatgtctttc |
| between AN1036 | aatttaatcaatttgtttttagaatgatgttgtctgcctggctatgtatctagatcctatacaagctctatcgactcgacctaac |
| andAN1035 | tactacgacttgaaagtcaagcgagaagtgatgatatgaacccatatgtcagacccgctaaatttattagtgataacaact |
| (named 1036T, | atattactcagagcttttctttctagagtatgttagaattgccctttctggctcagtgggaagctcgagacctagtccttagtc |
| 1768 bp) (SEQ | acgtgctgctacatcatgtaaatataagccctacatggctgtcttgtgcatgaggctaacaccattatctgtcactggtcct |
| ID NO: 3) | tttatttggttcttttctttactttctcgggcgggggggaaagccgctaacactgtctatcgcttggacagaaactcaccagt |
| | ttgttcgcaatcctgaagcgtatgggaagcttacagttaaggagtagctcgagtctggaccctgttttcgacttgtaccttt |
| | gatttggatgactggttaacctcagcttatgtatgatgtgctctcatggtgtcaatatctggtagtctgattctgagcaatttg |
| | atagtatctgatggctggcgagtaaggccagggcgatgactggtataaagtcagccctaaaacttccatccgagatgta |
| | aaaccatcgattcccctccaagatctcctgacgagactaaacaaagatcaagtggccttgtagtaactctagcaagcag |
| | cgacaaaatgcctcaacacgagatgaccaagtcagactcggaacgaatccagtcctcgcaggtaagagcatcagga |
| | catttgctaataccattccgccccgctaatctgcttgaatgcacacaggctaaaagcggaggggacatgtctcttggag |
| | gattcgcctcgcgcgccctgtctgccgggactgctgggtcaattcccagtcctcggccactgcttccggccacgcgga |
| | ctcgggtgccggatctgcaggcggatctcattcggccgcacctggcggtgatgcggggcagggaagaagataaaa |
| | gtaccctgttgtctttggggcgttgaggtataatggcatcgtggtagaccgactgggcttttttttttgatatagttgatcctg |
| | aagcggaggacagttggtaggataaatgaaagatactgaaccatgcccggattttgtgctcaaggacctaaaactgag |
| | aagctgaatctgttcttgtctgggagaaggcctgccagctgcatccgagtatctatcttgccaggaccaaaccgggtct |
| | gggctcagttcttctaacttcttagtggagttttgcagtgtagattcctttgcactatctggtatcctagtagcagcctacca |
| | ggaaataagagataaataaagtcttaattggcattattatgtttctcagaactatatatctcggaacaaagctgagcagac |
| | agaagtttaccctcacatatggacaaattgcgtgctcaggcataagtcggaaacagccttagccaggtcaacacttgta |
| | gccttcgctagacgacgccccagcttttcataatggccggcctggagggagatacggctatccacc |
| AN1035 | ctagaacctcggaataggtgtccccttcccaaagacccccttgggatcccactttctcttgagatacgacagctttggca |
| (complementary, | gattctccttgctccaccacgcctccggcccctcatcaccaaatgcataattgacgtagatatgtggctggtcagcagga |
| 1593 bp) | aacccgctggtggcatggagcttttcgcgcagtgagaccagcagctcgttcgttggagcctccagttccggattcaag |
| (SEQ ID NO: 4) | aatatattctcgtgcagccaaaacatcttcgtgtcgcgccagggatacacggccgtgtgcgcaggcgttttgagcgtgtt |
| | gttgttcgcgtatcgctggaacagactctgccccagataccccgggtactgctcgtagaacgcggtcatgtcgtcgaac |
| | acctcctgcatggtggccgcgtctgttcggcctagtcctacggtaccgccggagacgtaggctcccgtctggcagggt |
| | ccgtcgaggccagcgtacagctcgacaagagtgacgttcgatacgttccggctgatcgggccgagcgcctcggcgt |
| | gctcccagtggtcgacgaaagtggcccagggggcgaagtgcttgatgtccacggtcaagagggtctcgttgatggtg |
| | cggtcgtacccgatcgagagctgcactcccagttcaggagggaggacattatcgaggacagagaggtactcgaaga |
| | cgccgagactcttggatgagttatacacaaacgtgccgatcacggcgtcgccgttgttcggctggtcgaacatcttgaa |
| | tgtggcggcggtgatgatgccgaagtttgcaccggcgccgcggatagcccagaggagatcgctattgcaggtctcat |
| | tcgcagtgatcagctcgcccgtcgcagtgataatgcggacagagacgagtgcgtccacgccgaggccgaagagcc |
| | ctgtttcgtacccaattccgccgccgatagtggcgccaataaccccgacgcagggagagttgccgcgggctgtctcta |
| | ttagacggcatgcttaagaaggagaaggagagaatgagggggcatacggatggccttgcccgctttatagagcggct |
| | cagtgatatctcccagctttgcgcccgcaccaacggtgacggtgttggactccagatcgatgtccacgttgttaaagttg |
| | gccaggttgatatcaagccctttgacggtgccgtaaatcagactagtgccgtggccaccgctggtggccatgaagctg |
| | acattgttcgcgacggcgatgcggacctgcctcgtcagtacactatttccttaagaagcaacactacaaaggcaaaca |
| | gagaacaagaggcataagaagaagaagaagaagaagggggtatacaatctcctgtaaatcctcctcggtctgcggct |
| | tgatcgcgcctgtccaggtcggaggcctccattcggaccatctgggtgatacgacctcgtcaaaatccgcgtcgccaa |
| | cctcggcgatctctgtttcaggcgagacgtatgggccgaaaagagattcgaggtcgatgcttgccgcgcgcgccgca |
| | gcgactagtgttattgactgaagcagaaaccgcat |
| intergenic | cctggtgtgattgggctgattaggacaggccggatgggtgtgcaagataggaggagaggactggtacggcgaatga |
| region | gctttaatagccggtcagagattgcgcgtggctgcgcccagatccagcagctccagccatactccagcatactccggc |
| between AN1035 | cagccgggggcatatggcgtggtcactggagctggttaggatcaactgctggttaaggcttactgtgttgccatgctta |
| and AN 1034 | cggtgcaccgagagggaaggttggagttaacggagttgtaactccggggatccaattagggcttacagtctgcaaatc |
| (named 1035P, | catgcaaagtccgctgcgcccctgacacagcaaggaacagtgtagagtccgattggatagcggagttgaggtgactg |
| 527 bp) (SEQ ID | gctggttcctgttagcccctgcatcgacctgcaatgtattgcatcaaattagggctagcctctaactccgttagactatcc |
| NO: 5) | gcaacgcctgtcacacacgtggctaggcagcagatgatatacttttgaaagcagtact |
| AN1034 | ctaaatttgtggggtatatggtgtggctatgctggatcgtcgtctaaggcccattgttaccagcactatttaagttgtcgac |
| (complementary, | aagatctagtcacatactaccagcgagtgcatgcagggccgcaggatatagaccggactcagcattgagccatgtctt |
| 8931 bp) (SEQ | tacgtaccactgtagttagccactgagtgatagacacattgcagcttctctagactgatcagtaatgacgatctcgcttga |
| ID NO: 6) | tactgtctgcttatgcagtatttatatagtatagtgtagactacggacagattgcatctattccgtgaggaaagggtcttcaa |
| | gcatctataaggaataaaaactcgctgtcactgtacatgctctagctacctaaaagagatattgcaggtgcattgataaa |
| | ggactatgcagagagctagatctcatgtttctactcaagttacagggcatggcctagcctaatatgcagttgtcctatatgt |
| | gagctagctggagccgatgggaagtgtgtttgatgaaactgattggaataatatggaattgtaagcaaagtaacaacag |
| | tctagatacaatgaatcattcccaacaccagaatacgccagactaaaaccagagttagcgaaacaaagaatatctgtaa |
| | gctcaagcaatcaggcgaggtagcccatatccttccaagcctgcacatacaacctcgcaagctccgtgccaacaggc |
| | ccaacccccgccatagtggtcgagtgctccttcgccttgcttgtgtcaagcaccaggccgccacagctcatgcgctcg |
| | aaatggtcgtcgaggaaatccaccagccgcgccgccggattctccgtctccatcggcagcggagaccgccgcaccc |
| | ttgagatccacgtcttgaatgggatgatattcgatgcgggaatatcgagtgctgacgcaagcacatggttcatggcttgc |
| | cagttctgaccgacaggattgtccatatggtacactgggtatgcctcgtcgcctcgtgaggtgagatggagcaggtcca |
| | caacaccagcagcgcagtaatccacaggaatccactgcatctggccctgcaggtccggccaagcacgcagcgactg |
| | cgaagacttgactaagaaagcaaagtgctcgaccgggttccagaaaccgctcgtcgacgagcccgagatctggccg |
| | ggccgcacgaccatcgcccggaagagaccgggatgccggtgaagggtctcatcaaccatgcgctcacaaatccatt |
| | tcgcctcgccatatccggacggcagtgctgcagatagcgggacgcggtcctcgctcacgcgggactgcccgcagaa |
| | tccgacgacgccgatggaggagatgaattggaagcccacgcggctggaaccattgaagggccgttctgcaatgtca |
| | cgggcaagatcaagaagattccgcattgcctgtagctggggctcgaatgcggacactggccgtgtcccgctcatggg |
| | ccaggcgttgtggatgatatccgtcgcgttctcgaggagccagccgtactcaagcggcgggaggcccagctgtggct |
| | tagaagtgtctgtctctaaaacgcggagctttgcccgtgcgccgggggacagggtgatgccgcgggctgttagggct |
| | gcctgttggcgcttctctggggtggtgctgctgctgcgacggttgaggcacaccaccgtcgcaaccgacggtgtctcg |
| | gcgagtctctgaacgatatgtgagcctaggctgccagtcgcaccagtgacgatgacgacggcctcgtgcgctctgcg |
| | tcctggtgctgcgtgcggcgcctgtgttttgccagactccttctcggcccggctcgctaaagcacggagtttgggcgtct |
| | cccagccagccgtgtactttgcaactaggctctctgctgtcgctgtgcgcgcctcaacattctcccggttcaactcgggg |
| | atgagggtctgcacgggccctggcttgggcagacgggctccctgagcccccgacgcgagcgcgataatgactttct |
| | ggaaggtattttcaggcaggttgccgtctgtccagtcgacgtggccaaacccggccctgtgcagctcactctcccagt |
| | gctcggccggtacgacggcgtggtgccgcccgtcatcgaacagccaccacccctcgagcaggccgaaaacaagat |
| | cgacaaaggggaccacctcggtcatttccagcatcatcaaaaacccatcggggcggagtgcctgatggatgttggac |
| | agcgagaccccgagattgtgcgtggcatggatggcattgctggcgagcaccagatgctggttcctgagctcgtcggc |
| | cgggggcttctcgatatcgtgcacggcgaaacgcataaacgggtattgcttgctgaaccggcgacgggcgttggcga |
| | ccatgctgggggaaatgtctgtgaaagtgtattcaatgggcagggcgcccgattcagccagggtcgccaggaacgg |
| | cgccatgatgagcgtggtgcctcctgtgccggcgcccatctcgagaaccttgagcgtctctccggtgcggccaatccg |
| | ctcagcgaggaggttcgtgacttcacgcatctgtgcgtaactcatgcagttgaaggtatgctcgcagtacatggccgcg |
| | gtcagctctcttccctcagggctgccaaacagcacgcggatgccgtccgtcgagccgctcaagacgcccgccagct |
| | gctgcccggcgtagtaggctagtctgttggggactgcaaacccggggtctgatgccaggacttcctgcaggatcacct |
| | ggctggtcttgcgcggggccgtgatgtgcgtgcgtgtaatctggccgctggccgggtcgatgttgataaggcgtgcgt |
| | cacgctcaaggaattcgtagacccattgcatgaggcggccatgctgagggaggaaggcgacgcgggcgaggggct |
| | ggcctggtgatgccgtgcgaagggggcatccgagttcatccatcgcctcgacgacgagggcagtacagagtctgttg |
| | cttccagagagcatgacgccctcggtcttgtcgactccgtactccttcatgagggtgtcggtctgcatcttgacctgccc |
| | aaaggaggctagaatgtcggaagaggatagggcaagccgagactcgacgggaggtgcaatcgctgcgagcccag |
| | cagacttgtgaatggccactgccttgagaggcaaaggctgctcctcttcgccagtgggcgtgaggatgcccgtgtctg |
| | agctttcggagccggcgtcgtcgctctcagaggcagactcgctggacgagttgtcgctcttctcttcgtcttcgtcatctt |
| | ctgcctccgcaggacctgcgtttggaccaaagagcgcattcgagacgcactgcacgaacttgcgtaaactggttgctt |
| | ccatctgctcgttctggtcgagagtgcacttgaacgcggcctcgacctccttgcccagttccatgcccatcagactatcg |
| | atgccaaagtccgccatctcggcgtccagctcgagctcgctggcatcaatgccagagactgtagccacaaggttgcg |
| | cacttcctcggtaatatctcgccaaccagagggcttgctggacttggatttggccttcgtaacgggcttcttctctttcttct |
| | ctttcttcgaggtcttgctggcctttaccttcgccccaggttcagagctagctcttacctcaggagcagtctttagagcagc |
| | ctggaaggctgccgctggtgttggtcctggcaccagcgctttcgttctcaggaccgagtcgtccttggtcatccgtgcg |
| | agcatcatgctcatcgacgcctttgcgacacgcatatactgcacgcccagcatgatctccacgagctggccgcttacc |
| | gcatcaaatacaaacaggtccgtcatgatcgctttgtcgccttgtcttgaatggcgggcataaacatgccagacgtccg |
| | catcctctctcggcggtgctctaggcgagcgcatgctcagctcgcagcccgtcgcgatgaacatgtcgctgctcggca |
| | ggtccgtcatcaagtttacccagacaccgccgacctggctgaaactgtcgctgagcgggacatcgagccatgtatccc |
| | cgcgactggatctggggagttgcacacggcctgcgcactcagttcccttgccgacgacatacttgaccccgcggtag |
| | acctcgccgtagtcgacgatcgagctgaatgcacggtagacattgcggccctgcaggacctcgacaccttcgtcggt |
| | gtcctgatcgaggcttagacggagaagatcggtgcattgcttgtgtgagacgagccgctcaaagttggcgaactcgcg |
| | gacgtgcgcttggtcagaagaggagcgcatttcgaccgtggcttcggcgtgaatttctggtgttttcttggtcgcgtcatc |
| | atcaaggctgaagatcctgaccgtccagtttgtccgtctcttgtttgtcgccgtcaaatcgaggtatacgacccggctgg |
| | gatccttgcagatagggctgtggttgatcatctctcggacaacgggctgcaccccatcttgcctccaccctggctcgag |
| | actgaagagggcctcgataacgatgtcgcactcgagcgtccccgggcaaatgggcgcagtctgtgcgatgacgtga |
| | ctgagcacgtagcggttgtacttgtccgcggaggtattaacccggaatcgggcctgccttgtctcgtcgtcttgatagcc |
| | gacgaactcccacaccggcagcgtccgggggtcctgcggcgtgccggcctgctgaccctgcagccctgccccagc |
| | gagggagccgccgttggcagcgatcaaggcgagagcggcttccttaactttctcaacgggggacttcatcgggagc |
| | cagtggcgggaagaagtatcgaactggtatgggggtaggaggaggtgggcatactcagcggtctggacagcatcat |
| | gcgcccagaaggtaacgcggagaccctgcttccagagcgcggttgtggtatcggcgagagagtctagggctgtctc |
| | gttggtgatgctgacagcctggaagtagtggctctctgacgacgcctggccctgagcaatggcccggccggccatga |
| | cggtgatggtcgagctagagccggcttcgaggaagatcgcctgcgggtgtctctttgcgagacgctgcactgcgtggt |
| | tgaagaagacgggttggcgcatgtgctgcgagacgaaggaggcatctgtcgctctggcagaggccacctcagtggc |
| | tcgctcgacggggatgagggggctgttgaaggtcagcgtcttgccgatagagtccagcccgtcactgatcttgtcaac |
| | gagcgaggagtggaaggcgttcgtgacattgagacgcttgcccttgatcgagccgaattcgggccgcgagatcgtct |
| | gctggacctgatcgacagcactggtggacccagcaatcgtgaagctgcgcgggccattatagcaggcgatactcgc |
| | agagccatcagaccctgaagctccgttggcctcggacagtagctggtggactagtccctcatcgccttccagagccat |
| | catggcgccccggtcagcgccccagctgtcccggacgagcttcgcacgcgccgcaaccaaacggacggtctcatc |
| | caggctcagggtcccggcaacgcatagggccgtgatctctccaaagctgtggcccactagggcctggaccttgccgt |
| | tgaggccgcagtctatccaggtctgagcgcaggcgtactgcatcgcaaagagcatcgtctgaagcttaacggtatcttc |
| | aatgggctcgcggctgaatatatcgggcgcggcgtagatactgaccagcccctgcgccttaacaacagtatccaccg |
| | catctagatgcttgcgaaagagggcaactgcgtcaaagaggccccgatccagcccgacaaagcgcgagatctggcc |
| | gccgaagcagaggatgacgggtcgttcggccttgacgggggcaatgcccacactcgcggcggcatccttgctgctc |
| | ggagccgcggcaacggcctgttcgatcttctcgtggagttcggccagcgagcgggcattgaagatgaatccctgagg |
| | cagaccgcggttggattggcgactgaggttgaaggagatgtccgccagggtcggctcttcggcgcgcgagcgcaac |
| | cagggcccgagtttggcacaatacgccgttattgctcgagtatcgagcccaggaatccaaaaggggtagcgtgctcct |
| | gcaacagcgtggcttctcgagtgagggcctcggagatcgggctgggtgacgatcatgcttgcattcgacccgcaagc |
| | gccgtagttgttcagcaaggccgtcttcctctcctcctcccaggcccgtagtcttgtcacaacctcgatattgtcgtccgc |
| | cttgacggggatcttcttgttcatcgtcttgaaactcgcttgcggggggatgaacccctcgcgcatcatcatgattatcttg |
| | acgagcgcaatcgccccggacgcgccctctgtatgcccaatatggcctttgacagacccaattggcagcttcttcttgc |
| | ggcttggtccacccagtgcagcaaggatgctctcgtactctgcaggatcgccgacgggcgttccggtgccgtgggcc |
| | tcgaccagcgagacgtcgttagcagtgaccttggcctggcgcatgacgtccttgaacaggtgcgacagggacggcg |
| | agttcgggacgaacaggggcgtgcagttctcgttttggtacacggcgctcgcggcaatggttgcaataacctggttcc |
| | catcgcggagggcatcagacagacgcttgaggtagacgaatgcagcgccctcagcgcggcagtatccatcagcatc |
| | gtcgtcaaagggcttgcactggccagtaggagacacaaagctgcccgccgcgaggttctggaaccagttcatgtttgt |
| | gaccgtattggacccgcctgcaagcgcagccgtgcactctccagagagcaggttcctgcaggctgtatggatagcca |
| | ccgccgaggaggaacacgccgtatcaaaggtcatacaggggcccgtccacccgaaatggtggctgactcggccgg |
| | taatgaaactcttgagtgcaccagtcgccgtgaacgcgttcgggtcgtagcacgagatgttatgctcgtagtcgacacc |
| | gcatgaacccaagtagacaccaacatgcatcttgtcacgcccgtccggggtatacccgttatggtcttcgacaaagtac |
| | ccagactgctcaacagcctgatacgcagcctgcaggacgatgcgactctgcggatccatcgctgccgactcccgcg |
| | gcgagcgcttgaagaatttgtggtcaaaggcatcgccgtcgcggaagaagcacccgtagaatttgcgcttcgggtcg |
| | gcatctgcgttctcgcggaagagcatgtcgtgcatgagtctgtcccgggtgatggggatatgctgcgactggcccgtct |
| | tgagcatggcgacgaactcatctagatcgtcggctccggcggtcttgacggacatgccgacgatggcgatgggctca |
| | gactggggcgagacgggcatgactggctcgacgcgggtggtctgctgctgctgcagttgcaggaccggttgaagct |
| | ggggttgtgggggaggtgatgattgcggtgtaagccagaatgaaggcttctcagggtctttgggaaggtcttcgtaaa |
| | agacctgtcttcctccgagagttctcatcagagttggagggacacatctctccaggccaaaggtgaccacgtaagggt |
| | ctgggagggcatccgccacggccgagaaggtgtcaaaccaccggcattgctgcaccaggatcgaccgcaccacca |
| | tctcagtcatgttccctgagccagaaaccggaatgcccgatccctggttgtcgtaagtctgcagagcgagcttcgacac |
| | ctctgcatactgcagcccaggcagagaggcgcacagctccaccagggcattcgtatgttgtttccgatcagcattggg |
| | gctatggatctggcccttgattccaacctcggccaccgtgactcctgcagctctgaggcgcttcatgagcagtggcgca |
| | attgtctctgaggccgtcaccgttgcccgcgcctggtcataccggacagcaacatacgcgtcgtttgacagatccccaa |
| | tgattcggttcatctcgtcctcctgtttctggccgcgccaggcgacggcgtaggacgctgaactgcccttgccggatgc |
| | cttgtcccatacttcttgcgcgtcgatgagagcgccgatgagcatcgccagccggacggcgacggctccgtattcctc |
| | gaacccggcctggtttctggcgctagccactgaaagcgcagcgagcaggccagcgcagaagcccaggatgaccgt |
| | cggcctgctgccggactgtgtctgctgcaccagctccgcctgcagatctacggctggggcactgccgtccctgatcat |
| | ctccagatgccgccagtactgcgtcagctggattaacaccactaacgggccaaccaagatgctcggcagagactcgt |
| | cgtcagaaaccgagagcccggccgtgtcgaggctgtgccgaagccatctgtccagttcagacaaggaggtcggccc |
| | gtcgatatcgcgggctatatcaggcatcttggctgccaaggcatcccagtatgttggtaggtcggcgattgtgcgcaaa |
| | atccagtcgcgttgtggcgattgtgagagtggacgaacgagcttgtccatggatgcctttgtgaatgtaccgacatgcg |
| | ggccaaataggaagactgttgaggcctcgtggcctgacccagaggcgcttgctcgggtcat |
| intergenic | tgcgggagggtaggagggtaggagggtagctaggtagttgatagtgctaagtgctctgccgggtcaactgtgaatga |
| region | atgaggtgtagttgagacacttgaggttgactttccaggcgagcgagcgggtcaagagagcagagagaatatgatag |
| between AN1034 | actgggtgtctgtagtagatagacaagatgtatgtctgtcccttggggaagtagggctaatacttctaccttagcacatgtt |
| andAN1033 | gcgggaagccacgcactgaggaaacactgacatcgttggggcactctgattggagccggagattaaggtaagatgg |
| (named 1034P, | aatccttctggctgcagcgctgtaagccctaagcctggtggcgcttctggcggacttttcggactacaggactccatcc |
| 849 bp) (SEQ ID | aagactccagatcgagactcagcttcgctagtccggaagtccgctggctgatgcttgtctcagcttttcgtctcagctttg |
| NO: 7) | tcgtcttctgtagagcctttagggaaaccccaactcagcatatggatgcagggctggttgggctgattgggcgttgtctg |
| | gacttgtatctgggtatggctgccgtctggggatcaaaggtaaatggggcagaaattgcctgttgaaatagttattgcgg |
| | aggccaatgcaatatcccaagaatttcccaaaatgcaagctactatagatgctacatagccagatagaggttgataatg |
| | ccacattttcaatatatacacatacgtttgtgtgtataagtacataacacgactacagtggctgatatatatgcagtggacg |
| | cctttagacatgtttccatttatgattatagagcgatcctcaggcaagtggttata |
| AN1033 | ctagaccttcactacagcacgctcatacgcttctctcgcctggtcgaccatgccctgcacatcgaaatcccaaatcaccc |
| (complementary, | tgctcctcctctcccattcagccttacactttaccccgtcacccccaatgtcttcataccgccattcgtagagatctcccatc |
| 1452 bp) (SEQ | tcccttgagctcttcacaagccactggctacgctcaatcctcacatcgctgtaagtcttcagggcaagctcaatattagac |
| ID NO: 8) | ttcttctccttgaacgcggagccgttctggaccttctcaagcaactcagcgagaacaagcgcgtcctcaacgcccatac |
| | aggccccagccccgtggaacggactggacgcgtgcgcggcatcaccggccagcgccacccggccagcggcata |
| | gtaaggaagcgggtggtccgcctgatcgaagatggcgtacttgcttagctgttccgggaagaggctggcaagttcctt |
| | gatatgcgggccccagttctcgaccgccgagagtatctcctccttcgagctgggcactgtcatggtgtggccgtgagtc |
| | cactcgttcgagtcgtgcgtgaagaggaaaacattatagatctgggcgttgtttacctaggcacaatcagcgccttcttg |
| | cagaatagatgcggcatgctaggcctggaggtaaggtagggtaccggaaaagagacaatgtgcgcgtccggcccg |
| | caatgtgcgatctggacatgcgccttttcggtccccagcgcatcaattgctgctggcataggcacgagagcgcggtag |
| | acagctttgcgagagtacctggcgtttgcagcagggtgttctgcgccgaggaggactctgcgggccgtggagtggac |
| | gccatcgcatgcgatcacttcacccaccgcattagcattatgaaacgtccaatacccagctcagggaagaaaaccaac |
| | caatatctgcctcctccacctccccgtcctcgaacctcagcaccactttctggtccccaccatcctcatatgccaccagc |
| | ctcttgccaaacctcacaaccctctcgggcagcaaccgcgccatctccgcatgaaaaacacccctcaagcaagccca |
| | gtacgccatattcttctcctcgatctcaaacagcacgctcttctctggatcctgtgcctcctctttgcttttcgggtggaatcc |
| | gtcccagtaccgcactttatcatgcggattgcgctgcgcaactttggagagagcggatagaattgcgggatcaaggcg |
| | ctgcatgcactcgcgggcgattccggtgaaggcaaatgcggccccaatgtcgggccaagctgaggcgcgctcgtag |
| | attgtcaccttgccgatgttgcggtggagaagccccagggctgtcataaggccgatgatgccgccgcctatgatggcg |
| | atggagaggggttcctgttcctgctcgtggtctgccat |
| intergenic | cctgtttagagtggccagaaggtgtgtgtgttatctgcaggatgccggtaccagtagggctgtatgtaaatacggctgc |
| region | agtagtttcaagttctgcttcgatcaagcgttagacctaggattgagcgcggctctggcaatggcggcttttctcatggta |
| between AN1033 | tagcatggcatagcctgaggatataggtactccataccgaggtacgagtacatctatactaagaatagtgactcccagc |
| and AN1032 | ttgcctatcccctgcttatcccggagtttgcatctccgccaggaagcacgcggactgaggcggagtaattaacagaag |
| (named 1033P, | gcatggcaatgcttactgcgtggggcttaaaacctgacctgacctggcctggcctggcctgatctgatgtgaaactggt |
| 605 bp) (SEQ ID | tctccttctctatctccctctgtcagattgatcgtcaaaacctaaccctaagtcaaatttaaacgccacgcaccggatactc |
| NO: 9) | tcaactctgaatacggccttgatcagccaatcacagaagattgcgagctgacagttcgtattgattactttaaagcctggc |
| | atagacgatctgccattgatttgcaattctccggcccagttgcata |
| AN1032 (894 bp) | tgccggcgctcgatatcgcctcggccccggccgcagtctatcaacagcaactccatctcccacgcatcctctgcctcc |
| (SEQ ID NO: 10) | acggtggcggcaccaacgcccgaatttttaccgcgcaatgccgcgctctgcgaagacagctgacagacagctatcgt |
| | ctcgtttttgccgacgcgccatttctctcgtccgccgggccggatgtgacgtctgtctatggcgaatggggcccgtttag |
| | gagctgggttcctgttcctgcgggcgtggatatcagtgcatgggccgctgccggtgccgctagtaggatcgatatcga |
| | cgtggaggcgatcgatgagtgcatcgcagctgccatagcgcaggatgaccgggccggcgcgacaggggattgggt |
| | cggcctgctggggttcagtcagggggcgagggtcgctgccagtctgttgtaccggcagcagaaacagcagcgcatg |
| | ggtctgaccagttggagtaggggtagggatcgcaagcgaggtgcgacctctagcaccaattatcgcttcgctgtcttat |
| | ttgccggccgcggaccgctcctggacctaggctttgggtctggctctttagccggctcgagtgctgcttcttcgtctgcg |
| | tctgcgtctgtatctggatctgaatctgcgggtgaagaggaagaggacgggcacctcttaagcatcccaaccatacac |
| | gtccacgggctgcgagatccaggcctcgagatgcaccgggatctagtccggtcttgccggccctcgtctgtgaggatt |
| | gtcgagtgggaaggcgcccaccggatgccaataacgacgaaagatgtgggagcggtagtagcggagcttcgacac |
| | ttggcgataagccggaaatatgaaagcttgagatgttga |
| intergenic | attcagcctattgagattacagccacggaagtaatcctgtaaggatcaggatgcaactccatgcaaggcgctaaggatc |
| region | aggatccttttcttcaggattgtggcaacggcgccagcggccagcgggcgctatcgcgtcggtggtgatggcgttattt |
| between AN1032 | ggatttcggaggatagaatccggtcagcctaatcaagccaactccgtcggacttcggcgggactgtccggtcagttag |
| and AN1031 | agctagagaaggaaggaggtagagtcccagatagacaaaagacttggctgctatatatcttattattcaatcctcaatcc |
| (named 1031P, | cgctagctgtcaatagaatgatcctcagccgcacttgaagtcttgtctacatcccgaatccaggcgca |
| 384 bp) (SEQ ID | |
| NO: 11) | |
| AN1031 | atggctgagacggattcctcccacacccgtgggcccgtagactcaatccagaagaacgacgcctcaagcgacgatg |
| (2033 bp) | ccgaggcagagaccaagatccagtatccctcgggctggagggtcacgatgatcctgacttcggtgacattggcgtact |
| (SEQ. ID NO: 12) | ttcttttctttcttgacctagccgtgctgtcgaccgcgactcctgccattacctcgcagtttgactcgttagtcgatgttggat |
| | ggtgcgttatgtcccctactgcgctcttccctaggtacatatgtgctggatgctaaaacccaccttgccggcaggtatgg |
| | aggcgcctaccagcttggaagcgcagcgttccagcccctgacgggcaaaatctacagccagttctcgatcaaggtag |
| | ttctccctcaaccatttgacgcagttggaggcttgggtgctcatgaatagcagtggacattccttgtcttcttcattgtctttg |
| | aactcggctctgtcctgtgcgccgcagcacgcaactcgcccatgttcatcgttggtcgggtcattgcaggcgtagggtc |
| | ggccggcatgtccaacggcgccgtaaccacaatctccgcggtcctgccaacgcagaaacaggcgctcttcatgggc |
| | ctgaacatgggtatgggccagctcggtcttgcgacgggaccgattatcggaggcgcgttcacaacgaacgtttcgtgg |
| | cggtggtgttcgtccccctgctccctcctttcaaatcccacctactaggcgaccatgcagagaagatgcaccagctgat |
| | gacgacgcaggcttctacatcaacctccccctcggcgccgttgtcggcggcttcctcctcttcaacacgatccccgag |
| | ccgaaaccaaaggcccctccgttgcagatcctcggcaccgcaatcaggtccctcgatctgccgggattcatgctaatc |
| | tgccctgccgtggttatgttcctcctgggtctgcaattcgggggcaatgagcacccctgggacagctccgtcgtgatcg |
| | gcctcattgtcggaggaggtgccaccttcggtgtcttcctcgtgcaccagtggtggcgtggcgatgaggcaatggtcc |
| | cgtttgccctcttgaagcacaaggttatctggtctgcggccatgaccatgttcttctccctgtccagtgtgctcgtcgcgg |
| | acttctatatcgcgatatacttccaggctatccgggacgactcgccactcatgagtggtgtgcacatgttgcccatcacc |
| | ctaggtctggtcttgtttactgttgtttcaggggcgctgagtatggtcttttctcctgcgtgcttgaacaatggctaaccgtc |
| | cagtctccgtactgggctactacctgcccttccttcttgcaggcggcgccatctccgccgtcggctacggcctcctctcg |
| | acgctgagcccgaccacctctgtcgcgaaatgggtgggataccagatcctctacggcgtagccagtggctgcaccac |
| | cgccgctgtatgtcttcagttttacatacccccggaaccctttgccttcacctttaccaggtagaatgccgctgacaaggc |
| | cgaatgcagccctacgtcgcaatccagaacctcgttcccgcgccccaaatcccgcaagcaatggcaattatcatctttt |
| | ggcagaacattggcgccgccatatctctcattgcggcaaacgccatcttctccaactccctccgcgaccagctagccc |
| | agcgcgcgagtcagatcaccgtctccccgggcgcgattgttgcggccggtgtccggtccatccgggacctcgtctcc |
| | ggctctgcgcttgcggctgttctggaggcgtatgcggaggccatcgacagggtcatgtacttgggcatcgcggttagc |
| | gtgatggttattgtgttctcgcctggtctagggtggaaagatattcggaagacaaaagatctgcaagctctaactagcga |
| | tggagcgcagggtgaagcgacggagaaggagactgttccggttgccctgggttaa |
| intergenic | ggcatcgtctacaagcagatgctaggcacacatttctttctgccgctaaaaattgggtaatgcagagccacctcgcttttt |
| region | ttttttcgaacattttccatcttgtggtatttctgggttcatttcgctccatataacgaagattggccttggtacgggctagggt |
| between AN1031 | tcgcgggtgggatagttatagaatgagaaataatacttttatatgtaacaatttcaacttctcaagatgaatataccattcgg |
| and AN1030 | atagagcagcttctgagtatcgacagacttaggtaggcttatgggtatgctctgttgaatatcttgtagatgtgacaggca |
| (named 1031T, | atagattgttagattatagcctacaatccacagctcagctcagcacgagtttgattttttcattataattggaataagcactg |
| 591 bp) (SEQ ID | agctcagaatgaaaccaatagattactagggctatgcgtagacgttgaacgggatccatcaccaagcgcagtattagg |
| NO: 13) | gcaccttttgtcgtgggtatatagcaactaaacacattctcttcggtcctgttcggccctcttcggcctccattagccagtc |
| | aaaataaacagtaaccag |
| AN1030 | ctacaaagtgacaacaagcttctttcccgaaaccccctttcgctggatatccagcgcctcctggatcttctcgagcccctt |
| (complementary, | tccgacaacgagcggcggcggtgcaggcacaaactgccctctctcgagcgcttggggcagaaagtccatgtaaacc |
| 1218 bp) (SEQ | cggctgaccacactgtccgggtccaccagcccgtcaacaaggataaacttggcgatgacgcctgtgcggcgctgcc |
| ID NO: 14) | ggatgctcgatttcaccattcctcccagcatcccaatgaggtaagtccccttgccgacgaaggtggttagcttctcaggc |
| | gggatgatctcaccggcgacggcgatgaactttctcgtcagcgcaggatcatgcttgcgcatcacgagggtgcaggc |
| | ttccaccgcaccggcgccaatggtatatgcgccgacgagctctctgcccttgagggcggataagagatccttggcca |
| | ggaacttgctccggtagtcaaagacgtggctcgccccgagccccttgacatagtcgaagttcttgggcgacgaggtc |
| | gaaaggacctcgtagcctgctgcgacagcgagctggatcgcattgctgccaacgctgctggcgccgcccgtgatgat |
| | caccgcgcgcggggaccccgacctgccccgctgcacctctcccctgcccttttccgcaagctgcggcatatcgagg |
| | gccagatagtccttgtggaagagaccaaatgcggccgtacccagcccgagtccgagcacagatgcctgcgcatcgc |
| | tgatcccagcgggcaccggcgtgagcatatgcactcgcaggacggtatacagctggaacccaccctcggccgggtc |
| | gttcacctctttcgcaatcgccgtcgcgcttccacagacgcggtcgcccacggcgaaccgggtgacgcccggtccga |
| | cctcgacgacctcgcccgcaacatcagtcccaaagatgaacgggtagtggatatacccggccagcgcgggcccgat |
| | gaactgcaagacccagtcgaacgggttgatagctacggcgccgttcttgacgaccacctggccagggccagggcgc |
| | gtgtagggggcgtcgccgactttgaaggggatcacctttttggcggggatccacgcggcgcggtttttgggtttgggg |
| | gtcccgttgccgttggtagccggcgctgctgcggttgctgcggttgtatcttgagttgccat |
| intergenic | aacgaggtccaggtgacggtaacgtggttcagtgcagttccaatgtatggtagcgttgtaagctgacacggcgacggc |
| region | tgcgagaggggttggggggacggaaccagctgaaacaggactggcgaaagaaagctgctgtgttatatgtaggcag |
| between AN1030 | agctaaagaaccttgtggagcgacagaaccaaagtcagtctgggccatgggctatcttccataattttgggagctcgag |
| and PalcA- | gtccggattgcccgttaatactccgccagactagggcaagatagggctacgcggagttttaggtggacggatttcaac |
| AN1029 (named | cctccgaagtccgctcgaacttttgtcgacgagattaagccactagcctaaaggaatcagacctttaattcctcaggccg |
| 1029P, 1221 bp)* | agtcgggatcattgaaggcgagaatgaggtgaggttgtcagccacatcgtcagctcaatcctttagaccacgttcttatc |
| (SEQ ID NO: 15) | tcgcggccgttctccaatcgacgggcccgctggcccccagcgtgcagattacaccgtctcgctccgactgcaggatct |
| | ggcgtcttccatgcgcggacgtttcggacggcgatgactgtctgagtggttggcagggatgcacccctacctacccct |
| | gatcgaagctaatggtaatgcagaatacgaggttggttagactaagcgcttctgcagctgcagcgcatggaagctgttc |
| | tgtctggtggagagactaagcagtgctctgtgctcctctgtgctgctctgcattgcactgcactgtactgcattgtactgca |
| | ttgctgttctgcacggatcattcatccatctaccatggatccactactaacctcgcttactctagtcgatctggtcaagacg |
| | accaagacctcggagaattagatggccaaccaaggatagatgcgagatcaactgatccaccgctggcaaacttagtt |
| | gtgaatgtcgcgaacgcaaataccacggagatggcatgcagccgcacccgaaatggaatgctgtaggcctaatcaa |
| | gctcatcgattctcgcccccaaatctgggctgcgcggtcctgcaggtgagacggatcctggaggctccatgctggctg |
| | gctctgcctcctcgtggacgagggtacgatggcagccagtctgctggcgtgctggcgccgctggtagcacggccac |
| | gagcctattgattgcacgggcaaacgttcgtaactcgctcgtaa |
| PalcA (404 bp) | ctgaaaagctgattgtgatagttcccacttgtccgtccgcatcggcatccgcagctcgggatagttccgacctaggattg |
| (SEQ ID NO: 16) | gatgcatgcggaaccgcacgagggcggggcggaaattgacacaccactcctctccacgcaccgttcaagaggtac |
| | gcgtatagagccgtatagagcagagacggagcactttctggtactgtccgcacgggatgtccgcacggagagccac |
| | aaacgagcggggccccgtacgtgctctcctaccccaggatcgcatccccgcatagctgaacatctatataaagaccc |
| | ccaaggttctcagtctcaccaacatcatcaaccaacaatcaacagttctctactcagttaattagaactcttccaatcctatc |
| | acctcgcctcaaa |
| AN1029 | atggcgtgtcccaccagacgaggacgacagcagcccggctttgcatgcgaggagtgtcgccgccgcaaagcgcgc |
| (2354 bp) | tgtgatcgcgtgcgtccgaaatgcgggttctgcactgagaatgagctgcagtgtgtgttcgttgacaagaggcagcag |
| (SEQ ID NO: 17) | aggggtccgatcaaagggcagatcacctcgatgcagtcgcagctgggtaggtgtttgtcttgtctcattgtatctcgtctc |
| | gtctgcgcttttgtgattatggggctgccatgtttccggtccggacacaggcatctgcaaggcccgccgctgtgctccc |
| | ccgatctgcagggaccaatgcagctggttctggagcttgtgctgtgctgcttccctgtctttccacatggtcgagtcgag |
| | cgagctagctaacatgggatgcctcatgctttcagcaacgcttcgatggcagcttgatcgatacctgcgacatcgacct |
| | cccccgtccataaccatggccggcgagctcgatgagccaccagcggatatccagacgatgctggatgactttgatgta |
| | caggtcgccgcgctgaagcaggatgccacggcaaccaccacaatgtcgacgtcgacagctctcatgcctgccccag |
| | ccatctcatctaaagatgctgctcctgctggtgctggtttatcgtggcctgacccaacctggctggatcgccagtggcag |
| | gatgtcagcagtaccagcctcgtccctccatcagacctgacagtctcgtcggccactaccctaaccgaccctctcagct |
| | tcgaccttttgaacgagactcctcctcctccttctacgacgacaacaacgtcgacgacgaggcgagactcatgtactaa |
| | ggtcatgttaactgacctcatccgggctgaattgtacactacctaactgatttgtctaccatgacacctgactgacaatgtg |
| | cagagaccaactctacttcgaccgggtccacgccttctgccccatcatccaccggcgacggtactttgcgcgggtcgc |
| | ccgagatagccataccccagcacaggcatgtctgcagttcgccatgcgaacgctcgcagcggcaatgtctgctcact |
| | gccatcttagcgagcatctctatgccgagaccaaggccctcttggagacgcacagccagacgcccgccacaccgcg |
| | agacaaggtcccgctcgagcacatccaggcctggctgttgttaagccactacgagctgctgcggatcggcgtgcacc |
| | aggctatgctcacggctggccgggcctttcgtctcgtgcagatggcacgactgtcagagctggatgccgggtcagatc |
| | gacagctctcgccgccgtcttcgtcgccgccgtcttcgctaaccctatctccttcgggggagaatgctgagaacttcgtc |
| | gacgccgaagaaggccggcggacgttctggcttgcttattgctttgatcgtttgctttgcttgcagaatgagtggccgtta |
| | acgttacaagaagagatggtacgtcgcgcttcttttattctatttacctcagaatttatattcagttattttttattctaaccctgc |
| | tagatattaacccgcctcccctccctcgaacacaactaccagaacaatctccccgcacgcacgccctttctcactgaag |
| | ccatggcccagaccgggcagagcacaatgtccccgtttgccgaatgcattatcatggccacccttcacggccgatgta |
| | tgacgcaccgccgcttctacgcaaacagcaactcgactgcgtccggctccgagttcgagtctggcgccgcgacgcg |
| | agacttctgtatccgccagaattggctgtcgaatgcagtggaccggcgagtccagatgctacagcaggtctcctcgcc |
| | cgctgttgacagcgacccgatgctgctcttcacgcagacgctcggctaccgcgcgaccatgcacctgagcgataccg |
| | tccagcaagtctcctggcgggctctcgccagctcgcccgttgaccagcagctactgagcccgggcgcgacgatgtc |
| | gctgtcggccgccgcgtaccaccagatggccagccacgcagccggcgagatcgtccgcctggcgaaggccgtcc |
| | gtcccgatcccacgggcggcgagggggtgcagcatctgctacgagtgttaagcgagctgcgcgatacacacagcct |
| | ggcgcgggattatttgcaggggttgtcggtgcagacgcaggacgaagatcatagacaggatacgaggtggtattgta |
| | catag |

DNA sequence of the afo and other regulons are found at the Aspergillus Genome Database, for example, at www.fungidb.org/. This and other sequences also may be found using the NCBI database at, for example, www.ncbi.nlm.nih.gov/gene.
*Part of the intergenic region between AN1030 and AN1029 has been removed after replacing the native promoter of AN1029 with PalcA. The original intergenic region between AN1030 and AN1029 (1029P) is 1370 bp.

TABLE 8
Genomic DNA sequence of the afo locus in strain YM192.

| Region | DNA sequence |
|---|---|
| intergenic region | aatgactggtccgtccgtacttagaaagggtgtttctgtccggcagttatttaatgtcggctgtctgctcttgcaatttctctt |
| between AN1037 | ttgatttatctttcgtggtgtatctcgccggaacgaatggccacggttcgcgtttgcgttcatgttcatgttcatagagcagc |
| and ctvA (1036P, | tgcgaagtttcaaatgttcgttcgttcggctcggcttggctaggcgtatgatggtgttatgtttaggttgagaaggtattctt |
| 1487 bp) (SEQ | agttgggagctagagaaaagattatttgttccctgcaattttgctgtaccccggaaacatagaactgttactgtaccaata |
| ID NO: 1) | ctctgcgttccctccccaatgcaccccatacatatggagttggagcctgtacctttgtcgataagcttattctccaatcaac |
| | tctgctattgcagcttttcacttgagctttcttattcgtatgtgctctacggacgaaaaataagctttgttgcctgcagatcac |
| | cttggcagctgtgctgcgcctagacttataatgcaacgtttttaactttttgtttttcttttttctttcttttttaaactagtt |
| | ttcacatgagctacccgttcattataaccatcagctctagctaggacaggatcgcatgagtatatacctatttatattccttcc |
| | ctcccaactcggactcacgctttatatatatgtctactattactcgtgggtgaagagaagtttacgactatttagcctagatga |
| | aggataggttgtgcaatgctcgatagcgtagcatttaaccctacctagtaatgagctacttgggctgctagaataaatctccca |
| | atccaagctaatgtagtcagagctgaacgcaagtctcgtacatggccctacgaggcatcacaatagccctaaagagta |
| | tcacgtgaccatactagcaccgcaatgagttcaggatccgacaatagcgaggctgtatccaagtgcgccgaataatgt |
| | ctatcactgtagaaatatatctgattcgctcagctggtcgataggcgaagcatcggagttggcggagttggcggagttg |
| | caggacttgctggattagggctgaggtcagacggactctcactctccgctatagacactgggcgatgttgtaggcagc |
| | gatgggagaatgtgcattgcacatggtccggagatttctggagtcaggtcatgcagtctagatcctgactgcagtagaa |
| | tgtgcagattccggagcttggggagttaacctgcagtaagctcagctcaagcaatgatcggtaggtaggcctggtggc |
| | catatcagctatagatgcgatccgcgcctcaagcgcatttcaagccctccctcttcaatacgtttgcgataccttagagaa |
| | acaaatcaacatccatcaactggcacagattcatctaccaactcaacgtgattacccgtccagctttgacctaaacctcc |
| | ataatccccatccacaaggcacc |
| ctvA (7527 bp) | atggcacccatggagccgattgccatcgttggcactgcctgccgatttgccggctcgtcatccactccttccaggctttg |
| (SEQ ID NO: 18) | ggaacttctcttaaaccccaaggacgtggcatcagagccacccgcagatcgattcaatatcgatgctttctatgacccg |
| | gaaggctccaaccccatggcgaccaatgcccgccaggggtatttcctttctgacaacgtcaaagccttcgatgccccg |
| | ttcttcaatatctccgcagccgaagcactggcactcgacccacagcagcggatgctgctggaagtcgtctatgaatcac |
| | tggagactgctggcctgcgcttagacactctccgcggctcctcgacgggggtctactgcggtgtgatgaactccgact |
| | gggagggcatattcagcgtctcatgtgcagcaccgcagtatgggagtgttggggttgcccggaataacctcgctaacc |
| | gcatctcctacttcttcgactggcaaggcccgtccatgtccatcgataccgcctgctcagcgagcatggtagcattgcat |
| | gatgccgtctccgcactcactcgccacgactgcgacatggctgcagctctaggtgccaacctcatgttgtctccccaga |
| | tgttcatcgctgcatccaatttgcagatgttgtccccaaccagccgcagccgtatgtgggatgcgcaggctgatggttat |
| | gcgcgtggcgagggggtcgcatccgtgctcttgaaacggctttcagatgcagtggccgacggcgaccctatcgaatg |
| | tgttatccgagctgtcggcgtgaaccatgatggccgtagcatgggtttcaccatgccgtcgagtgatgcacaagtgcaa |
| | ctgatcaggtctacttatgcaaaagccggattggatcctcgctgcgcggaagatcgaccccaatatgtcgaggcccat |
| | ggtacaggcacgttggcgggtgatccccaggaagcatccgcccttcatcaggccttcttcagttcctcggacgaggac |
| | actgtactgcatgtcggttccatcaagacagtggtaggccacgcggaagggactgctggtctcgcgggtctcatcaag |
| | gcatccctgtgcattcagcatggcataatacccccgaatcttcttttcaatcgcttgaacccggctctggagccatatgca |
| | cggcaattgcgagttccagtagacgtgatcccctggccctcccttcctccaggcgttccccgacgtgtttcagtgaactc |
| | cttcggctttggtggcaccaatgctcatgttattctggagagctatgaacctgctagagacctcaccaaggacggcttca |
| | atcagaatgcggtgcttccgtttgtcttctctgcggagtcggattatagtcttgggtcggttctggagcagtattccagata |
| | tctctccagattttctgacgtggacgtacacgatctggcatggacgctaatcgagcgccgttccgcgctgatgcaccgt |
| | gtcgctttttgggcgccagatattgcacacctcaaaagaaggatccaggatgaggtcgccctccggaaagcagggac |
| | accctcgacagtcatctgccggccacatggcaagactaggaagcacattctgggcgtcttcactggtcagggtgccca |
| | atgggcgcagatgggacttgaactaatcaccgcgtccaccattgcgcgaggctggctggatgagctgcaacagtctct |
| | cgatactttgccggaggcgtatcgtccagagttctcgctctttcaagagcttgctgcggatccggccgcatcacgactat |
| | cggaggcccttctgtcgcagaccctctgcacagcaatgcagattatctgggtgaaggtgctctgggctctgaacatcca |
| | cttggaagctgtggtcggtcactcatctggcgagattgctgcggcctttgcggctggctttctgacagctgaggatgcc |
| | attcgcattgcctaccttcgaggtgtgttttgctcggcttcaggcagctcgggggaaggtgcgatgctggccgctggtct |
| | ttcgatggacgaagcgactgcactctgtgacgacgtatcctcgtctggggggcgaatcaacgtggcagcgtccaactc |
| | gcctgaaagcgtcacgctctctggagaccgagatgcaattctgcgagctgagcagcagttgaaggataggggagtct |
| | ttgcccgtctacttcgtgtcagtaccgcctaccactcccatcacatgcagccatgttcgcagccctatcagaacgcattg |
| | agtagttgcaacattcagattcaggccccggtgcccaccaccacctggtattcaagcgtctatgctgggtgccccctgg |
| | aggagccttcggtcatagagacgctcggtacaggagaatactgggcggaaaatctagtcagtcctgtgttgttctcgca |
| | ggcactaacggctgccatatccaccacaaacccttccctggtcgtcgaagttggacctcatccagctctgaaaggacct |
| | gccttacagacgatctcaggaataacgtcaggggagatcccttatatcggggtatcagcccggaacaattgtgcacttg |
| | agtccatagccacagccattggatctttctggacgcatcttggtccacaagtcatcaatccgcgagggtacctggctcttt |
| | tccggccgaatgtgaggtcttcagttgtccgtgggctgcctttgtatccctttgaccatcgccaagagcacggttatcag |
| | acccgcaaggctaatggttggctgtaccgacggtacacaccacaccctctgctgggttctctgagtgaagacctcggg |
| | gagggcgagttgcggtggaatcattacctctccccccgacggctcccatggctcgatggccaccgcgtccagggcc |
| | aaatcgtggtccctgccacagcttatatcgtgatggctctcgaggccgctcgcatactgaccgctgagaaacaaaaga |
| | gcttgcatctaatccgtatagacgacctagtcatcggtcaagctatctccttccaggatgaacgagatgaggttgagact |
| | ctgttccacctcgcccctatggtggagaccaaggatgacaacacagcagtcggccggttccgctgtcagatggctgct |
| | tccgggggtcacgtcaagacatgtgcggagggcatcctcacggtaacctggggctcgccgctggatgatgtcctccc |
| | ataccctaggtctccagcgcccgcagggctagcccatgtagccgacatagacgagtactatgcgtcgctccgaagctt |
| | gggttacgagtacaccggcgccttccagggaattttttctctctcccggaagatgggtatcgccacgggccaattgtgta |
| | accctgcattaaatggctttctgatccatccagcagttctcgacactggattacagggtcttctggccgcggtgggggag |
| | ggacacctcacgagcctacatgttccaacccgcattgatgcattcagcgtaaaccctgcagcctgtagtagcggttcgc |
| | tagcctttgaggctgccgtgactcggacaggattagacggtctcgtgggcgacgtggagttgtatacggataccaacg |
| | gccctggtgccgtcttctttgaaggagtgcacgtctccccactagtgccgccatccgcagcggatgatccgtcagtattt |
| | tgggtgcagcattggacaccccttagcctggatgtcaaccgttccaaatctcgactgtcgccggaatggatggccatgt |
| | tagaagggtatgagcgccgggcgttccttgcactgaaggacatcctccagcaggtcacaccagagcttcgtgccactt |
| | ttgactggcatcgtgaaagcgttgtcagttggattgagcacattatggaggaaacccgcgtgggtcggcacgccgtct |
| | gcaagcctgagtggctagaccaagagctagagaatctcggacacatatgggggcggccagacgcgcgcattgagg |
| | atcgaatgatgtatcgagtttaccggaacctgctacccttcctccgcggggaagcgaagatgctagatgctcttcggca |
| | ggacgaattgcttacacagttctatcgcgacgagcacgagctgcgcgatatcaaccgtcgactgggtcagttggttggt |
| | gacctagccgtgcgctttccacgtatgaaactccttgaagtcggcgccgggacaggctctgccactcgagaggtactc |
| | aaacatgtcggccgggcctaccattcctacacgttcacagacatctcggttggcttttttgaagacatgttggaaacaatt |
| | cccgagcacgcggaccgtctgctattccagaagctcgatgtcgggcaagacccattgcagcagggctttggtgaaca |
| | cacttacgatgtaatcatcgccgctaacgtacttcatgccacaccgacgctgcaagagactctgcgaaacgtgcgtcgt |
| | ctactcaagccaggagggtatctgatcgctctggagatcactaacattgatacaatccgcatcggcttcttgatgtgtgc |
| | ctttgacggctggtggcttggccgggaggatggccgtccatggggtccggtggtctctgcatcacagtgggatagcct |
| | actccgggagacgggattcggtggcatagacactatcactgatcgcgccgctgaccagctcaccatgtactctgtcttt |
| | gccgcccaagcggtggacgaccagatcactcgatgtcgagaacctctgacgccgctccctcctcaacctcctttctgc |
| | cggggagtgatcatcggaggctcgcctagtctggtgacaggcataagagtcattattcatcctttcttctcgactgttgaa |
| | catgtttctaccatcgagaacctgacggagggagcaccagctgttgtgttgatgttggctgacctgagcgacatcccct |
| | gcttcgaaaatctcaccgagtcaagactggccggactcaaagcactggtgcaaatggccgagaagacgctctgggtg |
| | accacgggctctgaagcggacaacccttatctctgcctcagcaagggctttctcacttcgatgaattatgaacatccagc |
| | tatcttccaatatctgaacatcatcgactcggctgacgtccaacccgtggtcttggccgagcatcttctgcgattggccta |
| | taccaaccaaaacaatgacttcgccctcacgaattgcgtccacagcacagagcttgagctgcgtctctaccagggcgg |
| | gattctgaagttcccacgcattaacgcgagcgatgtcctgaacagtcggtacgcggcagctcggcgcccagtcaccc |
| | attctgtcaccaacatgcaggacagcgtggttgtacttgaccaaagcccaagtgggaagcttcgactcgtgtttgggga |
| | ggagcttgcaggtgatcgcgcaaccgtcaccattaacgtccgatactcgacctctcgtgcaatccgcatcaatggtgct |
| | ggatatctggtccttgttctcgggcaggataaagttaccaaagcgcgtctggtggctctggcaggtcagtctgcgagcg |
| | tcgtctcgtcctcctgttattgggaggtcccagcagatatcttcgaggagcaggagcccgcgtatctgtacgccacagc |
| | aacagctttgctcgctgccagtttggtgcagtccaacggcaccacaatcctggtacatggcgctgacatggtcctacgc |
| | catgcaatcgccatagaggccgcttcacgggtcattcagcctatattcactaccacatctccctccgcagcatcatccgc |
| | gggtcttgggaagagcatcctcgtgcatgagaacgacacccggcgacaactggttcatcttctccctcgatatttcaca |
| | gctgctgtgaatttcgaccctagtgcccgccgactcttcgaccgaatgatgacagtcggtcatcaatcgggtgtcacag |
| | aagaacaccttcttaccactttgacagctgccctccctcgtccgtcagcatctctgctgccggcccagcctcaggctgc |
| | catggacactcttcgcaaagcctcattgactgcttatcagttcaccgtccagttgacagcaccaggacccatcatcgcac |
| | caatcgccgacatccaatcctgttcacaacagttagcagtcgtagactggaaaccatcttgcggctcggttccagtaca |
| | cctccaaccagccactgagctggttcgtctctctgctcaaaagacatatctcctggtgggtatgactggtgccctcggcc |
| | aatccatcacgcaatggctggtcacccgcggcgctcgcaatatcgtcctcaccagccgcaagccatcagtggacccc |
| | gcatggatcgcagagatgcagaccacaacaagcgcgcgtgtcctcgttacgccaatggatgtgacaagccgcgact |
| | cgatccttgtggtggcacacgccctgaaggccgactggccgccgctcggcggcgtcgtcaacggtgccatggtgct |
| | ctgggaccgtctcttcgtcgacgcacccctgtccgttctgacgggacagctcgccccaaaagtccaggggagccttct |
| | cctcgatgagatttttggccatgaaccgggccttgatttctttatcctcttcggtagcgctatcgccactattggaaatctgg |
| | gtcagtctgcctacacagccgccagtaacttcatggtcgcgcttgcggcgcaacgccgcgcccgagggcttgtcgca |
| | agcgtcctccagccggcgcaggtcgccggtgccatgggttatctcagggataaagacgacagcttctgggctcggat |
| | gtttgatatgattgggcgacatctcgtctccgaaccagatctgcacgaacttttggcccatgctatcttgtcgggtcgtgg |
| | ccctccagctgacgttggatacggaccaggcgaggatgagtgcatcattggcggactccgcgtccaagaccctgctg |
| | tatacccagatatcctctggttccgtacgcccaaagtctggccattcatccactatcaccacgagggaactggcccttca |
| | tctggggcggctggttcgatatcgctggtcgatcagctgaagtgtgcgactagcttagcccaagttggggacatggtg |
| | gaagctggcgttgcggccaaactgcaccatcgactccatctcccaggcgaggttggaggcgtcactggcgacacgc |
| | gtttgaccgagctgggggtggactcgttaattgcggtggacttgcgtcggtggtttgcgcaggagttggaggttgatatt |
| | cccgttctgcagatgctgagtgggtgttcagtaaaggagctggctgcttccgcgacggcgttgttgcatccgaaattcta |
| | tccggaggtggtggccgattctgacgtggggagtgagagggatggttcctcggactcccgtggtgatacctcttcctc |
| | ctcgtatcagctgatcactccggaggagggggaccatgactga |
| intergenic region | gctgcatcggtcatgttgttcttctatagagttgaagcaaggtttgtagtttgctctgggtgtctggagttgtctggagttgtc |
| between ctvA and | tggagttttgttatgatgttgatgggtacttcttcatactagcattttggcatgttataagaacatattatcagttaaatgtct |
| ctvB (1036T, | ttcaatttaatcaatttgtttttagaatgatgttgtctgcctggctatgtatctagatcctatacaagctctatcgactcgacc |
| 1768 bp) (SEQ | taactactacgacttgaaagtcaagcgagaagtgatgatatgaacccatatgtcagacccgctaaatttattagtgataacaact |
| ID NO: 3) | atattactcagagcttttctttctagagtatgttagaattgccctttctggctcagtgggaagctcgagacctagtccttagtc |
| | acgtgctgctacatcatgtaaatataagccctacatggctgtcttgtgcatgaggctaacaccattatctgtcactggtcct |
| | tttatttggttcttttctttactttctcgggcgggggggaaagccgctaacactgtctatcgcttggacagaaactcaccagt |
| | ttgttcgcaatcctgaagcgtatgggaagcttacagttaaggagtagctcgagtctggaccctgttttcgacttgtaccttt |
| | gatttggatgactggttaacctcagcttatgtatgatgtgctctcatggtgtcaatatctggtagtctgattctgagcaatttg |
| | atagtatctgatggctggcgagtaaggccagggcgatgactggtataaagtcagccctaaaacttccatccgagatgta |
| | aaaccatcgattcccctccaagatctcctgacgagactaaacaaagatcaagtggccttgtagtaactctagcaagcag |
| | cgacaaaatgcctcaacacgagatgaccaagtcagactcggaacgaatccagtcctcgcaggtaagagcatcagga |
| | catttgctaataccattccgccccgctaatctgcttgaatgcacacaggctaaaagcggaggggacatgtctcttggag |
| | gattcgcctcgcgcgccctgtctgccgggactgctgggtcaattcccagtcctcggccactgcttccggccacgcgga |
| | ctcgggtgccggatctgcaggcggatctcattcggccgcacctggcggtgatgcggggcagggaagaagataaaa |
| | gtaccctgttgtctttggggcgttgaggtataatggcatcgtggtagaccgactgggcttttttttttgatatagttgatcctg |
| | aagcggaggacagttggtaggataaatgaaagatactgaaccatgcccggattttgtgctcaaggacctaaaactgag |
| | aagctgaatctgttcttgtctgggagaaggcctgccagctgcatccgagtatctatcttgccaggaccaaaccgggtct |
| | gggctcagttcttctaacttcttagtggagttttgcagtgtagattcctttgcactatctggtatcctagtagcagcctacca |
| | ggaaataagagataaataaagtcttaattggcattattatgtttctcagaactatatatctcggaacaaagctgagcagac |
| | agaagtttaccctcacatatggacaaattgcgtgctcaggcataagtcggaaacagccttagccaggtcaacacttgta |
| | gccttcgctagacgacgccccagcttttcataatggccggcctggagggagatacggctatccacc |
| ctvB | ctagcgacgaggcttccgcgccttgaacataaggaccgttccaataatcacgctctccacctcctcgaactcgtcctcc |
| (complementary, | agcgcacggacaaagtcgtctggatagtctgaccgattctgaaacatgtccacagcattgtagatgcgctgaaggagc |
| 687 bp) (SEQ ID | caactgaaccaattctgccgaactccacggcacagcagcgtagacccgaagagagtgccattgtccttgaggagcg |
| NO: 19) | gcttcaagttggcaaacacgcgtcctttgtccttagaagtccccgggagacagtgcaggacgtacataagggatatgg |
| | agtcgaactgccgttcaggttgtatagggatgggctccaggatattggccagcacacactccgtgcgatccgctactcc |
| | aacgcggttggcagccttcctcaggcatcggatgtgaaaatccactagcgtcagcttctccggccaggacggccgac |
| | gcttccgcacagcagagagatagtagcccgtgcccacgccaacatcacagtgccgagatccaatgttggacaggaa |
| | aaaagggagaagaatgtccttagacgaacacttccaggcaaagagcgcgctgacccaatgaacccagaagtcgtac |
| | caccacaagagaagtggattgtagtagtngtcggcgccttcggcatcggaaagctggtaggaggtcat |
| intergenic region | cctggtgtgattgggctgattaggacaggccggatgggtgtgcaagataggaggagaggactggtacggcgaatga |
| between ctvB and | gctttaatagccggtcagagattgcgcgtggctgcgcccagatccagcagctccagccatactccagcatactccggc |
| ctvC (1035P, 527 | cagccgggggcatatggcgtggtcactggagctggttaggatcaactgctggttaaggcttactgtgttgccatgctta |
| bp) (SEQ ID NO: | cggtgcaccgagagggaaggttggagttaacggagttgtaactccggggatccaattagggcttacagtctgcaaatc |
| 5) | catgcaaagtccgctgcgcccctgacacagcaaggaacagtgtagagtccgattggatagcggagttgaggtgactg |
| | gctggttcctgttagcccctgcatcgacctgcaatgtattgcatcaaattagggctagcctctaactccgttagactatcc |
| | gcaacgcctgtcacacacgtggctaggcagcagatgatatacttttgaaagcagtact |
| ctvC | tcatacttccttgacattgaacaccacccagctaatccacaaaactatcacaagtccagagcaatacatcaccctctccc |
| (complementary, | caaattcctgccacttggacagaccccaagtattccaccactccaacttcggccagtacttccccgaacgttcagtcaac |
| 1611 bp) (SEQ | ggcaagaactgcagtacctcaccgttgtatgagttacgcaccacccccaacgcaaaaagatcgccacagtgtggcgc |
| ID NO: 20) | cacatatcgcgtgaaggccgtgaccatccgtccctggcaagcctccaatcgcgtgatccagcgcgactctagatggat |
| | agcatccagtcgtttgcggtttttctcgctgaaggcagcgagcgcctggtgaatggtgtcacttgacggctggtcatggt |
| | tttgtgcaatcgcatatatcacgttggccagtccagccgccgcttcaatggctgtattggcgccttgaccgatgttgggg |
| | gtcatctagagtcccagctatcagtatatgaaggggaaaaaaaggctccatgcaagacataccttgctgatactatccc |
| | cgatgcagatgatccggccatgatgccacgtacggaagagattctcctccaacgcaaccatccggaatccccggcgt |
| | tgagcccagatatcccggaattgtacttcctcccagataggctggctggcggctgcctcacagcgcgcaatggcgtcc |
| | tcctgcgagaatcgcggaacgtcagggtagatatacttgtgtggtagcttttcaatcagcacccagaaaaggctctccc |
| | cggtcgcagggaatatcaggatcgtgaaaccgggcccgatgcggatcacatgctgccaacgcttccgtccggggat |
| | ggggttggacatgccgaagacgcagctgaactcgacggacatgcctagccgcagagcatcatctattaatattggac |
| | ggaatcgataaggagtgtagcaacatactggcctgttctttgagcggaatcagccccggctgctctatattagcaatgc |
| | gccacatctctcgccgcgtcacactgtgcacgccgtccgcaccgacaaccagatctccctggaactcgtccccatctg |
| | cggtggtgaccgtcatcttgctgccatggggagtgatccggacgacgcctttgctcgtgaggaccctggacttgtcag |
| | gcaaatgggcgtacaggatctcgagtagctgagtccgttccaggcacgcgaatttcaagccaaacctgcgtacccgc |
| | cgtgttagcgtcttcttacgactttgttccaccctctcggggaccaaggaagcaccgacctcttcaagacgacactagg |
| | cgaaaggctatcatagtagaacccatcctgaaagcaaagatgcaccctttgaaatggctggcagcggtcttcaatgtgc |
| | cggaagatccccagctgctccatgatccgccctccattcggcaggatggccaccgcggcgccaatcggcggatgga |
| | cttcgtgatgcttctccagcaccacgtagtctattccggcccgatgcagacagtgggcgagggtcagacccgtgacgg |
| | atgccccgacgatgacgaccttgaactgagggtgctttccttccat |
| intergenic region | tgcgggagggtaggagggtaggagggtagctaggtagttgatagtgctaagtgctctgccgggtcaactgtgaatga |
| between ctvC and | atgaggtgtagttgagacacttgaggttgactttccaggcgagcgagcgggtcaagagagcagagagaatatgatag |
| ctvD (1034P, 849 | actgggtgtctgtagtagatagacaagatgtatgtctgtcccttggggaagtagggctaatacttctaccttagcacatgtt |
| bp) (SEQ ID NO: | gcgggaagccacgcactgaggaaacactgacatcgttggggcactctgattggagccggagattaaggtaagatgg |
| 7) | aatccttctggctgcagcgctgtaagccctaagcctggtggcgcttctggcggacttttcggactacaggactccatcc |
| | aagactccagatcgagactcagcttcgctagtccggaagtccgctggctgatgcttgtctcagcttttcgtctcagctttg |
| | tcgtcttctgtagagcctttagggaaaccccaactcagcatatggatgcagggctggttgggctgattgggcgttgtctg |
| | gacttgtatctgggtatggctgccgtctggggatcaaaggtaaatggggcagaaattgcctgttgaaatagttattgcgg |
| | aggccaatgcaatatcccaagaatttcccaaaatgcaagctactatagatgctacatagccagatagaggttgataatg |
| | ccacattttcaatatatacacatacgtttgtgtgtataagtacataacacgactacagtggctgatatatatgcagtggacg |
| | cctttagacatgtttccatttatgattatagagcgatcctcaggcaagtggttata |
| ctvD | tcagaattgagattcctcccgcagcaaccaaacagccgcaccgcagggccctgagatcagacaaagacctccaactt |
| (complementary, | tcagcgctagatagcaagtctgtgtgaatgacgactgcctctcaactgtccgccgcatatgcagtgcccacaggagaa |
| 1132 bp) (SEQ | agctccccattccaatgaggtgatcccactgtaggaaccacagcgccccttgggccatggattgaacctggaccgtac |
| ID NO: 21) | gacccccagcaaactgccaaggggatacatccgccaagagatttgtaggagccaaggtcagggaaagaccccagc |
| | tgattacatgggggataatcgcgcatacaaacgcaaaggtatacgcggtccggcatgcactcctcgtggagataccc |
| | gtgctcgctcttggtcggaaaaaggcccgtagaccccagtgacacagagctgcatagattggccagagctgccatgc |
| | ggcaatggccatctgtttgccgaacaagtcctggtgcgcggattccggaaggaccatggcgatagtcgggactccaa |
| | atcccaggatcatgctgatggggatgaggcgtatggaatgagctgctgatgccgagataacgcgcgccactgggcgt |
| | gatgatgatgatgacgacgacgacgacgacgaccagatgtggatcgcgcaccagaggggtacgacgacggcgat |
| | ggccacgacctgggatagcatggcgaaaagggttggactatgacggttggttcggaacatggccgacagatgaaga |
| | atagggatacgagggtagagacactcacgatagcagaacgctggtgcgggttggcgatctccagctctggacctgg |
| | atcgccacccacacggccacgattgcaccagagaagtgaaaggcctggacactgagaccaggatggcgtccgtcc |
| | agcacgggccagtagaagacaattagatttcccaagagttcgtcaaatcccgttccggtgatgttgccttgcaagggct |
| | ctgccgtgccggatagctttcgctctcggtagctgttggccatgagctcgaggaagccattccggaatcngaagccat |
| | agatggcgtctagtccaagtacggacaagcagagaagtatgtaggctgaaagggccat |
| intergenic region | cctgtttagagtggccagaaggtgtgtgtgttatctgcaggatgccggtaccagtagggctgtatgtaaatacggctgc |
| between ctvC and | agtagtttcaagttctgcttcgatcaagcgttagacctaggattgagcgcggctctggcaatggcggcttttctcatggta |
| the pyrG cassette | tagcatggcatagcctgaggatataggtactccataccgaggtacgagtacatctatactaagaatagtgactcccagc |
| (1033P, 605 bp) | ttgcctatcccctgcttatcccggagtttgcatctccgccaggaagcacgcggactgaggcggagtaattaacagaag |
| (SEQ ID NO: 9) | gcatggcaatgcttactgcgtggggcttaaaacctgacctgacctggcctggcctggcctgatctgatgtgaaactggt |
| | tctccttctctatctccctctgtcagattgatcgtcaaaacctaaccctaagtcaaatttaaacgccacgcaccggatactc |
| | tcaactctgaatacggccttgatcagccaatcacagaagattgcgagctgacagttcgtattgattactttaaagcctggc |
| | atagacgatctgccattgatttgcaattctccggcccagttgcata |
| pyrG cassette | caatgctcttcaccctcttcgcgggtctgaaataccctcacctggcaacagcaattggcgcttcatggctgtttttccgatc |
| (1885 bp) (SEQ | tctctacttgtacggctatgtgtactcgggtaagccacaaggcaagggcagattgctgggaggtttcttctggttttctca |
| ID NO: 22) | aggcgctctgtgggctctgagtgtgtttggtgttgccaaagacatgatctcttactgagagttattctgtgtctgacgaaat |
| | atgttgtgtatatatatatatgtacgttaaaagttccgtggagttaccagtgattgaccaatgttttatcttctacagttctg |
| | cctgtctaccccattctagctgtacctgactacagaatagtttaattgtggttgaccccacagtcggaggcggaggaatacag |
| | caccgatgtggcctgtctccatccagattggcacgcaatttttacacgcggaaaagatcgagatagagtacgactttaa |
| | atttagtccccggcggcttctattttagaatatttgagatttgattctcaagcaattgatttggttgggtcaccctcaattgga |
| | taatatacctcattgctcggctacttcaactcatcaatcaccgtcataccccgcatataaccctccattcccacgatgtcgtc |
| | caagtcgcaattgacttacggtgctcgagccagcaagcaccccaatcctctggcaaagagactttttgagattgccgaa |
| | gcaaagaagacaaacgttaccgtctctgctgatgtgacgacaacccgagaactcctggacctcgctgaccgtacgga |
| | agctgttggatccaatacatatgccgtctagcaatggactaatcaacttttgatgatacaggtctcggtccctacatcgcc |
| | gtcatcaagacacacatcgacatcctcaccgatttcagcgtcgacactatcaatggcctgaatgtgctggctcaaaagc |
| | acaactttttgatcttcgaggaccgcaaattcatcgacatcggcaataccgtccagaagcaataccacggcggtgctct |
| | gaggatctccgaatgggcccacattatcaactgcagcgttctccctggcgagggcatcgtcgaggctctggcccaga |
| | ccgcatctgcgcaagacttcccctatggtcctgagagaggactgttggtcctggcagagatgacctccaaaggatcgc |
| | tggctacgggcgagtataccaaggcatcggttgactacgctcgcaaatacaagaacttcgttatgggtttcgtgtcgac |
| | gcgggccctgacggaagtgcagtcggatgtgtcttcagcctcggaggatgaagatttcgtggtcttcacgacgggtgt |
| | gaacctctcttccaaaggagataagcttggacagcaataccagactcctgcatcggctattggacgcggtgccgacttt |
| | atcatcgccggtcgaggcatctacgctgctcccgacccggttgaagctgcacagcggtaccagaaagaaggctggg |
| | aagcttatatggccagagtatgcggcaagtcatgatttcctcttggagcaaaagtgtagtgccagtacgagtgttgtgga |
| | ggaaggctgcatacattgtgcctgtcattaaacgatgagctcgtccgtattggcccctgtaatgccatgttttccgccccc |
| | aatcgtcaaggttttccctttgttagattcctaccagtcatctagcaagtgaggtaagctttgccagaaacgccaaggcttt |
| | atctatgtagtcgataagcaaagtggactgatagcttaatatggaaggtccctcagggacaagtcgacctgtgcagaag |
| | agataacagcttggcatcacgcatcagtgcctcctctcagacag |
| intergenic region | attcagcctattgagattacagccacggaagtaatcctgtaaggatcaggatgcaactccatgcaaggcgctaaggatc |
| between the pyrG | aggatccttttcttcaggattgtggcaacggcgccagcggccagcgggcgctatcgcgtcggtggtgatggcgttattt |
| cassette and | ggatttcggaggatagaatccggtcagcctaatcaagccaactccgtcggacttcggcgggactgtccggtcagttag |
| AN1031 (1031P, | agctagagaaggaaggaggtagagtcccagatagacaaaagacttggctgctatatatcttattattcaatcctcaatcc |
| 384 bp) (SEQ ID | cgctagctgtcaatagaatgatcctcagccgcacttgaagtcttgtctacatcccgaatccaggcgca |
| NO: 11) | |
| AN 1031 (2033 | atggctgagacggattcctcccacacccgtgggcccgtagactcaatccagaagaacgacgcctcaagcgacgatg |
| bp) (SEQ ID NO: | ccgaggcagagaccaagatccagtatccctcgggctggagggtcacgatgatcctgacttcggtgacattggcgtact |
| 12) | ttcttttctttcttgacctagccgtgctgtcgaccgcgactcctgccattacctcgcagtttgactcgttagtcgatgttggat |
| | ggtgcgttatgtcccctactgcgctcttccctaggtacatatgtgctggatgctaaaacccaccttgccggcaggtatgg |
| | aggcgcctaccagcttggaagcgcagcgttccagcccctgacgggcaaaatctacagccagttctcgatcaaggtag |
| | ttctccctcaaccatttgacgcagttggaggcttgggtgctcatgaatagcagtggacattccttgtcttcttcattgtctttg |
| | aactcggctctgtcctgtgcgccgcagcacgcaactcgcccatgttcatcgttggtcgggtcattgcaggcgtagggtc |
| | ggccggcatgtccaacggcgccgtaaccacaatctccgcggtcctgccaacgcagaaacaggcgctcttcatgggc |
| | ctgaacatgggtatgggccagctcggtcttgcgacgggaccgattatcggaggcgcgttcacaacgaacgtttcgtgg |
| | cggtggtgttcgtccccctgctccctcctttcaaatcccacctactaggcgaccatgcagagaagatgcaccagctgat |
| | gacgacgcaggcttctacatcaacctccccctcggcgccgttgtcggcggcttcctcctcttcaacacgatccccgag |
| | ccgaaaccaaaggcccctccgttgcagatcctcggcaccgcaatcaggtccctcgatctgccgggattcatgctaatc |
| | tgccctgccgtggttatgttcctcctgggtctgcaattcgggggcaatgagcacccctgggacagctccgtcgtgatcg |
| | gcctcattgtcggaggaggtgccaccttcggtgtcttcctcgtgcaccagtggtggcgtggcgatgaggcaatggtcc |
| | cgtttgccctcttgaagcacaaggttatctggtctgcggccatgaccatgttcttctccctgtccagtgtgctcgtcgcgg |
| | acttctatatcgcgatatacttccaggctatccgggacgactcgccactcatgagtggtgtgcacatgttgcccatcacc |
| | ctaggtctggtcttgtttactgttgtttcaggggcgctgagtatggtcttttctcctgcgtgcttgaacaatggctaaccgtc |
| | cagtctccgtactgggctactacctgcccttccttcttgcaggcggcgccatctccgccgtcggctacggcctcctctcg |
| | acgctgagcccgaccacctctgtcgcgaaatgggtgggataccagatcctctacggcgtagccagtggctgcaccac |
| | cgccgctgtatgtcttcagttttacatacccccggaaccctttgccttcacctttaccaggtagaatgccgctgacaaggc |
| | cgaatgcagccctacgtcgcaatccagaacctcgttcccgcgccccaaatcccgcaagcaatggcaattatcatctttt |
| | ggcagaacattggcgccgccatatctctcattgcggcaaacgccatcttctccaactccctccgcgaccagctagccc |
| | agcgcgcgagtcagatcaccgtctccccgggcgcgattgttgcggccggtgtccggtccatccgggacctcgtctcc |
| | ggctctgcgcttgcggctgttctggaggcgtatgcggaggccatcgacagggtcatgtacttgggcatcgcggttagc |
| | gtgatggttattgtgttctcgcctggtctagggtggaaagatattcggaagacaaaagatctgcaagctctaactagcga |
| | tggagcgcagggtgaagcgacggagaaggagactgttccggttgccctgggttaa |

TABLE 9
Genomic DNA sequence of the afo locus in strain YM283.

| Region | DNA sequence |
|---|---|
| intergenic region | aatgactggtccgtccgtacttagaaagggtgtttctgtccggcagttatttaatgtcggctgtctgctcttgcaatttctctt |
| between AN1037 | ttgatttatctttcgtggtgtatctcgccggaacgaatggccacggttcgcgtttgcgttcatgttcatgttcatagagcagc |
| and Pl-ggs | tgcgaagtttcaaatgttcgttcgttcggctcggcttggctaggcgtatgatggtgttatgtttaggttgagaaggtattctt |
| (1036P, 1487 bp) | agttgggagctagagaaaagattatttgttccctgcaattttgctgtaccccggaaacatagaactgttactgtaccaata |
| (SEQ ID NO: 1) | ctctgcgttccctccccaatgcaccccatacatatggagttggagcctgtacctttgtcgataagcttattctccaatcaac |
| | tctgctattgcagcttttcacttgagctttcttattcgtatgtgctctacggacgaaaaataagctttgttgcctgcagatcac |
| | cttggcagctgtgctgcgcctagacttataatgcaacgtttttaactttttgtttttcttttttctttcttttttaaactagtt |
| | ttcacatgagctacccgttcattataaccatcagctctagctaggacaggatcgcatgagtatatacctatttatattccttcc |
| | ctcccaactcggactcacgctttatatatatgtctactattactcgtgggtgaagagaagtttacgactatttagcctagatga |
| | aggataggttgtgcaatgctcgatagcgtagcatttaaccctacctagtaatgagctacttgggctgctagaataaatctccca |
| | atccaagctaatgtagtcagagctgaacgcaagtctcgtacatggccctacgaggcatcacaatagccctaaagagta |
| | tcacgtgaccatactagcaccgcaatgagttcaggatccgacaatagcgaggctgtatccaagtgcgccgaataatgt |
| | ctatcactgtagaaatatatctgattcgctcagctggtcgataggcgaagcatcggagttggcggagttggcggagttg |
| | caggacttgctggattagggctgaggtcagacggactctcactctccgctatagacactgggcgatgttgtaggcagc |
| | gatgggagaatgtgcattgcacatggtccggagatttctggagtcaggtcatgcagtctagatcctgactgcagtagaa |
| | tgtgcagattccggagcttggggagttaacctgcagtaagctcagctcaagcaatgatcggtaggtaggcctggtggc |
| | catatcagctatagatgcgatccgcgcctcaagcgcatttcaagccctccctcttcaatacgtttgcgataccttagagaa |
| | acaaatcaacatccatcaactggcacagattcatctaccaactcaacgtgattacccgtccagctttgacctaaacctcc |
| | ataatccccatccacaaggcacc |
| Pl-ggs (1053 bp) | atgagaatacctaacgtctttctctcttacctgcgacaagtcgccgtcgacgccactctgtcatcttgctctggagtgaag |
| (SEQ ID NO: 23) | tcacgaaagccggtcattgcctatggctttgacgactcgcaagactctcgcgtcgatgagaatgacgaaaaaatattgg |
| | agccctttggctactatcgtcatcttctgaaaggcaagagcgccaggacggtgttgatgcactgcttcaacgcgttcctt |
| | ggactgcccgaagattgggtcattggcgtaacaaaggccattgaagaccttcataatgcatccctactaattgatgacat |
| | cgaagacgagtctgccctccgtcgtggttcaccagctgcccacatgaagtacgggattgcgctcaccatgaacgcgg |
| | ggaatcttgtctacttcacggtccttcaagacgtctatgaccttggcatgaagacaggtggcacacaggttgccaacgc |
| | aatggctcgcatctacactgaagagatgattgagctccatcgcggtcagggcatcgaaatctggtggcgtgaccagcg |
| | gtcccctccctccgtcgatcaatacattcacatgctcgagcagaaaaccggcggcctgctcaggcttggcgtacggct |
| | cttgcaatgccatcccggtgtcaatagcagggccgacctctccgacattgcgctccgtattggtgtctactaccaacttc |
| | gcgacgactacatcaacctcatgtccacaagctaccacgacgagcgtggatttgctgaggacattaccgaaggaaag |
| | tataccttcccgatgttgcactctctcaagaggtcacccgactctggactgcgtgaaatcttggaccttaagccggccga |
| | catcgccctgaaaaagaaagctatcgctatcatgcaagagacaggatcgcttgttgcaacccggaaccttctcggtgc |
| | agtcaggaatgatctcagtggattggttgctgaacagcgtggagacgactacgctatgagcgcgggtcttgaacgatt |
| | cttggaaaagttgtacatcgcagagtag |
| intergenic region | gctgcatcggtcatgttgttcttctatagagttgaagcaaggtttgtagtttgctctgggtgtctggagttgtctggagttgtc |
| between Pl-ggs | tggagttttgttatgatgttgatgggtacttcttcatactagcattttggcatgttataagaacatattatcagttaaatgtct |
| and Pl-cyc | ttcaatttaatcaatttgtttttagaatgatgttgtctgcctggctatgtatctagatcctatacaagctctatcgactcgacc |
| (1036T, 1768 bp) | taactactacgacttgaaagtcaagcgagaagtgatgatatgaacccatatgtcagacccgctaaatttattagtgataacaact |
| (SEQ ID NO: 3) | atattactcagagcttttctttctagagtatgttagaattgccctttctggctcagtgggaagctcgagacctagtccttagtc |
| | acgtgctgctacatcatgtaaatataagccctacatggctgtcttgtgcatgaggctaacaccattatctgtcactggtcct |
| | tttatttggttcttttctttactttctcgggcgggggggaaagccgctaacactgtctatcgcttggacagaaactcaccagt |
| | ttgttcgcaatcctgaagcgtatgggaagcttacagttaaggagtagctcgagtctggaccctgttttcgacttgtaccttt |
| | gatttggatgactggttaacctcagcttatgtatgatgtgctctcatggtgtcaatatctggtagtctgattctgagcaatttg |
| | atagtatctgatggctggcgagtaaggccagggcgatgactggtataaagtcagccctaaaacttccatccgagatgta |
| | aaaccatcgattcccctccaagatctcctgacgagactaaacaaagatcaagtggccttgtagtaactctagcaagcag |
| | cgacaaaatgcctcaacacgagatgaccaagtcagactcggaacgaatccagtcctcgcaggtaagagcatcagga |
| | catttgctaataccattccgccccgctaatctgcttgaatgcacacaggctaaaagcggaggggacatgtctcttggag |
| | gattcgcctcgcgcgccctgtctgccgggactgctgggtcaattcccagtcctcggccactgcttccggccacgcgga |
| | ctcgggtgccggatctgcaggcggatctcattcggccgcacctggcggtgatgcggggcagggaagaagataaaa |
| | gtaccctgttgtctttggggcgttgaggtataatggcatcgtggtagaccgactgggcttttttttttgatatagttgatcctg |
| | aagcggaggacagttggtaggataaatgaaagatactgaaccatgcccggattttgtgctcaaggacctaaaactgag |
| | aagctgaatctgttcttgtctgggagaaggcctgccagctgcatccgagtatctatcttgccaggaccaaaccgggtct |
| | gggctcagttcttctaacttcttagtggagttttgcagtgtagattcctttgcactatctggtatcctagtagcagcctacca |
| | ggaaataagagataaataaagtcttaattggcattattatgtttctcagaactatatatctcggaacaaagctgagcagac |
| | agaagtttaccctcacatatggacaaattgcgtgctcaggcataagtcggaaacagccttagccaggtcaacacttgta |
| | gccttcgctagacgacgccccagcttttcataatggccggcctggagggagatacggctatccacc |
| pl-cyc | tcaatggtggattccattgctcccgtttgctgtgaccttgatcccatttgtcgccgacccattagctttcttaaccccattggt |
| (complementary, | acctttggaaacctcctggttggcgttgctgatatcagcgcgagtgagacgaccaaggtcatcgtagagtgccgtgtgc |
| 2880 bp) (SEQ | aggtaggtgacccggatgatattgatataatcccgtgcacgtttggcaccgacatgtggagtgagttgcttgaccaagt |
| ID NO: 24) | actcgaacccatcgtcggtggccttgcgttcgaatttggtcaattcaagcagagcagcttcacgagccttctctgtatctg |
| | taccagactttggtcctgtgaattcggagaacatgatggagttgagattgacttcgttgaagtcgcgggagatactgtga |
| | agatcgttggcgagccttgagaatgtaccgaagtgcatgacgcagtcgttgaacaagtacttcaggactggggaggg |
| | gaaaacgtccaccaaatcgcgagagcctcgttcttcattgatctgatgaccaagaagacaaagggcgaagacgagg |
| | gcgatggtcccggcgacgttgtcagcgccaacgacatgtgtccagcgatagtgagaggttccgatgcgctccttgtcg |
| | agtccacgttcacgaaggagaatgttttcttcgcactgaccaatacctgccaggaaatagtgctcgatttcggagcgga |
| | ggagagccttatcgttatcgctggcgagctgtgcacggggatggttcaacagggaataggcaaagcgctcaatgacc |
| | tcgatgtgcgtaggcatccggtcatccgggacctcgctgagggtcgagaacgacttcggatccgcgaacaggtcgc |
| | ggatcttcttcttgaggtcgttcaagtcgtcattggtggccttgatgagggtcatatcgaggtagtcgtcggtgttgtaaag |
| | accgcggatgagaacgagcacgtccagcatcccttgtgaactgataggagtgccttccaagctgctcggagcgatgg |
| | tcatgtatggcaagaactcgaaccatttgcccgccgctcccttctctaccttggcgaacgtggacgatgggacacggtt |
| | gagctcggggcccatgagagtggcctcaatgccccacgtaagcttgcgccattcgggagcaggcttgaacatgtcaa |
| | gacgcccgaagaacttagagagttcctttggcaaggtttgcgagatagtaggaagagtgctgatggaagatggatcga |
| | agcgggggatgggtacgttgagagcagaaacgaggtaggcatcgcggaatgactcgacgctatatgtaaccttgtca |
| | atccagacacggtcctccggtttggcagcagggcgggcgtagaagatggaggtgaggtatgccttcgcggattcaat |
| | gactttgtacaggtggtcgcggatgaggtcgcaagtgggaagagaagcgacgttggcgagtgtaatgagagcgtatg |
| | aggtttcttcagcgcatccccaagagccatcgggcttctggctctggagaatacgactgatcattgtgaagcaggcgat |
| | ggacaccctggacagaagctcctcagatatggatttaaggttgccctttccgtgctcgaaaaggagacggacaagcg |
| | cctgtgaagacagcatagaggagtaccattctgatacattccatttgtctttgacgacacctgctgatgtccaccagacat |
| | cggcgacgtaggtggcgatcttgacgatttgggattcgtacatgttgacatcaggggcgtggaggagcgacataagg |
| | cagttggagttgacggtcacgcttgcgttcctttcgaaagagtagcaacggaagtaggtaggtgcctcaaactctgtga |
| | cgaattcgtcatgggcatatgggtggttgagaacttgcaagagcatcagggtcttcgagctcatgtcagcgtcgtgagt |
| | ggtgccgggaacgaagcctaagacaccttttcctgccacaaggaattcacgtagtttgagggcaatgcgatccaagca |
| | ttccggatccatttgtgcaaactccaggttgttgtcataaagggagctgagcgaccatacgatctcgaagaaggtcatc |
| | ggccagaggttaggaacaacatctcggccatggggtgcgtagacctcgataacgtggcgaaggtaatcctccgctcg |
| | gtcatcccacttggtggccttcatgaggtatgcagcggtggtagatggcgtagccatgaagttaccatcacgtaggaga |
| | tgaggcatgcgatcgaagtcgcagacaccaacgaatgcctccatgcagtgaagcaaggagctgttcttggcgtagat |
| | agcctcccagttaagcttcgccagttttccggcgtacatgttgtacagaaggtcatgatgggggaagctgaaggatacg |
| | ccaaaggcatcgagttgtttgaggaggcagggtacgatcatctcgtacgcgacacgctcagtctccatgatgtcccag |
| | cgctttagggcatcgtcgagataattttgagcggctctggcacgggcaggtatgtcgggttttgaggcgttgctctcgtg |
| | catcttgagagcgacaaggcaggccagagtgttgacgatggagtcgatgagtgacccatcccctgaccaactgccgt |
| | cggcctcctggtgctcgtagatgtaggtgaaggtctccgggaagacgaagacttgcttgccgtcgatctcacgggaga |
| | ccatggctacccaagcagtgtcgtagatagtcggattcgcggtgccaatacccctagaacctggcgtattgagcgcag |
| | actcgagagtctgcatgagggttcgggcgcgtgcatgaagatcttcagatagacccat |
| intergenic region | cctggtgtgattgggctgattaggacaggccggatgggtgtgcaagataggaggagaggactggtacggcgaatga |
| between pl-cyc | gctttaatagccggtcagagattgcgcgtggctgcgcccagatccagcagctccagccatactccagcatactccggc |
| and pl-p450-1 | cagccgggggcatatggcgtggtcactggagctggttaggatcaactgctggttaaggcttactgtgttgccatgctta |
| (1035P, 527 bp) | cggtgcaccgagagggaaggttggagttaacggagttgtaactccggggatccaattagggcttacagtctgcaaatc |
| (SEQ ID NO: 5) | catgcaaagtccgctgcgcccctgacacagcaaggaacagtgtagagtccgattggatagcggagttgaggtgactg |
| | gctggttcctgttagcccctgcatcgacctgcaatgtattgcatcaaattagggctagcctctaactccgttagactatcc |
| | gcaacgcctgtcacacacgtggctaggcagcagatgatatacttttgaaagcagtact |
| pl-p450-1 | ctacaacgcagcgaacgcttccttaatcaagtcttccttcatcttatctcgaggttcaattttgcatgcgaacggaagtgga |
| (complementary, | agagtctcaagagaaaccgacttgtcgtaacagtcctccatgttcatattcttcacagtgtccttgtttgaagaatctgggt |
| 1572 bp) (SEQ | aaaaattgaatgcccaacagagcctcatgatgaagagaccagttgatcttttcgcgagcttatcgcctgggcagactct |
| ID NO: 25) | acgtccagcaccgaaaaggaaatcgggattgacgtcttcagataagcctggcttcgtgccgtttggcgacaagaaata |
| | gcgttcaggcttgaaggcctcaggttcgtcgaagagctcggggtcgtggcccattccccagatgttcatgaagatcata |
| | cttccctctggtagtacgtaaccgccataagacaagctctcccgcgagacgtggggaagggctacagggccgactg |
| | gccgaatccgaaggacctcctgtaggaacgccttgagataaggcaaccgctctaaatcattgaagcacggcatggttt |
| | cggtccccaaaacattatccagctcgtcctgtatcttgcgctggcagtccgggtgggcgataagagcaagaatacacg |
| | attcgatgtacgatatcgtggtcttcgcgccggcatccaagaagccaccgctaaggtttgataactcaatccagctacg |
| | accatccggatggtcaatcacggactctgcaaaacatccggtcctgacaccggaatccatcgccttcttggcaccgtcc |
| | aagagagaattgtagacaccattacgaaaatccttgaattcgtccacaatagtcttccagccggccccggggaaaccg |
| | cgaggaatgtagtctaagaaggggaaagcgtcgaccgctgcaccattgtgagcgatttgaccaattctggtggcagct |
| | tcgtatgcattctcgataattgtgccatagtaactctcgcagcgtggctggccatacacaatgtgtaggagcagcgacat |
| | catagcgcgcctaatatggatcggccgattaggagcgtccatcaatagatcgcgcatgaggttcacagattcctcttctt |
| | gtcgcgctatgtagccactcaaggcacttggcgttaggtaattgtggatacctttgcgaccagtcttccatacagaagtgt |
| | ccatgctttccaccgtgagattcaagccttcagtataccgggcaatcatgggcgaaaatggccggtctcctgtgatatta |
| | ccctgcttgtcaagaatagtccgaacagcctttggactgttcaaaacaatcacagtgcgattcatcaatttgagagagta |
| | cacttcgccatactccctggcccactgtgtcaattgcattggaagccacatcttcgtcatgagatgagcatttccgagaa |
| | caggcttggtaggtggcccgggaggcaagaagttctccctggagcctagctgaaggagcttatagacggcaacagc |
| | ggatcctgcagcagcagccacgatcacgggatccaagttcgcaacagacgggaggtcgacggacagcat |
| intergenic region | tgcgggagggtaggagggtaggagggtagctaggtagttgatagtgctaagtgctctgccgggtcaactgtgaatga |
| between pl-p450- | atgaggtgtagttgagacacttgaggttgactttccaggcgagcgagcgggtcaagagagcagagagaatatgatag |
| 1 and pl-p450-2 | actgggtgtctgtagtagatagacaagatgtatgtctgtcccttggggaagtagggctaatacttctaccttagcacatgtt |
| (1034P, 849 bp) | gcgggaagccacgcactgaggaaacactgacatcgttggggcactctgattggagccggagattaaggtaagatgg |
| (SEQ ID NO: 7) | aatccttctggctgcagcgctgtaagccctaagcctggtggcgcttctggcggacttttcggactacaggactccatcc |
| | aagactccagatcgagactcagcttcgctagtccggaagtccgctggctgatgcttgtctcagcttttcgtctcagctttg |
| | tcgtcttctgtagagcctttagggaaaccccaactcagcatatggatgcagggctggttgggctgattgggcgttgtctg |
| | gacttgtatctgggtatggctgccgtctggggatcaaaggtaaatggggcagaaattgcctgttgaaatagttattgcgg |
| | aggccaatgcaatatcccaagaatttcccaaaatgcaagctactatagatgctacatagccagatagaggttgataatg |
| | ccacattttcaatatatacacatacgtttgtgtgtataagtacataacacgactacagtggctgatatatatgcagtggacg |
| | cctttagacatgtttccatttatgattatagagcgatcctcaggcaagtggttata |
| pl-p450-2 | ctaatagtctgcaacatcgtggatcacctgcacaactgactgactacgtggtaccatctcgcattcaaacggttttggcat |
| (complementary, | cgagaccggaccgggtacaacgacatcgtccttcattgacttggggctgttaggcaggggcttgatgtcgaatcccca |
| 1578 bp) (SEQ | gatgatgttcaaagatacagtgcgcttgaaaatttcagccatcttgagtccaggacagagcctgcgcccagcgccgaa |
| ID NO: 26) | agtgaaggtatgacggtagccagtcaggtcaacgcttggttttgtgccaaattcagactccatgtaccgttcggggcgg |
| | aaatcgtctggggcctcgaaaacatttgggtctcgttggatgccataaaggttcatcacgatgacggtacccttcgggat |
| | gaagtagccattgtattcgaaatcctctgtcgagtaatgaggcggtacgatgggactcggaggccagatgcgagttac |
| | ctctctgacgacgcaattgaagtatttcatcttcaatgcatcttgataagttggcaaacgcgagtcgtattcatcgcccatg |
| | acctccttcagctcatcacgaatcttctgctggcattcggggtgcatcgtcatcatgagcacgaagacacgagtgaaca |
| | tagcgagggtatcagttcctccgtcaatcatgacgcctccgtgataggcaataagatccctatccttgaatccaaactcat |
| | ccttcctctgaagaatggtctgcatgtgagacccgtcgaagacgccagcttccattctcttctcaacccttccgaggaaa |
| | tcattaaagataccaagttgcttgtccttgataccttgagccatgaccctccagccggccagactatcaggaagccactt |
| | ggcgagccaaggaattagagcggtgaagtgaacacctcggagacccatcatgttttcgaagtcgtgaagatattcttc |
| | gtggtagggaatgaatgggtctgaggaggtgaggacgcgttcaccataagcgatagcaacaatactggacatgctgg |
| | tgcggacgagatgcctaaagaattccttgggctcagccaacagctccttcatcagcacgatggtctccgtctcaatgttc |
| | tctgcatatcgatcaatactgtcgttgctaatgagcaacttaaaggccttgtggttgattcggaattcgtcggatttgtagg |
| | aggcgataggaaggaaacggtcgtctttgataggagcagggaggaaaccagtgggtctttcagcagtcttggcattca |
| | gcttgtcaagaatgccagtaacggaggctgagtctgttaggacgataacgttcttgaagaagatcttcaagctgtatattc |
| | ctccatattcttgtgcccatcggctaagctgaaggtgcatgtcgtccattgctggcatctggtggagattacccaacacc |
| | ggcttcgtaggtggcccaggaggtaacgtcttctccctcgaccccatacgaagcagcttgtagaccaagtagcatgcc |
| | aaagggatggccacaggtgcgatcatgttgctgtcaagcagagcagccttcagagcagaaagattcat |
| intergenic region | cctgtttagagtggccagaaggtgtgtgtgttatctgcaggatgccggtaccagtagggctgtatgtaaatacggctgc |
| between pl-p450- | agtagtttcaagttctgcttcgatcaagcgttagacctaggattgagcgcggctctggcaatggcggcttttctcatggta |
| 1 and pl-sdr | tagcatggcatagcctgaggatataggtactccataccgaggtacgagtacatctatactaagaatagtgactcccagc |
| (1033P, 605 bp) | ttgcctatcccctgcttatcccggagtttgcatctccgccaggaagcacgcggactgaggcggagtaattaacagaag |
| (SEQ ID NO: 9) | gcatggcaatgcttactgcgtggggcttaaaacctgacctgacctggcctggcctggcctgatctgatgtgaaactggt |
| | tctccttctctatctccctctgtcagattgatcgtcaaaacctaaccctaagtcaaatttaaacgccacgcaccggatactc |
| | tcaactctgaatacggccttgatcagccaatcacagaagattgcgagctgacagttcgtattgattactttaaagcctggc |
| | atagacgatctgccattgatttgcaattctccggcccagttgcata |
| pl-sdr (762 bp) | atggaaggcaaggtcgcaatcgtcactggcgcatccaatggtattggactcgccaccgtcaatctcctcctcgcagca |
| (SEQ ID NO: 27) | ggagcgtctgtctttggtgtagacctcgctccagcaccgccctcggtgacctccgagaaattcaaattcctacaactcaa |
| | catctgcgacaaggatgcacccgctaggatcgtatccggctccaaagaggcctttggcatcgagaggattgatgccct |
| | cttgaatgtcgctggtatttcggactacttccagactgcgttgaccttcgaggacgatgtatgggaccgagtcctcgatgt |
| | caacctggctgcacaagtgaggttgatgagagaggtattaaaggtcatgaaggtgcagaaatcggggagtatcgtga |
| | atgtcgtcagcaagctggccctcagcggtgcttgtggtggtgttgcatacgttgcgagtaaacatgccttgcttggcgtg |
| | acgaagaacacagcctggatgttcaaggatgacggcattcgatgcaatgcagtcgcacctggttcgactgacaccaa |
| | catccgaaacacgacagacccgtccaaaatagattacgacgccttctctcgagccatgcctgttatcggcgtacactgc |
| | aacttgcaaacaggtgagggcatgatgagccctgagcctgcagcccaagcgatcttcttcctagcttcagacttgagta |
| | agggcacgaacggtgtcgttattccagtcgataacgggtggagtgtcatttag |
| intergenic region | attcagcctattgagattacagccacggaagtaatcctgtaaggatcaggatgcaactccatgcaaggcgctaaggatc |
| between pl-sdr | aggatccttttcttcaggattgtggcaacggcgccagcggccagcgggcgctatcgcgtcggtggtgatggcgttattt |
| and the AfpyroA | ggatttcggaggatagaatccggtcagcctaatcaagccaactccgtcggacttcggcgggactgtccggtcagttag |
| cassette (103 IP, | agctagagaaggaaggaggtagagtcccagatagacaaaagacttggctgctatatatcttattattcaatcctcaatcc |
| 384 bp) (SEQ ID | cgctagctgtcaatagaatgatcctcagccgcacttgaagtcttgtctacatcccgaatccaggcgca |
| NO: 11) | |
| AfpyroA cassette | caatgctcttcaccctcttcgcgggtctgaaataccctcacctggcaacagcaattggcgcttcatggctgtttttccgatc |
| (2088 bp) (SEQ | tctctacttgtacggctatgtgtactcgggtaagccacaaggcaagggcagattgctgggaggtttcttctggttttctca |
| ID NO: 28) | aggcgctctgtgggctctgagtgtgtttggtgttgccaaagacatgatctcttactgagagttattctgtgtctgacgaaat |
| | atgttgtgtatatatatatatgtacgttaaaagttccgtggagttaccagtgattgaccaggacatcagatgctggattacta |
| | aggtaatgtaaggtcagttcgagaccatctgatattaccacaaatacaatggcgagagagtttttcgtaaaagccaatcc |
| | ttggcgtttccagctgttcctgacggttgtaggcccaagtccgcgggaaaccgcccacaaagcggcgtttttgcagatt |
| | ggcagatttatgctggaaacttactggggagatggaggggcacaagcgctgtgattggttttcaaagccgcggccgg |
| | atggaacgaagacataattcggcggggacatgaaaatgtgggtgatcgatacggaatttttggttcttcggaggcgac |
| | aaagggcgcaacggtcgaggttagtagttatcttgactcacacttacagggcccgtcttcggtcttcttaagaactgggt |
| | tttgctgggacttcccccccacctctcttttctactgtgtctcgtatctatttctatactcattctttcacttctcttagtac |
| | caccattcccttctaaatacacagaatggcttccaacggtaccaatggcgcctccgcctccaacagcttcactgtgaaggccg |
| | gcttggctcagatgctgaagggtggtgtgattatggacgtcgtcaacgcggagcaggtatgagcgattgtcatcagga |
| | tacttccagccctttgacgctaacatgacttctacaacaggcccgcattgcggaggaggccggtgccgctgccgtgat |
| | ggccctggagagagtccccgccgacatcagagcccagggtggcgttgcccgcatgtctgaccccagcatgatcaag |
| | gagatcatggctgctgttaccattcctgtcatggccaaagctcgtatcggacacttcgttgagtgccaggtaaggctgcc |
| | tttctcccgtggaaagcctgcattgcagctaacatgtgtaattgttagatcctcgaagccattggcgttgactacatcgac |
| | gagtccgaagtccttacccctgccgatgatgtctaccacgtgaagaagcacgactacaaggttcctttcgtctgtggttg |
| | ccgcaacctgggcgaggcccttcgtcggatcgccgagggtgccgctatgatccgtaccaagggtgaggccggtacc |
| | ggagatgttgttgaagccgtcaagcacatgcgcacggtcaactcccagatcgcccgcgcccgctccatcctccagaa |
| | ttccaccgaccccgagattgagctgcgtgcctacgctcgtgagcttgaggtcccttatgagcttctgcgcgagaccgcc |
| | gagaagggccgtcttcccgttgtcaacttcgccgccggcggtgttgccactcccgctgatgccgcactcatgatgcag |
| | ctgggctgcgacggagtgttcgtcggctctggtattttcaagtctggtgatgcgaagaagcgcgccaaggctattgtcc |
| | aggccgtgactcactacaaggaccccaaggtcctcgctgaagtcagcgagggtctgggtgaggccatggttggtatc |
| | aatgtctctcagatgcccgaggccgaccgattggccaagagaggatggtaattgcactactatctctacttgtgattcttc |
| | ttatgttcttgtcatgatatgggcgttggaaaagttgatatagcgttctttgatgcattttgcattcaagactttcaggttca |
| | ttcttgttagggtgttctgtgcatttgtccttcattatgtagacactcgcgaattctgaaaagctgattgtgagcatcagtgc |
| | ctcctctcagacag |
| intergenic region | ggcatcgtctacaagcagatgctaggcacacatttctttctgccgctaaaaattgggtaatgcagagccacctcgcttttt |
| between the | ttttttcgaacattttccatcttgtggtatttctgggttcatttcgctccatataacgaagattggccttggtacgggctaggg |
| AfpyroA cassette | ttcgcgggtgggatagttatagaatgagaaataatacttttatatgtaacaatttcaacttctcaagatgaatataccattcgg |
| and AN1030 | atagagcagcttctgagtatcgacagacttaggtaggcttatgggtatgctctgttgaatatcttgtagatgtgacaggca |
| (1031T, 591 bp) | atagattgttagattatagcctacaatccacagctcagctcagcacgagtttgattttttcattataattggaataagcactg |
| (SEQ ID NO: 13) | agctcagaatgaaaccaatagattactagggctatgcgtagacgttgaacgggatccatcaccaagcgcagtattagg |
| | gcaccttttgtcgtgggtatatagcaactaaacacattctcttcggtcctgttcggccctcttcggcctccattagccagtc |
| | aaaataaacagtaaccag |
| AN1030 | ctacaaagtgacaacaagcttctttcccgaaaccccctttcgctggatatccagcgcctcctggatcttctcgagcccctt |
| (complementary, | tccgacaacgagcggcggcggtgcaggcacaaactgccctctctcgagcgcttggggcagaaagtccatgtaaacc |
| 1218 bp) (SEQ | cggctgaccacactgtccgggtccaccagcccgtcaacaaggataaacttggcgatgacgcctgtgcggcgctgcc |
| ID NO: 14) | ggatgctcgatttcaccattcctcccagcatcccaatgaggtaagtccccttgccgacgaaggtggttagcttctcaggc |
| | gggatgatctcaccggcgacggcgatgaactttctcgtcagcgcaggatcatgcttgcgcatcacgagggtgcaggc |
| | ttccaccgcaccggcgccaatggtatatgcgccgacgagctctctgcccttgagggcggataagagatccttggcca |
| | ggaacttgctccggtagtcaaagacgtggctcgccccgagccccttgacatagtcgaagttcttgggcgacgaggtc |
| | gaaaggacctcgtagcctgctgcgacagcgagctggatcgcattgctgccaacgctgctggcgccgcccgtgatgat |
| | caccgcgcgcggggaccccgacctgccccgctgcacctctcccctgcccttttccgcaagctgcggcatatcgagg |
| | gccagatagtccttgtggaagagaccaaatgcggccgtacccagcccgagtccgagcacagatgcctgcgcatcgc |
| | tgatcccagcgggcaccggcgtgagcatatgcactcgcaggacggtatacagctggaacccaccctcggccgggtc |
| | gttcacctctttcgcaatcgccgtcgcgcttccacagacgcggtcgcccacggcgaaccgggtgacgcccggtccga |
| | cctcgacgacctcgcccgcaacatcagtcccaaagatgaacgggtagtggatatacccggccagcgcgggcccgat |
| | gaactgcaagacccagtcgaacgggttgatagctacggcgccgttcttgacgaccacctggccagggccagggcgc |
| | gtgtagggggcgtcgccgactttgaaggggatcacctttttggcggggatccacgcggcgcggtttttgggtttgggg |
| | gtcccgttgccgttggtagccggcgctgctgcggttgctgcggttgtatcttgagttgccat |
| intergenic region | aacgaggtccaggtgacggtaacgtggttcagtgcagttccaatgtatggtagcgttgtaagctgacacggcgacggc |
| between AN1030 | tgcgagaggggttggggggacggaaccagctgaaacaggactggcgaaagaaagctgctgtgttatatgtaggcag |
| and PalcA- | agctaaagaaccttgtggagcgacagaaccaaagtcagtctgggccatgggctatcttccataattttgggagctcgag |
| AN1029 (1029P, | gtccggattgcccgttaatactccgccagactagggcaagatagggctacgcggagttttaggtggacggatttcaac |
| 1221 bp)* (SEQ | cctccgaagtccgctcgaacttttgtcgacgagattaagccactagcctaaaggaatcagacctttaattcctcaggccg |
| ID NO: 15) | agtcgggatcattgaaggcgagaatgaggtgaggttgtcagccacatcgtcagctcaatcctttagaccacgttcttatc |
| | tcgcggccgttctccaatcgacgggcccgctggcccccagcgtgcagattacaccgtctcgctccgactgcaggatct |
| | ggcgtcttccatgcgcggacgtttcggacggcgatgactgtctgagtggttggcagggatgcacccctacctacccct |
| | gatcgaagctaatggtaatgcagaatacgaggttggttagactaagcgcttctgcagctgcagcgcatggaagctgttc |
| | tgtctggtggagagactaagcagtgctctgtgctcctctgtgctgctctgcattgcactgcactgtactgcattgtactgca |
| | ttgctgttctgcacggatcattcatccatctaccatggatccactactaacctcgcttactctagtcgatctggtcaagacg |
| | accaagacctcggagaattagatggccaaccaaggatagatgcgagatcaactgatccaccgctggcaaacttagtt |
| | gtgaatgtcgcgaacgcaaataccacggagatggcatgcagccgcacccgaaatggaatgctgtaggcctaatcaa |
| | gctcatcgattctcgcccccaaatctgggctgcgcggtcctgcaggtgagacggatcctggaggctccatgctggctg |
| | gctctgcctcctcgtggacgagggtacgatggcagccagtctgctggcgtgctggcgccgctggtagcacggccac |
| | gagcctattgattgcacgggcaaacgttcgtaactcgctcgtaa |
| PalcA (404 bp) | ctgaaaagctgattgtgatagttcccacttgtccgtccgcatcggcatccgcagctcgggatagttccgacctaggattg |
| (SEQ ID NO: 16) | gatgcatgcggaaccgcacgagggcggggcggaaattgacacaccactcctctccacgcaccgttcaagaggtac |
| | gcgtatagagccgtatagagcagagacggagcactttctggtactgtccgcacgggatgtccgcacggagagccac |
| | aaacgagcggggccccgtacgtgctctcctaccccaggatcgcatccccgcatagctgaacatctatataaagaccc |
| | ccaaggttctcagtctcaccaacatcatcaaccaacaatcaacagttctctactcagttaattagaactcttccaatcctatc |
| | acctcgcctcaaa |
| AN1029 (2354 | atggcgtgtcccaccagacgaggacgacagcagcccggctttgcatgcgaggagtgtcgccgccgcaaagcgcgc |
| bp) (SEQ ID NO: | tgtgatcgcgtgcgtccgaaatgcgggttctgcactgagaatgagctgcagtgtgtgttcgttgacaagaggcagcag |
| 17) | aggggtccgatcaaagggcagatcacctcgatgcagtcgcagctgggtaggtgtttgtcttgtctcattgtatctcgtctc |
| | gtctgcgcttttgtgattatggggctgccatgtttccggtccggacacaggcatctgcaaggcccgccgctgtgctccc |
| | ccgatctgcagggaccaatgcagctggttctggagcttgtgctgtgctgcttccctgtctttccacatggtcgagtcgag |
| | cgagctagctaacatgggatgcctcatgctttcagcaacgcttcgatggcagcttgatcgatacctgcgacatcgacct |
| | cccccgtccataaccatggccggcgagctcgatgagccaccagcggatatccagacgatgctggatgactttgatgta |
| | caggtcgccgcgctgaagcaggatgccacggcaaccaccacaatgtcgacgtcgacagctctcatgcctgccccag |
| | ccatctcatctaaagatgctgctcctgctggtgctggtttatcgtggcctgacccaacctggctggatcgccagtggcag |
| | gatgtcagcagtaccagcctcgtccctccatcagacctgacagtctcgtcggccactaccctaaccgaccctctcagct |
| | tcgaccttttgaacgagactcctcctcctccttctacgacgacaacaacgtcgacgacgaggcgagactcatgtactaa |
| | ggtcatgttaactgacctcatccgggctgaattgtacactacctaactgatttgtctaccatgacacctgactgacaatgtg |
| | cagagaccaactctacttcgaccgggtccacgccttctgccccatcatccaccggcgacggtactttgcgcgggtcgc |
| | ccgagatagccataccccagcacaggcatgtctgcagttcgccatgcgaacgctcgcagcggcaatgtctgctcact |
| | gccatcttagcgagcatctctatgccgagaccaaggccctcttggagacgcacagccagacgcccgccacaccgcg |
| | agacaaggtcccgctcgagcacatccaggcctggctgttgttaagccactacgagctgctgcggatcggcgtgcacc |
| | aggctatgctcacggctggccgggcctttcgtctcgtgcagatggcacgactgtcagagctggatgccgggtcagatc |
| | gacagctctcgccgccgtcttcgtcgccgccgtcttcgctaaccctatctccttcgggggagaatgctgagaacttcgtc |
| | gacgccgaagaaggccggcggacgttctggcttgcttattgctttgatcgtttgctttgcttgcagaatgagtggccgtta |
| | acgttacaagaagagatggtacgtcgcgcttcttttattctatttacctcagaatttatattcagttattttttattctaac |
| | cctgctagatattaacccgcctcccctccctcgaacacaactaccagaacaatctccccgcacgcacgccctttctcactgaag |
| | ccatggcccagaccgggcagagcacaatgtccccgtttgccgaatgcattatcatggccacccttcacggccgatgta |
| | tgacgcaccgccgcttctacgcaaacagcaactcgactgcgtccggctccgagttcgagtctggcgccgcgacgcg |
| | agacttctgtatccgccagaattggctgtcgaatgcagtggaccggcgagtccagatgctacagcaggtctcctcgcc |
| | cgctgttgacagcgacccgatgctgctcttcacgcagacgctcggctaccgcgcgaccatgcacctgagcgataccg |
| | tccagcaagtctcctggcgggctctcgccagctcgcccgttgaccagcagctactgagcccgggcgcgacgatgtc |
| | gctgtcggccgccgcgtaccaccagatggccagccacgcagccggcgagatcgtccgcctggcgaaggccgtcc |
| | cctcgctgagtccgttcaaggcgcacccgttcctacccgatacgttggcgtgcgccgccacgttcctctcgacgggca |
| | gtcccgatcccacgggcggcgagggggtgcagcatctgctacgagtgttaagcgagctgcgcgatacacacagcct |
| | ggcgcgggattatttgcaggggttgtcggtgcagacgcaggacgaagatcatagacaggatacgaggtggtattgta |
| | catag |

*Part of the intergenic region between AN1030 and AN1029 has been removed after replacing the native promoter of AN1029 with PalcA. The original intergenic region between AN1030 and AN1029 (1029P) is 1370 bp.

TABLE 10
Genomic DNA sequence of the afo locus in strain YM343.

| Region | DNA sequence |
|---|---|
| intergenic region | attcagcctattgagattacagccacggaagtaatcctgtaaggatcaggatgcaactccatgcaaggcgctaagg |
| between pl-sdr | atcaggatccttttcttcaggattgtggcaacggcgccagcggccagcgggcgctatcgcgtcggtggtgatggcgtt |
| and pl-atf | atttggatttcggaggatagaatccggtcagcctaatcaagccaactccgtcggacttcggcgggactgtccggtca |
| (1031P, 384 bp) | gttagagctagagaaggaaggaggtagagtcccagatagacaaaagacttggctgctatatatcttattattcaatc |
| (SEQ ID NO: 11) | ctcaatcccgctagctgtcaatagaatgatcctcagccgcacttgaagtcttgtctacatcccgaatccaggcgca |
| pl-atf (1134 bp) | atgaagcccttctcaccagaacttctggttctatctttcattctattggtactatcttgtgccatccggcctgctagagg |
| (SEQ ID NO: 29) | acgatgggttctctgggtcattattgttgggctcaacacctacctcaccctgactccgaccggcgattcgaccttggat |
| | tatgacattgccaataacctcttcgttattaccctcacggccacagattatattctcttgacggacgtccagagagagt |
| | tacaattccgcaaccagaaaggtgtcgagcaagcctcgttgcttgaacgcatcaagtgggcgacctggctggtgca |
| | aagtcggcgtggtgtgggctggaattgggagccgaagattttcgtccacaagtttgacccaaagacttcacgcctttc |
| | attcctcctccagcaactcgtcacaggttttcggcattaccttatttgcgatctagtctcgctatatagccgcagtccag |
| | tcgccttcatcgaacctcttgcttctcgccctctgatctggcggtgtgcagatattaccgcatggctcctgttcacgacg |
| | aaccaagtatcaattcttcttacggcattgagtgtcatgcaagttctctcaggttactcagaaccacaggactgggtc |
| | cccgtgtttggccgctggagagatgcttataccgttaggcggttctggggtcgatcgtggcatcaattggttcgcagat |
| | gcctatcagccccaggaaaacatctttccacgaagattctaggcttgaagtctggctctaacccggcgctttacgtac |
| | aactgtacaccgcattcttcctctcgggagttttgcatgcgattggggacttcaaggttcacgcagattggtacaaag |
| | ccgggactatggagttcttctgtgttcaagcggcgatcatacagatggaggatggggttctctgggtcggaaggaag |
| | cttggtatcaagccgacttcgtactggaaggcccttggacatctttggactgtggcatggttcgtctacagctgcccga |
| | attggctgggggcaactgtctcgggaaggggaaaggcctcaatgtcgttggagagtagtctcattcttggtctgtacc |
| | ggggggaatggaatccccctcgtgtagcacagtag |
| intergenic region | ggcatcgtctacaagcagatgctaggcacacatttctttctgccgctaaaaattgggtaatgcagagccacctcgctt |
| between pl-atf | tttttttttcgaacattttccatcttgtggtatttctgggttcatttcgctccatataacgaagattggccttggtacgggc |
| and pl-p450-3 | tagggttcgcgggtgggatagttatagaatgagaaataatacttttatatgtaacaatttcaacttctcaagatgaat |
| (1031T, 591 bp) | ataccattcggatagagcagcttctgagtatcgacagacttaggtaggcttatgggtatgctctgttgaatatcttgta |
| (SEQ ID NO: 13) | gatgtgacaggcaatagattgttagattatagcctacaatccacagctcagctcagcacgagtttgattttttcattat |
| | aattggaataagcactgagctcagaatgaaaccaatagattactagggctatgcgtagacgttgaacgggatccat |
| | caccaagcgcagtattagggcaccttttgtcgtgggtatatagcaactaaacacattctcttcggtcctgttcggccct |
| | cttcggcctccattagccagtcaaaataaacagtaaccag |
| pl-p450-3 | ctagccactagcaggcttcgtgaacgtcaacgggcaagcacggatgacctcctcagcttccttacttcttggcttgat |
| (complementary, | gcggcaagggaaatctagtggacgtgagatcatagcctggtgatactctctagtaggctcaatttctttgccattttcg |
| 1569 bp) (SEQ ID | tcaactgcttttaagagatcaaacagcgacagaacagaggccgcagccaaggtgatggtggaatgagcgaggtaa |
| NO: 30) | cgaccagcgcaaattcttctaccgaagccgaatgcgatatcaaaggggtctctgacagccttgttaggcttaccgtct |
| | tcggtcaagtatcgctcaggccggaattcgtctggctgggggtaatcggtctcgtcgttggacatcgcccattggttg |
| | gcaaacacgatggatcccttagggatgtggtattccctgtaaacgtcatctgagatggtttgatgaggtacgcccata |
| | ggagtcacaggtctccagcggtaaacctccttgatcacagcgttgaggtatgggaaagaggggaagtcggcgtgct |
| | cgggcatccttccattgagaacactatctaattctcgttgtgctttcttctgtacttcggggaaacagaccatggcgag |
| | gaagaaagtccccaaggcggatgcagtcgtatcagcaccagcaatgtagacttgaccagcaacatccttgaggtgc |
| | tccaaatctgcctcctggttttccgagttctgaagatctcggagagcgtcagatncaaaggagggctcataatcgcc |
| | agttttaatcatctcctgggcaactttgaatggctgttcacgaacatagtacgcatgacctcgcattaaggcagccttt |
| | tgatggaagatagtccctgggacccatggaggaatgtgtttcatcgcagggatgatgtcaacaagaaaggcgccag |
| | acgtcataatctcagacgctgcaaggacagctttctcgaccaggtcaacataggggtcgttataaggttcagtctca |
| | aggccataggtcattgaaagcgtcgtagagccgaccaagttccgtacatgatcgagaacgtcgtcgggcttctcgta |
| | aagctgcttgaggaaccgtttcacatatcgcaactcacgaggttggtttataccggggtttgaagagttgaagtgctt |
| | ggtgaagcttcttcgaccagcccgccatgactcgccgtatggcattaaggcccacgtaaagccccatcctgacagct |
| | cgtggtgcatcgtgctgtgtggtctgctcgagtagatcgccgacctcttcagcaacaagtcattggcggcgttggcag |
| | aattcagtattacgatcgaggttcccatggcgctaacatgtatgatatcagagttgtactctttaccccagcgagcat |
| | aggtttcccattcgaccttcgctggtaggtccatgacgttgccaataattggaagtttctttggcccaggcggcaggtg |
| | ctgctttttcttcttctgagaatctatccagtaggccaagcctatagcagtccatattacaaggactggtagagcacgt |
| | tccgttgacggagccat |
| intergenic region | aacgaggtccaggtgacggtaacgtggttcagtgcagttccaatgtatggtagcgttgtaagctgacacggcgacg |
| between pl- | gctgcgagaggggttggggggacggaaccagctgaaacaggactggcgaaagaaagctgctgtgttatatgtagg |
| p450-3 and | cagagctaaagaaccttgtggagcgacagaaccaaagtcagtctgggccatgggctatcttccataattttgggagc |
| PalcA-AN1029 | tcgaggtccggattgcccgttaatactccgccagactagggcaagatagggctacgcggagttttaggtggacggat |
| (1029P, 1370 bp) | ttcaaccctccgaagtccgctcgaacttttgtcgacgagattaagccactagcctaaaggaatcagacctttaattcc |
| (SEQ ID NO: 15) | tcaggccgagtcgggatcattgaaggcgagaatgaggtgaggttgtcagccacatcgtcagctcaatcctttagacc |
| | acgttcttatctcgcggccgttctccaatcgacgggcccgctggcccccagcgtgcagattacaccgtctcgctccga |
| | ctgcaggatctggcgtcttccatgcgcggacgtttcggacggcgatgactgtctgagtggttggcagggatgcacccc |
| | tacctacccctgatcgaagctaatggtaatgcagaatacgaggttggttagactaagcgcttctgcagctgcagcgc |
| | atggaagctgttctgtctggtggagagactaagcagtgctctgtgctcctctgtgctgctctgcattgcactgcactgt |
| | actgcattgtactgcattgctgttctgcacggatcattcatccatctaccatggatccactactaacctcgcttactcta |
| | gtcgatctggtcaagacgaccaagacctcggagaattagatggccaaccaaggatagatgcgagatcaactgatcc |
| | accgctggcaaacttagttgtgaatgtcgcgaacgcaaataccacggagatggcatgcagccgcacccgaaatgg |
| | aatgctgtaggcctaatcaagctcatcgattctcgcccccaaatctgggctgcgcggtcctgcaggtgagacggatc |
| | ctggaggctccatgctggctggctctgcctcctcgtggacgagggtacgatggcagccagtctgctggcgtgctggc |
| | gccgctggtagcacggccacgagcctattgattgcacgggcaaacgttcgtaactcgctcgtaacctataattacga |
| | tagctaaccacatcctggttctctctcataagaatgaatggcattcccgccttgatccgtcagcattgtcaacccggat |
| | agaccagtgcctcgtcattcaacatcacagatccagagactacaaagaccagcaatc |
| AfpyrG cassette | caatgctcttcaccctcttcgcgggtctgaaataccctcacctggcaacagcaattggcgcttcatggctgtttttccg |
| (1885 bp) (SEQ ID | atctctctacttgtacggctatgtgtactcgggtaagccacaaggcaagggcagattgctgggaggtttcttctggttt |
| NO: 31) | tctcaaggcgctctgtgggctctgagtgtgtttggtgttgccaaagacatgatctcttactgagagttattctgtgtctg |
| | acgaaatatgttgtgtatatatatatatgtacgttaaaagttccgtggagttaccagtgattgaccaatgttttatcttc |
| | tacagttctgcctgtctaccccattctagctgtacctgactacagaatagtttaattgtggttgaccccacagtcggag |
| | gcggaggaatacagcaccgatgtggcctgtctccatccagattggcacgcaatttttacacgcggaaaagatcgag |
| | atagagtacgactttaaatttagtccccggcggcttctattttagaatatttgagatttgattctcaagcaattgatttg |
| | gttgggtcaccctcaattggataatatacctcattgctcggctacttcaactcatcaatcaccgtcataccccgcatat |
| | aaccctccattcccacgatgtcgtccaagtcgcaattgacttacggtgctcgagccagcaagcaccccaatcctctgg |
| | caaagagactttttgagattgccgaagcaaagaagacaaacgttaccgtctctgctgatgtgacgacaacccgaga |
| | actcctggacctcgctgaccgtacggaagctgttggatccaatacatatgccgtctagcaatggactaatcaactttt |
| | gatgatacaggtctcggtccctacatcgccgtcatcaagacacacatcgacatcctcaccgatttcagcgtcgacact |
| | atcaatggcctgaatgtgctggctcaaaagcacaactttttgatcttcgaggaccgcaaattcatcgacatcggcaat |
| | accgtccagaagcaataccacggcggtgctctgaggatctccgaatgggcccacattatcaactgcagcgttctccc |
| | tggcgagggcatcgtcgaggctctggcccagaccgcatctgcgcaagacttcccctatggtcctgagagaggactgt |
| | tggtcctggcagagatgacctccaaaggatcgctggctacgggcgagtataccaaggcatcggttgactacgctcgc |
| | aaatacaagaacttcgttatgggtttcgtgtcgacgcgggccctgacggaagtgcagtcggatgtgtcttcagcctcg |
| | gaggatgaagatttcgtggtcttcacgacgggtgtgaacctctcttccaaaggagataagcttggacagcaatacca |
| | gactcctgcatcggctattggacgcggtgccgactttatcatcgccggtcgaggcatctacgctgctcccgacccggt |
| | tgaagctgcacagcggtaccagaaagaaggctgggaagcttatatggccagagtatgcggcaagtcatgatttcct |
| | cttggagcaaaagtgtagtgccagtacgagtgttgtggaggaaggctgcatacattgtgcctgtcattaaacgatga |
| | gctcgtccgtattggcccctgtaatgccatgttttccgcccccaatcgtcaaggttttccctttgttagattcctaccagt |
| | catctagcaagtgaggtaagctttgccagaaacgccaaggctttatctatgtagtcgataagcaaagtggactgata |
| | gcttaatatggaaggtccctcagggacaagtcgacctgtgcagaagagataacagcttggcatcacgcatcagtgc |
| | ctcctctcagacag |
| PalcA (404 bp) | ctgaaaagctgattgtgatagttcccacttgtccgtccgcatcggcatccgcagctcgggatagttccgacctaggat |
| (SEQ ID NO: 16) | tggatgcatgcggaaccgcacgagggcggggcggaaattgacacaccactcctctccacgcaccgttcaagaggta |
| | cgcgtatagagccgtatagagcagagacggagcactttctggtactgtccgcacgggatgtccgcacggagagcca |
| | caaacgagcggggccccgtacgtgctctcctaccccaggatcgcatccccgcatagctgaacatctatataaagacc |
| | cccaaggttctcagtctcaccaacatcatcaaccaacaatcaacagttctctactcagttaattagaactcttccaatc |
| | ctatcacctcgcctcaaa |
| AN1029 (2354 | atggcgtgtcccaccagacgaggacgacagcagcccggctttgcatgcgaggagtgtcgccgccgcaaagcgcgct |
| bp) (SEQ ID NO: | gtgatcgcgtgcgtccgaaatgcgggttctgcactgagaatgagctgcagtgtgtgttcgttgacaagaggcagcag |
| 17) | aggggtccgatcaaagggcagatcacctcgatgcagtcgcagctgggtaggtgtttgtcttgtctcattgtatctcgtc |
| | tcgtctgcgcttttgtgattatggggctgccatgtttccggtccggacacaggcatctgcaaggcccgccgctgtgctc |
| | ccccgatctgcagggaccaatgcagctggttctggagcttgtgctgtgctgcttccctgtctttccacatggtcgagtc |
| | gagcgagctagctaacatgggatgcctcatgctttcagcaacgcttcgatggcagcttgatcgatacctgcgacatc |
| | gacctcccccgtccataaccatggccggcgagctcgatgagccaccagcggatatccagacgatgctggatgacttt |
| | gatgtacaggtcgccgcgctgaagcaggatgccacggcaaccaccacaatgtcgacgtcgacagctctcatgcctg |
| | ccccagccatctcatctaaagatgctgctcctgctggtgctggtttatcgtggcctgacccaacctggctggatcgcca |
| | gtggcaggatgtcagcagtaccagcctcgtccctccatcagacctgacagtctcgtcggccactaccctaaccgacc |
| | ctctcagcttcgaccttttgaacgagactcctcctcctccttctacgacgacaacaacgtcgacgacgaggcgagact |
| | catgtactaaggtcatgttaactgacctcatccgggctgaattgtacactacctaactgatttgtctaccatgacacct |
| | gactgacaatgtgcagagaccaactctacttcgaccgggtccacgccttctgccccatcatccaccggcgacggtac |
| | tttgcgcgggtcgcccgagatagccataccccagcacaggcatgtctgcagttcgccatgcgaacgctcgcagcggc |
| | aatgtctgctcactgccatcttagcgagcatctctatgccgagaccaaggccctcttggagacgcacagccagacgc |
| | ccgccacaccgcgagacaaggtcccgctcgagcacatccaggcctggctgttgttaagccactacgagctgctgcg |
| | gatcggcgtgcaccaggctatgctcacggctggccgggcctttcgtctcgtgcagatggcacgactgtcagagctgg |
| | atgccgggtcagatcgacagctctcgccgccgtcttcgtcgccgccgtcttcgctaaccctatctccttcgggggaga |
| | atgctgagaacttcgtcgacgccgaagaaggccggcggacgttctggcttgcttattgctttgatcgtttgctttgctt |
| | gcagaatgagtggccgttaacgttacaagaagagatggtacgtcgcgcttcttttattctatttacctcagaatttata |
| | ttcagttattttttattctaaccctgctagatattaacccgcctcccctccctcgaacacaactaccagaacaatctccc |
| | cgcacgcacgccctttctcactgaagccatggcccagaccgggcagagcacaatgtccccgtttgccgaatgcatta |
| | tcatggccacccttcacggccgatgtatgacgcaccgccgcttctacgcaaacagcaactcgactgcgtccggctcc |
| | gagttcgagtctggcgccgcgacgcgagacttctgtatccgccagaattggctgtcgaatgcagtggaccggcgagt |
| | ccagatgctacagcaggtctcctcgcccgctgttgacagcgacccgatgctgctcttcacgcagacgctcggctaccg |
| | cgcgaccatgcacctgagcgataccgtccagcaagtctcctggcgggctctcgccagctcgcccgttgaccagcagc |
| | tactgagcccgggcgcgacgatgtcgctgtcggccgccgcgtaccaccagatggccagccacgcagccggcgagat |
| | cgtccgcctggcgaaggccgtcccctcgctgagtccgttcaaggcgcacccgttcctacccgatacgttggcgtgcgc |
| | cgccacgttcctctcgacgggcagtcccgatcccacgggcggcgagggggtgcagcatctgctacgagtgttaagc |
| | gagctgcgcgatacacacagcctggcgcgggattatttgcaggggttgtcggtgcagacgcaggacgaagatcata |
| | gacaggatacgaggtggtattgtacatag |

TABLE 11
Genomic DNA sequence of the afo locus in strain YM727.

| Region | DNA sequence |
|---|---|
| intergenic region | aatgactggtccgtccgtacttagaaagggtgtttctgtccggcagttatttaatgtcggctgtctgctcttgcaatttctctt |
| between AN 1037 | ttgatttatctttcgtggtgtatctcgccggaacgaatggccacggttcgcgtttgcgttcatgttcatgttcatagagcagc |
| and TC (1036P, | tgcgaagtttcaaatgttcgttcgttcggctcggcttggctaggcgtatgatggtgttatgtttaggttgagaaggtattctt |
| 1487 bp) (SEQ ID | agttgggagctagagaaaagattatttgttccctgcaattttgctgtaccccggaaacatagaactgttactgtaccaata |
| NO: 1) | ctctgcgttccctccccaatgcaccccatacatatggagttggagcctgtacctttgtcgataagcttattctccaatcaac |
| | tctgctattgcagcttttcacttgagctttcttattcgtatgtgctctacggacgaaaaataagctttgttgcctgcagatcac |
| | cttggcagctgtgctgcgcctagacttataatgcaacgtttttaactttttgtttttcttttttctttcttttttaaactagtt |
| | ttcacatgagctacccgttcattataaccatcagctctagctaggacaggatcgcatgagtatatacctatttatattccttcc |
| | ctcccaactcggactcacgctttatatatatgtctactattactcgtgggtgaagagaagtttacgactatttagcctagatga |
| | aggataggttgtgcaatgctcgatagcgtagcatttaaccctacctagtaatgagctacttgggctgctagaataaatctccca |
| | atccaagctaatgtagtcagagctgaacgcaagtctcgtacatggccctacgaggcatcacaatagccctaaagagta |
| | tcacgtgaccatactagcaccgcaatgagttcaggatccgacaatagcgaggctgtatccaagtgcgccgaataatgt |
| | ctatcactgtagaaatatatctgattcgctcagctggtcgataggcgaagcatcggagttggcggagttggcggagttg |
| | caggacttgctggattagggctgaggtcagacggactctcactctccgctatagacactgggcgatgttgtaggcagc |
| | gatgggagaatgtgcattgcacatggtccggagatttctggagtcaggtcatgcagtctagatcctgactgcagtagaa |
| | tgtgcagattccggagcttggggagttaacctgcagtaagctcagctcaagcaatgatcggtaggtaggcctggtggc |
| | catatcagctatagatgcgatccgcgcctcaagcgcatttcaagccctccctcttcaatacgtttgcgataccttagagaa |
| | acaaatcaacatccatcaactggcacagattcatctaccaactcaacgtgattacccgtccagctttgacctaaacctcc |
| | ataatccccatccacaaggcacc |
| TC (1233 bp) | atggaccgtgtgctatcgctggggaaactccccatcagttttttgaagacgttatatctgttcagcaagtctgacatccca |
| (SEQ ID NO: 32) | gcagcgactttaccttctgtatgtctggcgttcactctcgccccacgcaccggaagggtcactggctaatactgagagc |
| | agatggctgtagctcttgtgcttgctgccccgtgtagctttcacctaattataaagggatttctgtggaaccaattgcatctt |
| | ctcacatttcaggtgcgtctagaagcattctccttgaaccgaggccatcaagcgttgacctgagcaggtgaaaaatcag |
| | gttcgttagtccgagacacgacaggcaggtcgacaacgacatgcaatgcttaccgcagccgttagatcgatggtatcg |
| | acgaggatagcatagcaaagccacatcgacccttgccctctggccggatcacacctggacaagctaccctcctctatc |
| | gcgtcctcttcttcctgatgtgggttgccgccgtgtacaccaacacgatctcctgcacgttggtctattcgattgccatcgt |
| | agtgtacaatgagggtgggctggcagctattccggtagtcaagaatttgatcggagctatcggtctcggctgttactgct |
| | ggggaaccacgatcatctttggtatttagtctggcacggtccttctttttgtcaaggtacgcgctgacagatgatggttcaa |
| | gatggcggcaaagagttgcatggactgaaagccgtcgcggtactgatgatcgttggcattttcgctactacggtgagtt |
| | catccggtagagaggcaactacctgctaatatctttgtcacacctgcttagggccatgctcaagacttccgtgaccgga |
| | ctgcagacgcaacacgaggccgcaaaacaatcccgctactgctctcccagcctgtggctcgctggtcactagccacg |
| | ataacagcggcgtggactataggcttgattgccttgtggaagcccccggctatcgttactctggcatatgttgctgcgag |
| | tctccgctgtctggacgggtttctctccagctatgacgaaaaggacgattatgtgtcttattgctggtatggggtacgtcta |
| | tgctttttttcctatgtacgcctggcccatgtccgttgacccagattacagttctggcttcttgggagtaatatcctacccatc |
| | ttccctcgtttgagaggcgagcttccttag |
| intergenic region | gctgcatcggtcatgttgttcttctatagagttgaagcaaggtttgtagtttgctctgggtgtctggagttgtctggagttgtc |
| between TC and | tggagttttgttatgatgttgatgggtacttcttcatactagcattttggcatgttataagaacatattatcagttaaatgtct |
| P450 (1036T, | ttcaatttaatcaatttgtttttagaatgatgttgtctgcctggctatgtatctagatcctatacaagctctatcgactcgacc |
| 1768 bp) (SEQ ID | taactactacgacttgaaagtcaagcgagaagtgatgatatgaacccatatgtcagacccgctaaatttattagtgataacaact |
| NO: 3) | atattactcagagcttttctttctagagtatgttagaattgccctttctggctcagtgggaagctcgagacctagtccttagtc |
| | acgtgctgctacatcatgtaaatataagccctacatggctgtcttgtgcatgaggctaacaccattatctgtcactggtcct |
| | tttatttggttcttttctttactttctcgggcgggggggaaagccgctaacactgtctatcgcttggacagaaactcaccagt |
| | ttgttcgcaatcctgaagcgtatgggaagcttacagttaaggagtagctcgagtctggaccctgttttcgacttgtaccttt |
| | gatttggatgactggttaacctcagcttatgtatgatgtgctctcatggtgtcaatatctggtagtctgattctgagcaatttg |
| | atagtatctgatggctggcgagtaaggccagggcgatgactggtataaagtcagccctaaaacttccatccgagatgta |
| | aaaccatcgattcccctccaagatctcctgacgagactaaacaaagatcaagtggccttgtagtaactctagcaagcag |
| | cgacaaaatgcctcaacacgagatgaccaagtcagactcggaacgaatccagtcctcgcaggtaagagcatcagga |
| | catttgctaataccattccgccccgctaatctgcttgaatgcacacaggctaaaagcggaggggacatgtctcttggag |
| | gattcgcctcgcgcgccctgtctgccgggactgctgggtcaattcccagtcctcggccactgcttccggccacgcgga |
| | ctcgggtgccggatctgcaggcggatctcattcggccgcacctggcggtgatgcggggcagggaagaagataaaa |
| | gtaccctgttgtctttggggcgttgaggtataatggcatcgtggtagaccgactgggcttttttttttgatatagttgatcctg |
| | aagcggaggacagttggtaggataaatgaaagatactgaaccatgcccggattttgtgctcaaggacctaaaactgag |
| | aagctgaatctgttcttgtctgggagaaggcctgccagctgcatccgagtatctatcttgccaggaccaaaccgggtct |
| | gggctcagttcttctaacttcttagtggagttttgcagtgtagattcctttgcactatctggtatcctagtagcagcctacca |
| | ggaaataagagataaataaagtcttaattggcattattatgtttctcagaactatatatctcggaacaaagctgagcagac |
| | agaagtttaccctcacatatggacaaattgcgtgctcaggcataagtcggaaacagccttagccaggtcaacacttgta |
| | gccttcgctagacgacgccccagcttttcataatggccggcctggagggagatacggctatccacc |
| P450 | ctagactgtactcggtttgagaaggcttgcatggctgacctcgggtatctgctccgactcgatgcggcgcagaagatcg |
| (complementary, | atgtggtgctgactgcgaggctcgaccttgaactggaagggagcagggcgattgaccatgcccgggatcgcctgga |
| 1665 bp) (SEQ ID | gagtgacggggatctcgttgccttggtcatctcgagccttgcggacgttgaaaacagccagcagctggaccacggtg |
| NO: 33) | atgtagacactggcgtccgcaaagtaccgacccgcacaagatcggcggccgtaaccaaaagcaatttcgctcggatc |
| | agggtggttgaaaggctccatgtagcgctccggcttgaacactcgcggctctgggtactctttggggtcgttcaggaac |
| | caccatagagaaggcaggagataggaacccttggggatgagatattctccgcacactaaatcttcctcggacttgtgc |
| | gtcaatcccatgggtcccacgggattccatcgccaggcttccttgataatgccgtcgacataaggcaggttggttcgat |
| | cgtcaaagttggggagccgatcggagccgacaactcggtcgatttcttcctgcgcccttgtcacaacctcggggaaca |
| | tgacaagaccacagatgacgctgtggatgatggcgacggtactgtccgagccggcggcgtacaggctcacggcggt |
| | ccacttgatcgcctcttcgtcagccgcggaaacgttgatcttgttgtcctccgacttgatcatgtgcttctcgagaagattg |
| | gacacgtatgacggctggtgggctttgtgcgccatctggcgtttaacaaaatcgtaagggagttccgcagcggcctcat |
| | tgatagccctccatttccgcgccgtcttccggtacgacatgccggggaaccagtctggaaggtacttgatcgcaggtac |
| | ggagtccacggcccaagcgagaggcacaaatgcttgggacaggttttccatggcgtgttcgatcaactcgaccaacg |
| | ggtcctggccctttcgctcaatggagtatccataggtaattttcaaaacgatggcggcagccaacctacaacccatgag |
| | acagtgtagaagacatattaccacgtcgtagggcacttacgttttcaggtgctgcaagatgtcgtccggccggttgaac |
| | gtctgtaggatgaaccgaatggattcttgctcctgaatggggcggaaaccagcagagagccctttcgtcccaatctcct |
| | ggtgcaccattttccggtgcaggcggtacttgtcattgtactgatgggtaatgagaaagttctcgaacccacatagctgg |
| | gcaaagttgagctggggtctcgcggatgtcttttgggccttttttcccatcaccgcgtgggccgcgtccttgtcatggaa |
| | gatgacgagcgttgtccccatgacattgatcgaactgacgggaccataggcatctttgtgcttgaaccagtgcagatac |
| | tcgggctgccccttgggggggagatcaaagaaattcccaataattggcaatggccttggcccaggcgggacgttctgt |
| | ttgaggtttctggtacgagtccggaataccagaacggccatgaaggccacaaaggccacgcagctaagctgaaggg |
| | tagatagctcgtaggccat |
| intergenic region | cctggtgtgattgggctgattaggacaggccggatgggtgtgcaagataggaggagaggactggtacggcgaatga |
| between P450 | gctttaatagccggtcagagattgcgcgtggctgcgcccagatccagcagctccagccatactccagcatactccggc |
| and C6H (1035P, | cagccgggggcatatggcgtggtcactggagctggttaggatcaactgctggttaaggcttactgtgttgccatgctta |
| 527 bp) (SEQ ID | cggtgcaccgagagggaaggttggagttaacggagttgtaactccggggatccaattagggcttacagtctgcaaatc |
| NO: 5) | catgcaaagtccgctgcgcccctgacacagcaaggaacagtgtagagtccgattggatagcggagttgaggtgactg |
| | gctggttcctgttagcccctgcatcgacctgcaatgtattgcatcaaattagggctagcctctaactccgttagactatcc |
| | gcaacgcctgtcacacacgtggctaggcagcagatgatatacttttgaaagcagtact |
| C6H | tcaagcgctcaccgcagttgtacccttttcggaagggtatttctgagccatatacgtcagatcgcccttgacgacgtatcc |
| (complementary, | aatatggctgagtgcgagcagttccttcaactgcggactaagtgtctcttgaagctcctttggcagctttgcagaccagtt |
| 930 bp) (SEQ ID | tgtcaggggtcgcatgtgaggcgccgaataataggcaaacagaatggctcgatcttcatcctcagtcacgttggaacc |
| NO: 34) | agaggtgtgccacaggcgcccgtcaataacgacaatgtcgcccgcatccgcttcaaacgggaccagcagatccggt |
| | gcgttatcgggcacgtcctcccaggtggtccacttgttcgaaccggggatatacaaggtcgcaccgttctccttggtcat |
| | cctcgtcaggcaccagatcacgttgactgcccagacatccaaccacggcgctggaagaacgatgctctggtccgagt |
| | gcagggccatgctctccgcgccaggacgagcaatgttggccgagaagttgctgaccagcagctggtcgcccagga |
| | gggacttggccaggtctagtgcggtcgggttgaccagcatgtcgcgccagtatgcgtccaactcggggagatagaag |
| | acgcgcacgttcgccgggttgggatccaagatcggctggaaagtgcactcgccacgagcctccgaggcagctttcg |
| | cctcccagagacggctgagtgcatcctcagcttcagctttggagagaacggcagggatcttgacccagccatgctcttt |
| | tagatgagcttgggcgtcttccatgtttagtgtcatgtctcgaacaaggtcccttgatgttgagggtacaagggtgtattca |
| | ggctcttgagccgtaggatcaagagcgctgactgactcgctaatagtgcattcatgcctacccagcat |
| intergenic region | tgcgggagggtaggagggtaggagggtagctaggtagttgatagtgctaagtgctctgccgggtcaactgtgaatga |
| between C6H and | atgaggtgtagttgagacacttgaggttgactttccaggcgagcgagcgggtcaagagagcagagagaatatgatag |
| MT (1034P, 849 | actgggtgtctgtagtagatagacaagatgtatgtctgtcccttggggaagtagggctaatacttctaccttagcacatgtt |
| bp) (SEQ ID NO: | gcgggaagccacgcactgaggaaacactgacatcgttggggcactctgattggagccggagattaaggtaagatgg |
| 7) | aatccttctggctgcagcgctgtaagccctaagcctggtggcgcttctggcggacttttcggactacaggactccatcc |
| | aagactccagatcgagactcagcttcgctagtccggaagtccgctggctgatgcttgtctcagcttttcgtctcagctttg |
| | tcgtcttctgtagagcctttagggaaaccccaactcagcatatggatgcagggctggttgggctgattgggcgttgtctg |
| | gacttgtatctgggtatggctgccgtctggggatcaaaggtaaatggggcagaaattgcctgttgaaatagttattgcgg |
| | aggccaatgcaatatcccaagaatttcccaaaatgcaagctactatagatgctacatagccagatagaggttgataatg |
| | ccacattttcaatatatacacatacgtttgtgtgtataagtacataacacgactacagtggctgatatatatgcagtggacg |
| | cctttagacatgtttccatttatgattatagagcgatcctcaggcaagtggttata |
| MT | ctatggcagctctgcctcaatcacgctctcgtagccacgaccatcagggtaatacttgaccagcttgagcccggcatcc |
| (complementary, | ttgatcaccttgctccacacggcttcggttctttcattagctgaagcctgcaacacaagacagtccatggccgcttggtag |
| 1379 bp) (SEQ ID | caactggcacctgtcgatgggatcacaatgtcgttgatcagcaccttggagtagccgggcttcatcacagcggcaatct |
| NO: 35) | gccgaagaatcttgacggatgtctcatccgaccagtcatgcaggacggcatgcataaaatacgctcgcgctcctacat |
| | atcgctgttagtcccctaggcacctgtagtggcagcagaaccggtagcctacctttgatgggctgctcaactccctcctc |
| | aaagaggtcatgcgccacagttcggatcttgtccgtggtaaggtggacagcaccgacaacgtcgggcaggtcctcga |
| | gcacaagggacccagcggggagatcggggtgcttctcggccacgcgcatcaagtcgatgccgtggtgtccgccaa |
| | cgtccacaacgaaagggcttccattactgaggtcggcgccatcgagcagtgcttgggtgtcgtagaactccggccac |
| | ggtctctttcctttggcccacacgtccatgaaagatgagaagctctcctggtgcacggggttcgcgctgcaacgctcga |
| | aaaagctcttcttttctgggaaagtatcaatgtaacaactcgccttgtcgtcccgcggcttccgatagttggtcttggccag |
| | gaaatcgggccagtgcatggcacatggtgcgacatgatccgttctcatcggtgctcgttagtatggctcgcgttgatatc |
| | gaccttgtgcctaaagggggcagctcaccgaatgcgaagcgctggggcaacctttgtgctcttgtcgccgatagcga |
| | gggcatacggcgtaggtgcatagcggtcgttggccgtttccaggataatgtggttggcagccatcagtcgcagttgat |
| | gacctgggatcatcagcatccacgtgggatggcactctcagcagagagaaacctacgtaatagctcgggttccacgtc |
| | tctcttgctgagcttggccaactcggtcacatctctctcgccgcccccggccgcagcccagccttcgaacagaccggt |
| | gtcgatgagagcttggagcacggagaacatgactggttcctcgatagcgagccgcatggtcttttcttctttcgtttccag |
| | cgtgtggaagagtttgcgggccgccagagccagcttctgtcgcgtggcatcctggccctcaaagatgctcgtctccaa |
| | cgtctggagcttttcgattaattgttcggcaatgtcagccat |
| intergenic region | cctgtttagagtggccagaaggtgtgtgtgttatctgcaggatgccggtaccagtagggctgtatgtaaatacggctgc |
| between MT and | agtagtttcaagttctgcttcgatcaagcgttagacctaggattgagcgcggctctggcaatggcggcttttctcatggta |
| KR (1033P, 605 | tagcatggcatagcctgaggatataggtactccataccgaggtacgagtacatctatactaagaatagtgactcccagc |
| bp) (SEQ ID NO: | ttgcctatcccctgcttatcccggagtttgcatctccgccaggaagcacgcggactgaggcggagtaattaacagaag |
| 9) | gcatggcaatgcttactgcgtggggcttaaaacctgacctgacctggcctggcctggcctgatctgatgtgaaactggt |
| | tctccttctctatctccctctgtcagattgatcgtcaaaacctaaccctaagtcaaatttaaacgccacgcaccggatactc |
| | tcaactctgaatacggccttgatcagccaatcacagaagattgcgagctgacagttcgtattgattactttaaagcctggc |
| | atagacgatctgccattgatttgcaattctccggcccagttgcata |
| KR (3155 bp) | atgctaggattgcctaacgagctgtcggggagccaagtcccaggtgctacagaatatgagccaggatggcgacgcgt |
| (SEQ ID NO: 36) | cttcaaggtagaagacttgcctgggctaggggattaccacatagacaatcaaaccgctgtccctacgtctatagtctgc |
| | gtgattgcccttgcagccgccatggatatcagcaatggcaaacaagcaaacagcatcgagctctatgacgttaccatc |
| | ggacgaccgatccacttaggaacatctccagtggagattgagaccatgatcgccatagagcctggtaaggatggagc |
| | tgactccatccaggccgagttcagtctgaacaagagcgccgggcatgacgaaaacccggtcagtgtagccaacgga |
| | cggttacgcatgactttcgcaggccacgagctagaattattgtcctccagacaagcgaagccgtgcgggttgaggcct |
| | gtgagcatcagcccattctatgattccctcagggaagtcgggctgggatacagtggacctttccgagctttaacttctgct |
| | gagcggcgaatggactatgcatgcggcgtcatcgcgccgacgactggtgaagcatcaaggacaccagccctacttc |
| | accccgccatgctcgaggcctgcttccagacgcttcttcttgccttcgccgcccctcgagatggttcgttatggacgattt |
| | tcgtgcctacccagatcggtcgactcacgatatttccgaattcatccgttggcatcaatacgccagcctcggtaactatc |
| | gatacgcacctacatgaatttactgcagggcataaagcagatttacccatgatcaaaggagacgtcagcgtctacagct |
| | cagaggctgggcagttgcggatacgcctcgaaggcctcacgatgagccccatagcgccctctaccgagaagcagg |
| | acaaacggctgtacttgaaaaggacatggctgccagatattctctcgggcccagtactcgagcgagggaagccagttt |
| | tctgttacgaactcttcggcctgtcgctcgctcctaagtcgatactggccgccacccgactgctctcgcatcgctacgca |
| | aagttaaaaattctccaggttggaacttcttccgtacatctggtacattctttatgtcgcgagctaggaagttccatggactc |
| | ttacacgattgcctgtgaatcggacagttccatggaagatatgaggcggaggttgctatcggacgccctgcctatcaag |
| | tatgtagtcctcgacatcggaaagagtcttacagaaggggacgaacctgccgccggtgagccaaccgacctcggctc |
| | tttcgacttgataattcttctaaaagcctctgccgatgattctcccattttgaaacgtacccgaggtctcataaagccaggg |
| | gggtttctactgatgactgtggcggcaacagaggccattccgtgggaagcaagagacatgacccgaaaggcaatac |
| | atgatacgctgcagagcgttgggttttcgggagtcgatttattgcagagggacccagaaggcgattcgtctttcgtgatc |
| | ctgtcacaggccgtcgatcatcaaatcagatttcttagggctccgtttgactcgactccaccatttccgactcgagggac |
| | gcttcttgttataggcggcgcctcgcacagggccaaacggcccattgagacgatccagaatagtttgaggcgtgtctg |
| | ggctggggagatcgtcttaattaggtccctgaccgacttgcagacccggggccttgaccacgtggaagctgtgctgag |
| | cctgaccgagcttgatcagtcggtcctggaaaatctcagtcgcgatacctttgacggcctacatcgactgctccaccag |
| | tccaagatagtcctgtgggtcacatacagcgcaggaaatctgaacccccaccaaagcggtgcaattgggctggttcga |
| | gccgtccaggctgaaacccccgacaaggttctgcagctccttgatgtggatcagattgatggcaacgacggtcttgtg |
| | gcggagagcttccttcggcttatcgggggcgtcaagatgaaggatggcagctcgaatagcttgtggacggtcgaacc |
| | agagctctccgtccaaggagggagacttcttatcccgagggtgcttttcgacaagaagcgcaacgatcgtctcaactgt |
| | ttacgccggcagctgaaagcaaccgattcctttgagaagcagtcggctctggctcgtcccattgatccttgcagcctgtt |
| | ctcgccgaacaagacgtatgttctcgccggtctgagcgggcagatgggccagtccatcaccagatggatagtacaga |
| | gtggtgggcgccacattgtgatcacaagccggtgcgaacagacacgtctgtgatgtggataagtactgacagtaatag |
| | caatcccgacaaggacgatctctggacaaaagagctagaacagcgcggtgctcacattgagatcatggccgctgatg |
| | tgaccaagaagcaagaaatgatcaacgtccgcaaccagatcctaagtgctatgccccccatcggaggcgtggcaaa |
| | cggtgcaatgcttcagtcgaattgtttcttctctgatctgacgtacgaggccctacaggatgtcctgaagcccaaggtgg |
| | atgggtcgctggttctcgatgaggtcttctctagtgatgacctcgacttttttctgttgttctcgtccatctcggcggtggttg |
| | ggcagccattccaagcaaactacgatgcggcgaataacgttaagtttggccaatctgccgcagtgcggacctactgac |
| | tgaccactttgtagtttatgaccggcttggtgttgcagagacgcgctcgtaacctgcctgcgtcggtcatcaaccttggc |
| | ccgatcatagggctcgggttcattcagaacatagatagtggtggaggttccgaggctgtgattgctacattgcgaagtct |
| | ggattacatgcttgtctccgagcgtgagcttcatcacatattggccgaagcaatcctcatcggcaagagcgatgagact |
| | ccggaaataatcactgggttagagacggtctcggacaatccagcacctttctggcacaagagcttgctcttttcacatat |
| | catatag |
| intergenic region | attcagcctattgagattacagccacggaagtaatcctgtaaggatcaggatgcaactccatgcaaggcgctaaggatc |
| between KR and | aggatccttttcttcaggattgtggcaacggcgccagcggccagcgggcgctatcgcgtcggtggtgatggcgttattt |
| CPA (1031P, 384 | ggatttcggaggatagaatccggtcagcctaatcaagccaactccgtcggacttcggcgggactgtccggtcagttag |
| bp) (SEQ ID NO: | agctagagaaggaaggaggtagagtcccagatagacaaaagacttggctgctatatatcttattattcaatcctcaatcc |
| 11) | cgctagctgtcaatagaatgatcctcagccgcacttgaagtcttgtctacatcccgaatccaggcgca |
| CPR (2145 bp) | atggcgcaacttgacacgctcgatattgttgtcctggtagtgctcttggtgggtagcgttgcctacttcaccaagggctcc |
| (SEQ ID NO: 37) | tactgggccgttcctaaagacccctatgccgcagcgaattccgcaatgaatggcgccgccaaaacaggtaaaactcg |
| | ggacatcatccaaaagatggaagaaaccgggaagaattgtgttattttctacggttctcagactggaactgccgaagatt |
| | acgcgtcccggctagcaaaggaaggttcccagcgtttcggcttgaagaccatggtcgctgatctcgaagattacgact |
| | atgaaaatcttgataagttccccgaggataagatcgctttctttgttttggctacctacggtgagggcgagccaaccgata |
| | acgccgtcgagttttaccagtttatcaccggtgaggacgtcgctttcgagagtggtgcctccgctgaggaaaagccact |
| | ctcctccctcaagtatgttgctttcggccttggtaacaatacctacgagcactacaatgctatggttcgccacgtcgatgct |
| | gctcttacaaagcttggtgcgcaacgcatcggaaccgctggtgagggtgatgacggcgctggtacaatggaggagg |
| | acttcttggcatggaaggagcccatgtgggccgcgctgtcggaatctatgaacctgcaagagcgcgaggctgtctatg |
| | aacctgttttctctgtgattgaagatgaatctttgagccccgaggacgatagcgtctaccttggcgagccgactcagggt |
| | catctcagcggcagccccaagggtccctactcggcacacaatccttacatcgctcccatcgttgagtcccgtgaattgtt |
| | tacggccaaggatcgtaattgccttcacatggagatcggcattgctggcagtaacctcacttatcagactggtgaccac |
| | atcgctatctggcctaccaacgcgggtgttgaagtcgatcgtttcctcgaggtctttggcattgaaaagaagcgccatac |
| | agttattaacatcaaaggtcttgatgtcactgccaaggttcccattccgaccccaaccacatacgacgcggccgttcgct |
| | tctacatggaaatttgcgcacctgtttcgcgtcagttcgtgtcctctttggtgccattcgcccccgacgaagaaagcaaa |
| | gccgagatcgtgcgccttggtaatgataaggattactttcacgagaagatcagcaaccaatgcttcaacatcgctcagg |
| | ctcttcagaatatcacctcgaagccgttctctgctgtcccgttttcgctccttatcgaaggcctcaacaggcttcagcctcg |
| | ttactactccatctcgtcttcctcccttgttcaaaaggataagattagtatcacagctgtcgttgaatctgtccgcttgcctgg |
| | tgcgtcccatatcgtcaagggcgtgaccacgaattacctactcgccctcaagcagaagcagaacggtgatccctcacc |
| | cgatccccatggtttgacatatgctattactggtcctcgcaacaagtacgatggaattcacgtcccagtccatgttcgcca |
| | ctccaatttcaagttgccttctgatccttccaaacccatcatcatggtcggacccggtactggcgttgcgcctttccgtgg |
| | cttcatccaggaacgagctgctctggctgaaagtggcaaggacgttggacctacgattctgttctttggttgccgtaata |
| | gaaatgaggacttcttgtacaaggaggagtggaaggtatgtttgcagtcttcttatgagcacattcggagccgtttgtctg |
| | actcttaataggtctatcaagagaaacttggagacaagctcaagatcatcactgccttctctcgtgagaccgccaagaa |
| | agtatacgtccagcaccgactgcaagagcatgccgaccttgttagcgatctcctcaagcagaaggctaccttttacgtct |
| | gtggagatgcagccaacatggctcgggaagtcaatcttgtccttggtcaaatcattgccaagtctcgtggcttgcccgct |
| | gagaagggtgaggaaatggtgaagcacatgcggagcagcggcagctaccaggaggatgtctggtcgtga |
| intergenic region | ggcatcgtctacaagcagatgctaggcacacatttctttctgccgctaaaaattgggtaatgcagagccacctcgcttttt |
| between the CPR | ttttttcgaacattttccatcttgtggtatttctgggttcatttcgctccatataacgaagattggccttggtacgggctaggg |
| cassette and fpaII | ttcgcgggtgggatagttatagaatgagaaataatacttttatatgtaacaatttcaacttctcaagatgaatataccattcgg |
| (1031T, 591 bp) | atagagcagcttctgagtatcgacagacttaggtaggcttatgggtatgctctgttgaatatcttgtagatgtgacaggca |
| (SEQ ID NO: 13) | atagattgttagattatagcctacaatccacagctcagctcagcacgagtttgattttttcattataattggaataagcactg |
| | agctcagaatgaaaccaatagattactagggctatgcgtagacgttgaacgggatccatcaccaagcgcagtattagg |
| | gcaccttttgtcgtgggtatatagcaactaaacacattctcttcggtcctgttcggccctcttcggcctccattagccagtc |
| | aaaataaacagtaaccag |
| fpaII | ctagtagtcgtcgccacgactaatcacctctttgacggtgggtcgaagcagaatggtcttttcccaccattagtgcatatt |
| (complementary, | cttcttgaggaatcaaccggacttacgtgttcgaactgggccgtatacgtccctggcttctcgttcagagggggatagtc |
| 1937 bp) (SEQ ID | ttccacaatgccggatttcaccaggtaattcagctatcgccggttagccgagagctctgattattgcccttggtaatggac |
| NO: 38) | ctcatacaccgaggagatacttctcttggccaatccggtccaaatagcgccggcagaaggggatcgtgctaaagttctt |
| | cttgatggctgtcaagagggacctggccgaagacaacgtcaagtcttttcggtccgcgtccccgcgcagggcgtaat |
| | gggagacctcgcctccttcgacgtatcggccactgccggtactcccaaaggtttcgatggcaaagacgtccccttcctc |
| | catcttggtcatgtcgttcgacttgacaaaggggacattcttggtgccatggatggagtacggcaggatcgtgtggcca |
| | cacaggttgcgaatcgccttgatcgggtacgtcttgccgcggatttcgcactcgtagctttccatcgcctcctggatgta |
| | gccgcctagttcgcccacacggacatcgatgcccgcttcgcgcacccccgtgttggtggcatccttgaccgccgcga |
| | gcaggttatcgtacatgggatcaaacgccatggtgaaggcactgtcgacaatgcgaccgccgacatgaatgccaatat |
| | cgaccttgaggacattgttctgcgccaggacggtcttgcagccggcattggggctgtagtgggcgacaatgttatcgat |
| | gttcaaccccgtgggaaaccccatgccggcgatcaaggagtccccctccgttaagccgtcatggcccaccaggcatc |
| | gcgcgctctcctcgatgccattcgcaatctccagcagcgtttgaccgggcttgatgtttctctgcgcccactgacggacc |
| | tggcgatgcgcttccgctgcctgacggtagtccgagaggaagtcgctattcaggttgtcgaggtgacgtttctcctcgct |
| | cgtcgtgcgatagcgattctcgtccttgtactcgacctcttcacccttgggataggagttgttcgggaatagctgcgaga |
| | ggggaatcgatggcggatcggtctggaccttgggttgcttcttcttgggcttcctctttttgttcttctttttcttcgcagtg |
| | ctgtgctcggcagctactgcgggggggttttcagtgccatcgtcgtccgagccgtcgtcaacttcctttccagtcccgtttg |
| | ctgcggccgaagtcgaacttgacatgtcggctccattggcaccagcatctgtgagttgcatccagtatgagctgggatc |
| | atcgtataggttgggaacctgatgctggactcttaccggtgattctcagcttctcaagaagctctggcgcgtcgacagtc |
| | atatctagcaagggaggacaccaggagaaaagggacggtcgcaagtctgtgggaaccaaatgatatgtaacttagcc |
| | aagcacaccaataccaacgaaacgcgagagggcttcggagtgtgcagtcctggacctcggatgtgcggcgtactcc |
| | gtagcgtggacaacgcagtgagtgagatccagcgcgaggcggggctggaggggcaataacacagaagcagcgc |
| | agtgccaggagacgacgactgcagttgcacggtgggcaccaagggtacgtgctaggcgctggccctggtccaccgt |
| | ttgacagggaaagatttggaaacttgggtatccagcatgtagatgcaagtcgggtatacgctatccctctgctttcgaca |
| | acgagcaaaatccaatcgagtccacgtctttggctttgaagcat |
| intergenic region | aacgaggtccaggtgacggtaacgtggttcagtgcagttccaatgtatggtagcgttgtaagctgacacggcgacggc |
| between fpaII | tgcgagaggggttggggggacggaaccagctgaaacaggactggcgaaagaaagctgctgtgttatatgtaggcag |
| and PalcA- | agctaaagaaccttgtggagcgacagaaccaaagtcagtctgggccatgggctatcttccataattttgggagctcgag |
| AN1029 (1029P, | gtccggattgcccgttaatactccgccagactagggcaagatagggctacgcggagttttaggtggacggatttcaac |
| 1370 bp) (SEQ ID | cctccgaagtccgctcgaacttttgtcgacgagattaagccactagcctaaaggaatcagacctttaattcctcaggccg |
| NO: 15) | agtcgggatcattgaaggcgagaatgaggtgaggttgtcagccacatcgtcagctcaatcctttagaccacgttcttatc |
| | tcgcggccgttctccaatcgacgggcccgctggcccccagcgtgcagattacaccgtctcgctccgactgcaggatct |
| | ggcgtcttccatgcgcggacgtttcggacggcgatgactgtctgagtggttggcagggatgcacccctacctacccct |
| | gatcgaagctaatggtaatgcagaatacgaggttggttagactaagcgcttctgcagctgcagcgcatggaagctgttc |
| | tgtctggtggagagactaagcagtgctctgtgctcctctgtgctgctctgcattgcactgcactgtactgcattgtactgca |
| | ttgctgttctgcacggatcattcatccatctaccatggatccactactaacctcgcttactctagtcgatctggtcaagacg |
| | accaagacctcggagaattagatggccaaccaaggatagatgcgagatcaactgatccaccgctggcaaacttagtt |
| | gtgaatgtcgcgaacgcaaataccacggagatggcatgcagccgcacccgaaatggaatgctgtaggcctaatcaa |
| | gctcatcgattctcgcccccaaatctgggctgcgcggtcctgcaggtgagacggatcctggaggctccatgctggctg |
| | gctctgcctcctcgtggacgagggtacgatggcagccagtctgctggcgtgctggcgccgctggtagcacggccac |
| | gagcctattgattgcacgggcaaacgttcgtaactcgctcgtaacctataattacgatagctaaccacatcctggttctct |
| | ctcataagaatgaatggcattcccgccttgatccgtcagcattgtcaacccggatagaccagtgcctcgtcattcaacat |
| | cacagatccagagactacaaagaccagcaatc |
| PalcA (404 bp) | ctgaaaagctgattgtgatagttcccacttgtccgtccgcatcggcatccgcagctcgggatagttccgacctaggattg |
| (SEQ ID NO: 16) | gatgcatgcggaaccgcacgagggcggggcggaaattgacacaccactcctctccacgcaccgttcaagaggtac |
| | gcgtatagagccgtatagagcagagacggagcactttctggtactgtccgcacgggatgtccgcacggagagccac |
| | aaacgagcggggccccgtacgtgctctcctaccccaggatcgcatccccgcatagctgaacatctatataaagaccc |
| | ccaaggttctcagtctcaccaacatcatcaaccaacaatcaacagttctctactcagttaattagaactcttccaatcctatc |
| | acctcgcctcaaa |
| AN1029 (2354 | atggcgtgtcccaccagacgaggacgacagcagcccggctttgcatgcgaggagtgtcgccgccgcaaagcgcgc |
| bp) (SEQ ID NO: | tgtgatcgcgtgcgtccgaaatgcgggttctgcactgagaatgagctgcagtgtgtgttcgttgacaagaggcagcag |
| 17) | aggggtccgatcaaagggcagatcacctcgatgcagtcgcagctgggtaggtgtttgtcttgtctcattgtatctcgtctc |
| | gtctgcgcttttgtgattatggggctgccatgtttccggtccggacacaggcatctgcaaggcccgccgctgtgctccc |
| | ccgatctgcagggaccaatgcagctggttctggagcttgtgctgtgctgcttccctgtctttccacatggtcgagtcgag |
| | cgagctagctaacatgggatgcctcatgctttcagcaacgcttcgatggcagcttgatcgatacctgcgacatcgacct |
| | cccccgtccataaccatggccggcgagctcgatgagccaccagcggatatccagacgatgctggatgactttgatgta |
| | caggtcgccgcgctgaagcaggatgccacggcaaccaccacaatgtcgacgtcgacagctctcatgcctgccccag |
| | ccatctcatctaaagatgctgctcctgctggtgctggtttatcgtggcctgacccaacctggctggatcgccagtggcag |
| | gatgtcagcagtaccagcctcgtccctccatcagacctgacagtctcgtcggccactaccctaaccgaccctctcagct |
| | tcgaccttttgaacgagactcctcctcctccttctacgacgacaacaacgtcgacgacgaggcgagactcatgtactaa |
| | ggtcatgttaactgacctcatccgggctgaattgtacactacctaactgatttgtctaccatgacacctgactgacaatgtg |
| | cagagaccaactctacttcgaccgggtccacgccttctgccccatcatccaccggcgacggtactttgcgcgggtcgc |
| | ccgagatagccataccccagcacaggcatgtctgcagttcgccatgcgaacgctcgcagcggcaatgtctgctcact |
| | gccatcttagcgagcatctctatgccgagaccaaggccctcttggagacgcacagccagacgcccgccacaccgcg |
| | agacaaggtcccgctcgagcacatccaggcctggctgttgttaagccactacgagctgctgcggatcggcgtgcacc |
| | aggctatgctcacggctggccgggcctttcgtctcgtgcagatggcacgactgtcagagctggatgccgggtcagatc |
| | gacagctctcgccgccgtcttcgtcgccgccgtcttcgctaaccctatctccttcgggggagaatgctgagaacttcgtc |
| | gacgccgaagaaggccggcggacgttctggcttgcttattgctttgatcgtttgctttgcttgcagaatgagtggccgtta |
| | acgttacaagaagagatggtacgtcgcgcttcttttattctatttacctcagaatttatattcagttattttttattctaaccc |
| | tgctagatattaacccgcctcccctccctcgaacacaactaccagaacaatctccccgcacgcacgccctttctcactgaag |
| | ccatggcccagaccgggcagagcacaatgtccccgtttgccgaatgcattatcatggccacccttcacggccgatgta |
| | tgacgcaccgccgcttctacgcaaacagcaactcgactgcgtccggctccgagttcgagtctggcgccgcgacgcg |
| | agacttctgtatccgccagaattggctgtcgaatgcagtggaccggcgagtccagatgctacagcaggtctcctcgcc |
| | cgctgttgacagcgacccgatgctgctcttcacgcagacgctcggctaccgcgcgaccatgcacctgagcgataccg |
| | tccagcaagtctcctggcgggctctcgccagctcgcccgttgaccagcagctactgagcccgggcgcgacgatgtc |
| | gctgtcggccgccgcgtaccaccagatggccagccacgcagccggcgagatcgtccgcctggcgaaggccgtcc |
| | cctcgctgagtccgttcaaggcgcacccgttcctacccgatacgttggcgtgcgccgccacgttcctctcgacgggca |
| | gtcccgatcccacgggcggcgagggggtgcagcatctgctacgagtgttaagcgagctgcgcgatacacacagcct |
| | ggcgcgggattatttgcaggggttgtcggtgcagacgcaggacgaagatcatagacaggatacgaggtggtattgta |
| | catag |

TABLE 12
Genomic DNA sequence of the mdp locus in strain YM727.

| Region | DNA sequence |
|---|---|
| AN10039 | tcaaacatgctcgcgaggcctgacgggcgcagtatcgtgaaggtcccattcctcttccagctcatccgcaagagacg |
| (complementary, | atggcccaacagtctgctcgacaagccgtggagggcatttgttcttcatctcccagctgccatgccgctcatgtccttt |
| 1713 bp) (SEQ | ggctgaagcggtgggagcctttattgcatccccccattgcggattcttaaaggtcaggtccgaatcacttgccatcttg |
| ID NO: 39) | ctatccctcttgaagcctccaagaccgggattccgcaaccgcttggtgcgcagacataaaaagaatccgacaacac |
| | cgatcagtgctaaaaccgcgatagtaacgacagatccgataactccagcaacagcagcactgattccaccgcggtc |
| | ctttcgcgccttgctggcggccgactgtcgtggcagtggttttacaacccccgagcagaacacagcggacgagttac |
| | aacgacggcaccaatcttcggttgaccctaatgccattctttgcatctcagcttggaactcactgaatgggatggccg |
| | tattgctcgggccatgtccaaacagcggataaggcacgaattcagcccgtgttccattgtgcaggaggaatcgaacg |
| | tagagctgcgccgggtcggggtacgtagggtatctctcgctctccaggctgaacagttccaggacaagggacgatcc |
| | tggacgcggcagggacgaagtaaagtttggacttgtggtggccagctgcaaaagtgaggtgaaggacacagcggt |
| | ttggtagttgccgaactgaagtgtcatcttcccattagcgcctcggttctggatggttgtctcgaaagcgtttagaaca |
| | cttgatgcgaggccttggcctgcgattgtccggataccgccgtcaacggttgtgccggatgccgacgtgtttccattcg |
| | tcgcaaagacatatcggccagcataccaacgtgcgcgcttgatgtcctctgcacttagactatgtaaaagggattca |
| | ttatgcaccacctggtagtccagaaattcggcaatcgcggttgcgttcgcatagctggatgcctttgtatcaaagtga |
| | ccggacaaagatagagtatgaagacggttgtaaaatactgctgactcttcataggtccgccagaactctttgctggc |
| | aatgtactccagatttgcggcagcgtgccttgaacaaagcgcctggccggacaccatgagggactgagggtcttccg |
| | cgccgacggtaacaatcgaagggtactgatatccacccagcggaggagagataagagacccatcggccaattcca |
| | gttggttgtcgaagaaagtggtgttgaacgactcattcaggggcgggtaaacaccctgcatgaatgcctgggcggat |
| | gcgagcacggccacatcaggcgtagaggtgattttgatgtcgtcgttgtccaccagataaggagagagattctcgat |
| | ccttgcatcgggtctatcggcgccggcttttacagagacatatcgacctcggaatgccgagccggcttcatgaagttg |
| | gtatgctccgtacggtgtcaatgcccttgaacgcgggaagactcgtggtatggtttctccgttgatagtatatgcatat |
| | actgcccagacccgcgccgtctggccgcttgctgcagctgcgacaactcccagaaggctcaggagtgcgaaagaca |
| | gcccaaccatcat |
| intergenic region | gttgccggtgcggtgggatgcattcttcacgtttcttccgctgggactggtcgacctaataagaataagaaggtcgat |
| between | ttactttcgcaaggatatcgcgacatgacgacatgatacggtcgtaaccatgttccaagattcaacttactttgcccta |
| AN10039 and | ttccggctggcggggtgaattttccgccgcaatcaacacgaattaggtcagagtgtagatagagccacatagattcc |
| AN10021 | gagcgtattactgttcggaaatcacgggcctgtatagaaaattctgctaatggacttcactttcgatttctaggattgt |
| (10039P, 653 bp) | atgacgtgaagacagagcaaggttacattctaactctcagtagtggagttctacctagcccggcccggcgcgcccta |
| (SEQ ID NO: 40) | gataaccctaaatcaaagataattggcctgccttcgacgtttctcaacgagctatgtccgaaattttatctttaccaag |
| | gtcgaagtttcgtaggaactcaggcccattttgtgcgacatgagctgcttgttcggaactgtatccgctcgttccaaac |
| | cgttccatccgggcagttgcggaatcagtcttaggacctgatagatgcatgaaatagatggaccatcctgaacatct |
| | cacaaactcaaaaaaaaatttccaaccg |
| AN10021 | tcacctgttataggcctggtaccgaatctccaacaaaactaccgtgctttctctggactgaatcttattcaccaccacc |
| (complementary, | aaccggcccatactatcactgacattgctcagcagattaatccactccgccagctcaatctcacgctcatttgccaac |
| 1534 bp) (SEQ | tgcatcagcgtcaagtcgcgcagtcgcgcgcttgcttcgacctcgctatgcacagctgagggttcaggcaagggccg |
| ID NO: 41) | cggggtgagaatcagggtcgccgatgggtttgagcggaggatgtcgagatgtgagcggagttctgcgaggatgtgc |
| | gttgcaagggaggcgaaaggaactgttggtgagggagaggggaggtgtaggatgtagagatttgctgaggtaatg |
| | ggttgcggtgctgttggaagccggtgttggatcgttatgttggaggcctggggtatgctattggtggtatgcgtatggg |
| | tgtggttgtggctagaggccggtgttgtactggccgtgcttgcagtgagtgcgcgaaggtcgtcgtgtttgtggctacc |
| | gccgggagttggggggcggatgggattggggtgtgctggtgaccaggcagttgggcctgctggggatgcgatttgg |
| | actgtgatgttgatggatgggtagaggtttgcaagggttgttgcgcggtcgagggagcgggcgctgacctataattt |
| | gggcaattggttagacaatcgtatggatgatctcaaaatgaagatggattggatgaagtacctcaacgacagatat |
| | acttcctcttcgaaaatggtccagcctcgacaacagatccgtcactcgatcgtcggtatcactggtcccatactgaag |
| | aaaagcaggccactggcgttgaagctttggccgttgctcagacgtactggcgaatgtcgctgggttatttaaagcta |
| | ggttgtacgcggtctcgttcggacgcaaactcgcgccaaatcgctgcgttgcagtaggcatctgcaaagcagaagg |
| | ggcaatggtgccagccaaaaacatcacagcgtcaagataagacggtttggtgacgaaaggagcggagagcgcgc |
| | tgtgagcgacttgaccggggtctggctcatccaggaagccagcggtggctgtcatccggataatacgtgagagatg |
| | agtctctgggacaccggccagctcagcgacatcttttatgggaacggtgccggtgaggggaatgcaagcgaggact |
| | tggaactctccgagccattgtaggcaggcaagcagctggttctgaacggcgaggtggtggaggaagtcggttgggc |
| | tggtgaggagcttctggaggccagaaatggttgataagatcgattgttgggctcgatgcgcttccttggaagcgcta |
| | gaggtgatgaggggttgagttctgctgcgagaggcggcattttggcgagggcattgcgagatgatcgtcttgacagc |
| | gcttgtgagctcactggcgtgggtttcaaggtcggatagactagacatcatgtcctggaagtcccttgacaccat |
| intergenic region | atattggtgggcagtatatattagtagaatcacatcaggaaaggttctgagctatataagcacaaccgatagagcct |
| between | gaacctcactcgggatatttcaggcaacacagcagaagaatgcatatgcagccgaacatgaccgcgaacagtgaa |
| AN10021 and | gcaacacgaataacggccttacacaaaccccgatggggagcaagaggcgattccgacgcagaaactacctttcctc |
| AN10049 | agtaccaagatatatggaactaattacccgataggttgtaggcgatattatatagtttatggatataccagccgtcta |
| (10021P, 314 bp) | acacatga |
| (SEQ ID NO: 42) | |
| AN10049 | tcagacggacctgacctcaaccgctttgttcaccacatgaccattcctctcctctacctcactcgaaccattcgagttc |
| (complementary, | atcacttggtcgtctgcagcgactccgttctcttccttctcagggggtccaaagatcccctctccaccaaactcagtcc |
| 692 bp) (SEQ ID | atcgtatattcggttcaattccggcaaacttccactcgccattgatcttgcggtacgtcaccgtcgctgagccatgacc |
| NO: 43) | gtgaccttttgcaacgacttctttcatctgagaatcaaggtgtttctgatgggcgactctcatctgatgatacccaact |
| | attttcgagtcgtcgaccttctcccatttcattgttcccacaaagtgctgcgttttgaggagtgggttacccaagaagtg |
| | gggatgagagaccatagccacgaattcttcggccggcatcttctcccagagcttgtccaagaaggctctgtaatcga |
| | tctggtaccgtcagcactgattatataggacgagactgggaagctgacgcgaaggaaaggggcgatgcattgtttta |
| | agcgatcccagtctttgctgtcgtagctctctgcccattcgaacagggcagcttgacagcccgtaatgtctggatggct |
| | atcagtatgcacattcaaacactgttcaggggtcctaccttcaaatgttggctgcagcgtcat |
| intergenic region | tgtgccgtccctgtttctctacaagatgggacaaacggagaaaaggtagactcaaaagcaatattttaagtcgatcc |
| between | caactcacaagacagtgtctaggacgggaagaccatgcaagggtacttcaggtcggtgacttgctaagtaccgtat |
| AN 10049 and | gaaggcgggttttacttggtccccgaccttcggtgtccggtacctatatttgagtggaacccatttcaatgcagcctag |
| ANO146 (10049P, | atcatcaacgcaatgtgccattttattgttctggctacgacttagctactaaatctagcagaa |
| 295 bp) (SEQ ID | |
| NO: 44) | |
| AN0146 | ctaagcagcgcctccgtcgacggtaatgatctttccgttcacccactccgcttctctactcgccagaaaaccgacaac |
| (complementary, | ctttgcaatatccactggaaacccattccgcttcaacggcgataccgttgccgccattttttgcagctcttccgcgctgt |
| 925 bp) (SEQ ID | gtttttcgccgtttggaatataatgctgcgccacgtcgtaaaacatgtccgtcaccgttcccccgggggcgacagcatt |
| NO: 45) | gacggtaatctgcttgtcgccgcagtctttagccatcacacgcacaaaggactcaattgcgcccttggagccagagt |
| | acacggagtgccgggggacgctgaactctttagcagtgttggaggacatgaggattatgcggccgtgggtgttgag |
| | gtggcggtaagcttcacgcgcaacgaagaactgggcgcgggtgttcagactgaagacccggtcgaattcctcctat |
| | gaaaaatcgtcaatacctcaaccaggagtcgaatgaaaagcgggttcatacctctgtgacctcgcccagatgccca |
| | aaactaacaacccccgcattactacaaacaatatccaggcccccaaaatgcgccactgcatcatccatgaccctcac |
| | aatctcgctcacgttgcggatgttggcttgcagcgcgatcgcatcggtacccagctctttaatctcctggactaatttct |
| | cagcgggttcacgggagttagcgtaattcaccacgacctttgcaccgagtcgtcctagttcaagggccattgctgcgc |
| | cgattccccggccggagccagtcacaagggcaactttgccttcgaggcggtatggggcgtgcgtggttgcggtcattt |
| | ttgggagcgcgctgacgttggaattgaggtgggaggacacgagggagagacgttggattgctggagacat |
| intergenic region | tggtgctttcctacctaccttatgtatcttgcgctcaggtttcttagaaacggatgattagagccctaagttcgtaagca |
| between AN0146 | catggtgtgcaagggtacggtgcccgagtctcgatcgggatatgtaacttgggcgcaggggataagagagaggttt |
| and AN0147 | cggtgacttagatgcattatgcgagtacggacagcgatgttttacctgcatataatactattacttctgccttgaggat |
| (0146P, 558 bp) | gggcatgagcgtgttgcaacacgagctgtgaatatgtgatcaatttggcccgaccaagagaatataagagttaccat |
| (SEQ ID NO: 46) | tattgctgagtagcactcgttaagtatccatggttgagaagaatgactttgatatcagtagatcagaatcattgtctct |
| | taatcaaggatgaactgctagctaggtcgccctacttagattttctgggaaatacgaatatcaaaccatttatgaatc |
| | tagccttgagcgccagctttaagctcaatcacattgcgactgatgatatccaaatcaatatatattctaaatctttgga |
| | gaaaaggtaa |
| AN0147 | ctaagaccaatcaccatccaacaaatcctccactctcttcccatctgcaatattcctccaaacctcctccaccgtccaa |
| (complementary, | gccctaaactcatggcccggaggatagttcgtattcacaaactctctcccatcaagtaaatgcgcaaacgcctcgcc |
| 1644 bp) (SEQ | gaacttttcatatgcatacgcctccggatcatgctgaaagatccacttaggaaaccttgtcctgatcttcgccggatcc |
| ID NO: 47) | ttccagatcgcatcccagtccgtgcccgtcttcagctgagaattcacgaacgacattttttgtgcacaagagacccgc |
| | tcataccggagaagattgtagatcttggtcccaagatatgcacgctgcgagcttccggctaattggaggcatgttgca |
| | agcgtgattgcgtcttccaaggcctgcgagcctccgtttcctgaggtaggaatgaagctgtgcgcgctgtcgccgact |
| | tgcactacccgtccggcaggtgaggtccactcgcggcgaaggtcccgccagaggagaggccaatgaacaattgcg |
| | cctttcggcgcgcttcgaatgagcgctagcacagcgggatcccagtctcctgcaccggagagcatagcctgcgccac |
| | agtctcgggatctgtatcaggctcccatgattcagtggctgtgccttcaacgatgtcatcacggggcgtgaatccgaa |
| | ggagataatatcgtcgccgacgaagacaccaagatacatgcccggtccaagccagtattcccagatgggtggacta |
| | tcgctccatcgcttccgtacgagctcattctgcattgctaaatctttcggaaatgcagtgcgatagatactcagcccgc |
| | ttgatcttggaggaacatgctgaccggctatcaatatctctgaaggagatttgaggccgtccgctgcaacgacgatat |
| | cagccactctgacctctgcttctcctgttgttgcgattataacgccgcccttgccatccttttcatcttcaaaatagctc |
| | ttcaccgtctttccatattcaacgcggagcccgcaccttgcgacctggcgcaggagcatgcggtagaatttccggcga |
| | acctgagcgggggcaacaaatggacctttgcgtgtttccaggtgctcggggtcattgaacgaggggacggttgggc |
| | cgtaaatgtgccgtccatcatgagtttcgtagctaacgacggcgtggacttgctccgctttcatatcatggagcatgtc |
| | gggccagtgccggattatagatacggcagaaggctgcatgacaatgatatctcctatctcgagaaagtatagagtta |
| | actactcggtttctcatgctctgggggaatacacgagacgtatcaacaaaatacctgaatacacaggtccctcactc |
| | cgttctagaattcccgcaacatcatggccctttctccagcattctaacgccgtcatcagtccacccattccagcaccga |
| | caatgaggacggagattccggtcgaggggtgccgagatggaagaccagaggtaggagcagtgccattctcgccgt |
| | taacactgctttcggtagtaggcgtctttgcccagcgctctggatcaaattcctgcttgtcactggcgatgttgacggg |
| | gaaatgggtcat |
| intergenic region | tgtgactctcagtgctggtggtgtttggggacctgggccgagtaggtagtgcgttgggtagggtcattgaagcaccga |
| between AN0147 | gccggtggtctagggctacctgtgttgattgagggagcactagatgatagaaactgtcactgaagcttggctattgtg |
| and AN0148 | ctcgatactttctagtacaactagttaatatctagactagaagatcgcagcggatagagccattgaaagtcacagac |
| (0147P, 526 bp) | gctgacataacacatttggattccaactaggagagctgatatgctcggggatataaatttagttcttgaacgggactg |
| (SEQ ID NO: 48) | cccagtccaattgggaacttaatagccttaatccaaattacccctctatacgctggtcataatatggatactattacgg |
| | cactgataagcacgggaaaaagactccgaccactcatatgctaggtcttattgtaacaactaagttgcaaatacaac |
| | gcgcgcacgaaacgcaatggaacagggtatatggattccggtacgataatgtttgacaa |
| AN0148 | tcaacccctccgcaatcggtcgacaatctcacttgacaggctccgaagttgagctccgagatcaaccgccagcctttc |
| (complementary, | cagcagatgaaaagggagagagtaatgattgctgtcattgaccccagtggtactcaacctagcaggcttcccgcctg |
| 1308 bp) (SEQ | agacttggtctttcagtcgctgatagagattgcccactaatcgttgaacgcgatgcagttcgctgagaactagctgtg |
| ID NO: 49) | cggccatgcggccttgatcttcaccatcgatattgtagcccctgacaacagccggtgtcctgtcgatctcttctaatgc |
| | ttggctgtcttcagatataggggagatatgcgctacggcgctataccaggctagtactttgaaggcagcaagagtta |
| | tgattgtgatggtgtagccgtcttccgagcaggagcactctatgatctcagtaatatctcgtaaagtctgctcattttta |
| | gtgatgacttgttgaactgtaggaggacttgcactgccactttcacttgagggagtcacgcatgatagtgaagggttt |
| | ggaaagagttcgcgcagcagtgtcagtgcacgtgggaaacagaaacattgccgtggtgtcccaacactggttacat |
| | ctggtgtaggaggaactgaaggcgaatttgccggaacgggagaatccgcgaaactggtcttcaagatattctcttgg |
| | agcgttggtatcggttctccagatgggaagaatgacggaggatcaggaaaaccatccatgacattcgcgctcatatc |
| | agctccagggaagtaatccatgtcaggcacatcgagaagcgatagagatataggcgaagcaagataaccgtcgta |
| | gtcagggggccccaaggtaagagggcttgtagctgaggttccggggccggttgatgagagaagacttggtatactc |
| | tctggatagcttggtgtgcgctggtgatattgatttcttcggtatacttcgaggcttcggtcctgctgaagagcgtattg |
| | catgagctctgtcgacacctccatgagctccctccgatcatcatctttgttgatagacgtggaatagtctgtcttcatat |
| | tgtagaatgacttgaagctgcctgttttactgccttgtttgcgacctgcgcgcttgctggcgagatactgacatgctgt |
| | acctcttttgacgcatcgtgagcaagtaggtttatcttgactgcacttcaatttggacagggcacatgcgtgacagctt |
| | cctcgcagcttgactggcggagttttgatagcggggatacctggaccctctgaagatgtcat |
| PalcA | tttgaggcgaggtgataggattggaagagttctaattaactgagtagagaactgttgattgttggttgatgatgttgg |
| (complementary, | tgagactgagaaccttgggggtctttatatagatgttcagctatgcggggatgcgatcctggggtaggagagcacgt |
| 404 bp) (SEQ ID | acggggccccgctcgtttgtggctctccgtgcggacatcccgtgcggacagtaccagaaagtgctccgtctctgctct |
| NO: 16) | atacggctctatacgcgtacctcttgaacggtgcgtggagaggagtggtgtgtcaatttccgccccgccctcgtgcgg |
| | ttccgcatgcatccaatcctaggtcggaactatcccgagctgcggatgccgatgcggacggacaagtgggaactatc |
| | acaatcagcttttcag |
| intergenic region | agtgggagtgaggcgatatcaatcgggggattacagcgtgggaaaatgagggggcccaggcttaaagtaagaga |
| between PalcA | gcatctgcaggaaggattcgactccatgctcgcatggccaccgcttggttcattggctttgatagcaccaggccagct |
| and AT (0148P, | gctggatgtcagcttacagttggataccattggagtctctaaactccatccggggcctgagctgatgcccagagtgg |
| 1478 bp) (SEQ | gatccgggaaacagcccctggcaatgctcatgatccttttgtttcgggcgggtcaagtcttgctgtccccgacagtga |
| ID NO: 50) | tggtgatcagccagagtggcctgggagccgcaatccattcatatgcactatagtgctagcaacaaccgattttatcat |
| | gcatttgccggagtcaggtctcggatttaacggaggagaaggactttgctcatcgcagttaatcccattcgaccgata |
| | actccatctcaacgaaactataaatcaagcattaaccaagccaggcgccctactcgtacctacttcggagacgagta |
| | cagatgtacgcttacgggtaacggaatagatgtggagactttcggacccaggttaaccggcccccacgtcgttcccg |
| | gtgaccgacatcaccgccgctgtccggtcattagcagttgtcatcgcaaaaggcgattcgaagatgaccgcttcatc |
| | aacgggaaaccggataggaaactttcaaaaagccaacgggaatgtttggaatccgcaaaagagagggtcggaag |
| | gtatctcgcgtggcttgctcagtgccgttgagctgatcggaaactatccatagtataacccaatcggctagtactgca |
| | ctgcagatccacccgcaactatcggcacgctattcgcaaccggtcttagtccagcttagcgggcatgctaaattcgac |
| | cttattttgtcgtcactcgtcactttggcagagttcggggtggtatagcccgtcaagaatgggtttatggaatttgtctg |
| | ttgcctcgtgtcgcagaaagcagttcccctgtcaacggcgcatatctgaagtagagacggcctagccatcgtcttatc |
| | tacttcggctacaacgcgcaattggacgctcacggtctatctgttgacacgaaccgatcagcttggtcatcaatacag |
| | tgtatatggtgaatagtagagtcgagactgcgagcagttgacggttagatgtgtattaccgtacgtcgatgaatccac |
| | gccaaggacaaagacgcgcgtcaacagaggactgaagtagactgtaatctgcgtttagttgataatcttagagtga |
| | caatctaggcagcagcaaaatcgtttgataaatctagtgaacaggttgtcggcaatcgtagaaatccgtttaatgtgt |
| | tgttggagagcgaaggtggagtatgaaagaaagtgaaagcttcaggcttggcatcccaacctcactccatccaatg |
| | cctcgcttaa |
| AT | ctaaaagtcgaggtgtttcctcataaaggcaagctgcctctgcaggacttcttcggctccctctccgttaattacatcc |
| (complementary, | atatgacctcggccagcgacgagatcgaactccttgtttggctccggaatcatgtcgaaaacagctctttgatccgcc |
| 926 bp) (SEQ ID | ggcgacgagatggtgtcctcggcgggagtcaccatcatgactggcgtccctttgatgaggcgcatcgctccgtacgg |
| NO: 51) | ctgccacgccagcacatggtagtagctctgcacggtcgtccggttggtaaagaacgaggtccccaggaatgctcgg |
| | aactgctccatgttgtactgattgccccaaccagcggggttgtagccgtcatccccgacaaacgggatgtataccgg |
| | gtcgttgcccgcgagagtggagacacgatcctgcattgccagcgccatgacgttgttcttttccttctctcgaaagtcg |
| | tagttggcaattggggtcaccgagatggcggctccaacacggtggtcgagaccagccgcaacaagcgccgtcatgg |
| | cactgaaggagtagccatagagaatgatcttgtcctcatcgaccatgggatgacgagccataaaggtcagagcatc |
| | gtgaaagtcctccaccagcttggccggcttgacatcattgcgcggttcgccatcactggcaccaatgcaacgattatc |
| | atacaagaggaccgttactccctgttgctggaaccagacggcaacatccggtaacaagatctccttgggggtgttga |
| | actggaccaatcagcctacgaatacagagacaaatcaatcagacgtactccctgattcatgacaatggccggccca |
| | cgaattgtcccagggtacagccagcctcgcaatatcaacccatcacaggtcggaaactcgacatcctcgcggttcat |
| intergenic region | tgtgtctggttagaaaatgcacaaccccaagtctagccgatgcttttgcaccttattgagagcagtggaaaaaagct |
| between AT and | ggaatcatctgggacatatcaagctgaactgggcgaaataaacattacaacacttccatactatcggcattgctaat |
| PKS (0149P, 468 | aatagccccgtcagccgcaaatcgactggactccgaccggggatctagtattccgagtacgagtacgagtccagag |
| bp) (SEQ ID NO: | tactcatcgccgaatgccgccccggtcaaattggccgatctgacgcttgtcacttggcagcctgatagcagtctttatt |
| 52) | gatcacaataaagctgacctggtgcaacaaaaatctgtcttgcacttgattccaattttgcagactgctctccttatta |
| | tctcaggccgagtctgcattttcctgtcttttttttttttgttgttttccaccttctcttggtggttccatcgcctcaga |
| PKS (7603 bp) | atgaccctcacatatggccataagcgcctccaggatgccccagagcctatcgcgatcgtttctgcagcatgtcgatta |
| (SEQ ID NO: 53) | ccggggcatgtgaatggcccgcacaaactatgggaactccttcagtcgggaggcactgccgtttccaatgaggtgcc |
| | ccaatctcgatttagttccgagggccatttcgacgggtcaggccggccgggcaccatgaaagcgctgagcggcatgt |
| | tcatcgaggatatcgatcctgccgcctttgatgcggcctttttcaacctcacccgggctgacgcgattgccatggaccc |
| | ccagcagcgtcagcttcttgaagtggtatacgagtgctttgaaaacggcggcataccgattgagaaagtgaggggg |
| | aaacaaatcggctgctacgttggcagtctcaacggcggtaagagcctctggatgtcgcggtggtccgttgcagacat |
| | aattcggattctcattgatgcagattaccacgacatgcagatgcgagacccggagcaaagggtgtcgggtcatgca |
| | gttggcacgggtcgagccatactgagtaacagaattagccacttcttcgacctaagaggatcgaggtgagtttccaa |
| | gacactcgatggtctcttcggcagtgactgagatcgactccatgcagtttcacaattgacacagcgtgctcgagcgg |
| | ccttgtgggagtagacgtcgcctgcaagaatctccgcgcgggaacactgaccggagcagtcgtggctggtgtcaatc |
| | tgtggctatcaccagaacacaccgaagaaaggggcaccatgcgggcagcgtactcagcgagcggcaagtgtcaca |
| | ccttcgatgctaaggctgacggatactgccgcgcggaggccgttaatgctgtgtacctgaagcgtctatcagatgctg |
| | tgagggacggcgatcctatccgcgcagtgattcggggaaccgcgagtaacagcgacgggtggacccccgggatca |
| | acagccctagcgcccaagctcaagcggcgatgattcgcgaagcttatgcaaatgctggtatcgacagcagcgagta |
| | cgccgagacgggatacctcgagtgtcatggaacgggtaccccggcgggagaccctactgaagtcaaaggcgcggc |
| | gtcagtgcttgctcacatgcgcccaccggcgagccccttgatcatcggatcggtgaagagcaacattgggcactcgg |
| | agccaggagcaggtctctctggcctcatcaaggcgatgctggtggtcgaggagggcgaaatccccggcaatcccac |
| | gtttctcaacccaaatccagccatcgatttcgataacctccgggtatatgccacccggataaggattccatggcccaa |
| | agaatcaagccactacagacgtgcaagcgtcaactcgtttggctttggaggctccaatgcacatgctgtactagaca |
| | atgcggagcactaccttgggaagtactgggcatccctcgagataccccgatctcacctcagctcatatatcaatctgt |
| | ccgacatgctgtccttgtttgacggacggcgatcatccaaaacagtcactcggcggccccaagtactggttttctcgg |
| | ccaacgacatggattcgctcaaacgccagatatcgacgctttcagcccatctcctcaacccccgagtcaaagtcaag |
| | ctttcagatctcagctatacactctcggagcggcgatcccgtcatttttgccgcgcattcctgctaagctaccccgcga |
| | agagtggacatgccagtaagatcgccgtggaggaggctcagttttccaagatctcgcaagaggcaaccagaatcg |
| | gctttgttttcaccggccaaggcgcgcagtggtcacaaatggggctggagctggtcagaacgttcccaggggtagtg |
| | aagcccattctggagcagctcgacaacgtgctacaggagctgccagcagacctcaagtcagagtggtcgctgctgc |
| | aagagcttacggaagctcgctcgtctgagcatctgagcaggccggaattctcgcaacctctcgtgaccgcgctccag |
| | ctggcacaactagcggtattgcaatcctggggtgtgcgggcagaagccgtgataggtcattcttcaggtgaaatagc |
| | agccgcgtgcagcgcaggactccttacaccccggcaggctattctgaatgcgtatttcagaggactcgcagggaaa |
| | agtgctctggcaactagtccgaagggcatgatggctgtgggactcggtgcacaggatgtccagccgtacctcgagg |
| | gcgtaagtgccgacgtggtaatcgcatgccacaacagcccagctagtgtcacgctgtccggttcggcctccacatta |
| | gcggagctggaagggaccatcaaagccgctggacactttgcccgaatgttgcgagtggaggtcgcgtaccactcgc |
| | ctcacatggccaagatagccaaccgttacgaagagctgctgaaggagcacggaaggctggacgatggcagtaaaa |
| | ccaataagagatcgaatcgtatgatctccaccgtgaccgaagatgaggttactggagctcaagtctgtgacgcggc |
| | atattggaaagcgaacatgctgtcgcccgttcgattcgacggcgcatgcaacaagctgttaacgaacacgcaactc |
| | gctcccaatttcctcatagaactggggcccagcaacacgctcgcaggaccagtcactcagattgccagagcagcca |
| | aggtggacaacctcacgtatgctgccgcgaataagcgtggccccgacgagagctcccgcgcaatcttcgacgttgc |
| | aggccacctgttcctgcagaatgccgacatctcacttgacaaggtgaacctcggcgacaatacaccagataaggcg |
| | aagcccgcggtgatcgttgatctgcccaactaccagtggaagcattctacccactactggcacgagagtctggccag |
| | caaggattggagattcaagaagttcccgtcccatgacttgcttgggagcaaggttatcggcacgctgtggcagagcc |
| | cgtcctggcacaagatgctgcgtctgtccgacgtgccctggctgcgggaccaccggattggatcagagatactctttc |
| | ccgctgctggctatctggccatggccatggaagctgttcgccaagccgctttgtcgactgcaacagctgaagctcga |
| | gagctcctgaagacgagacactaccgctactgcctccgggatgtacaatttccgcgaggactggtgctcgaggatga |
| | tgccgaagttcatattatgcttttactggtacccatggcaaagctcgggcagggatggtgggaatataagatcacctc |
| | tctcgcggaatcggattcagtagcatcgtcatcatcgtcaaccttgtccccggagaagtggaacatcaactccaccg |
| | gattggttcgactagagacaatcctagaggcatcatcgtctcgagcaccagagcacacctgcagcttgcctttggat |
| | aacccgacacctggacagatgtggtacaagtctctcagggacgccggatactcttacggtccaagtttccagagact |
| | ggtagccgtcgagagcacggagggaaagtcagccacgcgctctcttatctctttggaaccgccacgatccaagtgg |
| | gagccgcagtcagaatacccactgcacccagctcctctggacagcgtcctccagagcatgttcccctcgcttcatcgt |
| | ggaaatcgaactaaactagaccagctactcgtcccaagaggaatcggtgagctgaccgtctctggagacatctgga |
| | agtccggagaagcaatttctgtgaccacctggaacaaggtgtccggagacgcgtctttgtacgatcctgccagtcga |
| | tcgctaatcatgcagctcaacagcgtgtcgttctctcccatgctggatggtcgagacagtctttacatgtcccatgtct |
| | atactcaattgacgtggaagccagatttccaacttctggatactgatgagaagctccaacaggccctcagcggtggt |
| | gatggcgctgcgtcttcccttgtccaggatcttctcgacctcgccgctcacaaggcgcctaatttgagggttctcgagt |
| | tcaatctcgttcccggaagctcggaatccctgtggcttgccggacatccaacaccgcgtgctgttcgcacggccctta |
| | ctgaattccactttgctgccaacagcgctgatactgcgctcgccgcccaagaggaatatgcagagtggccggcggca |
| | cgaaccgcccgcttcagtgtgcttgatcctttcagcaaagcccttgctgtacccgcaggaagttcccagttcgatcttg |
| | tgataatcaggcggcctcagcatgcagacttgggcgagctcgacattctcgtcggcaacttgcgccgtctgacttccg |
| | acggcggcagtgtaatattctatgattccaaacagtccagtctgtcagggggtcgaggtttggcgaatgggcacaac |
| | catttccccgctgcactgcaacgctttggtctcactaaggttcgccagacgagggatgggagctgcattgtggcaga |
| | ggtcagcccagcacagaatctctctctccgcaatgatttcagagtcgttattgtgcggttctcaactgcgcggtccact |
| | attatcgatcacaccatttcgcagctgcgccaatttgggtggaccttgacggagatttgcatctacaatgaatccggc |
| | actgggcttccacaacttcctcccaaatcaacggtgctcgttctcgacgaattggaccggcctttgctggccaccgcg |
| | accgaccatgaatggacggcgctccaggcgataatacagtcagaatgtaacttactttgggtgactgagggctcgc |
| | aagttaggcctactgcgccgctcaaggccgttgcgcatgggatctttcgtactgtccgcgccgaggtacccatgatgc |
| | gcatagtgactctggacgtcgagtcagccacaactgagagtttgggcacaaacgcgtcggccatcaatatggctctg |
| | agagagataactttagcggacagatcgtccctccccattgagtgcgagattgcggaacgaggtggtctgttgcatgt |
| | cagccggatatggccggatgctggcgtgaataaacgcaaggtggaagacaacgcaggaggcgcaccacctgtgct |
| | aaccaatctgcatgattcaaagtctaccattcgcttgatggcaagcagacctggtagtttggaggcgctgcatttcgc |
| | cgagcaaggtcgagatgtgtgcagtaggcaagatatgggaccggatgatgttgaggtcgagatcttcgccgctggtt |
| | gcaactccagagacattgatgtggctatgggcgatatctctggggatttggatggactcggcttggaaggtgctggc |
| | gtggtcgtccgcgtcggcgcctgtgtcagcgctcgctgtgttggccagcgggtggcagtgtttggcaaaggctgcttt |
| | gcgaaccgagtcaccgtctcatgcaaagccacctttcctttgcctgatgccatgtcgtttgagcaggctgcgacgctg |
| | ccaatcgccttgctcaccgctttatacgccgttggtcgtctcgcacatgtacagggagatgatcgtgttttagtccattc |
| | accttgtactgatgttgggatcgcttgcatccgactctgccagcgctcggggtcgactcccttcgcgacggtggacaa |
| | cctggagcagcgccattttctgactcacgagcttggactaccggaagatcatatcttcatgtcggagcctgcagcatt |
| | tcctcgcgctctccgccacgcaaccaagggccatgggcttgacgtgattatcagtcagcctgcaaatcgcaatctcg |
| | acaatgaaaacatgcggctacttgcccctggtggacgacttatcgggatagcaaacggaggcgccgatgttggaaa |
| | tttgctgcccacgggatctctcgctcccaactgttctttccagaggttggatgtaacagctttaccggagaaaaccatt |
| | gaatcgtaagtaaacgttggagaaatattggcttatcttttatcgagagtggaaactcatttgacagtgtgttcttgga |
| | gctttctcggctcgtcacagatggcagtgtgcagcccctgtcaccaagcacactcttgggttatgaagagatacccaa |
| | ggccctgcagcttcttcgagaaggcacccacatcggaaagatcgttatttcagacccccgtggcacgaagcttgctg |
| | ttctggtaagagtttgaacttgacgtgtctgaatcggattctaacctgtccagacccgacctgcaacaaccctggcac |
| | agagtatgattaaccctagccactgttatctcttggtgggtggtttgaaagggatctgcggtagtcttgccatccattt |
| | agcctcccacggggccaagaacattgccgtcatgtcccgcagtggtggtggagaccaggtgtctcagggcatcgctc |
| | gaaacatcagagcactggggtgttctcttgacctgcttcaaggcgatgtcacttctatcagcgacgtcaggcgggcct |
| | ttagccagatctcggttcctctgggtggaatcatccaaggagccgccgtattccgagtaagacagcactcccgaagc |
| | cattctctgctattcatttcgttctgacctagaaaccatcaggatcggacgtttgaatccatgtctcacgaagactacc |
| | acgccgctgtgtcgagcaaggtgacgggcacatgcaacctacatacggtctccctcgaaacaaatcaaccgatctc |
| | attcttcaccatgctgtcttccatttcaggcgtcataggccagaagggacaagccaactacgctggtggcaatgcatt |
| | ccaagacgcctttgcagagtatcgccgcgcattggggctgcccgccatcagtattgacctcggacccgtagaagacg |
| | tcggagtcattcacggtaacgaagacctccagaataggttcgacggtagcactctgctcagcatcaatgagggcctg |
| | ctgcgccgaatctttgactactcaatccttcagcagcatccggatccacagcaccgtctgaacgtcacgagccaagg |
| | ccagatgattaccagtatactcgttccccagcctgaagacagcgatctgctcagagattgccgctttcgaggcttgcg |
| | agcccttggagaacatagtccacgctcacggcgggaccctaccaaagataaagagatccagagcctcttgtttctgg |
| | cccaatcccaggatcccgatcgtgcagccctgcgcgccgccgctatcacggtcgtgggtgcgcggctggcaaagca |
| | gcttcgcttaacggatgcagtcgacccggcacgtcccttgtcctactacgggttagactctctggcggctgtcgagct |
| | acggacctgggtgcgtatgacactggcgatagagctcaccactttggatgtgatgaatgcagccagcctgggagaa |
| | ttgtgtgagaaggtgattgggaaaatgggatttggcatgtag |
| intergenic region | gcagtatgttaaccggtagtgaaagggctgcgctgttgctttcggttgttagagttatggtatataggtacagatgaa |
| between PKS and | aacactggtctatgcatatttcactatccttgacgcgacgaagtaagcctcgatgtgatctatcgtcgtagataacag |
| ABM (10022P, | cttaatgacccgatctgtgcttaatttcccgccgctgtccggatctcgtctcgggtcattttgcattatatagggagcct |
| 305 bp) (SEQ ID | ccactcgcccatcctcactcatcaaccacatcgaccagctcagaattcacccgcatcaattcaaagaaa |
| NO: 54) | |
| ABM (895 bp) | atggatcagtcgatgaagccccttctctcacccacagaacgaccacgtcggcatctgacagcgtccgtcatctccgta |
| (SEQ ID NO: 55) | agcccctcctcaaccatgcagaagtaggatctaatgaagcaaccgctaacgccatggtaaaaagttcttcctcccaa |
| | atcaattccgtctcagcacgatcctttgcattggtgctctcctgcagaccatcctctgcgccgtcctccccctccgctac |
| | gccgccgtcccatgtgtaactgttctcctcatatccgttctcaccacaatccaagagtgcttccaaccgaacacgaatt |
| | ctttcatggccgatgtcattcgcggaagaactaccgcgcagatcccaggcaaagatggaacacacggccgggagcc |
| | ggggaagggctcggtggtagtgttccaccttggaatacaatacaatcaccccctcggagtttttgcaccgcacatgc |
| | gcgaaatctcgaaccggtttctcgccatgcagcaggacatactccgccgcaaggatgagctcggcctgctggcggtt |
| | cagaactggcgagggagcgagcgcgactccggtaacaccacgctgatcaagtatttcttcaaagacgtggaaagta |
| | ttcataaatttgcccacgaaccgctacataaggagacttggacgtactataaccagcatcaccctggtcatgtgggc |
| | atctttcatgagacatttatcaccaaggatggcggatatgagagcatgtatgtaaattgccatccaattctacttggg |
| | agaggcgaggtcaaggtcaataatcggaaagacggcacagaggagtgggtggggacactggtcagtgctgatac |
| | gcctgggttgaagtcttttaaagcaaggttgggtagagatgactga |
| intergenic region | caatttttttatcattttctggctattcgttcaaataacagggtttctttggtctgggtaatggtttctgtcctaaggctta |
| between ABM | cggtcagggagcagttagttacctagagtcgcttcgggacatcaaccgtatctgtttgttgatatgacaactattactt |
| and AN10035 | gattacttttgtttttcttggtcgtcttctttatttatctgattactgagttccagatgcacaccggaccccgacagttcca |
| (10035P, 374 bp) | ctgaaacccgagctcggatagcacgacgctgacgctgacgctgcatgtccagtcaccacggctcgtattttgaaaca |
| (SEQ ID NO: 56) | gtcaaagcagtgaccagagtctacagtggagtattcaagcacctatcaaacaga |
| AN10035 (1857 | atgtcggtttcacgctcgtgcttcaggcctttcctcccagcagaaatcgatggtgggcacctacccgttgacccttcgg |
| bp) (SEQ ID NO: | tctttacacacattgagcgtggcctccatcagaatccacagggttttgctattcagagtacccatcaacaaccgtgtc |
| 57) | atttctctgcgcttgttcagacaggaagtgggactgaaaatggcggtgcgccaaactatgatgcggtcgagagaga |
| | accggggacatgcctcgcctggacatatacacaactccaccacgctgcgttacggattgcggcggggctgctggcg |
| | agaaatgcccagccaagcacgagaatgctcttgctcatccccaacggcgccgagttctgtcttctgctttggactgcg |
| | gttgttctccgcgtgacgattgtctgtctcgatgaggaactgcttaacgttgagcagcatgatgagttacgcagaatg |
| | ctaaagactatcaatccaagggttattgttgtgcaagacgtaaaaggcgcggatgtgatcgatgtcgcgttgcggaa |
| | tctaccgcttgacccggatatcctcaagatcactctatccgagcttgcgggaagtcaaccagactcagcctggagat |
| | cccttctgtccctatctctgacaccagctctttcagcttctgaaaccgagtctcttctatcttctgctcgctgggactcttc |
| | caacgcagcccgtacatactccatcctctatacgtcaggaacatccggggtccctaaagggtgcccgttgcatatttc |
| | gggaatgagctacgttctccaatcccagtcgtggctggtcaacgcagagaactgcacgcgggcactgcaacaagcg |
| | catccgtgtcggggcattgccattgcacagacactccagacatggagggaaggtgggacagtagtcatgacgggga |
| | atggcttcaatgcgggcgatttggtgcatgcggtaaaaaggcacgcggttagtttcgtggtgctcacgccggcgatg |
| | gttcatccagttgcagacgagttgaagggtagaaatggcgcagctgattctgtcaggacagttcaaatcggtggcga |
| | tgcggtgacaagaggcgcacttgagatatgtacgcgattgtttccgaaagcgagagttgtcgtgaatcacgggatga |
| | cggagggtggaggggcgtttgtttggcctttcaacaggcccagagatattccgttctatggtgagatgagtcctgttg |
| | gatccgttgcacgaggcgctgctgtcaggatccgtggcgcaaacgcgacagtggcaagaggagagctgggcgagc |
| | tccatgtctcctgcccaagtattatcccggggtatctgggtggagtttcagcccagtcgtttcacgacgaggatgggc |
| | gaagatggttcaaaacaggtgatgtgggcttgatggacaagcagggcgttgtttttatccttggccggatgaaggat |
| | atgattaatgggaaagtgatgcctgccccgattgagagttgccttgagaaatatacttctgttcaggtatgttttctttc |
| | tttattcttcccccatacctccaccacatttgcctcagatctgagatctaaacaagcataccagacatgtgtggtaaat |
| | gctggcggcccctttgctgtcctggcacgatataccggcaagaaagaagcccagatcagaagacatgttgtgcggg |
| | cacttgggaagagcaatgcgttgaacggagtaatttatctgcaccagttgggactggaaaggtttccggttaatggg |
| | acgcataagattgctcgtggggatgtggagggggctatgctggcctatttgcagactgagcctaccagtagatag |
| intergenic region | aaccctacctatagatggattgtgtgctgagggcgtctcaatatgctattcttaacgccaccgaaatcgtacatcaga |
| between | tcactcaagacgtcaagacatggctccaactagccgactcgggttgtcccattagacattctaatca |
| AN10035 and | |
| AN10038 | |
| (10035T, 145 bp) | |
| (SEQ ID NO: 58) | |
| AN10038 | ttaccattttatatcctctggaatctctaactcaagtcccaaatccgggacacctcccgcaaccttcttaaaccagcca |
| (complementary, | atctcaaggaccccatcataccagctgcacagtgctccaaacctctcctgcatggatctcctaaacgccgcaaacgc |
| 799 bp) (SEQ ID | tcccatgagcagactcgcggcgaaaaaatccgcaatcgttatactttcccccacaagatatctgctgcgcttcagatg |
| NO: 59) | ctcatctaggtacttgcaccgctgcagcatcgcacgcagtgagtccccgtcatcttgctggattatttgccgttgccca |
| | atgcgtgggaggaagacgccgccgactgctggaaagaggtcggagtttgcaaaagacatccattggaggatcctg |
| | agcgaggagcgttcgtcattgcctaggagggattttgttatcgggtcctgactctgggatgcaactgctcagcaggac |
| | ttttcagtcctctttcattaaccagggagtgtcggggctgagtacagtaaagagtcaatggaatacattcactcagca |
| | cgaacccgtctgcgcctacaaaagtagggacttgcccgagtggattatatctgcaaagctcctcaaatgcctctttat |
| | tcttcttttctgcgtgtatgattttgacgtcgaggttgtggagctttgctagagcgatgagggtcgtcgagcgaggcgtt |
| | ggctgcaagattcaaggttagctaaacccccaattctaattctgggccctgaggtgtaagaacatacgttatgggtgt |
| | agagtgttccgaatgacat |
| intergenic region | ttgtgcggtctggtctgtttggaaatgataatgcgggtgggtatgggctgtcggtgattatatctactccgtcgaaccg |
| between | gaacccgggggtctgcgactgcgatacgctcgatgaactccgagatttcgggggccgggggttgaggttgcactgc |
| AN10038 and | agatcttgatatccagcatctagcacggtatagttcgtatcttgagatatttgagacattgaagtctgaaaacgacgg |
| AN10044 | tttaggctacggtacccgactgccatagctctctatacgagtgctttataaacacccaaccaccatcaaccataatcc |
| (10038P, 364 bp) | tcacggcaccgtattggttacgaaatactaaattctgaatatcatcaatcgaa |
| (SEQ ID NO: 60) | |
| AN10044 (798 | atgcctctggccacttacgccgttctgggcgcaaccggcaatactggcacggctctgatccagaatctgctctcgcca |
| bp) (SEQ ID NO: | ccatcttcagaaatgcacataaacgcctactgtcgaaacaagcccaaactcttaaacctcttgcccgaactcaacga |
| 61) | cacgaaaaatgtgactatctttgaaggctccatcaccgacttatccctcatcaccgcatgcatacgcaacacacgtgc |
| | ggtcttcttgaccgtcacttcaaacgacaatattcccggttgccgactgagtcaggactcggtgcagacggttctcga |
| | ggcactcaagcagattcgtacagcggaaccgaatgcagttgtgccgaaactggtccttctctcctccgcgacgatag |
| | atccgcacctaagccgcaaaatgccctcgtggttcttaccgattatgaaaacagctgcgagtaacgtctacgccgac |
| | ctgatcaaggcagaggagatgctgcgagcgaacgagtcctgggtcacaagcattttcatcaagcctgccggcttga |
| | gcgtcgacattcagcgtggtcacaaactcgactttgacgagcaggagtcgttcatctcgtacctggatctggcggctg |
| | ccatgcttgaggcggcaaatgatacagatgggaggtatgatgggaggaacgtctctgtggttaatacggggggcaa |
| | ggcgaggttcccgcctggaactccgaaatgtatcattgttggcttgctcaggcatttcttcccggggttgcatcgatttc |
| | tgccaacaacggggccttcctaa |
| intergenic region | tggcctgggattgtagcctggggtatgtaatattgggtctctaggaggacgttttggttattagatgggtcaattttatg |
| between | gattcccaacaccgcaaaacgtagccctgatcgaggttaaggcctcagtcactcattcgtactagtcacgctcggcg |
| AN 10044 and | tacctttgccatttgctagatatagagaaccagtccagtcgacaatatgtgaatatggctgctcggtcatcgggcttc |
| AN10023 | gaggtctcgttatccgaagctagctgtgcagtatatatctttgggctcaggacattaaaccagtcagcaaaacccaa |
| (10023P, 360 bp) | ccatctaccataccaagtcaacaagaaagcacgaatacggcgtcaaaa |
| (SEQ ID NO: 62) | |
| AN10023 (1341 | atgtcctcttcgatcaatattctctcaaccaaactcggccagaacatctacgcccaaactcccccctcccagactctca |
| bp) (SEQ ID NO: | ctctgacaaatcacctcctacaaaagaaccacgacacgctgcacatctttttccgcaatctaaacggccacaaccac |
| 63) | ctggtccataaccttctcactcggctagtgctgggtgcaaccccagagcaactccaaaccgcctacgacgatgacct |
| | ccctactcagcgcgccatgccgcctctcgtcccttctatcgtggaaaggttatctgacaactcctacttcgagtcccaa |
| | attacacagattgaccagtatacaaacttcctacgtttcttcgaagcggagatcgaccgacgagactcatggaagga |
| | cgtcgtgatagagtacgtcttctcgcgctcgcccattgctgagaagatcctcccgcttatgtacgacggcgcctttcac |
| | tcaattattcatctcgggcttggagtcgagttcgaacagccggggatcatcgctgaggcattggcgcaggcggccgc |
| | gcacgactcttttgggaccgattactttttcctcacggccgaaaagcgagctgctgggcgaaacgaagagggagag |
| | actctcgtgaaccttttacagaaaatcagggacacacccaaacttgtcgaagccggacgcgtccagggcctcattgg |
| | gacgatgaagatgagaaagtctattctcgtcaatgcagctgatgaaataatagacattgcgtcgcggtttaaagtca |
| | ccgaggaaacgctcgcgagaaagactgccgagatgctaaacctctgtgcttacttggctggtgcgtcgcagaggac |
| | gaaggacgggtatgagccaaagattgactttttcttcatgcactgcgtaacaagcagtatcttcttctctattctcggg |
| | cgtcaggactggatttccatgcgggatagagtaaggttagtcgagtggaagggccggctggatctgatgtggtatgc |
| | tctctgcggtgtacccgagcttgatttcgaatttgtgagaacctacaggggggagagaacggggactatgtcctgga |
| | aggaattgtttgcgattgttaatgagcagcatgatgatgggcatgtggcgaagtttgtgcgagcgctgaagaacggg |
| | caggaggtttgcgggcagtttgaggatggagaggagtttatggtcaagggggatatgtggttgaggattgcgagga |
| | tggcgtatgagacgacgattgagacgaacatgcaaaatcggtgggtggttatggcaggcatggacggggcttgga |
| | aggacttcaaagtgcagtcgtctgattga |
| intergenic region | ttagatatacgcagtgctgtatatgggtcttggccatctagtacgatcaacaagccaagagtgactctactctctactc |
| between | tttacaggtctatcgatagcagtcaatctatgcatcgacaagagttcaatttgacttcccgatttcgactcagagaatc |
| AN10023 and | ctaggcccatgccaggacttataaatgcctatccatgattgcatgaagtcctttctccaaacacctcaaagaccattg |
| AN0153 (0153P, | cttgtgagcgtcagtttacctttttgactatgtcgggtcctcaggctggatcatagcgctattccatattcagcttggcg |
| 459 bp) (SEQ ID | tagaatggtttacgctagcccactccggctagacggcctgaacgccgggatatttccacgtgacggcattcttttcaa |
| NO: 64) | cttcaagccctacaagcgcgccctacccctaagccctcattgctgatcctggaagcatcatcttc |
| AN0153 (2778 | atgtcagcgccaactcctcccgtcatggccgatgccagtgcatcaggaccctccgttgacacgcagggagcgtccga |
| bp) (SEQ ID NO: | cctccctgcctcgccggtgcccaaggaggagggtcaccatggtaagccacctagccgcattcactgcctgactccgg |
| 65) | cagtaacaccaccccaagtctattcactcaacccaatgacttactcttgtcacactagaactccccaagctgtttcatc |
| | ccatcgaggatgattctctttcgccgcgggcatccaaaaaacgtcggcttgatgaaccggaggactccgtagcgga |
| | aacgacaacgacaacaccaccgtcccagcaacctcaagagcaaacccgggaaccgtcgcagcaaacggagcag |
| | agccagttccagcaacaacacacgaatcttcttcctggtgctggagaccagattgaagaagaattggcatcggccct |
| | tgccgcgggggtcgtcgattcggtggaaactgcggatagcaagaatggtcagaccgagatcggagcaagtcctgtg |
| | caagagcaaaacacgaatatcgactcggacgtagctactgtcatctcgaacatcatgaatcattccgagcgtgtcga |
| | ggagcagtgcgccatgggtccccagcagttgccggatttgtccggtcagggcgctcccaaggggatggtttttgtca |
| | aggccaattcgcatctaaaaattcagagtttacccattcttgataatctggtgagttctctaattcaggctcagagtttt |
| | ggttaggaagctaatttgcagtccacgcaaattctgtcgctgctggccaagtccacgtaccaagatattacctccttc |
| | gtatctgagccggagtcggagaatggtcaggcgtacgctacgatgcggtcactgtttgaccacacaaaaaaggtct |
| | attcaaccaagaaatcgttcctctcgcccacggagctcgagctcactgaaccttcgcaagtcgacatcatccgcaaa |
| | gcaaacctggcatcgtttgtctccagcatctttggtactcaggagatcagcttctctgagctcaatgataactttctcg |
| | acgtatttgtccccgaaggtggacggcttctcaaacagcaaggtgccctttttcttgagatgaagactcaagcgttca |
| | tcgcgtcgatgaacaacaccgaacgtacccgcaccgaattgctttatactttgttcccagataatcttgagcagcaac |
| | tccttgacagacgacccgggacgcgtcagctggctccgagcgaaaccgactttgtcaaccgtgcacattcgcgccgt |
| | gagatattgcttaatgatatcaacaatgaggaggccatgaaagctttaccagacaaataccactgggaggactttct |
| | ccgggacctcagctcgtatattacaaagaactttgataccatcaacaaccaacaggttagactctacatatggtttta |
| | aacaaatagatcgctaatgcggattagtcaaagaagatcacaaaaggacggcaaccatcttcatcaaatggtgatt |
| | ctgagccgcctagtgcgcctcttcagagccagtttcctgtcgccacgcaggcgccggaggtcccagtcgataaaaac |
| | atgcacggtgacctggttgcccgtgccgccagagctgcgcagattgcgctgcagggtcacgggctcagacgttctca |
| | gcagcaggcacagcaggcccagcagcaacaagcccagcagcaacaagcccagcagcaggcccagcagcaggcc |
| | cagcagcagcaacaggctcggcagcaggctcagcaatatcagcagcagcagcaacagcaacagcaacagcaac |
| | aacagcaacaacaggctcagcagcaggcgccccagcagggcatccagattctacaaggatatacccccgcgcagc |
| | aaccctaccagagcagcccagctccttcaggatatcaacagtctcagacatataacttccaacagagcccaatgca |
| | gacaaacttccagcagtacaaccacccctcgccgtcgccaatacccggtcgacctaactcgtctactgccaaccacg |
| | gctacatgcccggcattccccactactctcaatctcagccgacacaagttctctatgagcgggctcggatggccgcat |
| | ccgccaaatcctcgcccagcagccgcaagtctggccttcccagtcaacgccgcccatggacgactgaagaagaaaa |
| | cgccctcatggctggccttgaccgcgtcaagggaccccactggagtcagatcctggccatgttcggccccggcggta |
| | cgattagcgaagctctcaaggatcgcaaccaggtacaacttaaagataaagctcgaaacctgaagctcttctttctt |
| | aagagtgggattgaggtgccatactacctcaaattcgtcacgggtgagttgaaaacgcgtgctccagcacaagccg |
| | ccaaacgtgaggcccgcgagcgccagaagaaacaaggggaggaggataaggcacatgtcgaggggatcaaggg |
| | catgatggccctggcgggggcgcatccgcagcaggtcggccatcctcatcatggagttcctggagttccgcaccacg |
| | gccacgagagcatgtctgcgtcgccgatgccgccagatccaaactttgatcagacggcggagcaaaatctcatgca |
| | gacgctgggaaaggaagtccatggagagtcattcgggcagcctgggcagcctgggcacccggggcatcatcctga |
| | gaatatgcatatggggcaatga |

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method of producing a target compound in a host cell comprising:
a) amplifying i) one or more polynucleotide sequences from a first target sequence, the first target sequence comprising one or more genes of an exogenous biosynthetic gene cluster for producing the target compound, and ii) amplifying one or more polynucleotide sequences from a second target sequence, the second target sequence comprising one or more intergenic regions of an endogenous biosynthetic gene cluster of a host cell, wherein the one or more intergenic regions comprise a promoter sequence for at least one gene of the endogenous biosynthetic gene cluster, and wherein the promoter sequence is controlled by a positive activator protein;
b) assembling the amplified one or more polynucleotide sequences of the first target sequence and the amplified one or more polynucleotide sequences of the second target sequence in vitro to provide assembled sequences;
c) using the assembled sequences as a template for a second amplification step to produce one or more final polynucleotide sequences; and
d) transforming the one or more final polynucleotide sequences into the host cell wherein the one or more final polynucleotide sequences induce one or more homologous recombination events at an integration site of the host cell, wherein expression of one or more genes of the one or more final polynucleotide sequences causes production of the target compound.

2. The method of claim 1 wherein the host cell is a species of Aspergillus fungi selected from the group consisting of Aspergillus nidulans, Aspergillus fumigatus, Aspergillus oryzae, Aspergillus clavatus, Aspergillus flavus, Aspergillus niger, Aspergillus terreus, and Aspergillus sojae.

3. The method of claim 1 wherein the integration site is one or more of an asperfuranone (afo) biosynthetic gene cluster and an monodictyphenone (mdp) biosynthetic gene cluster of Aspergillus nidulans.

4. The method of claim 1 wherein the one or more intergenic regions of the endogenous biosynthetic gene cluster comprise intergenic regions of the asperfuranone (afo) biosynthetic gene cluster of Aspergillus nidulans or the monodictyphenone (mdp) biosynthetic gene cluster of Aspergillus nidulans.

5. The method of claim 4 wherein the one or more intergenic regions of the afo gene cluster are present and is at least 85% identical to one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15.

6. The method of claim 4 wherein the one or more intergenic regions of the mdp gene cluster are present and comprise and is at least 85% identical to one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64.

7. The method of claim 1 wherein a polynucleotide sequence of the positive activator protein is operably linked to an inducible promoter or a constitutive promoter.

8. The method of claim 7 wherein the inducible promoter is present and comprises the PalcA promoter sequence and the polynucleotide sequence of the positive activator protein comprises a polynucleotide sequence of afoA, a polynucleotide sequence of mdpE, or a combination thereof.

9. The method of claim 8 further comprising contacting the host cell with an agent to cause induction of the inducible promoter.

10. The method of claim 1 wherein the assembling step comprises Gibson assembly of the amplified one or more polynucleotide sequences of the first target sequence and the amplified one or more polynucleotide sequences of the second target sequence.

11. The method of claim 1 wherein the exogenous biosynthetic gene cluster comprises a citreoviridin biosynthetic pathway, a mutilin biosynthetic pathway, a pleuromutilin biosynthetic pathway, or a fumagillin biosynthetic pathway.

12. A method of producing a target compound in a recombinant Aspergillus nidulans host cell comprising:
a) amplifying i) one or more polynucleotide sequences from a first target sequence, the first target sequence comprising one or more genes of an exogenous biosynthetic gene cluster for producing the target compound, and ii) amplifying one or more intergenic regions of an endogenous biosynthetic gene cluster of a host cell, wherein the one or more intergenic regions comprise a promoter sequence for at least one gene of the endogenous biosynthetic gene cluster, the one or more intergenic regions comprising one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15, one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64, or combinations thereof, and wherein the promoter sequence is controlled by a positive activator protein;
b) assembling the amplified one or more polynucleotide sequences of the first target sequence and the amplified one or more polynucleotide sequences of the second target sequence in vitro using Gibson assembly to provide assembled sequences;
c) using the assembled sequences as a template for a second amplification step to produce one or more final polynucleotide sequences; and
d) transforming the one or more final polynucleotide sequences into the host cell wherein the one or more final polynucleotide sequences induce one or more homologous recombination events at an integration site of the host cell, wherein expression of one or more genes of the one or more final polynucleotide sequences causes production of the target compound.

13. The method of claim 12 wherein a polynucleotide sequence of the positive activator protein is operably linked to an inducible promoter.

14. The method of claim 13 wherein the positive activator protein comprises the polynucleotide sequence of afoA, the polynucleotide sequence of mdpE, or a combination thereof.

15. The method of claim 13 wherein the inducible promoter comprises a PalcA promoter sequence.

16. The method of claim 15 wherein the integration site is one or more of an asperfuranone (afo) biosynthetic gene cluster and an monodictyphenone (mdp) biosynthetic gene cluster.

17. A transgenic Aspergillus nidulans cell for producing a target compound comprising:
a recombinant biosynthetic pathway comprising:
one or more genes of an exogenous biosynthetic gene cluster operably linked to a polynucleotide sequence of an intergenic region of a gene of an endogenous asperfuranone (afo) gene cluster and/or a gene of an endogenous monodictyphenone (mdp) gene cluster, wherein the intergenic region comprise a promoter sequence of the gene of the endogenous afo gene cluster and/or the endogenous mdp gene cluster; and
a gene encoding a positive activator protein operably linked to an inducible promoter sequence wherein the positive activator protein is configured to bind to the promoter sequence of the gene of the endogenous afo gene cluster and/or the endogenous mdp gene cluster, thereby enabling expression of the one or more genes of the exogenous biosynthetic gene cluster and production of a target compound.

18. The recombinant Aspergillus nidulans cell of claim 17 wherein the gene encoding the positive activator protein is afoA, mdpE, or a combination thereof.

19. The recombinant Aspergillus nidulans cell of claim 17 wherein the polynucleotide sequence of the intergenic region of a gene of the endogenous afo gene cluster is present and comprises one or more of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15.

20. The recombinant Aspergillus nidulans cell of claim 17 wherein the polynucleotide sequence of the intergenic region of a gene of the endogenous the mdp gene cluster is present and comprises one or more of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, and SEQ ID NO: 64.
